(19) United States
(12) Patent Application Publication
(10) Pub. No.: US 2014/0298539 A1
(43) Pub. Date: Oct. 2, 2014

(54) SPATIALLY MODIFIED GENE EXPRESSION IN PLANTS

(71) Applicants: Dominique Loque, Albany, CA (US); Henrik Vibe Scheller, Millbrae, CA (US)

(72) Inventors: Dominique Loque, Albany, CA (US); Henrik Vibe Scheller, Millbrae, CA (US)

(73) Assignee: THE REGENTS OF THE UNIVERSITY OF CALIFORNIA, Oakland, CA (US)

(21) Appl. No.: 13/982,231

(22) PCT Filed: Jan. 30, 2012
(86) PCT No.: PCT/US12/23182

§ 371 Date: Jan. 15, 2014

Related U.S. Application Data
(60) Provisional application No. 61/437,569, filed on Jan. 28, 2011.

(51) Int. Cl. C12N 15/82

(52) U.S. Cl. CPC C12N 15/8255; C12N 15/8247
USPC 800/281; 435/99; 435/419; 800/278; 800/285; 800/287; 800/298

(57) ABSTRACT

The invention provides methods of engineering plants having lignin deposition or xylan deposition that is substantially localized to the vessels of xylem tissue in the plant. The invention also provides methods of engineering plants to increase production of a desired biosynthetic product, e.g., to have increased secondary cell wall deposition or increased wax/cutin accumulation. The engineered plants of the present invention have use in bioenergy production, e.g., by improving the density and the digestibility of biomass derived from the plant and to improve water usage requirements.

### CROSS-REFERENCE TO RELATED APPLICATIONS

This application claims benefit of U.S. provisional application No. 61/437,569, filed Jan. 28, 2011, which is herein incorporated by reference for all purposes.

### STATEMENT AS TO RIGHTS TO INVENTIONS MADE UNDER FEDERALLY SPONSORED RESEARCH AND DEVELOPMENT

This invention was made with government support under Contract No. DE-AC02-05CH11231 awarded by the U.S. Department of Energy. The government has certain rights in this invention.

### BACKGROUND OF THE INVENTION

Plant cell wall is the only source of cellulose for the paper industry and is a promising source of sugar for lignocellulosic biofuels. The utilization of plants to convert solar energy into transportable and storable energy will have a positive impact on the environment, since using plants can help to drastically reduce the utilization of fossil-derived fuels, can reduce carbon emission into the atmosphere, and even can contribute to carbon sequestration. However, even if lignocellulosic biofuels will be beneficial for the environment, the cost to produce them is still not cost-effective, mainly due to the expensive raw sugar derived from plant cell wall. The low density, recalcitrance to enzymatic hydrolysis, and medium content in cellulose are the main contributors to the sugar cost because they impact transportation cost and require high amount of energy and chemicals. Therefore, improving the density and the digestibility of the raw biomass will have an important beneficial impact on the cost of lignocellulosic biofuels production.

Cell wall recalcitrance is mainly caused by the presence of lignin, which embeds the polysaccharide polymers and reduces their extractability and accessibility to hydrolytic enzymes. Lignin content and saccharification efficiency of plant cell wall usually are highly negatively correlated (Vinzant et al., 1997; Chen et al., 2007; Jorgensen et al., 2007). Unfortunately, most attempts at reduction of plant lignin content resulted in severe biomass yield reduction (Voelker et al., 2010; Shadle et al., 2007; Franke et al., 2002) and therefore, crops with significant lignin reduction are not readily available. This cell wall-growth relation is not unique to lignin; it is commonly observed and correlated with vessel collapse and occurs most of the time when secondary cell wall genes involved in hemicellulose or cellulose biosynthesis are defective (Voelker et al., 2010; Anterola and Lewis, 2002; Brown et al., 2005). These vessels are essential to feed above-ground tissues with water and nutrients absorbed by the root system (Gomez et al., 2008, Boyce et al., 2004). Hence, silencing strategies, which compromise between the level of the enzymatic step inhibition and biomass yield, are used to reduce lignin in plants.

In woody tissues, a new cell wall, so-called secondary cell wall, is produced and is the main component contributing to biomass density when water is removed. Optimizing cell wall deposition would increase biomass density and therefore energy density. This improvement would be beneficial in reducing the transportation cost of biomass, a significant component in the price of the biomass delivered at the gate of the biorefinery (Searcy et al., 2007; Aden et al., 2002; Kumar et al., 2005). Therefore, developing strategies allowing the thickening of cell wall of woody tissues or pith without altering plant growth can increase biomass and energetic density and would be favorable to the cost-effectiveness of lignocellulosic bioenergy production.

There is an additional need to engineer various biosynthetic pathways in path in a manner such that the production of biosynthetic product can be targeted in a tissue of interest.

This invention addresses these needs.

### BRIEF SUMMARY OF THE INVENTION

Various biological processes exist in organisms from prokaryotes to eukaryotes that are regulated by a small number of transcription factors. In one aspect, this invention provides a positive feedback loop to increase expression of desired products in an organism, e.g., a plant. An artificial positive feedback loop (AFPL) in accordance with the invention employs a transcription factor/promoter construct, typically where the transcription factor is a “master” transcription factor that modulates expression of all or most of the components of a targeted biosynthetic pathway. A promoter from a gene that is downstream in the pathway, where the transcription factor induces or increases expression of the gene, is operably linked to a nucleic acid encoding the transcription factor such that increased expression of the transcription factor results. An AFPL can be used in any biosynthetic process in plants, e.g., to control cell wall deposition, wax/cutin accumulation, or lipid accumulation, and the like.

In one aspect, the invention provides a method of engineering a plant to increase the production of a biosynthetic product in a desired tissue, the method comprising: introducing an expression cassette into the plant, wherein the expression cassette comprises a polynucleotide encoding a transcription factor that regulates production of the biosynthetic product operably linked to a heterologous promoter, wherein the heterologous promoter is a promoter that induces gene expression of a gene that is a downstream target of the transcription factor in the desired tissue; and culturing the plant under conditions in which the transcription factor is expressed. The method may be applied to any plant, including monocots and dicots. In some embodiments, the plant is Arabidopsis, poplar, eucalyptus, rice, corn, switchgrass, sorghum, millet, miscanthus, sugarcane, pine, alfalfa, wheat, soy, barley, turfgrass, tobacco, hemp, poppy, bamboo, rape, sunflower, willow, or Brachypodium.

In some embodiments, the promoter is a tissue-specific secondary wall promoter and the transcription factor induces expression of secondary wall biosynthetic products. For example, the transcription factor may be NAC secondary wall-thickening promoting factor 1 (NST1), NST2, NST3, secondary wall-associated NAC domain protein 2 (SND2), SND3, MYB domain protein 103 (MYB103), MBY85, MYB46, MYB83, MYB58, or MYB63. In some embodiments, the tissue-specific secondary wall promoter is an IRX1, IRX3, IRX5, IRX8, IRX9, IRX14, IRX7, IRX10, GAUT13, GAUT14, or CESA4 promoter.

In some embodiments, of the methods of engineering a plant to increase production of a biosynthetic product in a desired tissue, the transcription factor induces expression of wax and/or cutin. In some embodiments, the transcription factor is a shine (SHN) transcription factor selected from SHN1 (also known as WIN1), SHN2, SHN3, SHN4, or SHN5; or MYB 96. In some embodiments, the promoter is a CER1, CER2, CER3, CER4, CER5, CER6, CER10, WSD1, Mah1, WBC11, KCS1, KCS2, FATB, LACS1, LACS2, CYP864A, CYP86A7, CYP86A5, KCS10, or KCS5 promoter.

In a further aspect, the invention provides a plant comprising an expression cassette that comprises a polynucleotide encoding a transcription factor that regulates production of a biosynthetic product operably linked to a heterologous promoter, wherein the heterologous promoter is a promoter that induces gene expression of a gene that is a downstream target of the transcription factor in the desired tissue; and culturing the plant under conditions in which the transcription factor is expressed. The plant may be any plant, including monocots and dicots. In some embodiments, the plant is Arabidopsis, poplar, eucalyptus, rice, corn, switchgrass, sorghum, millet, miscanthus, sugarcane, pine, alfalfa, wheat, soy, barley, turfgrass, tobacco, hemp, poppy, bamboo, rape, sunflower, willow, or Brachypodium.

In some embodiments, the plant comprises an expression construct in which the promoter is a tissue-specific secondary wall promoter and the transcription factor encoded by the construct induces expression of secondary wall biosynthetic products. For example, the transcription factor may be NAC secondary wall-thickening promoting factor 1 (NST1), NST2, NST3, secondary wall-associated NAC domain protein 2 (SND2), SND3, MYB domain protein 103 (MYB103), MBY85, MYB46, MYB83, MYB58, or MYB63. In some embodiments, the tissue-specific secondary wall promoter is an IRX1, IRX3, IRX5, IRX8, IRX9, IRX14, IRX7, IRX10, GAUT13, GAUT14, or CESA4 promoter.

In some embodiments, the transcription factor encoded by the expression construct induces expression of wax and/or cutin. In some embodiments, the transcription factor is a shine (SHN) transcription factor selected from SHN1 (also known as WIN1), SHN2, SHN3, SHN4, or SHN5; or MYB 96. In some embodiments, the promoter is a CER1, CER2, CER3, CER4, CER5, CER6, CER10, WSD1, Mah1, WBC11, KCS1, KCS2, FATB, LACS1, LACS2, CYP864A, CYP86A7, CYP86A5, KCS10, or KCS5 promoter.

In one aspect, the present invention provides methods of engineering a plant having lignin deposition that is substantially localized to the vessels of xylem tissue of the plant. In some embodiments, the method comprises:
introducing an expression cassette into the plant, wherein the plant is modified to have a reduced level of expression of a lignin biosynthesis enzyme; and further, wherein the expression cassette comprises a polynucleotide encoding the lignin biosynthesis enzyme operably linked to a heterologous vessel-specific promoter; and
culturing the plant under conditions in which the lignin biosynthesis enzyme is expressed.

In some embodiments, the lignin biosynthesis enzyme is PAL, C4H, 4CL, HCT, C3H, or CCR1. In some embodiments, the lignin biosynthesis enzyme is C4H.

In some embodiments, the promoter is a VND1, VND2, VND3, VND4, VND5, VND6, VND7, VNI2, REF4 or RFR1, e.g., a promoter substantially identical to a VND1, VND2, VND3, VND4, VND5, VND6, VND7, VNI2, REF4 or RFR1 promoter; or a native VND1, VND2, VND3, VND4, VND5, VND6, VND7, VNI2, REF4 or RFR1 promoter.

In some embodiments, the level of activity of the lignin biosynthesis enzyme in the modified plant is reduced by contacting the plant with an antisense oligonucleotide that silences expression of the gene encoding the lignin biosynthesis enzyme. In some embodiments, the modified plant in which the polynucleotide operably linked to the heterologous promoter is expressed has a mutation in the gene encoding the lignin synthesis enzyme that decreases expression of the enzyme.

In some embodiments, the plant is selected from the group consisting of Arabidopsis, poplar, eucalyptus, rice, corn, switchgrass, sorghum, millet, miscanthus, sugarcane, pine, alfalfa, wheat, soy, barley, turfgrass, tobacco, hemp, bamboo, rape, sunflower, willow, and Brachypodium.

In some embodiments, the present invention provides plants, plant cells, seeds, flowers, leave, fruit, or biomass comprising plant tissue engineered to have lignin deposition that is substantially localized to the vessels of xylem tissue of the plant.

In another aspect, the present invention provides methods of obtaining an increased amount of soluble sugars from a plant in a saccharification reaction. In some embodiments, the method comprises subjecting a plant engineered to have lignin deposition that is substantially localized to the vessels of xylem tissue of the plant to a saccharification reaction, thereby increasing the amount of soluble sugars that can be obtained from the plant as compared to a wild-type plant.

In still another aspect, the present invention provides methods of engineering a plant having increased secondary cell wall deposition. In some embodiments, the method comprises:
introducing an expression cassette into the plant, wherein the expression cassette comprises a polynucleotide encoding a transcription factor that regulates the production of secondary cell wall in woody tissue operably linked to a heterologous promoter, wherein the promoter is substantially identical to the native promoter of a gene that is a downstream target of the transcription factor; and
culturing the plant under conditions in which the transcription factor is expressed. In some embodiments, the promoter and the transcription factor, or either the promoter or the transcription factor are from a different plant species than the host cell in which the artificial positive feedback loop is created. In further embodiments, the transcription factor and the promoter are from different plant species.

In some embodiments, the transcription factor is NST1, NST2, NST3, MYB103, MYB85, MYB46, MYB83, MYB58, or MYB63. In some embodiments, the transcription factor is NST1.

In some embodiments, the promoter is an IRX1, IRX3, IRX5, IRX8, IRX9, IRX14, IRX7, or IRX10 promoter. In some embodiments, the promoter is a native IRX1, IRX3, IRX5, IRX8, IRX9, IRX14, IRX7, or IRX10 promoter.

In some embodiments, the plant in which the polynucleotide operably linked to the heterologous promoter is expressed is a wild-type plant. In some embodiments, the plant in which the polynucleotide operably linked to the heterologous promoter is expressed is an engineered plant having lignin deposition that is substantially localized to the vessels of xylem tissue of the plant.

In some embodiments, the plant is selected from the group consisting of Arabidopsis, poplar, eucalyptus, rice, corn, switchgrass, sorghum, millet, miscanthus, sugarcane, pine, alfalfa, wheat, soy, barley, turfgrass, tobacco, hemp, bamboo, rape, sunflower, willow, and Brachypodium.

In some embodiments, the present invention provides plants, plant cells, seeds, flowers, leave, fruit, or biomass comprising plant tissue engineered to have increased secondary cell wall deposition.

In yet another aspect, the present invention provides methods of increasing bioenergy production from biomass derived from a plant. In some embodiments, the method comprises harvesting biomass from a plant engineered to have increased secondary cell wall deposition; and subjecting the biomass to a conversion reaction, thereby increasing bioenergy production as compared to a wild-type plant.

Ina further aspect, the present invention provides methods of increasing stem/straw/timber strength, which can reduce lodging, and increase wood density from a plant. Thus, the invention provides a method of increasing stem, straw or timber strength from plants during growth, the method comprising: cultivating plants engineered to have increased secondary cell wall deposition, thereby improving resistance lodging as compared to a wild type plants. Plants having increased secondary wall deposition may also be cultivated to provide plants, or biomass from such plants that have increased resistance to mechanical stress compared to a wildtype plant.

In yet another aspect, present invention provides methods of engineering a plant having xylan deposition that is substantially localized to the vessels of xylem tissue of the plant. In some embodiments, the method comprises:
introducing an expression cassette into the plant, wherein the plant is modified to have a reduced level of activity of a xylan biosynthesis enzyme; and further, wherein the expression cassette comprises a polynucleotide encoding the xylan biosynthesis enzyme operably linked to a heterologous vessel-specific promoter; and
culturing the plant under conditions in which the xylan biosynthesis enzyme is expressed. In some embodiments, the plant into which the expression cassette is introduced is modified to have a reduced leve of expression of a xylan biosynthesis enzyme.

In some embodiments, the xylan biosynthesis enzyme is irregular xylem 8 (IRX8), IRX14, IRX14-like, IRX9, IRX9-like, IRX7, IRX10, IRX10-like, IRX15, IRX15-like, F8H, or PARVUS.

In some embodiments, the promoter is a VND1, VND2, VND3, VND4, VND5, VND6, VND7, VNI2, REF4 or RFR1, e.g., a promoter substantially identical to a VND1, VND2, VND3, VND4, VND5, VND6, VND7, VNI2, REF4 or RFR1 promoter; or a native VND1, VND2, VND3, VND4, VND5, VND6, VND7, VNI2, REF4 or RFR1 promoter.

In some embodiments, the level of activity of the xylan biosynthesis enzyme in the modified plant is reduced by contacting the plant with an antisense oligonucleotide that silences expression of the gene encoding the xylan biosynthesis enzyme. In some embodiments, the modified plant in which the polynucleotide operably linked to the heterologous promoter is expressed has a mutation in the gene encoding the xylan synthesis enzyme that decreases expression of the enzyme. In some embodiments, the activity of the xylan biosynthesis enzyme in the modified plant is reduced by contacting the plant with a mutated xylan biosynthesis gene that encodes a protein with a dominant negative mutation and causes a decrease in xylan biosynthesis.

In some embodiments, the plant is selected from the group consisting of Arabidopsis, poplar, eucalyptus, rice, corn, switchgrass, sorghum, millet, miscanthus, sugarcane, pine, alfalfa, wheat, soy, barley, turfgrass, tobacco, hemp, bamboo, rape, sunflower, willow, and Brachypodium.

In some embodiments, the present invention provides plants, plant cells, seeds, flowers, leave, fruit, or biomass comprising plant tissue engineered to have xylan deposition that is substantially localized to the vessels of xylem tissue of the plant.

In yet another aspect, the present invention provides methods of obtaining an increased amount of soluble sugars from a plant in a saccharification reaction. In some embodiments, the method comprises subjecting a plant engineered to have xylan deposition that is substantially localized to the vessels of xylem tissue of the plant to a saccharification reaction, thereby increasing the amount of soluble sugars that can be obtained from the plant as compared to a wild-type plant.

In still another aspect, the present invention provides methods of engineering a plant having xylan O-acetylation that is substantially localized to the vessels of xylem tissue of the plant. In some embodiments, the method comprises:
introducing an expression cassette into the plant, wherein the plant is modified to have a reduced level of expression of an enzyme responsible for xylan O-acetylation; and further, wherein the expression cassette comprises a polynucleotide encoding the xylan O-acetylation enzyme operably linked to a heterologous vessel-specific promoter; and culturing the plant under conditions in which the xylan O-acetylation enzyme is expressed.

In some embodiments, the xylan O-acetylation enzyme is an RWA protein.

In some embodiments, the xylan O-acetylation enzyme is a member of the Trichome Birefringence Like family of proteins (PF03005 family also known as Domain of Unknown Function 231).

In some embodiments, the promoter is a VND1, VND2, VND3, VND4, VND5, VND6, VND7, VNI2, REF4 or RFR1, e.g., a promoter substantially identical to a VND1, VND2, VND3, VND4, VND5, VND6, VND7, VNI2, REF4 or RFR1 promoter; or a native VND1, VND2, VND3, VND4, VND5, VND6, VND7, VNI2, REF4 or RFR1 promoter.

In some embodiments, the level of expression of the xylan O-acetylation enzyme in the modified plant is reduced by contacting the plant with an antisense oligonucleotide that silences expression of the gene encoding the xylan O-acetylation enzyme. In some embodiments, the modified plant in which the polynucleotide operably linked to the heterologous promoter is expressed has a mutation in the gene encoding the xylan O-acetylation enzyme that decreases expression of the enzyme.

In some embodiments, the plant is selected from the group consisting of Arabidopsis, poplar, eucalyptus, rice, corn, switchgrass, sorghum, millet, miscanthus, sugarcane, pine, alfalfa, wheat, soy, barley, turfgrass, tobacco, hemp, bamboo, rape, sunflower, willow, and Brachypodium.

In some embodiments, the present invention provides plants, plant cells, seeds, flowers, leave, fruit, or biomass comprising plant tissue engineered to have xylan deposition that is substantially localized to the vessels of xylem tissue of the plant.

In yet another aspect, the present invention provides methods of obtaining an increased amount of soluble sugars from a plant in a saccharification reaction. In some embodiments, the method comprises subjecting a plant engineered to have xylan O-acetylation that is substantially localized to the vessels of xylem tissue of the plant to a saccharification reaction, thereby increasing the amount of soluble sugars that can be obtained from the plant as compared to a wild-type plant.

### BRIEF DESCRIPTION OF THE DRAWINGS

FIG. 1. Phenylalanine ammonia-lyase (PAL) alignment. The protein sequences for PAL from Arabidopsis thaliana (“AtPAL1”), Physcomitrella patens (moss) (“PpPAL3”), Oryza sativa (rice) (“OsPAL”), Zea mays (maize) (“ZmPAL”), Sorghum bicolor (sorghum) (“SbPAL”), Pinus massoniana (pine) (“PlPAL”), Medicago sativa (alfalfa) (“MsPAL”), Triticum aestivum (wheat) (“TaPAL”), Glycine max (soybean) (“GmPAL2”), Beta vulgaris (sugar beet) (“BvPAL”), Nicotiniana tabacum (tobacco) (“NtPAL1”), Solanum tuberosum (potato) (“StPAL1”), Bambusa oldhamii (bamboo) (“BoPAL”), Brassica rapa (“BnPAL1”), Helianthus annuus (sunflower) (“HaPAL”), Ricinus communis (“RcPAL”), Vitis vinifera (grape) (“VvPAL”), Jatropha curcas (“JcPAL”), Euphorbia pulcherrima (poinsettia) (“EpPAL”), Trifolium pratense (clover) (“TpPAL”), Lotus japonicus (“LjPALS”), and Selaginella moellendorffii (spike moss) (“SmPAL”) were aligned using ClustalW.

FIG. 2. Cinnamate 4-hydroxylase (C4H) alignment. The protein sequences for C4H from Arabidopsis thaliana (“AtC4H”), Pinus taeda (pine) (“PtC4H”), Oryza sativa (rice) (“OsC4H”), Zea mays (maize) (“ZmC4H”), Sorghum bicolor (sorghum) (“SbC4H”), Medicago truncatula (“MtC4H”), Triticum aestivum (wheat) (“TaC4H”), Glycine max (soybean) (“GmC4H”), Nicotiniana tabacum (tobacco) (“NtC4H”), (“StC4H”), Solanum tuberosum (potato) (“StC4H”), Bambusa oldhamii (bamboo) (“BoC4H”), Brassica napus (“BnC4H1”), Helianthus annuus (sunflower) (“HaC4H”), Ricinus communis (“RcC4H”), Vitis vinifera (grape) (“VvC4H”), Euphorbia pulcherrima (poinsettia) (“EpC4H”), Trifolium pratense (clover) (“TpC4H”), and Selaginella moellendorffii (spike moss) (“SmC4H”) were aligned using ClustalW.

FIG. 3. 4-coumarate-CoA ligase (4CL) alignment. The protein sequences for 4CL from Arabidopsis thaliana (“At4CL2” and “At4CL1”), Nicotiniana tabacum (tobacco) (“Nt4CL1” and “Nt4CL2”), Eucalyptus camaldulensis (“Ec4CL”, “Ec4CL1”, and “Ec4CL2”), Pinus taeda (pine) (“Pt4CL” and “Pt4CL1”), Glycine max (soybean) (“Gm4CL1”), Oryza sativa (rice) (“Os4CL3” and “Os4CL4”), Sorghum bicolor (sorghum) (“Sb4CL”), Zea mays (maize) (“Zm4CL”), Panicum virgatum (switchgrass) (“Pv4CL”), Lolium perenne (ryegrass) (“Lp4CL3”), Selaginella moellendorffii (spike moss) (“Sm4CL1”), and Physcomitrella patens (moss) (“Pp4CL1”) were aligned using ClustalW.

FIG. 4. Hydroxycinnamoyl CoA: shikimate hydroxycinnamoyl transferase (HCT) alignment. The protein sequences for HCT from Arabidopsis thaliana (“AtHCT”), Arabidopsis lyrata (“A1HCT”), Pinus taeda (pine) (“PtHCT”), Ricinus communis (“RcHCT”), Coffea canephora (“CcHCT”), Vitis vinifera (grape) (“VvHCT”), Nicotiniana tabacum (tobacco) (“NtHCT”), Trifolium pratense (clover) (“TpHCT”), Oryza sativa (rice) (“OsHCT” and “OsHCT3”), Sorghum bicolor (sorghum) (“SbHCT”), Zea mays (maize) (“ZmHCT” and “ZmHCT2”), Avena sativa (oat) (“AsHCT”), and Selaginella moellendorffii (spike moss) (“SmHCT1” and “SmHCT2”) were aligned using ClustalW.

FIG. 5. Coumaroyl shikimate 3-hydroxylase (C3H) alignment. The protein sequences for C3H from Arabidopsis thaliana (“AtC3H”), Eucalyptus globulus (“EgC3H”), Ricinus communis (“RcC3H”), Vitis vinifera (grape) (“VvC3H”), Glycine max (soybean) (“GmC3H”), Trifolium pratense (clover) (“TpC3H”), Medicago truncatula (“MtC3H”), Coffea canephora (“CcC3H”), Osimum basilicum (basil) (“ObC3H”), Pinus taeda (pine) (“PtC3H”), Nicotiniana tabacum (tobacco) (“NtC3H”), Ginkgo biloba (“GbC3H”), (“SbC3H”), Zea mays (maize) (“ZmC3H”), Oryza sativa (rice) (“OsC3H”), Triticum aestivum (wheat) (“TaC3H”), Selaginella moellendorffii (spike moss) (“SmC3H”), and Physcomitrella patens (moss) (“FpC3H”) were aligned using ClustalW.

FIG. 6. Cinnamoyl-CoA reductase (CCR) alignment. The protein sequences for CCR from Arabidopsis thaliana (“AtCCR1”), Solanum lycopersicum (tomato) (“SlCCR”), Euphorbia pulcherrima (poinsettia) (“EpCCR”), Solanum tuberosum (potato) (“StCCR”), Eucalyptus gunnii (“EgCCR”), Vitis vinifera (grape) (“VvCCR”), Ricinus communis (“RcCCR”), Pinus taeda (pine) (“PtCCR”), Glycine max (soybean) (“GmCCR”), Picea abies (spruce) (“PaCCR”), Pinus massoniana (pine) (“PmCCR”), Oryza sativa (rice) (“OsCCR”), Lolium perenne (ryegrass) (“LpCCR”), Panicum virgatum (switchgrass) (“PvCCR”), Sorghum bicolor (sorghum) (“SbCCR”), Saccharum officiunarum (sugarcane) (“SoCCR”), Hordeum vulgare (barley) (“HvCCR”), Zea mays (maize) (“ZmCCR”), and Selaginella moellendorffii (spike moss) (“SmCCR”) were aligned using ClustalW.

FIG. 7. IRX8 sequence alignment. Alignment of amino acid sequences of Arabidopsis IRX8 (GAUT12) and homologous proteins. The alignment was made with COBALT (Papadopoulos J S and Agarwala R (2007) COBALT: constraint-based alignment tool for multiple protein sequences, Bioinformatics 23:1073-79). Proteins are identified by their GenBank protein IDs. gi15239707: IRX8 from Arabidopsis thaliana; gi2241262287: homolog from Populus trichocarpa, gi224117396: homolog from P. trichocarpa, gi224141469: homolog from P. trichocarpa, gi224077712: homolog from P. trichocarpa; gi302803855: homolog from Selaginella moellendorffii; gi30678270: GAUT13 from A. thaliana; gi30685369: GAUT14 from A. thaliana; gi115489272: homolog from Oryza sativa; gi224131384: homolog from P. trichocarpa; gi22331857: GAUT15 from A. thaliana.

FIG. 8. IRX14 alignment. Alignment of amino acid sequences of Arabidopsis IRX14 and homologous proteins. The alignment was made with COBALT (Papadopoulos J S and Agarwala R (2007) COBALT: constraint-based alignment tool for multiple protein sequences, Bioinformatics 23:1073-79). Proteins are identified by their GenBank protein IDs. gi|30690793: IRX14 from A. thaliana; gi|15240245: IRX14-like from A. thaliana; gi|224096716 and gi|224081752: homologs from P. trichocarpa; gi|302797519: homolog from S. moellendorffii; gi|115469624: homolog from O. sativa.

FIG. 9. IRX9 alignment. Alignment of amino acid sequences of Arabidopsis IRX9 and homologous proteins. The alignment was made with COBALT (Papadopoulos J S and Agarwala R (2007) COBALT: constraint-based alignment tool for multiple protein sequences, Bioinformatics 23:1073-79). Proteins are identified by their GenBank protein IDs. gi|15228084: IRX9 from A. thaliana; gi|224140167 and gi|224069352: homologs from P. trichocarpa; gi|297600755 and g|115461821: homologs from O. sativa; gi|224092304: homolog from P. trichocarpa; gi|302759368: Homolog from S. moellendorffii; gi|42571663: IRX9-like from A. thaliana; gi|224063335: homolog from P. trichocarpa; gi|115439133, gi|115474279, gi|115465403 451, gi|115481434 and gi|115456794: homologs from O. sativa.

FIG. 10. IRX7 alignment. Alignment of amino acid sequences of Arabidopsis IRX7 (FRA8) and homologous proteins. The alignment was made with COBALT (Papadopoulos J S and Agarwala R (2007) COBALT: constraint-based alignment tool for multiple protein sequences, Bioinformatics 23:1073-79). Proteins are identified by their GenBank protein IDs. gi|42570324: IRX7 from A. thaliana; gi|224106838: homolog from P. trichocarpa; gi|42568020: IRX7-like (F8H) from A. thaliana; gi|115450193: homolog from O. sativa; gi|302786830 and gi|302826405: homologs from S. moellendorffii.

FIG. 11. IRX10 alignment. Alignment of amino acid sequences of Arabidopsis IRX10 and homologous proteins. The alignment was made with COBALT (Papadopoulos J S and Agarwala R (2007) COBALT: constraint-based alignment tool for multiple protein sequences, Bioinformatics 23:1073-79). Proteins are identified by their GenBank protein IDs. gi|18424516: IRX10-like (GUT1) from A. thaliana; gi|224119858: homolog from P. trichocarpa; gi|15223522: IRX10 (GUT2) from A. thaliana; gi|224053575 and gi|224075447: homologs from P. trichocarpa; gi|115441967: Os01g0926600 from O. sativa; gi|302783378: GT47D1 from S. moellendorffii; gi|115458146: Os04g0398600 from O. sativa; gi|115441965: Os01g0926400 from O. sativa; gi|115481310: Os10g0180000 from O. sativa; gi|224106838: homolog from P. trichocarpa.

FIG. 12. Parvus sequence alignment. Alignment of amino acid sequences of Arabidopsis PARVUS (GATL1) and homologous proteins. The alignment was made with COBALT (Papadopoulos J S and Agarwala R (2007) COBALT: constraint-based alignment tool for multiple protein sequences, Bioinformatics 23:1073-79). Proteins are identified by their GenBank protein IDs. gi|18394719: PARVUS from A. thaliana. The other proteins are some of the homologs from A. thaliana, P. trichocarpa, and O. sativa, and the single homolog from S. moellendorffii (gi|302807664).

FIG. 13. NAC secondary wall-thickening promoting factor (NST) alignment. The protein sequences for NST from Arabidopsis thaliana (“AtNST1”, “AtNST2”, and “SND”), Pinus taeda (pine) (“PtNAC023”, “PtNAC065”, and “PtNAC”), Medicago truncatula (“MtNAC1”), Glycine max (soybean) (“GmNAM1”), Vitis vinifera (grape) (“VvNST”), Ricinus communis (“RcNST”), Eucalyptus gunnii (“EgNST”), Zea mays (maize) (“ZmNST”), Sorghum bicolor (sorghum) (“SbNST”), Oryza sativa (rice) (“OsNAC7” and “OsNST”), Picea sitchensis (spruce) (“PsNST”), apple (“AppleT”), and Selaginella moellendorffii (spike moss) (“SmNST1”) were aligned using ClustalW.

FIG. 14. Transcriptional network regulating secondary cell wall biosynthesis. Major transcription factors regulating secondary cell wall deposition in tracheary elements and fibers are presented as well as several of the downstream target genes that are induced during secondary cell wall biosynthesis. The transcription factors presented are able to induce expression of genes involved in cellulose, hemicellulose and/or lignin biosynthesis. The drawing is adapted from Zhong et al., 2007.

FIG. 15. Lignin analysis of cell wall of engineered plant lines. A. Lignin quantification using the acetyl bromide method on senesced stems from wild-type (W) and engineered (“Eng Lig I”) (ref3-2+pVND6:C4H) plants. B. Bright-light images of stem cross-sections stained with phloroglucinol of same-age wild-type (W) and two engineered Eng Lig I plants from left to right respectively.

FIG. 16. Analysis of the Eng Lig I line. A. Plant growth phenotype of Eng Lig I compared at two different growth stages. The top panel represents the vegetative stage and the bottom panel represents the adult stage (bolting stage). Wild-type plants are shown on the left and the engineered Eng Lig I plants are shown on the right in A-D. B. Sugar released from dry stems pretreated with NaOH and incubated with a cellulase cocktail for 0, 24, or 48 hrs. C. Sugar released from dry stems pretreated with hot water and incubated with a cellulase cocktail for 0, 24, or 48 hrs. D. Sugar released from dry stems pretreated with dilute acid and incubated with a cellulase cocktail for 0, 24, or 48 hrs.

FIG. 17. Analysis of the Eng Lig II line. A. Plant growth phenotype of Eng Lig II (ref3-2+pVND6:C4H+ pIRX8:NST1) compared at two different growth stages. The top panel represents the vegetative stage and the bottom panel represents the adult stage (bolting stage). Wild-type plants are shown on the left and the engineered Eng Lig II plants are shown on the right. B. Bright-light images of stem cross-sections stained with phloroglucinol of same-age wild-type (W), ref3-2 mutant, and the engineered Eng Lig II plants from left to right respectively. C. Lignin quantification using the acetyl bromide method on senesced stems from wild-type (W), engineered Eng Lig I, and engineered Eng Lig II plants.

FIG. 18. Transmission electron micrographs of cross-sections through wild-type (A, C) and engineered (ref3-2+pVND6:C4H+pIRX8:NST1) (B, D) plants. A-B. Xylem tissues of the plants. C-D. Interfascicular tissues of the plants. “Ve,” “Xf,” and “If” stand for vessels, xylery fibers, and interfascicular fibers, respectively.

FIG. 19. Saccharification efficiency of the Eng Lig I and Eng Lig II lines. A. Sugar released from dry stems pretreated with hot water and incubated with a cellulase cocktail for 0 to 144 hrs. Stems are from wild-type (wt; blue) plant, engineered Eng Lig I (orange) plants, or Eng Lig II (red) plants. B. Sugar released from dry stems pretreated with NaOH and incubated with a cellulase cocktail for 0 to 144 hrs. Stems are from wild-type (wt; blue) plant, engineered Eng Lig I (orange) plants, or Eng Lig II (red) plants.

FIG. 20. Promoter activity characterization. A. Bright-field image of stem cross-section from the base of 5-10 cm stems from wild-type (WT), cadc/d mutant, cadc/d mutant transformed with pVND6:CADc, and cadc/d mutant transformed with pC4H:CADc, from left to right respectively. The redness is generated by the lack of CAD activity. B. Bright-field image from Maule stained stem cross-section from the base of 5-10 cm stems from wild-type (WT), f5h mutant, f5h mutant transformed with pVND6:F5H, and f5h mutant transformed with pC4H:F5H, from left to right respectively. The redness is generated by the presence of Sinapyl alcohol and is representative of the amount of Sinapyl alcohol in the lignin that reacts during the Maule staining reaction. The production of Sinapyl alcohol is restored in the f5h mutant by the expression of the native F5H gene.

FIG. 21. Xylem collapse. A. Same-age adult ref3-2 mutant (homozygote c4h mutant) and wild-type plants (wt) (right and left, respectively). B. Same vegetative age ref3-2 mutant (homozygote c4h mutant) and wild-type plants (right and left, respectively). C. Top and bottom panels represent a bright-field image of phloroglucinol-stained stem cross-sections, magnified 20 and 40× fold respectively, from wild-type and ref3-2 (left and right respectively) sampled at the same age as presented on A. The yellow arrows point to some collapse vessels in the ref3-2 mutant.

FIG. 22. Expression analysis of NST1. NST1 expression was analyzed by semi-quantitative RT-PCR. pIRX8:NST1: specific NST1 primers were used to verify the expression of NST1 driven by pIRX8 promoter. NST1: specific NST1 primers were used to verify the expression of both NST1 genes each driven by pIRX8 and pNST1 promoters. pVND6:C4H: specific C4H primers were used to verify the expression of the C4H genes driven by pVND6. C4H: specific C4H primers were used to verify the expression of the C4H genes driven by pVND6 or pC4H promoters (wild-type and ref3-2 mutant alleles). Tubulin: specific tubulin primers was used to verify the quality and quantity of the RNA used for the RT-PCR. Lanes 1 to 4 show independent Eng Lig II (ref3-2+pVND6:C4H+pIRX8:NST1) plants; lane 4 shows a wild-type plant; lanes 5 and 6 show independent Eng Lig I (ref3-2+pVND6:C4H) plants; and lane 7 shows a ref3-2 mutant plant.

FIG. 23. Cell wall thickness. A-D. Cell wall thickness and cell diameters were measured on 20 independent fiber cells from the intrafascicular regions in Col0 (WT) (A), ref3-2 (c4h mutant) (B), Eng Lig I (C), and Eng Lig II (D) plants. Cell wall ratio was measured by the sum of the cell wall thickness (μm) divided by the cell diameter (μm). E. Cell wall thickness and cell diameter measurement method. The green bar (a) and yellow bar (b) each represent cell wall thickness measurements and the pink bar represents the cell diameter. Cell wall ratio was measured by the sum of the cell wall thickness (μm) divided by the cell diameter (μm), (a+b)/cell diameter.

FIG. 24. Sugar release from cell wall after chemical hydrolysis. A-B. Hemicellulose composition after TFA hydrolysis. A. Quantification (mg of sugar/mg dry cell wall) of the major sugar released). B. Percentage of each sugar in the total released. C. Total sugar released after H2SO4 hydrolysis.

FIG. 25. Alignment of SHN protein sequences. The protein sequences for SHN polypeptides from Arabidopsis thaliana (“At”), Populus trichocarpa (“Pt”), Medicago truncatula (“Mt”), Oryza sativa (“Os”), Brachypodium distachyon (“Bd”), Zea mays (“Zm”), Sorghum bicolor (“Sb”), Hordeum vulgare (“Hv”), Picea sitchensis (“Ps”), Selaginella moellendorffi (“Sm”), and Physcomitrella patens (“Pp) were aligned using ClustalW.

FIG. 26. Alignment of Myb96 protein sequences. The protein sequences for Myb96 polypeptides from Arabidopsis thaliana (“At”), Thellungiella halophila (“Th), Medicago truncatula (“Mt”), Populus trichocarpa (“Pt”), Vitis vinifera (“Vv”), Citrus macrophylla (“Cm”), Brachypodium distachyon (“Bd”), Triticum aestivum (“Ta”), Oryza sativa (“Os”), and Zea Mays (“Zm”) were aligned using ClustalW.

FIG. 27. Representation of cell wall artificial positive feed back loop. FIG. 27 depicts an illustrative cell wall densification strategy.

FIG. 28. Induction of wax biosynthetic pathways in target tissues. FIG. 28 depicts an illustrative artificial positive feed back loop to induce a wax biosynthetic pathway in target tissues.

FIG. 29. Plant growth phenotype of engineered cell wall plant lines. Growth comparison of wildtype, c4h mutant plants and engineered plant lines in which the ref3-2 mutation is complemented with either pREF4::C4H (A) or pRFR1::C4H (B) DNA construct.

FIG. 30. Lignin distribution and content of engineered cell wall plant lines. Lignin distribution is shown in the upper panel. Lignin quantification is shown in the lower panel.

FIG. 31. Saccarificaton efficiency of lignin engineered plant lines. Panels A and B show sugar released form dry stems using hot-water (Panel A) or alkali (Panel B) pretreatment follow by incubation was a cellulase cocktail. Panel C provides a summary of the saccharification results.

FIG. 32. Cell wall densification feed back loop. Panel A illustrates cell wall densification in Arabidopsis wildtype plants containing a DNA construct pCesA4::NST1. Panel B shows cell wall densification in Brachypodium wildtype plants using pAtlRX8::AtNST1 DNA construct where the promoter and transcription factor are both from Arabidopsis.

FIG. 33. Examples of xylan engineering. Comparison of growth in wildtype, mutant, and mutant plants complemented with the wildtype version of the mutated IRX7, IRX8, or IRX9 gene drive by pVND6 or pVND7.

FIG. 34. Growth of offspring of transformants. Growth of offspring of four individual transformants made by transforming irx7 mutant with a pVND7::IRX7 expression construct.

FIG. 35. Growth of offspring of transformants. Growth of offspring of two individual transformants made by transforming irx9 mutant with a pVND7::IRX9 expression construct.

FIG. 36 Non-cellulosic monosaccharide composition prepared from transformants. Non-cellulosic monosaccharide composition of cell walls prepared from four individual transformants made by transforming irx7 mutant with a pVND7::IRX7 expression construct.

FIG. 37 Non-cellulosic monosaccharide composition prepared from transformants. Non-cellulosic monosaccharide composition of cell walls prepared from four individual transformants made by transforming irx8 mutant with a pVND6::IRX8 expression construct.

FIG. 38. Noncellulosic monosaccharide composition of stem cell walls prepared from individual transformants. Non-cellulosic monosaccharide composition of stem cell wall prepared from offspring of four individual transformants made by transforming irx9 mutant with a pVND7::IRX9 expression construct.

FIG. 39. Saccharification analysis of cells walls. Saccharification analysis of cell walls prepared from offspring of two individual transformants made by transforming irx9 mutant with a pVND6::IRX9 expression construct.

FIG. 40. Wax deposition in plants transformed to create an artificial positive feedback loop. Visual analysis of the Arabidopsis plant transformed with the different constructs showed increased shininess of the leaves compared with control plants.

### DETAILED DESCRIPTION OF THE INVENTION

### I. Definitions

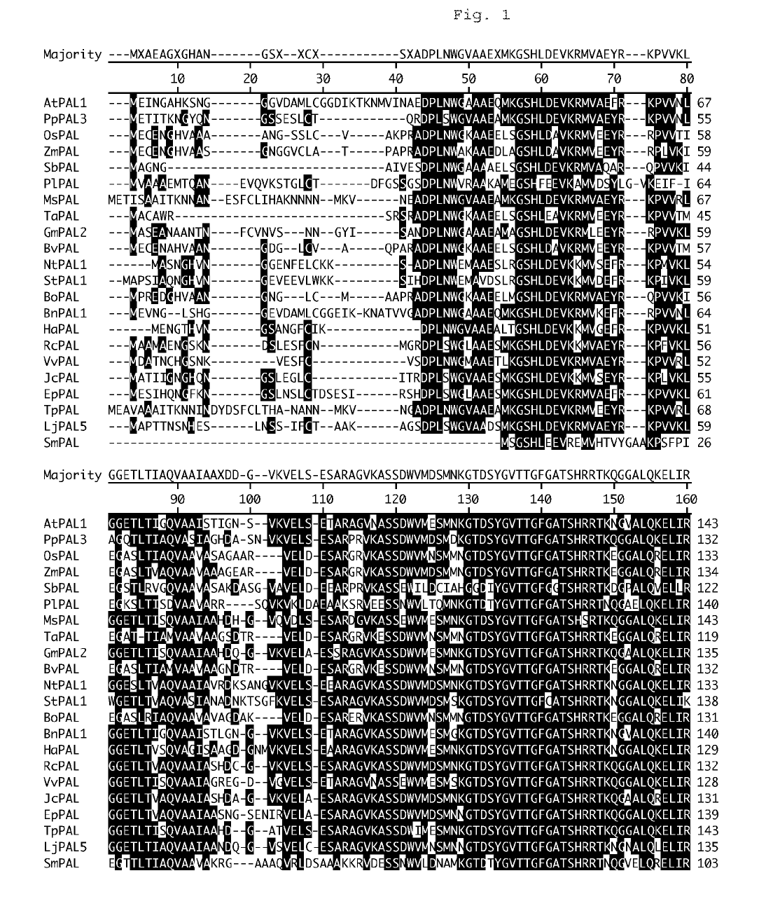

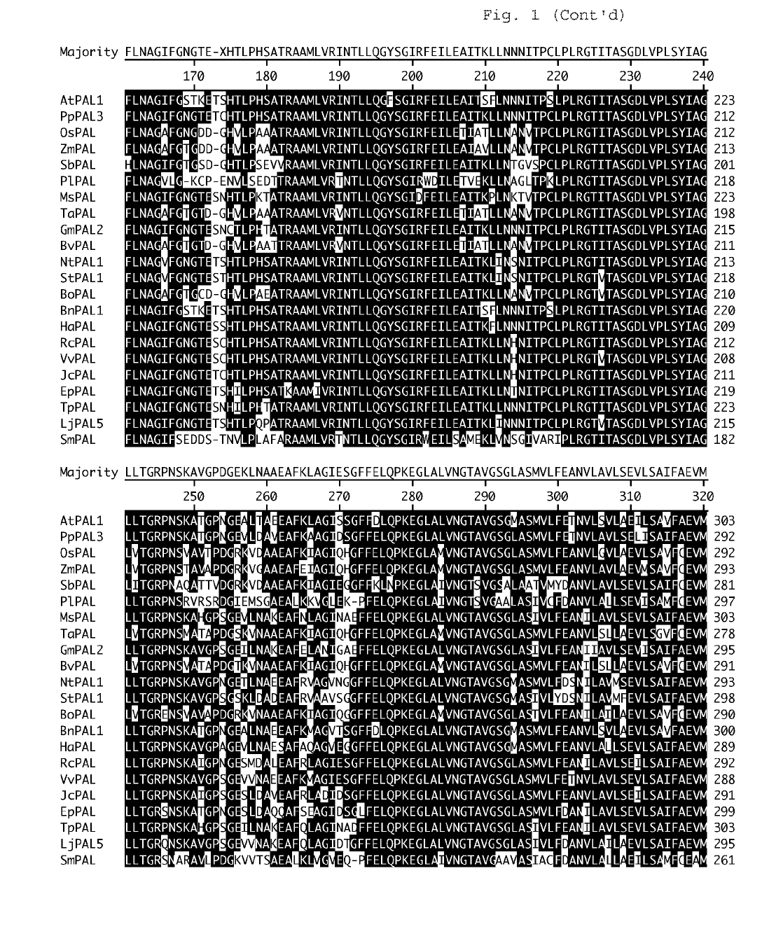

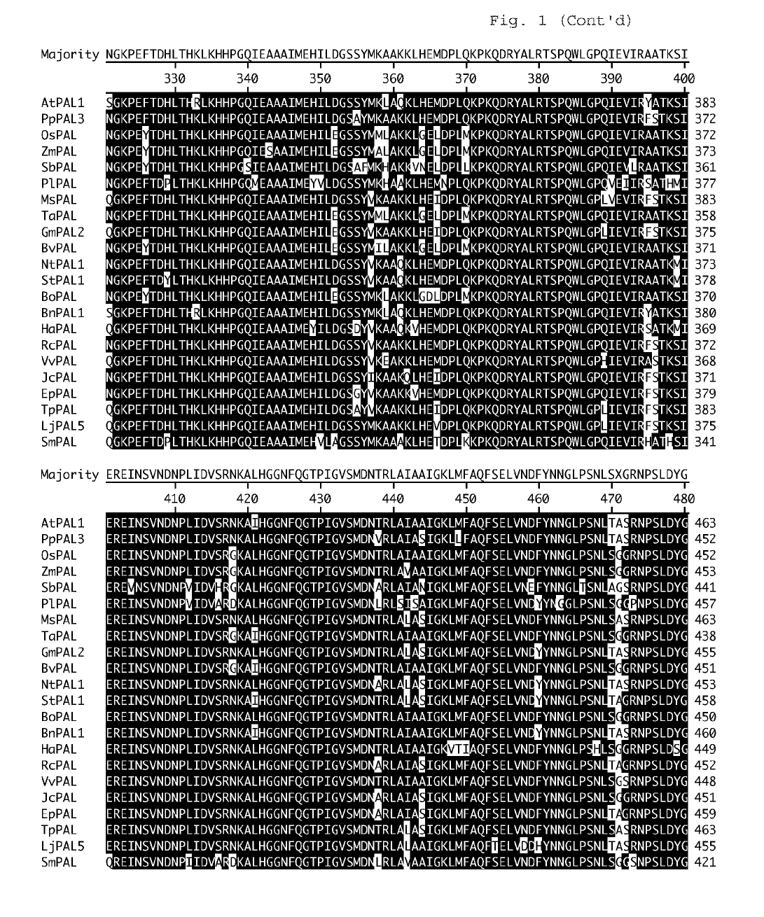

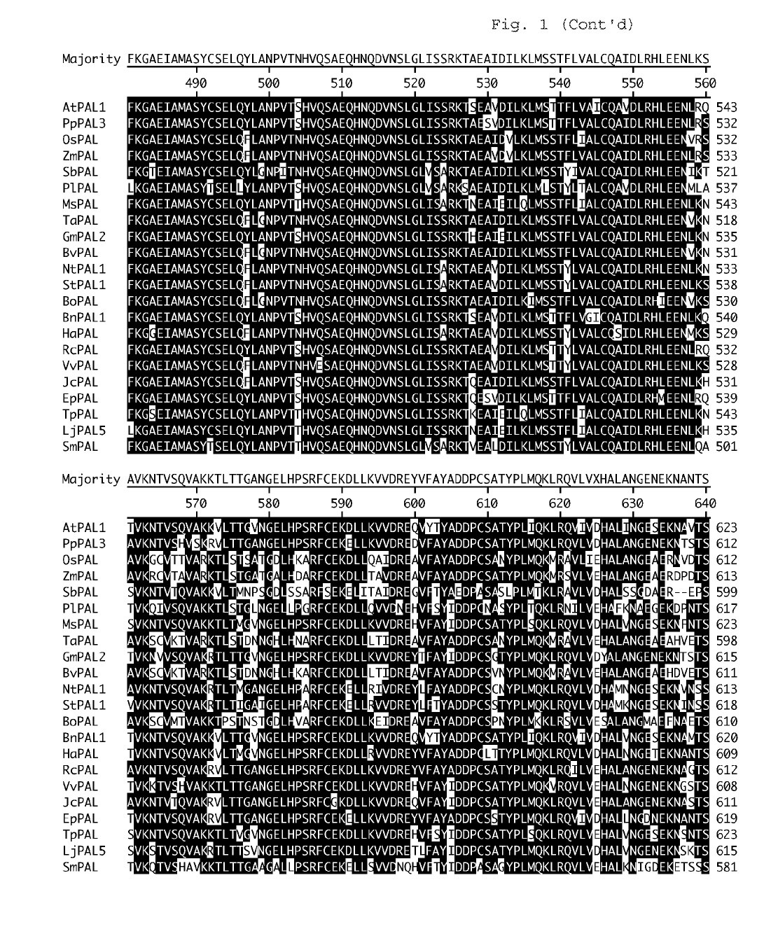

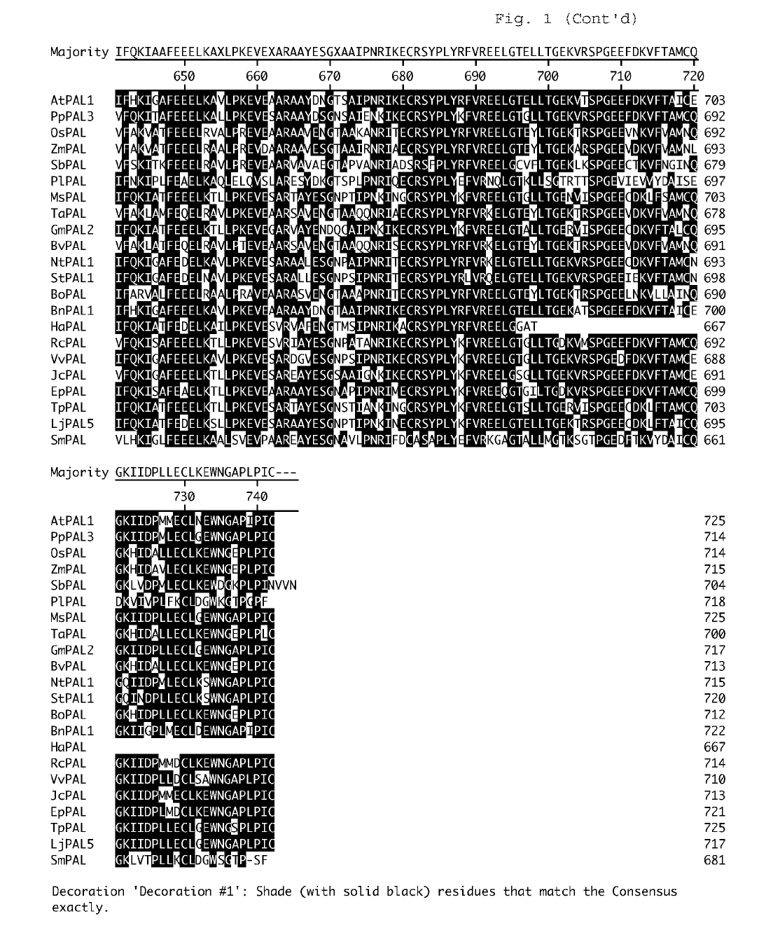

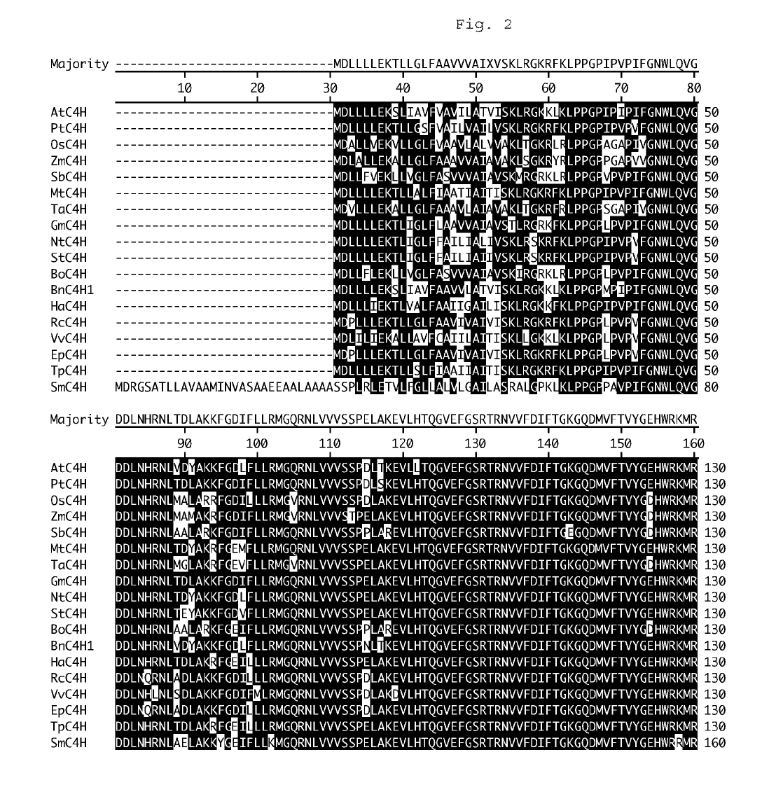

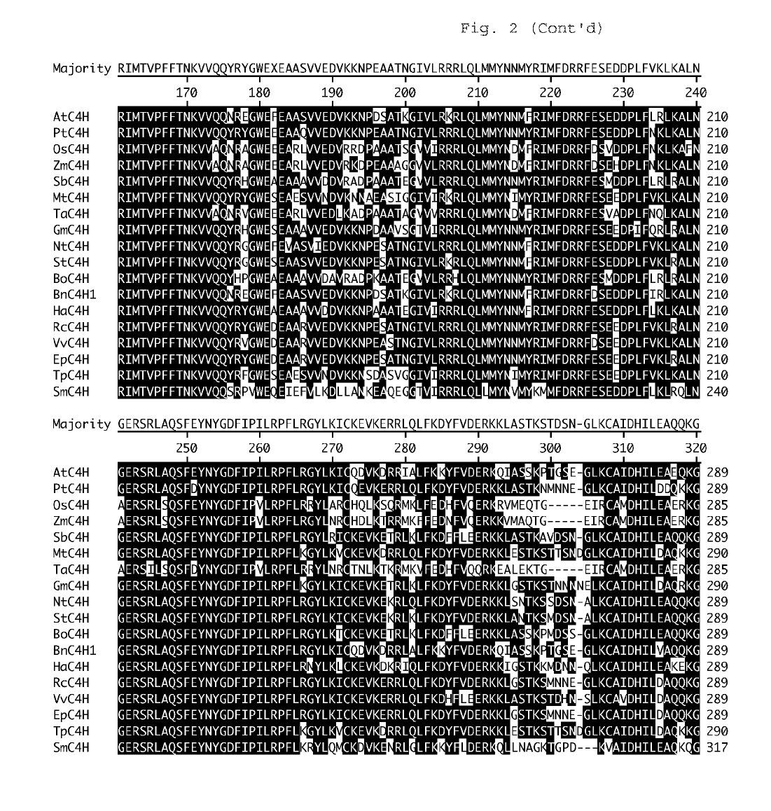

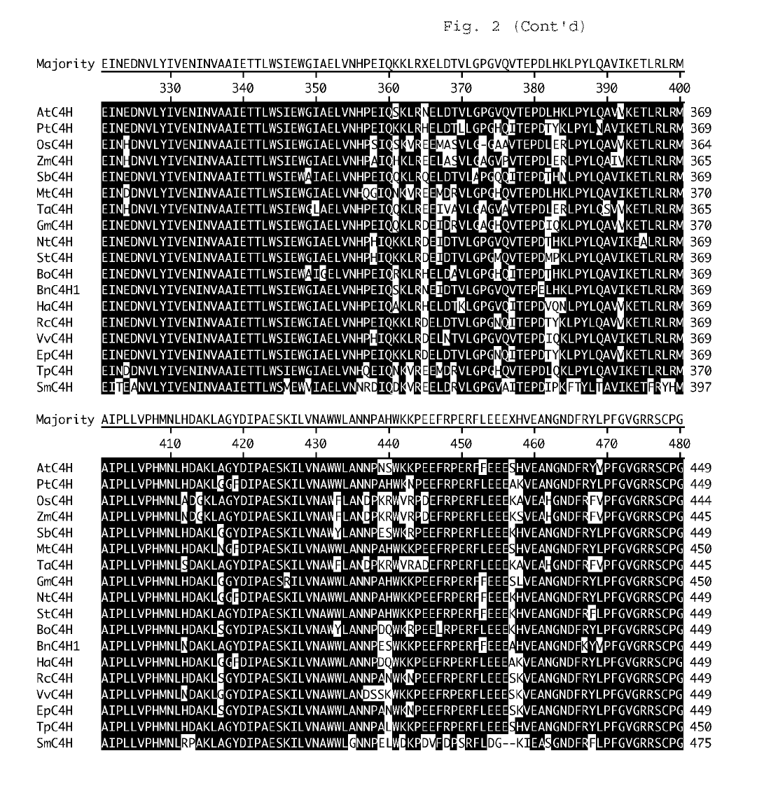

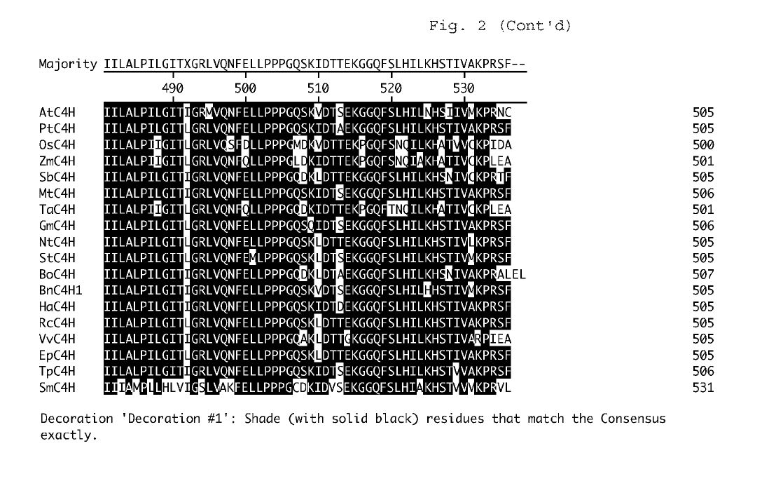

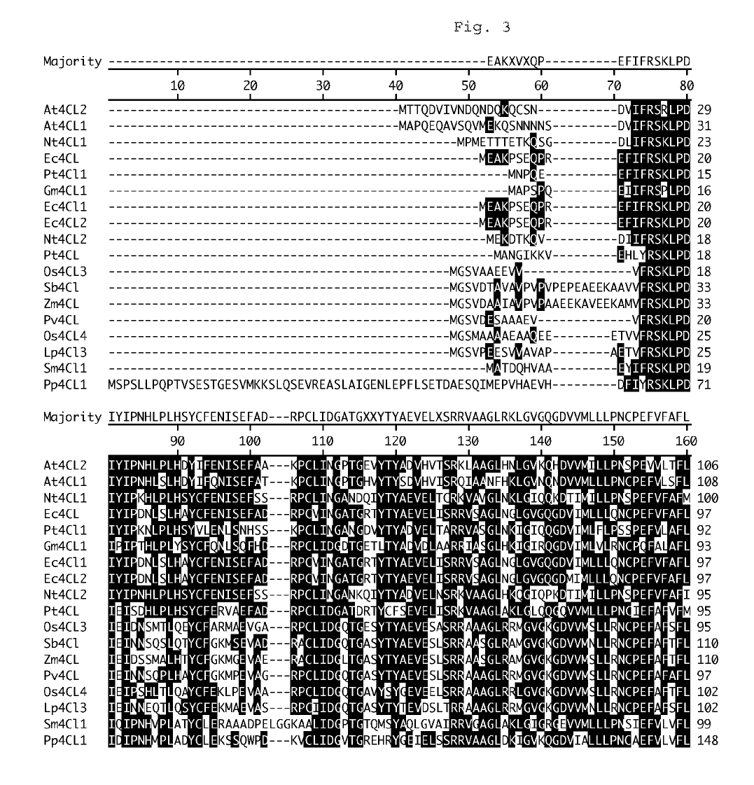

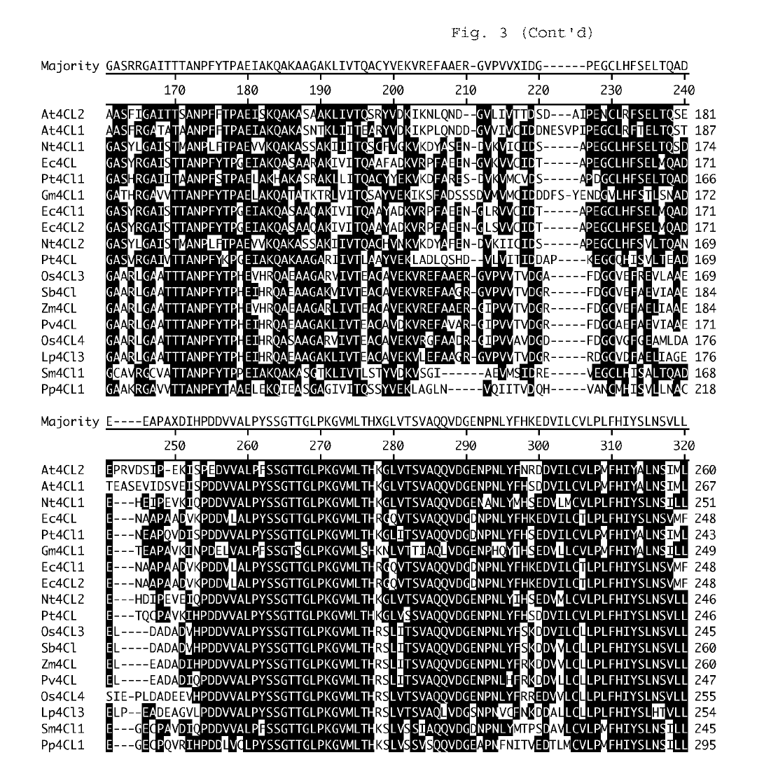

As used herein, the term “lignin biosynthesis enzyme” refers to a protein that regulates the synthesis of lignin monomers (p-coumaryl (4-hydroxycinnamyl) alcohol, coniferyl (3-methoxy 4-hydroxycinnamyl) alcohol, and sinapyl (3,5-dimethoxy 4-hydroxycinnamyl) alcohol) in plants. The term includes polymorphic variants, alleles, mutants, and interspecies homologs to the specific enzymes described herein. A nucleic acid that encodes a lignin biosynthesis enzyme refers to a gene, pre-mRNA, mRNA, and the like, including nucleic acids encoding polymorphic variants, alleles, mutants, and interspecies homologs of the particular sequences described herein. Thus, in some embodiments a lignin biosynthesis nucleic acid (1) has a nucleic acid sequence that has greater than about 50% nucleotide sequence identity, 55%, 60%, 65%, 70%, 75%, 80%, 85%, 90%, preferably 91%, 92%, 93%, 94%, 95%, 96%, 97%, 98% or 99% or higher nucleotide sequence identity, preferably over a region of at least about 10, 15, 20, 25, 50, 100, 200, 500 or more nucleotides or over the length of the entire polynucleotide, to a nucleic acid sequence of any of SEQ ID NOs:1, 3, 5, 7, 9, or 11; or (2) encodes a polypeptide having an amino acid sequence that has greater than about 50% amino acid sequence identity, 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200 or more amino acids or over the length of the entire polypeptide, to a polypeptide encoded by a nucleic acid sequence of any of SEQ ID NOs:1, 3, 5, 7, 9, or 11 or to an amino acid sequence of any of SEQ ID NOs:2, 4, 6, 8, 10, or 12 or to any one of the sequences shown in any of FIGS. 1-6. In some embodiments, a lignin biosynthesis enzyme, or a lignin biosynthesis polypeptide has an amino acid sequence having greater than about 50% amino acid sequence identity, 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200 or more amino acids or over the length of the entire polypeptide, to an amino acid sequence of any of SEQ ID NOs:2, 4, 6, 8, 10, or 12 or to any one of the amino acid sequences shown in any of FIGS. 1-6.

Lignin biosynthesis enzymes can be identified by name (e.g., cinnamate 4-hydroxylase); gene symbol (e.g., C4H); or accession number (e.g., NM_{—}128601 for nucleic acid or NP_{—}180607 for protein). It is understood that all of these identifiers reference the same biomarker and thus are equivalent. In some embodiments, the lignin biosynthesis enzyme is phenylalanine ammonia lyase (PAL) (accession number NM_{—}129260 or NP_{—}181241), cinnamate 4-hydroxylase (C4H) (accession number NM_{—}128601 or NP_{—}180607), 4-coumarate-CoA ligase (4CL) (accession number NM_{—}113019 or NP_{—}188761), hydroxycinnamoyl CoA:shikimate hydroxycinnamoyl transferase (HCT) (accession number NM_{—}124270 or NP_{—}199704), coumaryol shikimate 3-hydroxylase (C3H) (accession number NM_{—}119566 or NP_{—}850337), or cinnamoyl-CoA reductase 1 (CCR1) (accession number NM_{—}101463 or NP_{—}173047).

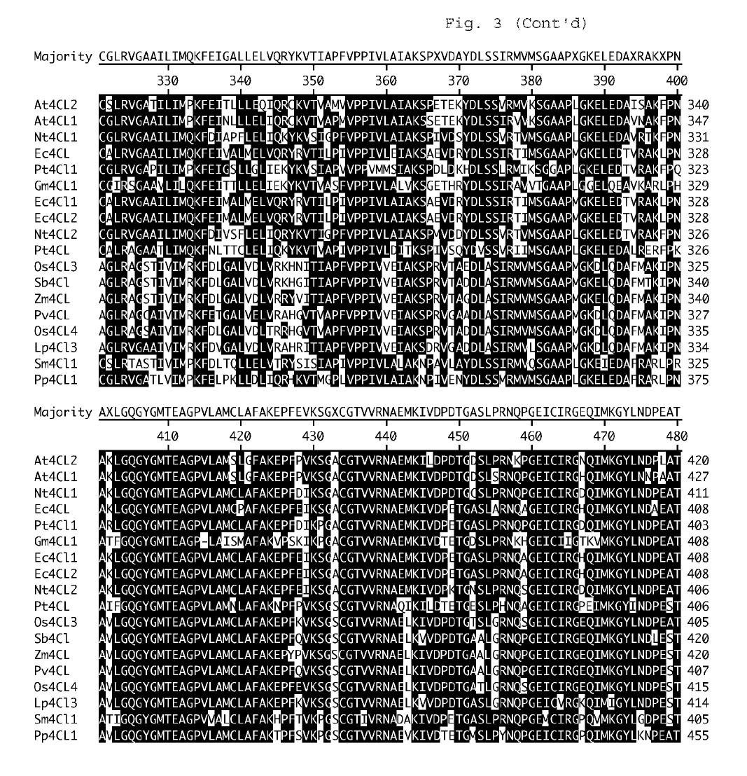

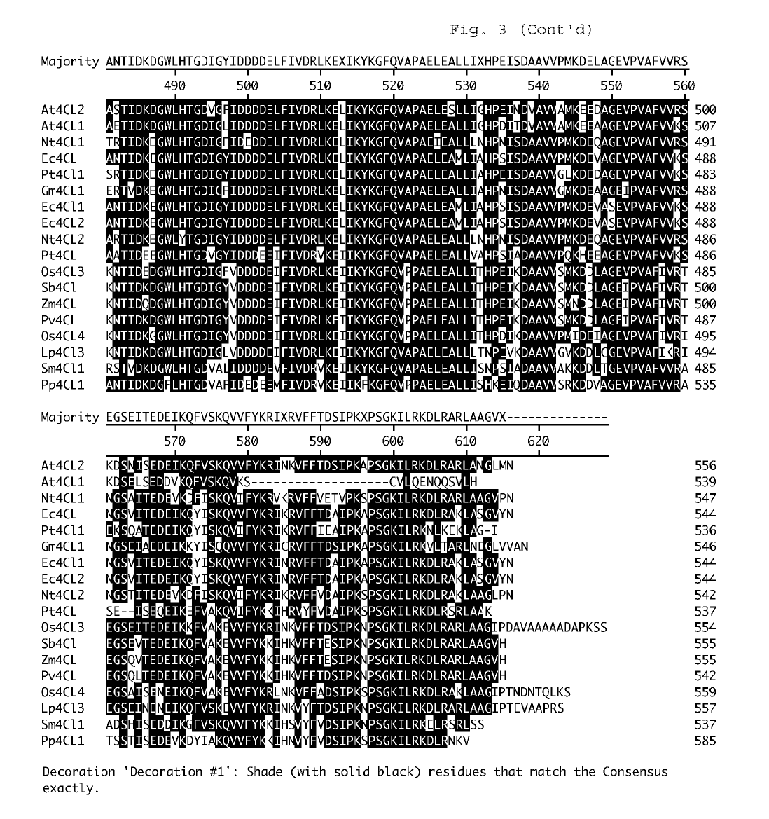

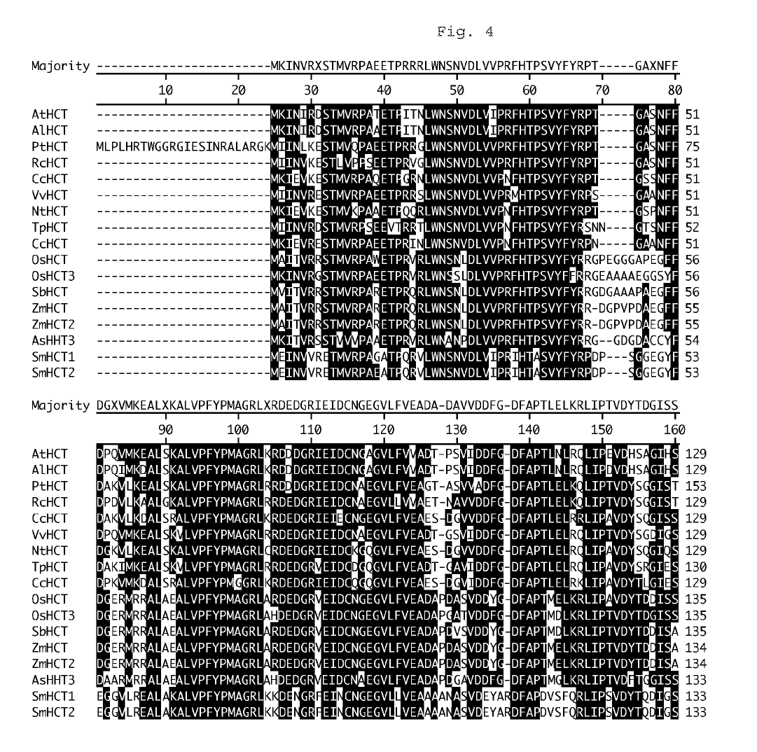

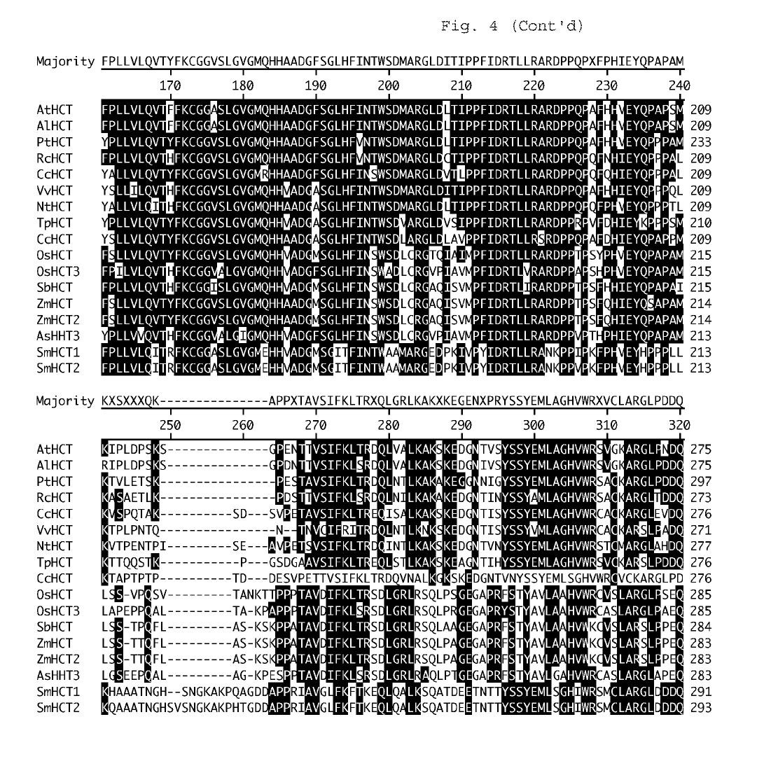

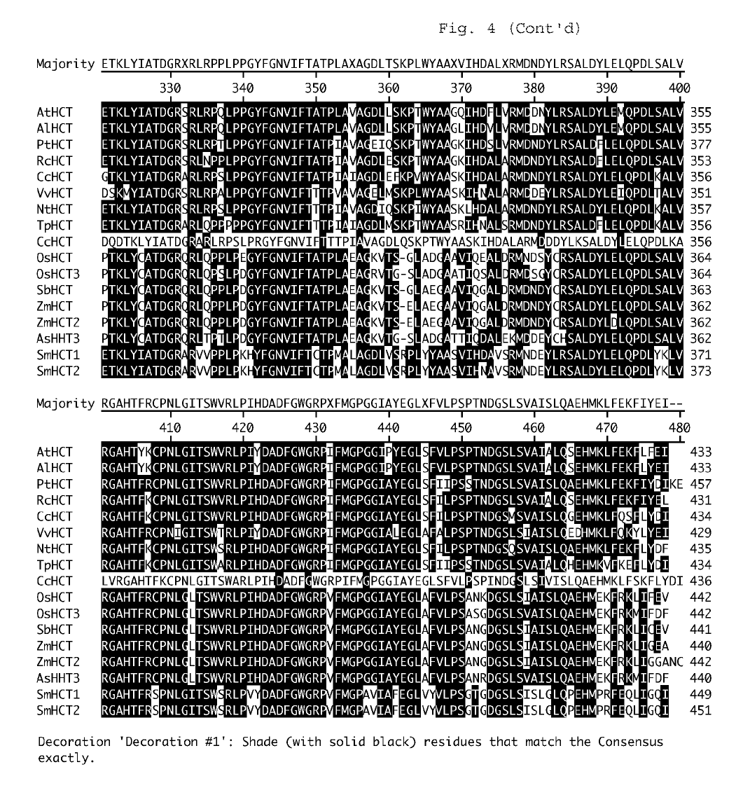

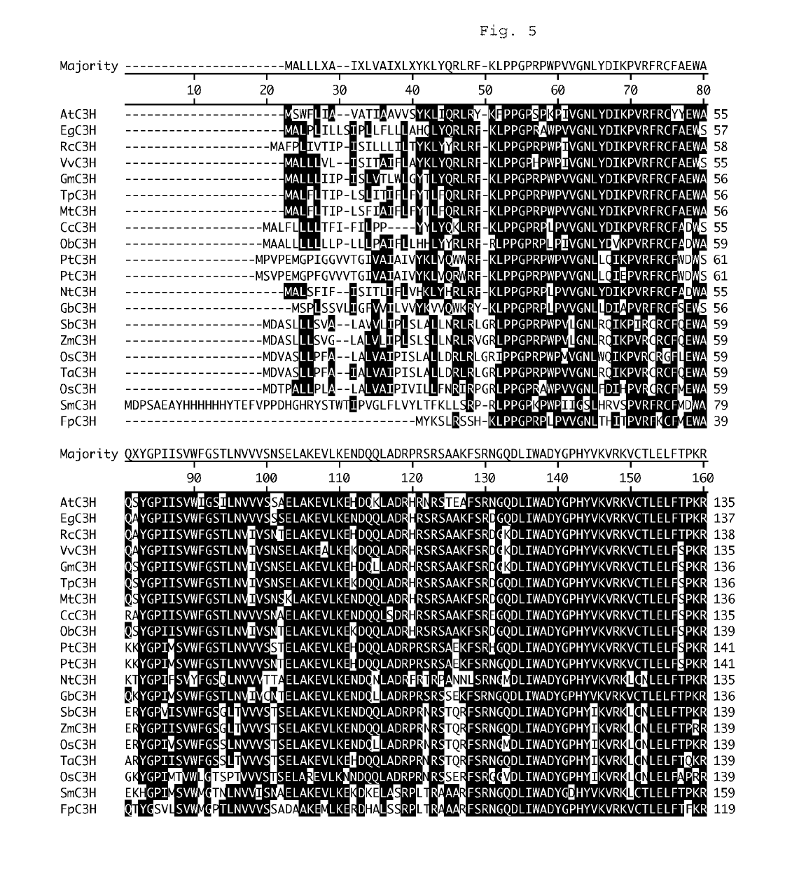

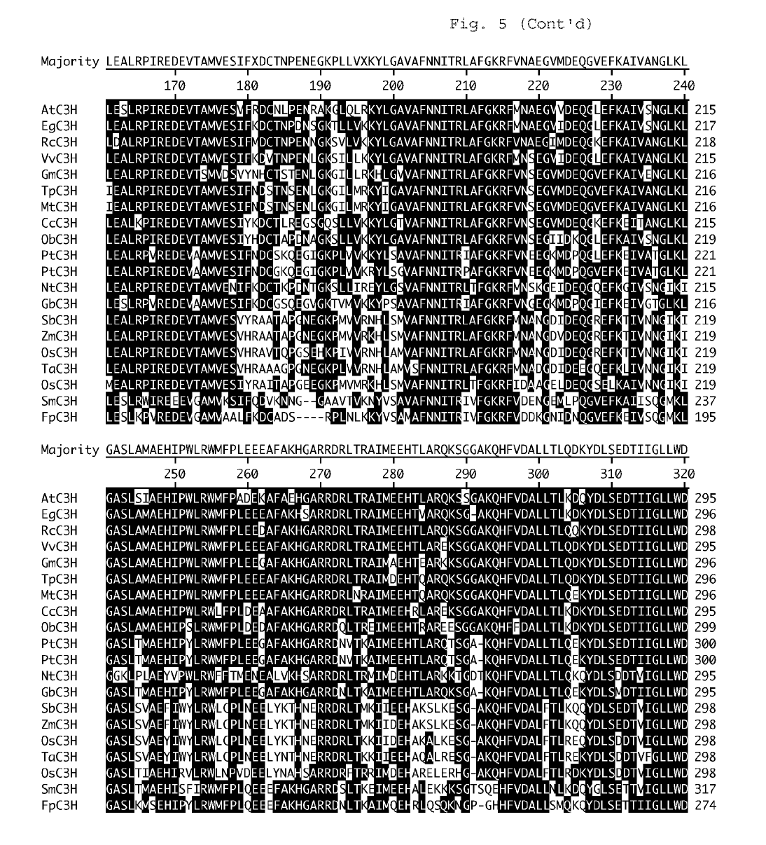

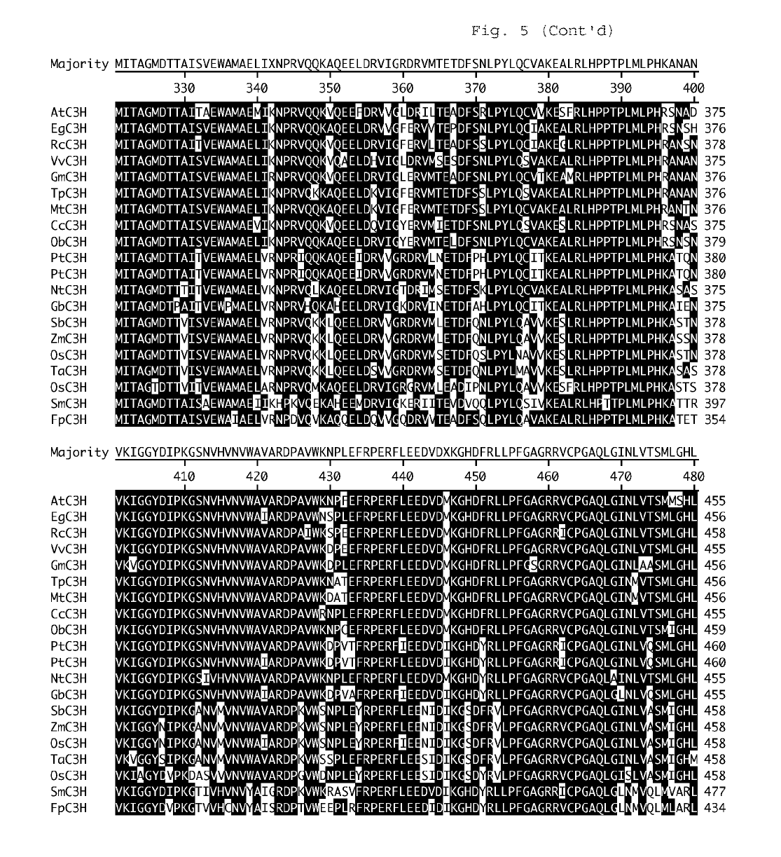

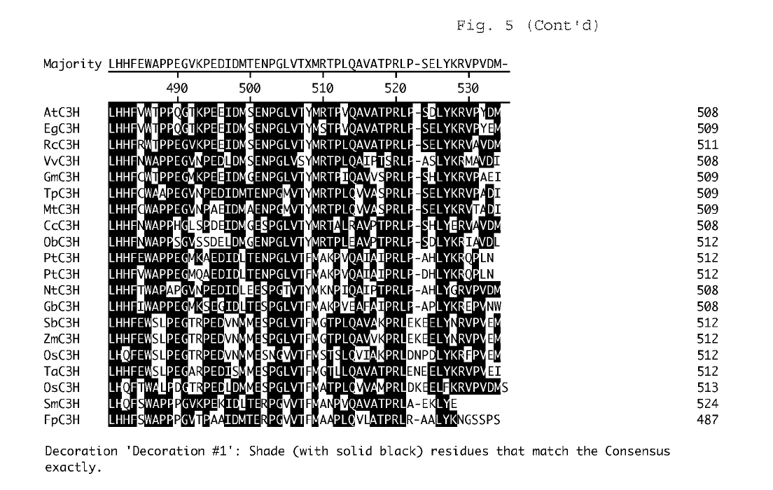

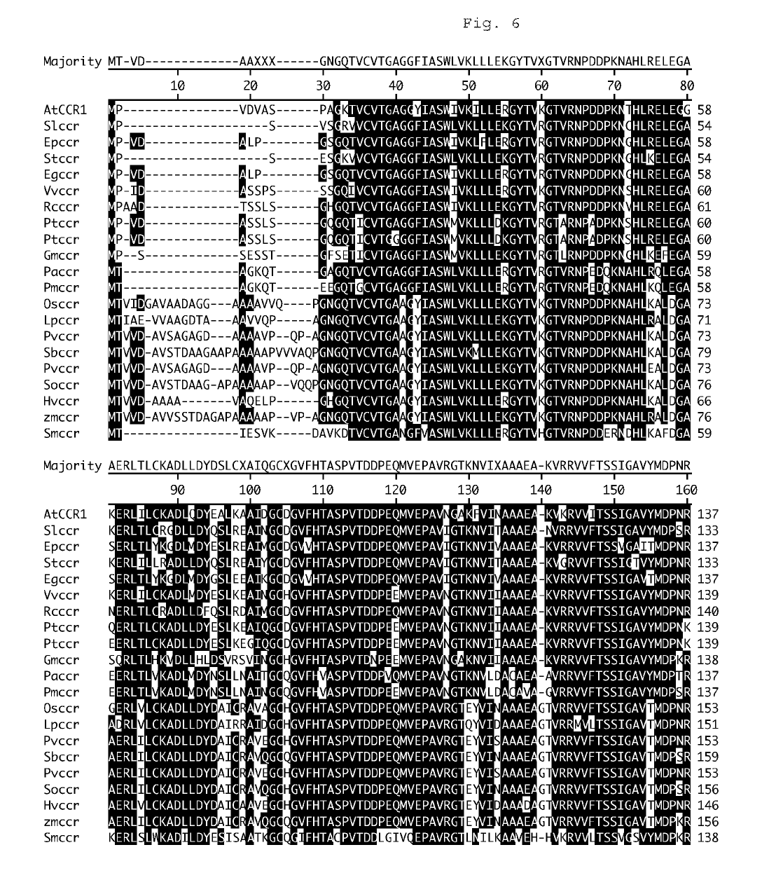

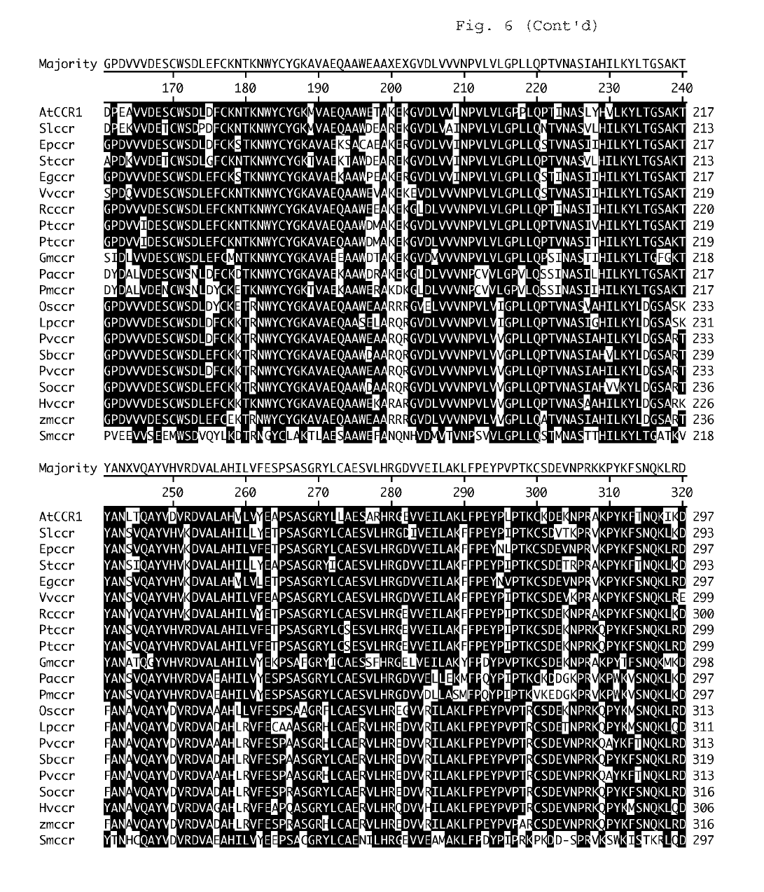

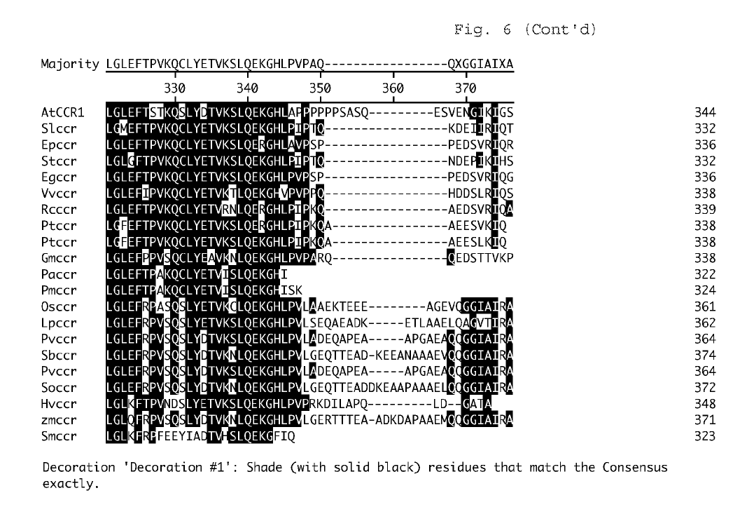

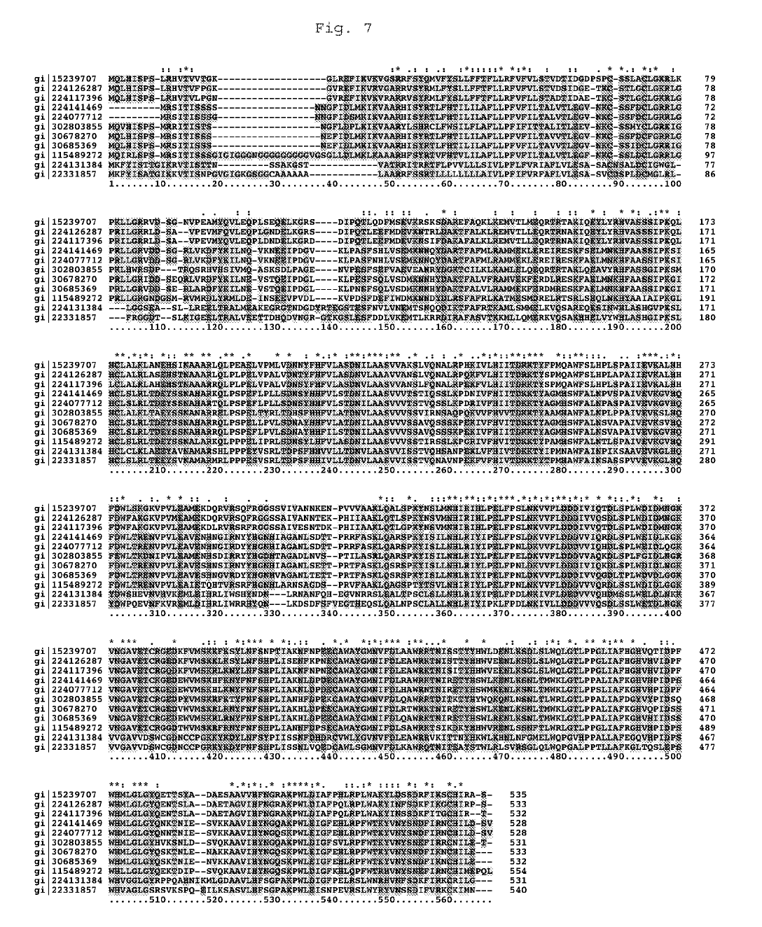

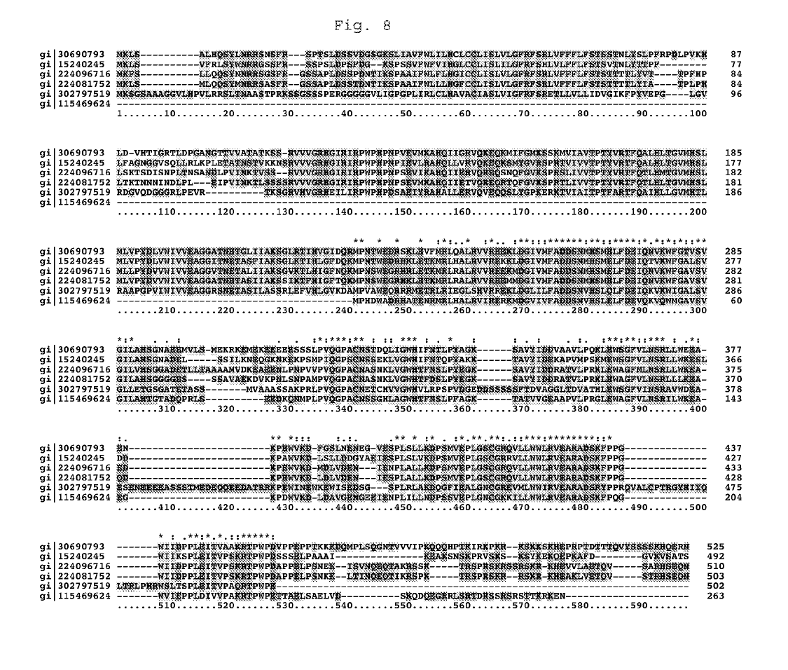

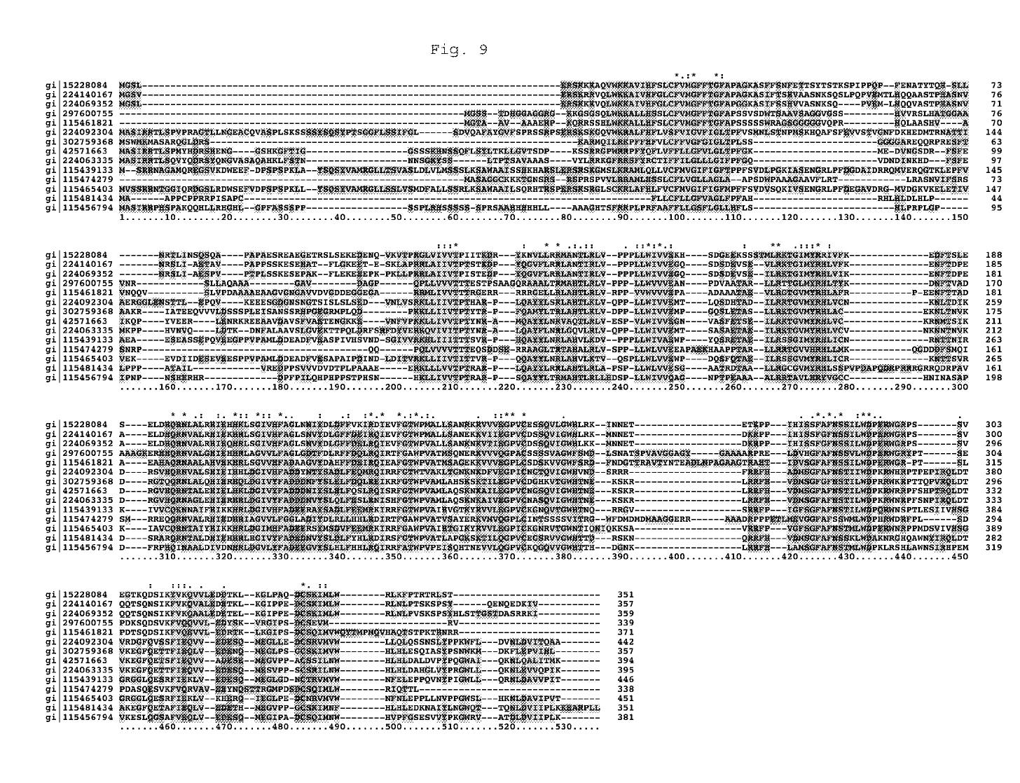

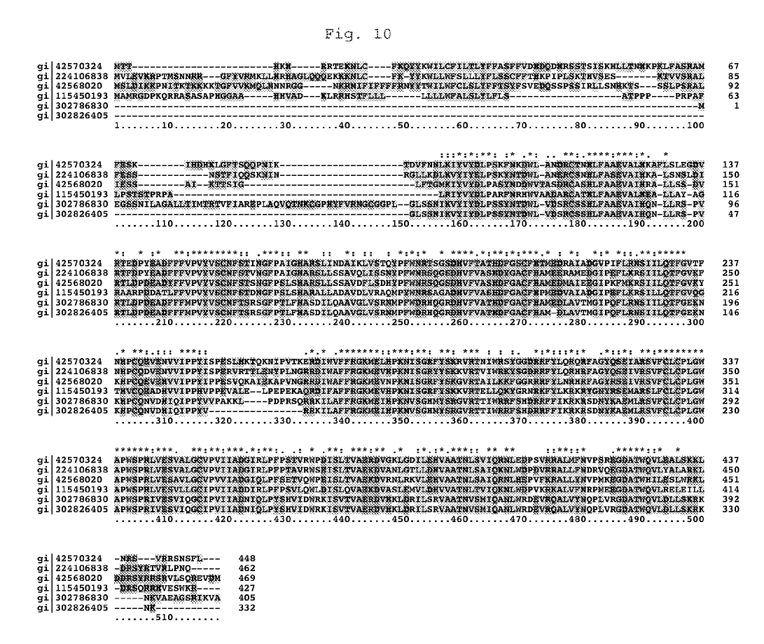

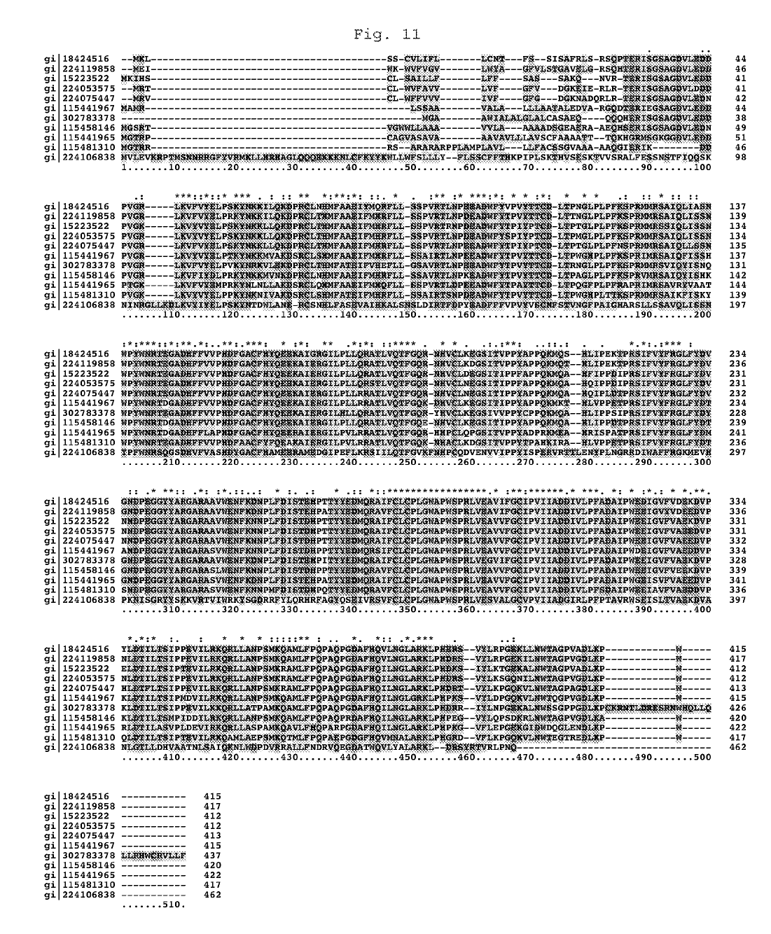

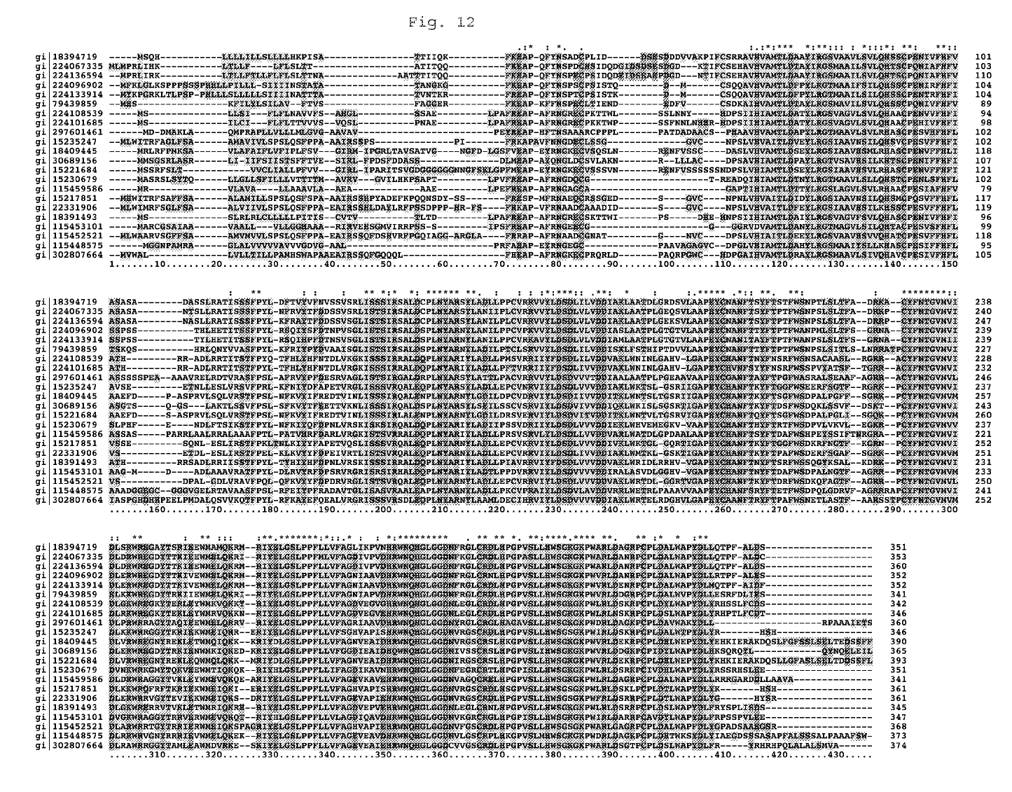

As used herein, the term “xylan biosynthesis enzyme” refers an enzyme that is involved in xylan synthesis. The term as used herein can also relate to an enzyme that modifies xylan, e.g., enzymes that acetylate xylan. The term encompasses polymorphic variants, alleles, mutants, and interspecies homologs to the specific polypeptides described herein. A nucleic acid that encodes a xylan biosynthesis enzyme refers to a gene, pre-mRNA, mRNA, and the like, including nucleic acids encoding polymorphic variants, alleles, mutants, and interspecies homologs of the particular amino acid sequences described herein. Thus, in some embodiments, a xylan biosynthesis enzyme encodes a polypeptide having an amino acid sequence that has greater than about 50% amino acid sequence identity, 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200 or more amino acids or over the length of the entire polypeptide, to any one of the amino acid sequences shown in any of FIGS. 7-12. Nucleic acid sequence of examples of xylan biosynthesis enzymes are available under the accession numbers provided in FIGS. 7-12. In some embodiments, a xylan biosynthesis enzyme has an amino acid sequence having greater than about 50% amino acid sequence identity, 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200 or more amino acids or over the length of the entire polypeptide, to any one of the amino acid sequences shown in any of FIGS. 7-12. In some embodiments, the xylan biosynthesis enzyme is irregular xylem 8 (IRX8), IRX14, IRX14-like, IRX9, IRX9-like, IRX7, IRX10, IRX10-like, F8H, PARVUS, or RWA1, RWA2, RWA3, or RWA4.

The term “substantially localized,” when used in the context of describing a plant having lignin deposition and/or xylan deposition that is substantially localized to a particular tissue, refers to lignin deposition and/or xylan deposition that is produced in substantially higher amounts in the particular cell type of interest as compared to other cell types that normally have a high content of lignin and/or xylan, such as interfascicular fibers or phloem fibers. In some embodiments, lignin deposition and/or xylan deposition is substantially localized to a particular cell type of interest when the amount of lignin deposition and/or xylan deposition in the particular cell type of interest is at least 2-fold, 3-fold, 4-fold, 5-fold, 6-fold, 7-fold, 8-fold, 9-fold, 10-fold higher or more as compared to the amount of lignin deposition and/or xylan deposition in other cell types that normally have a high content of lignin and/or xylan. In some embodiments, lignin deposition and/or xylan deposition is substantially localized to a particular cell type of interest when the amount of lignin deposition and/or xylan deposition in the particular cell type of interest is at least 2-fold, 3-fold, 4-fold, 5-fold, 6-fold, 7-fold, 8-fold, 9-fold, 10-fold higher or more as compared to the amount of lignin deposition and/or xylan deposition in interfascicular fibers or phloem fibers. In some embodiments, lignin deposition and/or xylan deposition is substantially localized to a particular cell type of interest when there is no detectable lignin deposition and/or xylan deposition in cell types other than the particular cell type of interest. In some embodiments, xylan O-acetylation is similarly substantially localized to specific cell types, while the content of xylan in general is not necessarily substantially localized in a way different from the natural (i.e., wild-type) situation. Lignin deposition and/or xylan deposition can be assessed using any method known in the art, including but not limited to spectrophotometry using acetyl-bromide reagent, histochemical staining (e.g., with phloroglucinol), and immunohistochemistry (e.g., with LM10 monoclonal antibody). Xylan O-acetylation can be assessed using immunohistochemistry (e.g., with LM23 monoclonal antibody), with biochemical assays for acetyl esters, or by determining the effect of hydrolytic enzymes.

As used herein, the term “transcription factor that regulates the production of components of a biosynthetic pathway” or “master transcription factor” refers to a transcription factor that regulates expression of one or of multiple genes in a biosynthetic pathway.

As used herein, the term “transcription factor that regulates the production of secondary cell wall” refers to a polypeptide, and variants, mutants, and homologs of the polypeptide, that regulates the expression of one or more genes involved in lignin biosynthesis and/or polysaccharide (cellulose and hemicellulose) biosynthesis by modulating transcription. In some embodiments, nucleic acids that encodes such a transcription factor: (1) have a nucleic acid sequence that has greater than about 50% nucleotide sequence identity, 55%, 60%, 65%, 70%, 75%, 80%, 85%, 90%, preferably 91%, 92%, 93%, 94%, 95%, 96%, 97%, 98% or 99% or higher nucleotide sequence identity, preferably over a region of at least about 10, 15, 20, 25, 50, 100, 200, 500 or more nucleotides or over the length of the entire polynucleotide, to a nucleic acid sequence of any of SEQ ID NOs:13, 15, 17, 19, 21, 23, 25, 27, 29, 31, or 33; (2) encode a polypeptide having an amino acid sequence that has greater than about 50% amino acid sequence identity, 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200 or more amino acids or over the length of the entire polypeptide, to a polypeptide encoded by a nucleic acid sequence of any of SEQ ID NOs:13, 15, 17, 19, 21, 23, 25, 27, 29, 31, or 33 or an amino acid sequence of any of SEQ ID NOs:14, 16, 18, 20, 22, 24, 26, 28, 30, 32, or 34 or to any one of the amino acid sequences shown in FIG. 13. In some embodiments, a transcription factor polypeptide that regulates the production of secondary cell wall: (1) has an amino acid sequence having greater than about 50% amino acid sequence identity, 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200 or more amino acids or over the length of the entire polypeptide, to an amino acid sequence of any of SEQ ID NOs:14, 16, 18, 20, 22, 24, 26, 28, 30, 32, or 34 or to any one of the amino acid sequences shown in FIG. 13.

In some embodiments, the transcription factor is NAC secondary wall-thickening promoting factor 1 (NST1) (ANAC043; accession number NM_{—}130243 or NP_{—}182200), NST2 (ANAC066; accession number NM_{—}116056 or NP_{—}191750), NST3 (SND1/ANAC012; accession number NM_{—}103011 or NP_{—}174554), secondary wall-associated NAC domain protein 2 (SND2) (ANAC073; accession number NM_{—}118992 or NP_{—}194579), SND3 (ANAC010; accession number NM_{—}102615 or NP_{—}564309), MYB domain protein 103 (MYB103) (accession number NM_{—}105065 or NP_{—}176575), MBY85 (accession number NM_{—}118394 or NP_{—}567664), MYB46 (accession number NM_{—}121290 or NP_{—}196791), MYB83 (accession number NM_{—}111685 or NP_{—}187463), MYB58 (accession number NM_{—}101514 or NP_{—}173098), or MYB63 (accession number NM_{—}106569 or NP_{—}178039).

The term “downstream target,” when used in the context of a downstream target of a transcription factor that regulates a component of a biosynthetic pathway of interest refers to a gene or protein whose expression is directly or indirectly regulated by the transcription factor. In some embodiments, the downstream target is a gene or protein that is directly or indirectly upregulated by the transcription factor. In some embodiments, the downstream target is a gene or protein that is directly or indirectly downregulated by the transcription factor.

In the context of secondary wall production, a downstream target can be, for example, IRX1, IRX3, IRX5, IRX8, IRX9, IRX14, IRX14-L, IRX7, or IRX10. See, for example, FIGS. 7-12 for examples of accession numbers and sequences for downstream targets. Downstream target genes are also described in the art; see, for example, Oikawa et al., 2010, PLoS ONE 5(11):e15481. As understood in the art, and further explained hereinbelow, some of the downstream targets (e.g., IRX9-Like and RWA2) may not be expressed in secondary wall tissue per se, but can be linked to a secondary wall-specific promoter or a vessel-specific promoter that is regulated by a transcription factor that regulates secondary wall production and can then serve to substantially localize xylan or xylan acetylation to the secondary wall.

As used herein, the term transcription factor that regulates the production “wax and/or cutin” components (e.g., wax ester, alkane, fatty alcohol and fatty esters) refers to a polypeptide, and variants, mutants, and homologs of the polypeptide, that regulates the expression of one or more genes involved in wax and/or cutin biosynthesis by modulating transcription. In some embodiments, nucleic acids that encodes such a transcription factor: encode a polypeptide having an amino acid sequence that has greater than about 50% amino acid sequence identity, 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200 or more amino acids or over the length of the entire polypeptide, to a polypeptide encoded by a nucleic acid sequence of any one of SEQ ID NOs:80-93, or an amino acid sequence of any of any one of SEQ ID NOs:80-93.

When used in the context of a transcription factor that regulates wax/cutin production, “downstream target” refers to a non-coding RNA, gene, or protein involved in wax/cutin production whose expression is directly or indirectly regulated by the transcription factor. In some embodiments, the downstream target is a non-coding RNA, gene, or protein that is directly or indirectly upregulated by the transcription factor. In some embodiments, the downstream target is a non-coding RNA, gene, or protein that is directly or indirectly downregulated by the transcription factor. Examples of such genes include the following (synonyms for the gene are listed in parenthesis): CER1, aldehyde decarbonylase; CER2 (VC2), BAHD-type acyl-transferase; CER3 (WAX2), sterol desaturase; CER4 (FAR3), fatty acyl-CoA reductase; CER5 (WBC12), ABC transporter; CER6 (CUT1), very long chain fatty acid condensing enzyme; CER10 (ECR), enoyl-CoA reductase; WSD1, wax ester synthase; MAH1, mid-chain alkane hydrolase; WBC11 (ABCG11, DSO, COF1), ABC transporter; KCS1, very long chain fatty acid condensing enzyme; KCS2 (DAISY), very long chain fatty acid condensing enzyme; FATB, acyl carrier; LACS1, long chain acyl-CoA synthase; LACS2, long chain acyl-CoA synthase; CYP86A4, cytochrome P450-dependent fatty acid hydroxylase; CYP86A7, cytochrome P450-dependent fatty acid hydroxylase; LCR (CYP86A5), cytochrome P450-dependent fatty acid hydroxylase; KCS10 (FDH), very long chain fatty acid condensing enzyme; and CER60 (KCS5), very long chain fatty acid condensing enzyme. Examples of accession numbers are provided in the Listing of Illustrative Wax/Cutin genes.

The terms “reduced level of activity,” “reduced activity” and “decreased activity” refer interchangeably to a reduction in the amount of activity of a protein, e.g., a cell wall biosynthesis enzyme of interest or a xylan biosynthesis enzyme gene or protein of interest in an engineered plant as compared to the amount of activity in a wild-type (i.e., naturally occurring) plant. In some embodiments, reduced activity results from reduces expression levels. A reduced level of activity or a reduces level of expression can be a reduction in the amount of activity or expression of a protein, e.g., a cell wall biosynthesis enzyme gene or protein or a xylan biosynthesis enzyme gene or protein, of at least 10%, 20%, 30%, 40%, 50%, 60%, 70%, 80%, or 90% or greater. In some embodiments, the reduced level of activity or reduced level of expression is a reduction in the amount of activity or expression of the enzyme, e.g., a cell wall biosynthesis enzyme gene or protein of interest or a xylan biosynthesis enzyme gene or protein of interest, throughout all the tissues of the engineered plant. In some embodiments, the reduction in the amount of activity or expression of the protein or gene, e.g., a cell wall biosynthesis enzyme gene or protein of interest or a xylan biosynthesis enzyme gene or protein of interest, is localized to one or more tissues of the engineered plant. In some embodiments, the biosynthetic enzyme is not reduced in amount but is modified in amino acid sequence so that the enzymatic activity is reduced directly or indirectly (e.g., expression of inhibitory protein). Reduction in the amount of expression of a gene or protein can be assessed by measuring decreases in the level of RNA encoded by the gene of interest and/or decreases in the level of protein expression or activity for the protein of interest.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “substantially identical,” used in the context of two nucleic acids or polypeptides, refers to a sequence that has at least 50% sequence identity with a reference sequence. Percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein; preferably BLAST using standard parameters, as described below. For example, a polynucleotide encoding a lignin biosynthesis enzyme may have a sequence that is at least 50%, 55%, 60%, 65%, 70%, 75%, 80%, 85%, 90%, 91%, 92%, 93%, 94%, 95%, 96%, 97%, 98%, or 99% identical to a sequence of SEQ ID NO:1, SEQ ID NO:3, SEQ ID NO:5, SEQ ID NO:7, SEQ ID NO:9, or SEQ ID NO:11.

Two nucleic acid sequences or polypeptide sequences are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

Nucleic acid or protein sequences that are substantially identical to a reference sequence include “conservatively modified variants.” With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

(see, e.g., Creighton, Proteins (1984)).

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C. For example, stringent conditions for hybridization, such as RNA-DNA hybridizations in a blotting technique are those which include at least one wash in 0.2×SSC at 55° C. for 20 minutes, or equivalent conditions.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular plants species, but also encompasses a promoter from a corresponding gene in other plant species.

A “constitutive promoter” in the context of this invention refers to a promoter that is capable of initiating transcription in nearly all cell types, whereas a “cell type-specific promoter” or “tissue-specific promoter” initiates transcription only in one or a few particular cell types or groups of cells forming a tissue. In some embodiments, a promoter is tissue-specific if the transcription levels initiated by the promoter in a particular cell-type or tissue are at least 2-fold, 3-fold, 4-fold, 5-fold, 6-fold, 7-fold, 8-fold, 9-fold, 10-fold, 50-fold, 100-fold, 500-fold, 1000-fold higher or more as compared to the transcription levels initiated by the promoter in non-vessel tissues. In some embodiments, the promoter is vessel-specific. As used herein, a “vessel-specific” promoter refers to a promoter that initiates substantially higher levels of transcription in vessels as compared to other non-vessel cells of the plant. As used herein, the term “vessel” refers to xylem vessels, a conductive component of the vascular tissues in plants that function in the transport of water, nutrients, and signaling molecules throughout the plant. In some embodiments, a promoter is vessel-specific if the transcription levels initiated by the promoter in vessel tissues are at least 2-fold, 3-fold, 4-fold, 5-fold, 6-fold, 7-fold, 8-fold, 9-fold, 10-fold, 50-fold, 100-fold, 500-fold, 1000-fold higher or more as compared to the transcription levels initiated by the promoter in non-vessel tissues. Non-limiting examples of vessel-specific promoters include the native promoter of any of the genes encoding Vascular-Related NAC-Domain Protein 1 (VND1), VND2, VND3, VND4, VND5, VND6, VND7. See, e.g., Kubo et al., Genes Dev. 19:1855-1860 (2005), which is incorporated by reference herein. Another example of a vessel-specific promoter includes the native promoter of REF4 and RFR1 (see, e.g., Bonawitz et al., “The REF4 and RFR1 subunits of the eukaryotic transcriptional coregulatory complex Mediator are required for phenylpropanoid homeostasis in Arabidopsis.” doi:10.1074/jbc.M111.312298 (2012)).

In the context of an artificial positive feedback loop, an “induced” promoter from a downstream gene in a biosynthetic pathway of interest refers to a promoter where expression of the gene is enhanced, i.e., expression may be directly or indirectly activated (turned on and/or increased) by the transcription factor employed in the artificial positive feedback loop. Thus, in when referring to a promoter employed in an artificial feedback loop construct, it is understood that the promoter is “induced” by the transcription factor regardless of whether it is explicitly stated that the promoter is an induced promoter.

A polynucleotide is “heterologous” to an organism or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “expression cassette” or “DNA construct” or “expression construct” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, RNAi, or sense suppression) one of skill will recognize that the inserted polynucleotide sequence need not be identical, but may be only substantially identical to a sequence of the gene from which it was derived. As explained herein, these substantially identical variants are specifically covered by reference to a specific nucleic acid sequence. One example of an expression cassette is a polynucleotide construct that comprises a transcription factor operably linked to a heterologous promoter that is a promoter from a gene that is regulated by the transcription factor.

The term “plant” as used herein can refer to a whole plant or part of a plant, e.g., seeds, and includes plants of a variety of ploidy levels, including aneuploid, polyploid, diploid and haploid. The term “plant part,” as used herein, refers to shoot vegetative organs and/or structures (e.g., leaves, stems and tubers), branches, roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, and plant tissue (e.g., vascular tissue, ground tissue, and the like), as well as individual plant cells, groups of plant cells (e.g., cultured plant cells), protoplasts, plant extracts, and seeds. The class of plants that can be used in the methods of the invention is generally as broad as the class of higher and lower plants amenable to transformation techniques, including angiosperms (monocotyledonous and dicotyledonous plants), gymnosperms, ferns, bryophytes, and multicellular algae.

The term “biomass,” as used herein, refers to plant material that is processed to provide a product, e.g., a biofuel such as ethanol, or livestock feed, or a cellulose for paper and pulp industry products. Such plant material can include whole plants, or parts of plants, e.g., stems, leaves, branches, shoots, roots, tubers, and the like.

The term “increased secondary cell wall deposition” refers to an increased amount of secondary cell wall that is produced in an engineered plant of the present invention as compared to a wild-type (i.e., naturally occurring) plant, e.g., an increased density or thickness and/or an increased ratio between the cell diameter and cell wall thicknesses. “Secondary cell wall” is mainly composed of cellulose, hemicellulose, and lignin and is deposited in some, but not all, tissues of a plant, such woody tissue. Secondary cell wall deposition is said to be increased in an engineered plant as compared to a wild-type plant when the amount of one or more components of secondary cell wall (e.g., cellulose, hemicellulose, or lignin) in the engineered plant, or the ratio between the cell diameter and cell wall thickness, is increased by at least 10%, at least 20, 30%, 40%, 50%, 60%, 70%, 80%, 90% or more relative to the amount of the one or more components of secondary cell wall in a wild-type plant. The amount of a component of secondary cell wall that is present can be assessed using any method known in the art, including but not limited to microscopy (e.g., electron-microscopy, RAMAN-microscopy), histochemical staining (e.g., phloroglucinol) and enzymatic or chemical reaction (e.g., polysaccharide hydrolysis or TFA hydrolysis).

The term “saccharification reaction” refers to a process of converting biomass, usually cellulosic or lignocellulosic biomass, into monomeric sugars, such as glucose and xylose.

The term “soluble sugar” refers to monomeric, dimeric, or trimeric sugar that is produced from the saccharification of biomass.

The term “increased amount,” when referring to an amount of sugar or soluble sugar obtained from an engineered plant of the present invention, refers to an increase in the amount or yield of sugar that is obtained from saccharification of biomass per amount of starting material, in comparison to corresponding biomass from a wild-type (i.e., naturally occurring) plant. In the context of the present invention, “corresponding biomass from a wild-type plant” refers to plant material that is from the same part of the plant as the biomass from a plant having a reduced level of expression of a lignin biosynthesis enzyme and/or xylan biosynthesis enzyme. As understood in the art, increased amount or increased yield is based upon comparisons of the same amount of corresponding plant material.

The term “conversion reaction,” as used herein, refers to a reaction that converts biomass into a form of bioenergy. Examples of conversion reactions include, but are not limited to, combustion (burning), gasification, pyrolysis, and polysaccharide hydrolysis (enzymatic or chemical).

The term “increased production,” when referring to an amount of bioenergy production obtained from an engineered plant of the present invention, refers to an increased amount of bioenergy that is produced from subjecting biomass from an engineered plant to a conversion reaction (e.g., combustion, gasification, pyrolysis, or polysaccharide hydrolysis) as compared to the amount of bioenergy that is produced from corresponding biomass from a wild-type (i.e., naturally occurring) plant.

### II. Introduction

In one aspect, the present invention relates to the discovery that an artificial positive feedback loop (APFL) can be created in plants to regulate gene expression in desired biosynthetic pathways, for example, to modulate gene expression in one or more desired tissues. Accordingly, the invention provides an APFL in plants wherein the APFL comprises a gene encoding a transcription factor that controls expression of a biosynthetic pathway of interest operably linked to a promoter from an induced downstream gene in the biosynthetic pathway where the expression of the downstream gene is controlled by the transcription factor. Examples of biosynthetic pathways that can be regulated by such a system include secondary cell wall deposition, wax/cutin biosynthesis, lipid biosynthesis, alkaloid biosynthesis and terpenoid biosynthesis. Thus, one example of an APFL in accordance with the invention relates to increasing cell wall deposition in specific tissues whereby a nucleic acid encoding a transcription factor as described herein that controls the biosynthesis of secondary cell wall is operably linked to a promoter from a downstream induced gene involved in secondary wall biosynthesis where expression of the downstream gene is induced by the transcription factor. A second example of an APFL of the invention comprises a nucleic acid encoding a transcription factor as described herein that controls expression of wax and/or cutin biosynthesis operably linked to a promoter from a downstream induced gene involved in wax and/or cutin biosynthesis where expression of the downstream gene is induced by the transcription factor. A further example of an APFL of the invention comprises a nucleic acid encoding a transcription factor as described herein that regulates lipid biosynthesis and, e.g., accumulation in seed and other tissues, operably linked to a promoter from a downstream induced gene involved in lipid biosynthesis where expression of the downstream gene is induced by the transcription factor.

In various embodiments, the invention provides nucleic acids, expression constructions, and plants comprising AFPLs of the invention and methods of using such compositions.

In one aspect, the present invention is based, in part, on the discovery that focusing lignin deposition in the vessels of plants while reducing lignin and/or xylan content elsewhere in the plant overcomes problems typically associated with plants having reduced lignin or xylan content, specifically vessel collapse and stunting of plant development. Although cell wall components such as lignin and xylan are beneficial to plants for purposes such as providing structural support to the vessels which supply water and nutrients throughout the plant, these cell wall components (e.g., lignin and xylan) also account for much of the recalcitrance of cell walls to enzymatic degradation and polysaccharide extractability. Therefore, specific localization of lignin and xylan in vessels represents a method by which the cell walls of plants can be made more susceptible to enzymatic degradation and polysaccharide extractability, thus improving saccharification and, e.g., biofuel production from plants; and also providing for improved substrates for the paper and pulp industry.

Accordingly, in one aspect the present invention provides methods of engineering a plant having lignin and/or xylan deposition and/or xylan O-acetylation that is substantially localized to the vessels of xylem tissue of the plant. Vessel-specific lignin and/or xylan deposition and/or xylan O-acetylation is accomplished by reducing a lignin and/or xylan biosynthesis enzyme and/or xylan O-acetylation enzyme and expressing a substantially identical enzyme (e.g., an ortholog or a paralog of the enzyme reduced in the plant, or an enzyme that has the same biochemical function) under the control of a vessel-specific promoter that is not the native promoter of the lignin and/or xylan biosynthesis enzyme and/or xylan O-acetylation enzyme. Plants of the present invention or biomass comprising the plants of the present invention are suitable for use in a saccharification reaction to obtain an increased amount of soluble sugars than can be obtained from wild-type plants, or in the paper industry.

The present invention is also based, in part, on the discovery that increasing cell wall deposition specifically in woody tissues results in plants having cells that are filled with cell wall polymers. Increased cell wall deposition is beneficial because it increases the biomass density of the plant, which in turn can increase the amount of bioenergy production that can be obtained from the plant. Accordingly, in another aspect the present invention provides methods of engineering a plant having increased cell wall deposition using an AFPL. A transcription factor that regulates secondary cell wall production is expressed in a plant under the control of a promoter from an induced gene that is a downstream target of the transcription factor. The expression of the transcription factor increases the expression driven from the downstream promoter, which in turn, because it is operably linked to a gene encoding the transcription factor, increases the expression of the transcription factor, thus generating a positive feedback loop that enhances the expression of the downstream genes of the secondary cell wall pathway and consequently increases secondary cell wall deposition. The transcription factor and promoter may both be from a different plant species that the host plant, or either the transcription factor or promoter may be from a different plant species. Similarly, the transcription factor and promoter need not be from the same plant species. Plants of the present invention or biomass comprising the plants of the present invention are suitable for use in a biomass conversion reaction to increase bioenergy production as compared to the bioenergy production of wild-type plants.

The methods of the present invention can further be used in combination with one another. Thus, in some embodiments, the present invention provides methods of making plants having increased lignin deposition that is substantially localized to the vessels of xylem tissue of the plant and having increased secondary cell wall deposition. In some embodiments, the present invention provides methods of making plants having increased xylan deposition that is substantially localized to the vessels of xylem tissue of the plant and having increased secondary cell wall deposition. In some embodiments, the present invention provides methods of making plants having increased xylan O-acetylation deposition that is substantially localized to the vessels of xylem tissue of the plant and having increased secondary cell wall deposition. In some embodiments, the present invention provides methods of making plants having increased lignin deposition that is substantially localized to the vessels of xylem tissue of the plant and having increased xylan deposition that is substantially localized to the vessels of xylem tissue of the plant. In some embodiments, the present invention provides methods of making plants having lignin deposition that is substantially localized to the vessels of xylem tissue of the plant and having increased xylan O-acetylation deposition that is substantially localized to the vessels of xylem tissue of the plant.

In another aspect, the invention provides a method of increasing wax/cutin production in a desired tissue. A transcription factor that regulates wax/cuticle production is expressed in a plant under the control of a promoter from an induced gene that is a downstream target of the transcription factor. The expression of the transcription factor increases the expression driven by the downstream promoter, which in turn, because it is operably linked to a gene encoding the transcription factor, increases the expression of the transcription factor, thus generating a positive feedback loop that increases wax/cutin production. The transcription factor and promoter, or the transcription factor or promoter, can be from a different species than the host plant cell in which the artificial positive feedback loop is created. In some embodiments, the transcription factor and promoter are from different species. Plants generated in accordance with this aspect of the invention have increased drought tolerance and reduced water consumption.

### III. Plants Having Spatially Modified Gene Expression

A. Modification of Expression of a Lignin or Xylan Biosynthesis Enzyme

In one aspect, the present invention provides methods of engineering a plant having lignin deposition that is substantially localized to the vessels of xylem tissue of the plant. In some embodiments, the method comprises:
introducing an expression cassette into the plant, wherein the plant is modified to have a reduced level of expression of a lignin biosynthesis enzyme; and wherein the expression cassette comprises a polynucleotide encoding the lignin biosynthesis enzyme operably linked to a heterologous vessel-specific promoter; and
culturing the plant under conditions in which the lignin biosynthesis enzyme is expressed.

In another aspect, the present invention provides methods of engineering a plant having xylan deposition that is substantially localized to the vessels of xylem tissue of the plant. In some embodiments, the method comprises:
introducing an expression cassette into the plant, wherein the plant is modified to have a reduced level of expression of a xylan biosynthesis enzyme; and wherein the expression cassette comprises a polynucleotide encoding the xylan biosynthesis enzyme operably linked to a heterologous vessel-specific promoter; and
culturing the plant under conditions in which the xylan biosynthesis enzyme is expressed.

The expression cassette as described herein, when introduced into a plant that is modified to have a reduced level of expression of the lignin or xylan biosynthesis enzyme, results in a plant having fine-tuned lignin or xylan deposition in which lignin is still expressed in vessel tissues, thus preventing vessel collapse, but in which lignin or xylan is not highly expressed in other tissues, thus reducing cell wall recalcitrance.

One of skill in the art will understand that the lignin biosynthesis enzyme and/or xylan biosynthesis enzyme that is introduced into the plant by an expression cassette does not have to be identical to the lignin biosynthesis enzyme and/or xylan biosynthesis enzyme that was modified in the plant before introduction of the expression cassette. In some embodiments, the lignin biosynthesis enzyme and/or xylan biosynthesis enzyme that is introduced into the plant by an expression cassette is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to the lignin biosynthesis enzyme and/or xylan biosynthesis enzyme that was modified in the plant before introduction of the expression cassette. In some embodiments, the lignin biosynthesis enzyme and/or xylan biosynthesis enzyme that is introduced into the plant by an expression cassette is a homolog (e.g., a homolog as shown in any of the alignments of FIGS. 1-12 or an enzyme with the same biochemical function, e.g., paralog) of the lignin biosynthesis enzyme and/or xylan biosynthesis enzyme that was modified in the plant before introduction of the expression cassette.

1. Lignin Biosynthesis Enzymes

In some embodiments, the expression cassette comprises a polynucleotide encoding a lignin biosynthesis enzyme. A lignin biosynthesis enzyme may be selected for use in the present invention on the basis that regulates the production of monolignols and therefore lignin biosynthesis. In some embodiments, the lignin biosynthesis enzyme is phenylalanine ammonia lyase (PAL), cinnamate 4-hydroxylase (C4H), 4-coumarate-CoA ligase (4CL), hydroxycinnamoyl CoA:shikimate hydroxycinnamoyl transferase (HCT), coumaryol shikimate 3-hydroxylase (C3H), or cinnamoyl-CoA reductase 1 (CCR1).

The lignin biosynthesis enzymes PAL, C4H, 4CL, HCT, C3H, and CCR1 have been characterized in Arabidopsis and have been shown to mediate the synthesis of lignin monomers (monolignols) from phenylalanine. See, e.g., Bonawitz and Chapple, Annu. Rev. Genet. 44:337-63 (2010). Thus, in some embodiments, the polynucleotide encoding a lignin biosynthesis enzyme is substantially identical to any of the polynucleotide sequences of SEQ ID NOs:1, 3, 5, 7, 9, or 11. In some embodiments, the lignin biosynthesis enzyme is substantially identical to any of the polypeptide sequences of SEQ ID NOs:2, 4, 6, 8, 10, or 12. Additionally, many of the enzymes involved in lignin biosynthesis are conserved among species. Thus, in some embodiments, the polynucleotide encoding a lignin biosynthesis enzyme comprises a homolog of any of the polynucleotide sequences of SEQ ID NOs:1, 3, 5, 7, 9, or 11. In some embodiments, the lignin biosynthesis enzyme comprises a homolog of any of the polypeptide sequences of SEQ ID NOs:2, 4, 6, 8, 10, or 12 or any of the polypeptide sequences shown in any of FIGS. 1-6.

In some embodiments, the polynucleotide encoding a lignin biosynthesis enzyme comprises a polynucleotide sequence that is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to any of SEQ ID NOs:1, 3, 5, 7, 9, or 11. In some embodiments, the polynucleotide encoding a lignin biosynthesis enzyme comprises a polynucleotide sequence that encodes a polypeptide sequence that is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to any of SEQ ID NOs:2, 4, 6, 8, 10, or 12 or any of the polypeptide sequences shown in any of FIGS. 1-6. In some embodiments, the lignin biosynthesis enzyme comprises an amino acid sequence that is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to any of SEQ ID NOs:2, 4, 6, 8, 10, or 12 or any of the polypeptide sequences shown in any of FIGS. 1-6.

Gene and protein sequences and/or accession numbers for PAL, C4H, 4CL, HCT, C3H, and CCR1 are described in the Sequence Listing herein. Amino acid sequence alignments for lignin biosynthesis enzymes showing the amino acid sequences for each of these proteins from multiple plant species are shown in FIGS. 1-6. Additionally, gene and protein sequences for these proteins, and methods for obtaining the genes or proteins, are known and described in the art. See, for example, Schilmiller et al., 2009, Plant J., doi: 10.1111/j.1365−313X.2009.03996.x. One of skill in the art will recognize that these gene or protein sequences known in the art and/or as described herein can be modified to make substantially identical lignin biosynthesis enzymes, e.g., by making conservative substitutions at one or more amino acid residues. One of skill will also recognize that the known sequences (e.g., the alignments provided herein) provide guidance as to what amino acids may be varied to make a substantially identical lignin biosynthesis enzyme. For example, using any of the alignments shown in FIGS. 1-6, one of skill will recognize which amino acid residues are not highly conserved and thus can likely be changed without resulting in a significant effect on the function of the lignin biosynthesis enzyme.

2. Xylan Biosynthesis Enzymes

The methods of the invention can also employ xylan biosynthesis enzymes. Several enzymes involved in xylan biosynthesis are known. Glycosyltransferases (GTs) belonging to the family GT43 (known as IRX9, IRX9-like, IRX14 and IRK14-like) have been demonstrated to be involved in xylan biosynthesis. The nomenclature for GT families used here is according to the CAZy database (www.cazy.org) (Cantarel et al., 2009). Other GTs in the GT47 family have also been shown to be involved in xylan biosynthesis: IRX10, IRX10-like, IRX7 and F8H. In addition GTs in GT8 have been shown to be involved in xylan biosynthesis: IRX8 (GAUT12) and PARVUS (GATL1). All the mentioned enzymes are known to be involved in xylan biosynthesis because plants where the genes have been mutated are deficient in xylan. (Brown, 2009; Wu et al., 2010) (Lee et al., 2009) (Pena et al., 2007; Persson et al., 2007; Liepman et al., 2010; Scheller and Ulvskov, 2010). Proteins belonging to the DUF579 family (also known as IRX15) are also involved in xylan biosynthesis although they do not appear to be GTs (Brown et al., 2011). The GTs responsible for adding glucuronic acid residues to the xylan backbone have been identified and are known as PGSIP or GUX, however, inactivation of these genes does not lead to xylan deficiency (Mortimer et al., 2010; Oikawa et al., 2010). GTs involved in adding arabinose residues to the xylan backbone have been identified in the literature as members of the GT61 family of enzymes (Anders et al. 2012). Proteins involved in O-acetylation of polysaccharides, including xylan, have been identified and designated as RWA proteins (Manabe et al., 2011), and proteins involved in O-acetylation of xyloglucan and mannan have been shown to be members of the DUF231 family (Gille et al. 2011). Most likely other members of the large DUF231 family are required for xylan O-acetylation.

Protein sequences and accession numbers for various IRX proteins and Parvus proteins are shown in FIGS. 7-12. FIGS. 7-12 provide amino acid sequence alignments of the indicated proteins. Additionally, gene and protein sequences for these proteins, and methods for obtaining the genes or proteins, are known and described in the art. One of skill in the art will recognize that these gene or protein sequences known in the art and/or as described herein can be modified to make substantially identical lignin biosynthesis enzymes, e.g., by making conservative substitutions at one or more amino acid residues. One of skill will also recognize that the known sequences (e.g., the alignments provided herein) provide guidance as to what amino acids may be varied to make a substantially identical lignin biosynthesis enzyme. For example, using any of the alignments shown in FIGS. 7-12, one of skill will recognize which amino acid residues are not highly conserved and thus can likely be changed without resulting in a significant effect on the function of the lignin biosynthesis enzyme.

In addition to the xylan synthesis genes (e.g., those listed hereinabove) a similar strategy may also be used to regulate polysaccharide O-acetylation expression patterns via RWA gene expression. RWA proteins function in acetylation in general, including in xylan O-acetylation. Thus, combining specific expression of RWA with the RWA knock-out/downregulation plants that have very low acetate content but still have excellent growth properties can also be produced using the techniques described herein. In Arabidopsis there are 4 RWA genes and three (RWA1, RWA3 and RWA4) are predominantly expressed in tissues with secondary walls (Manabe et al., 2011;). Downregulation or inactivation of two or more of these RWA genes results in decreased xylan O-acetylation and impaired function of vascular tissues (Scheller et al., 2010; WO/2010/096488). Thus, RWA may be downregulated in plants, e.g., using methods and compositions described in WO2010/096488 and an RWA gene then reintroduced into the plant where the RWA gene is under the control of a promoter/transcription factor as described herein. Alternative to targeting RWA proteins, one or more DUF231 proteins involved in xylan O-acetylation can be targeted.

Although the genes and proteins used as illustrations above have been studied primarily using Arabidopsis thaliana, orthologs are easily identified in other plant species. For example, for many genes, it has been demonstrated by complementation experiments, silencing, or RNAi that orthologs from other plants have the same function as the A. thaliana proteins (Zhou et al., 2006; Zhou et al., 2007; Lee et al., 2009).

Gene and protein sequences and/or accession numbers for IRX8, IRX14, IRX14-like, IRX9, IRX9-like, IRX7, IRX10, IRX10-like, IRX15, IRX15-like, F8H, and PARVUS are described herein. Amino acid sequence alignments for xylan biosynthesis enzymes showing the amino acid sequences for each of these proteins from multiple plant species are also shown in FIGS. 7-12. Additionally, gene and protein sequences for these proteins, and methods for obtaining the genes or proteins, are known and described in the art as discussed above. One of skill in the art will recognize that these gene or protein sequences known in the art and/or as described herein can be modified to make substantially identical xylan biosynthesis enzymes, e.g., by making conservative substitutions at one or more amino acid residues. One of skill will also recognize that the known sequences (e.g., the alignments provided herein) provide guidance as to what amino acids may be varied to make a substantially identical xylan biosynthesis enzyme. For example, using any of the alignments shown in FIGS. 7-12, one of skill will recognize which amino acid residues are not highly conserved and thus can likely be changed without resulting in a significant effect on the function of the xylan biosynthesis enzyme.

3. Vessel-Specific Promoters

In some embodiments, the polynucleotide encoding the lignin biosynthesis enzyme or xylan biosynthesis enzyme is operably linked to a vessel-specific promoter. The vessel-specific promoter is heterologous to the polynucleotide encoding the lignin biosynthesis enzyme or xylan biosynthesis enzyme (i.e., is not the native promoter associated with the lignin biosynthesis enzyme or xylan biosynthesis enzyme). A promoter is suitable for use as a vessel-specific promoter if the promoter is expressed strongly in vessel cells of the plant but is expressed at lower levels in fiber cells of the plant as compared to the level of expression of the native promoter of the lignin biosynthesis enzyme or xylan biosynthesis enzyme whose expression is to be modified.

In some embodiments, the promoter is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to the native promoter of a gene encoding vascular-related NAC-domain 1 (VND1), VND2, VND3, VND4, VND5, VND6, VND7, or VND-interacting 2 (VNI2). In some embodiments, the promoter is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to the native promoter of a gene encoding REF4 or RFR1.

In some embodiments, the vessel-specific promoter comprises SEQ ID NO:36, 94, or 95. In some embodiments, the vessel-specific promoter comprises a subsequence of SEQ ID NO:36, 94, or 95 or a variant thereof. In some embodiments, the vessel-specific promoter comprises a subsequence of SEQ ID NO:36, 94, or 95 comprising about 50 to about 1000 or more contiguous nucleotides of the sequences. In some embodiments, the vessel-specific promoter comprises a subsequence of SEQ ID NO:36, 94, or 95 comprising 50 to 1000, 50 to 900, 50 to 800, 50 to 700, 50 to 600, 50 to 500, 50 to 400, 50 to 300, 50 to 200, 50 to 100; 75 to 1000, 75 to 900, 75 to 800, 75 to 700, 75 to 600, 75 to 500, 75 to 400, 75 to 300, 75 to 200; 100 to 1000, 100 to 900, 100 to 800, 100 to 700, 100 to 600, 100 to 500, 100 to 400, 100 to 300, or 100 to 200 contiguous nucleotides of the sequence.

Vessel-specific promoters are also described in the art. See, for example, Yamaguchi et al., 2010, Plant Cell; Kubo et al., 2009, Genes Dev.; and Yamaguchi et al., 2008, Plant J.; each of which is incorporated by reference herein in its entirety.

It will be appreciated by one of skill in the art that a promoter region can tolerate considerable variation without diminution of activity. Thus, in some embodiments, the vessel-specific promoter is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to SEQ ID NO:36, SEQ ID NO:94, or SEQ ID NO:95.

4. Genetic Background of Plants

In some embodiments, a plant in which an expression cassette comprising a lignin or xylan biosynthesis enzyme is to be introduced has a genetic background that is modified to have a reduced level of activity of the lignin or xylan biosynthesis enzyme. In some embodiments, the plant is modified to have a level of activity of the lignin or xylan biosynthesis enzyme that is reduced throughout the entire plant. In some embodiments, the plant is modified to have a level of activity of the lignin or xylan biosynthesis enzyme that is reduced only in a subset of cells or tissues of the plant. The genetic background of the plant can be modified according to any method known in the art, such as antisense, siRNA, microRNA, dsRNA, sense suppression, mutagenesis, or use of a dominant negative inhibition strategy. In some embodiments, the level of expression of the protein is reduced. In some embodiments, the modified plant having the reduced level of activity, or expression, of a lignin and/or xylan biosynthesis enzyme is then used to express an expression cassette expressing that same lignin and/or xylan biosynthesis enzyme, but under the control of a vessel-specific promoter rather than its native promoter. In some embodiments, the lignin and/or xylan biosynthesis enzyme that is introduced into the plant by expression cassette is substantially identical, but not completely identical, to the lignin and/or xylan biosynthesis enzyme that is reduced in the plant, in order to avoid silencing of the lignin and/or xylan biosynthesis enzyme that is introduced by the expression cassette (e.g., silent nucleotide changes can be made in the lignin and/or xylan biosynthesis enzyme that is introduced by the expression cassette such that the amino acid sequence, but not the nucleotide sequence, is identical to the lignin and/or xylan biosynthesis enzyme being reduced in the plant).

a) Gene Silencing Techniques

In some embodiments, expression of the lignin or xylan biosynthesis enzyme is inhibited by an antisense oligonucleotide. In antisense technology, a nucleic acid segment from the desired gene is cloned and operably linked to a promoter such that the antisense strand of RNA will be transcribed. The expression cassette is then transformed into plants and the antisense strand of RNA is produced. In plant cells, it has been suggested that antisense RNA inhibits gene expression by preventing the accumulation of mRNA which encodes the enzyme of interest, see, e.g., Sheehy et al., Proc. Nat. Acad. Sci. USA, 85:8805-8809 (1988); Pnueli et al., The Plant Cell 6:175-186 (1994); and Hiatt et al., U.S. Pat. No. 4,801,340.

The antisense nucleic acid sequence transformed into plants will be substantially identical to at least a portion of the endogenous gene or genes to be repressed. The sequence, however, does not have to be perfectly identical to inhibit expression. Thus, an antisense or sense nucleic acid molecule encoding only a portion of the lignin or xylan biosynthesis enzyme-encoding sequence can be useful for producing a plant in which expression of the lignin or xylan biosynthesis enzyme is inhibited. For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. In some embodiments, a sequence of at least, e.g., 20, 25, 30, 50, 100, 200, or more continuous nucleotides (up to mRNA full length) substantially identical to an endogenous lignin or xylan biosynthesis enzyme mRNA, or a complement thereof, can be used.

Catalytic RNA molecules or ribozymes can also be used to inhibit expression of a gene encoding a lignin or xylan biosynthesis enzyme. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff et al. Nature, 334:585-591 (1988).

Another method by which expression of a gene encoding a lignin or xylan biosynthesis enzyme can be inhibited is by sense suppression (also known as co-suppression). Introduction of expression cassettes in which a nucleic acid is configured in the sense orientation with respect to the promoter has been shown to be an effective means by which to block the transcription of target genes. For an example of the use of this method to modulate expression of endogenous genes, see Napoli et al., The Plant Cell 2:279-289 (1990); Flavell, Proc. Natl. Acad. Sci., USA 91:3490-3496 (1994); Kooter and Mol, Current Opin. Biol. 4:166-171 (1993); and U.S. Pat. Nos. 5,034,323, 5,231,020, and 5,283,184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity can exert a more effective repression of expression of the endogenous sequences. In some embodiments, sequences with substantially greater identity are used, e.g., at least about 80%, at least about 95%, or 100% identity are used. As with antisense regulation, further discussed below, the effect can be designed and tested to apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. Furthermore, the introduced sequence need not have the same intron or exon pattern, and identity of non-coding segments will be equally effective. In some embodiments, a sequence of the size ranges noted above for antisense regulation is used, i.e., 30-40, or at least about 20, 50, 100, 200, 500 or more nucleotides.

Endogenous gene expression may also be suppressed by means of RNA interference (RNAi) (and indeed co-suppression can be considered a type of RNAi), which uses a double-stranded RNA having a sequence identical or similar to the sequence of the target gene. RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA. Although complete details of the mechanism of RNAi are still unknown, it is considered that the introduced double-stranded RNA is initially cleaved into small fragments, which then serve as indexes of the target gene in some manner, thereby degrading the target gene. RNAi is known to be also effective in plants (see, e.g., Chuang, C. F. & Meyerowitz, E. M., Proc. Natl. Acad. Sci. USA 97: 4985 (2000); Waterhouse et al., Proc. Natl. Acad. Sci. USA 95:13959-13964 (1998); Tabara et al. Science 282:430-431 (1998); Matthew, Comp Funct Genom 5: 240-244 (2004); Lu, et al., Nucleic Acids Res. 32(21):e171 (2004)).

Thus, in some embodiments, inhibition of a gene encoding a lignin or xylan biosynthesis enzyme is accomplished using RNAi techniques. For example, to achieve suppression of the expression of a DNA encoding a protein using RNAi, a double-stranded

RNA having the sequence of a DNA encoding the protein, or a substantially similar sequence thereof (including those engineered not to translate the protein) or fragment thereof, is introduced into a plant of interest. As used herein, RNAi and dsRNA both refer to gene-specific silencing that is induced by the introduction of a double-stranded RNA molecule, see e.g., U.S. Pat. Nos. 6,506,559 and 6,573,099, and includes reference to a molecule that has a region that is double-stranded, e.g., a short hairpin RNA molecule. The resulting plants may then be screened for a phenotype associated with the target protein, for example, screening for an increase in the extractability of sugar from the plants as compared to wild-type plants, and/or by monitoring steady-state RNA levels for transcripts encoding the protein. Although the genes used for RNAi need not be completely identical to the target gene, they may be at least 70%, 80%, 90%, 95% or more identical to the target gene sequence. See, e.g., U.S. Patent Publication No. 2004/0029283. The constructs encoding an RNA molecule with a stem-loop structure that is unrelated to the target gene and that is positioned distally to a sequence specific for the gene of interest may also be used to inhibit target gene expression. See, e.g., U.S. Patent Publication No. 2003/0221211.

The RNAi polynucleotides may encompass the full-length target RNA or may correspond to a fragment of the target RNA. In some cases, the fragment will have fewer than 100, 200, 300, 400, 500 600, 700, 800, 900 or 1,000 nucleotides corresponding to the target sequence. In addition, in some embodiments, these fragments are at least, e.g., 50, 100, 150, 200, or more nucleotides in length. In some cases, fragments for use in RNAi will be at least substantially similar to regions of a target protein that do not occur in other proteins in the organism or may be selected to have as little similarity to other organism transcripts as possible, e.g., selected by comparison to sequences in analyzing publicly-available sequence databases.

Expression vectors that continually express siRNA in transiently- and stably-transfected have been engineered to express small hairpin RNAs, which get processed in vivo into siRNAs molecules capable of carrying out gene-specific silencing (Brummelkamp et al., Science 296:550-553 (2002), and Paddison, et al., Genes & Dev. 16:948-958 (2002)). Post-transcriptional gene silencing by double-stranded RNA is discussed in further detail by Hammond et al. Nature Rev Gen 2: 110-119 (2001), Fire et al. Nature 391: 806-811 (1998) and Timmons and Fire Nature 395: 854 (1998).

Yet another way to suppress expression of an endogenous plant gene is by recombinant expression of a microRNA that suppresses a target (e.g., a gene encoding a lignin or xylan biosynthesis enzyme). Artificial microRNAs are single-stranded RNAs (e.g., between 18-25-mers, generally 21-mers), that are not normally found in plants and that are processed from endogenous miRNA precursors. Their sequences are designed according to the determinants of plant miRNA target selection, such that the artificial microRNA specifically silences its intended target gene(s) and are generally described in Schwab et al, The Plant Cell 18:1121-1133 (2006) as well as the internet-based methods of designing such microRNAs as described therein. See also, US Patent Publication No. 2008/0313773.

Another example of a method to reduce levels of a gene expression product of a gene or gene of interest employ riboswitch techniques (see, e.g., U.S. Patent Application Publication Nos. US20100286082, and US20110245326).

Methods of inhibiting plant gene expression for one or more lignin and/or xylan biosynthesis enzymes, including plants that have inhibited RWA expression, have been described in the art. See, for example, Coleman et al., Plant Physiol. 148:1229-37 (2008) (C3′H RNAi in poplar); Kitin et al., Plant Physiol. 154:887-98 (2010) (4CL antisense in poplar); Coleman et al., Proc. Acad. Natl. Sci. USA 105:4501-06 (2008) (C3′H RNAi in poplar); and Voelker et al., Plant Physiol. 154:874-86 (2010) (4CL antisense in poplar), and WO2010/096488 (RWA inhibition), each of which is incorporated by reference herein in its entirety.

As appreciated by one of skill in the art, the isoforms that are highly expressed in xylem and fibers are targeted. For example, using Arabidopsis for illustration purposes, IRX7, IRX8, IRX9, PARVUS, IRX15 are highly expressed in xylem and fibers and would therefore be targeted. For IRX10 and IRX14, both isoforms (Arabidopsis has 2 isoforms) would be typically targeted since they both have expression in xylem and fibers. Similarly, for making plants that are inhibited in Rwa expression, the isoforms that are expressed in xylem and fibers are targeted. For example, again using Arabidopsis for illustration, one of, typically two or more of, RWA1, RWA3 and RWA4 are targeted (RWA2 is not expressed in xylem and fibers).

As further understood in the art, in the steps of the methods of the invention in which the activity is introduced back into the xylan-deficient or lignin-deficient plant using a vessel specific promoter (e.g. VND6), it is not necessary to express the same isoform as the one that was targeted for inhibition. For example, an irx9 mutant plant may be employed that has very little xylan, but it is not necessary to express the tissue specific IRX9 isoform in the plant, rather a IRX9 homolog that is not normally expressed in those tissues may also be readily employed. Many plants, including Arabidopsis, have a second IRX9-like gene which is mostly expressed in tissues apart other than xylem and fibers. Similar relationships are true for IRX7/F8H, IRX14/IRX14-like, and IRX15/IRX15-like. Likewise, RWA1/RWA3/RWA4 mutants can be engineered to express Rwa2 under control of the vessel-specific promoter, e.g., a VND6 promoter.

b) Plants Having Mutant Backgrounds

In some embodiments, the level of expression of the lignin or xylan biosynthesis enzyme is reduced by generating a plant that has a mutation in a gene encoding the lignin or xylan biosynthesis enzyme. One method for abolishing or decreasing the expression of a gene encoding a lignin or xylan biosynthesis enzyme is by insertion mutagenesis using the T-DNA of Agrobacterium tumefaciens. After generating the insertion mutants, the mutants can be screened to identify those containing the insertion in the gene of interest. Mutants containing a single mutation event at the desired gene may be crossed to generate homozygous plants for the mutation (Koncz et al. (1992) Methods in Arabidopsis Research. World Scientific).

Alternatively, random mutagenesis approaches may be used to generate new alleles that will generate truncated or defective (non-functional or poorly active) enzymes or unstable RNA, or to disrupt or “knock-out” the expression of a gene encoding a lignin or xylan biosynthesis enzyme using either chemical or insertional mutagenesis or irradiation. One method of mutagenesis and mutant identification is known as TILLING (for targeting induced local lesions in genomes). In this method, mutations are induced in the seed of a plant of interest, for example, using EMS treatment. The resulting plants are grown and self-fertilized, and the progeny are assessed. For example, the plants may be assessed using PCR to identify whether a mutated plant has a mutation in the gene of interest, or by evaluating whether the plant has reduced lignin content in a part of the plant that expressed the gene of interest. TILLING can identify mutations that may alter the expression of specific genes or the activity of proteins encoded by these genes (see Colbert et al (2001) Plant Physiol 126:480-484; McCallum et al (2000) Nature Biotechnology 18:455-457).

Methods of making plants having a mutant background for one or more lignin and/or xylan biosynthesis enzymes have been described in the art. See, for example, Schilmiller et al., Plant J. 60:771-82 (2009) (Arabidopsis mutant for C4H); and Weng et al., Plant Cell 22:1033-45 (2010) (Selaginella mutant for F5H), each of which is incorporated by reference herein in its entirety. Methods of making plants that have an RWA mutant background are described, e.g., in WO2010/096488.

In some embodiments, where expression cassettes comprising a lignin biosynthesis enzyme and a xylan biosynthesis enzyme are to be introduced into a plant, the plant has a genetic background that is modified to have reduced levels of expression of both the lignin biosynthesis enzyme and the xylan biosynthesis enzyme. Such plants can be generated using known methods as described herein sections of the application describing modification of plants to suppress or reduce expression of a desired product.

B. Modification of Expression Using a Transcription Factor that Regulates the Production of Secondary Cell Wall

In another aspect, the present invention provides methods of engineering a plant having increased secondary cell wall deposition. In some embodiments, the method comprises:
introducing an expression cassette into the plant, wherein the expression cassette comprises a polynucleotide encoding a transcription factor that regulates the production of secondary cell wall in woody tissue operably linked to an induced heterologous promoter, wherein the promoter is substantially identical to the native promoter of a gene that is a downstream target of the transcription factor in the biosynthetic pathway; and
culturing the plant under conditions in which the transcription factor is expressed. The downstream target may be a direct or indirect target of the transcription factor.

The expression cassette as described herein, when introduced into a plant, generates a positive feedback loop that allows the maintenance of expression or the overexpression of genes involved in secondary cell wall biosynthesis, due to the transcription factor directly or indirectly inducing expression of the promoter from the downstream target gene, which in turn is operably linked to the polynucleotide encoding the transcription factor, resulting in increased expression of the transcription factor. This positive feedback loop results in the continued production or overproduction of secondary cell walls components such as cellulose, hemicellulose, and lignin.

1. Transcription Factors that Regulate the Production of Secondary Cell Wall

In some embodiments, the expression cassette comprises a polynucleotide encoding a transcription factor that regulates the production of secondary cell wall. A transcription factor may be selected for use in the present invention on the basis that it induces one or more genes involved in lignin biosynthesis and/or polysaccharide (cellulose and hemicellulose) biosynthesis. Alternatively or additionally, the transcription factor may be selected for use on the basis of an overexpression or loss-of-function phenotype in a plant (e.g., a plant overexpressing that transcription factor that exhibits a phenotype of increased cell wall thickening or secondary cell wall deposition, or a plant having a dominant repression or loss-of-function mutation of that transcription factor that exhibits a phenotype of decreased cell wall thickening or secondary cell wall deposition). In some embodiments, the transcription factor is NAC secondary wall-thickening promoting factor 1 (NST1), NST2, NST3, secondary wall-associated NAC domain protein 2 (SND2), SND3, MYB domain protein 103 (MYB103), MBY85, MYB46, MYB83, MYB58, or MYB63.

The transcription factors NST1, NST2, NST3, SND2, SND3, MYB103, MBY85, MYB46, MYB83, MYB58, and MYB63 have been characterized in Arabidopsis and have been shown to regulate secondary cell wall production in that species. See, e.g., Mitsuda et al., Plant Cell 17:2993-3006 (2005); Mitsuda et al., Plant Cell 19:270-80 (2007); Ohashi-Ito et al., Plant Cell 22:3461-73 (2010); Zhong et al., Plant Cell 20:2763-82 (2008); Zhong et al., Plant Cell 19:2776-92 (2007); Ko et al., Plant J. 60:649-65 (2009); and McCarthy et al., Plant Cell Physiol. 50:1950-64 (2009). Thus, in some embodiments, the polynucleotide encoding a transcription factor that regulates the production of secondary cell wall is substantially identical to any of the polynucleotide sequences of SEQ ID NOs:13, 15, 17, 19, 21, 23, 25, 27, 29, 31, or 33. Additionally, these transcription factors have been identified in a variety of other plants, including rice, sorghum, poplar, grape, moss, maize, and switchgrass. Furthermore, the general mechanism of secondary cell wall biosynthesis is conserved not only between monocots and dicots, but also within these groups. Thus, in some embodiments, the polynucleotide encoding a transcription factor that regulates the production of secondary cell wall comprises a homolog of any of the polynucleotide sequences of SEQ ID NOs:13, 15, 17, 19, 21, 23, 25, 27, 29, 31, or 33 or any of the amino acid sequences of SEQ ID NOs:14, 16, 18, 20, 22, 24, 26, 28, 30, 32, or 34 or any of the amino acid sequences of FIG. 13.

In some embodiments, the polynucleotide encoding a transcription factor that regulates the production of secondary cell wall in woody tissue comprises a polynucleotide sequence that is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to any of SEQ ID NOs:13, 15, 17, 19, 21, 23, 25, 27, 29, 31, or 33. In some embodiments, the polynucleotide encoding a transcription factor that regulates the production of secondary cell wall in woody tissue comprises a polynucleotide sequence that encodes a polypeptide sequence that is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to any of SEQ ID NOs:14, 16, 18, 20, 22, 24, 26, 28, 30, 32, or 34. In some embodiments, the transcription factor that regulates the production of secondary cell wall in woody comprises an amino acid sequence that is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to any of SEQ ID NOs:14, 16, 18, 20, 22, 24, 26, 28, 30, 32, or 34 or to any of the amino acid sequences of FIG. 13.

Gene and protein sequences and/or accession numbers for NST1, NST2, NST3, SND2, SND3, MYB103, MBY85, MYB46, MYB83, MYB58, and MYB63 are described in the Sequence Listing herein. Additionally, amino acid sequence alignments for the transcription factors, showing the amino acid sequences for each of these proteins from multiple plant species, are shown in FIGS. 1-6. Gene and protein sequences for these proteins, and methods for obtaining the genes or proteins, are also known and described in the art. See, for example, Goiocoechea et al., 2005, Plant J. 43:553-67; McCarthy et al., 2009, Plant Cell Physiol. 50:1950-64; Shen et al., 2009, Bioenerg. Res. 2:217-32; and Zhong et al., 2010, Trends in Plant Sciences, http://dx.doi.org/10.1016/j.tplants.2010.08.007. One of skill in the art will recognize that these gene or protein sequences known in the art and/or as described herein can be modified to make substantially identical transcription factors, e.g., by making conservative substitutions at one or more amino acid residues. One of skill will also recognize that the known sequences (e.g., the alignments provided herein) provide guidance as to what amino acids may be varied to make a substantially identical transcription factor. For example, using any of the alignments shown in FIGS. 1-6, one of skill will recognize which amino acid residues are not highly conserved and thus can likely be changed without resulting in a significant effect on the function of the transcription factor.

2. Promoters from Downstream Targets of the Transcription Factors that Regulate the Production of Secondary Cell Wall

In some embodiments, the polynucleotide encoding the transcription factor that regulates secondary cell wall production is operably linked to a promoter that is a downstream target of the transcription factor. The promoter is heterologous to the polynucleotide encoding the transcription factor that regulates secondary cell wall production (i.e., is not the native promoter associated with the transcription factor that regulates secondary cell wall production). A promoter is suitable for use with the transcription factor that regulates secondary cell wall production if expression of the promoter is induced, directly or indirectly, by the transcription factor to be expressed, and if the promoter is expressed in the desirect location, e.g., the stem of the plant but not strongly expressed in leaves of the plant.

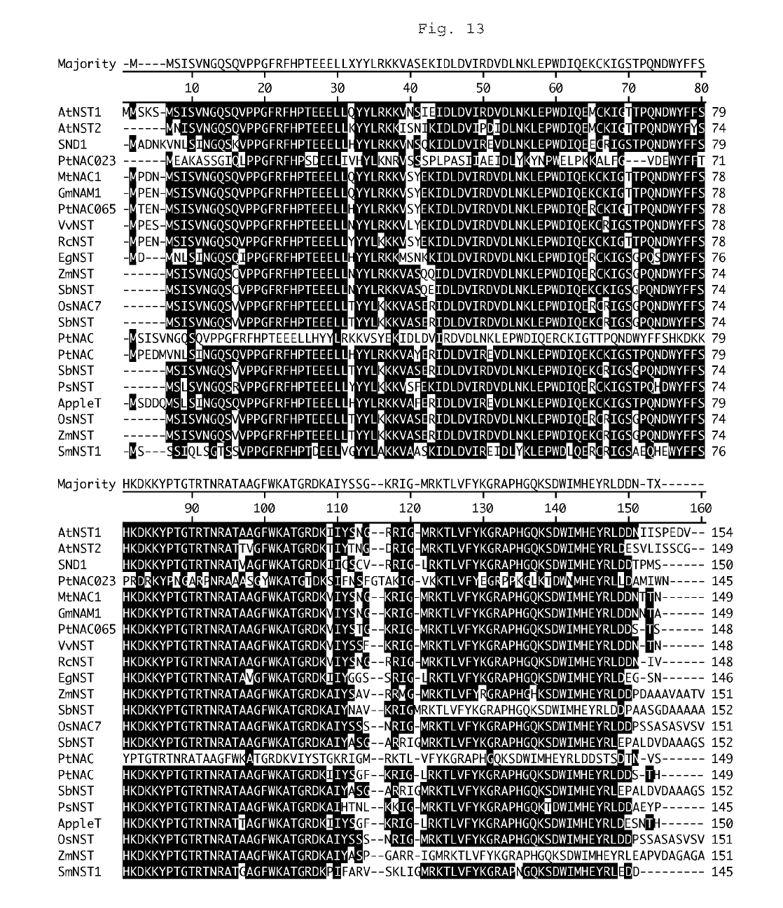

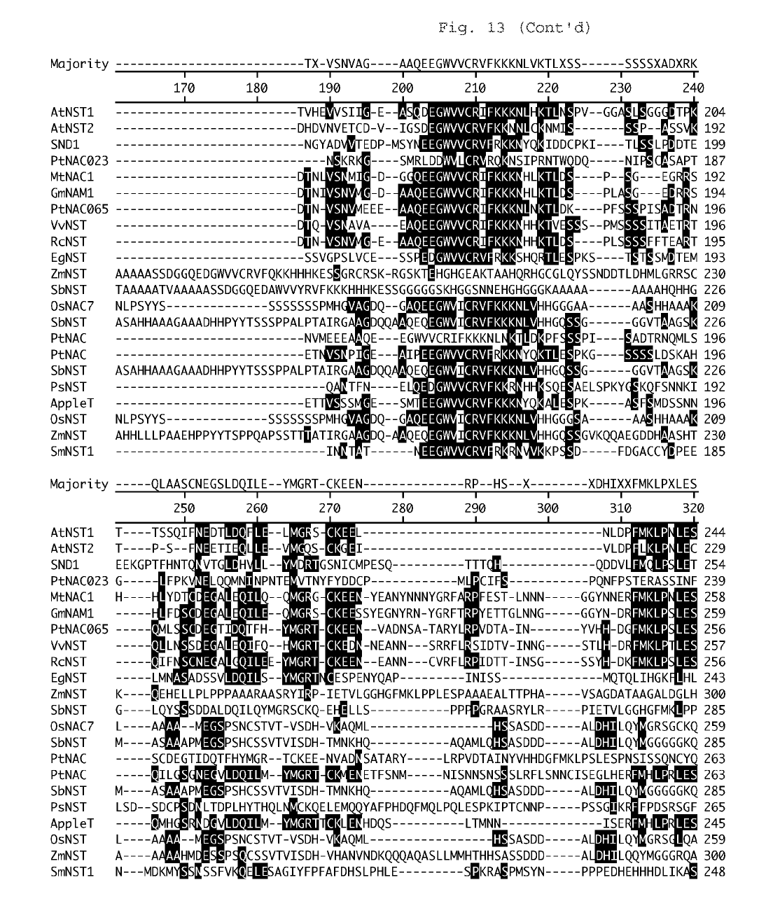

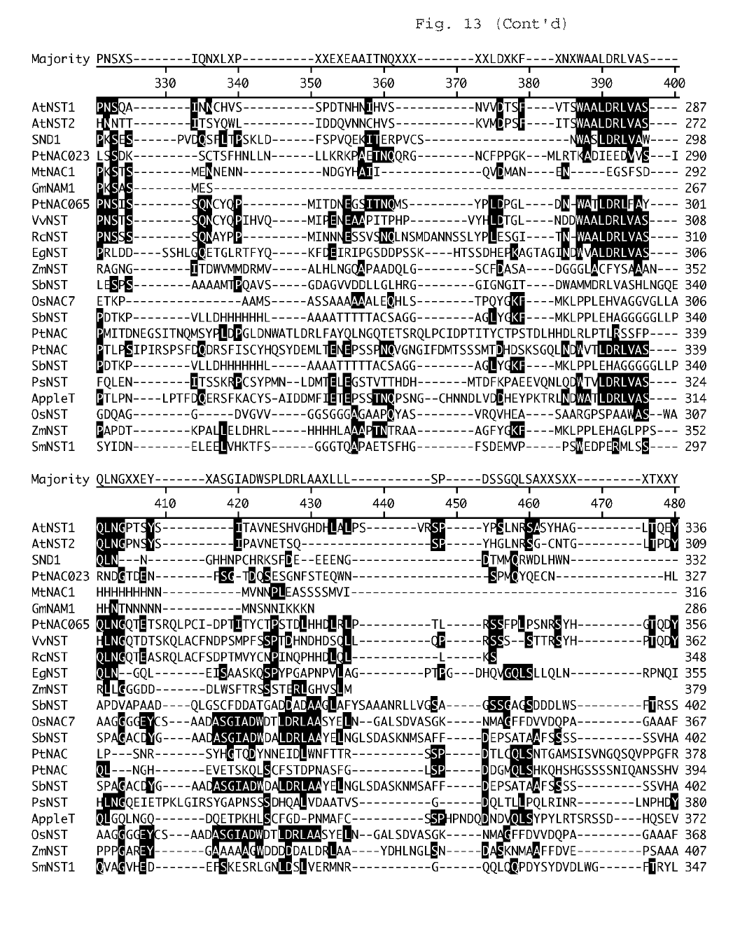

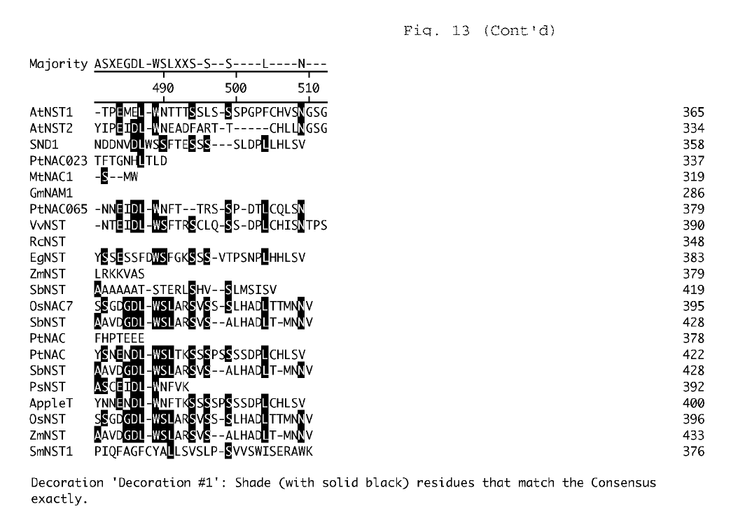

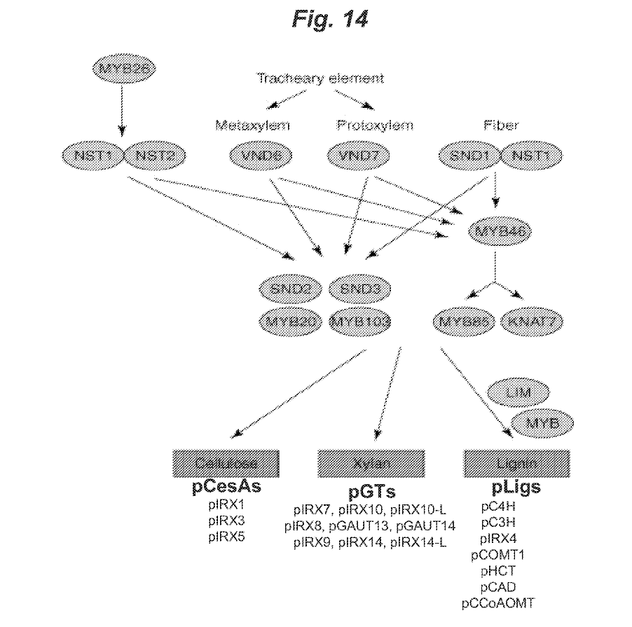

In some embodiments, the promoter is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to the native promoter of a gene that is a downstream target of the transcription factor. In some embodiments, the promoter is substantially identical to the native promoter of IRX1, IRX3, IRX5, IRX8, IRX9, IRX14, IRX7, or IRX10. In some embodiments, the transcription factor is selected from NST1, NST2, NST3, SND2, SND3, MYB103, MBY85, MYB46, MYB83, MYB58, and MYB63 and the promoter is substantially identical to a native promoter selected from IRX1, IRX3, IRX5, IRX8, IRX9, IRX14, IRX7, IRX10, GAUT13, or GAUT14. See FIG. 14. Alternative promoters may also be used. For example, alternative promoters can be identified by coexpression analysis, e.g., using Atted II database and known promoters as bait; or by identifying functional motifs of interest in the promoters of candidate genes. Promoters from other genes that are regulated by the transcription factor may also be used.

In some embodiments, the promoter comprises a subsequence of SEQ ID NO:35 or a variant thereof. In some embodiments, the promoter comprises a subsequence of SEQ ID NO:35 comprising about 50 to about 1000 or more contiguous nucleotides of SEQ ID NO:35. In some embodiments, the promoter comprises a subsequence of SEQ ID NO:35 comprising 50 to 1000, 50 to 900, 50 to 800, 50 to 700, 50 to 600, 50 to 500, 50 to 400, 50 to 300, 50 to 200, 50 to 100; 75 to 1000, 75 to 900, 75 to 800, 75 to 700, 75 to 600, 75 to 500, 75 to 400, 75 to 300, 75 to 200; 100 to 1000, 100 to 900, 100 to 800, 100 to 700, 100 to 600, 100 to 500, 100 to 400, 100 to 300, or 100 to 200 contiguous nucleotides of SEQ ID NO:35.

Promoters that are downstream targets of the transcription factors described herein are also described in the art. See, for example, Oikawa et al, 2010, PLoS ONE; Taylor et al., 2000, Plant Cell; Betancur et al., 2010, J. Integrative Plant Biol.; Persson et al., 2007, Plant Physiol.; Wu et al., 2010, Plant Physiol.; Zhong et al., 2005, Plant Cell; and Wu et al., 2009, Plant J.; each of which is incorporated by reference herein in its entirety.

It will be appreciated by one of skill in the art that a promoter region can tolerate considerable variation without diminution of activity. Thus, in some embodiments, the promoter is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to SEQ ID NO:35.

C. Modification of Expression Using a Transcription Factor that Regulates Wax/Cutin Production

Improving plant water use efficiency is an important priority to reduce water consumption by plant per ton of CO₂ fixed and improve plant drought stress tolerance. It would improve or maintain biomass yields under water limiting conditions by reducing cellular oxidative stresses, which also cause a reduction of photosynthesis efficiency. Developing strategies that can reduce water losses by plants without reducing biomass yield reduces water needs, improves drought stress tolerance and is compatible with drought stress tolerance technologies already developed. Part of the water that is lost by plants occurs by water evaporation through the cuticle on the surface of leaf epidermis, also called epicuticle. Transcription factors to control wax/cutin biosynthesis have been identified. Although overexpression in plants of some of these in plants improved resistance to drought-stress and reduced water losses, the expression strategies used to increase the expression of these transcription factors also caused deposition of wax or/and cutin in sensitive tissues generating undesired effects on plant growth and development (Aharoni et al., The Plant Cell 16:2463-2480, 2004; Zhang et al., Plant J. 42:689-797, 2005). Beyond water use efficiency, modifying epicuticular wax composition and content has several other potential advantages since the epicuticle is the first barrier for many pathogens, insects and chemicals. The invention thus provides an artificial positive feedback loop system to increase wax and/or cutin deposition on the epidermis of plants in order to improve plant water use efficiency and drought-stress tolerance.

Thus, in another aspect, the present invention provides methods of engineering a plant having modified, e.g., increased, wax and/or cutin production. In some embodiments, the method comprises:
introducing an expression cassette into the plant, wherein the expression cassette comprises a polynucleotide encoding a transcription factor that regulates the production of wax/cutin components linked to a heterologous induced promoter, wherein the promoter is substantially identical to the native promoter of a gene that is a downstream target of the transcription factor; and
culturing the plant under conditions in which the transcription factor is expressed. The downstream target may be a direct or indirect target of the transcription factor.

The expression cassette as described herein, when introduced into a plant, generates a positive feedback loop that allows the maintenance of expression or the overexpression of genes involved in wax and/or cutinbiosynthesis, due to the transcription factor directly or indirectly inducing expression driven by the promoter from the downstream target gene, which in turn is operably linked to the polynucleotide encoding the transcription factor, resulting in increased expression of the transcription factor. This positive feedback loop results in the continued production or overproduction of wax and/or cutin.

1. Transcription Factors that Regulate the Production of Wax/Cutin

In some embodiments, the expression cassette comprises a polynucleotide encoding a transcription factor that regulates the production of wax and/or cutin components for the production of wax (and/or cutin). A transcription factor may be selected for use in the present invention on the basis that it induces one or more genes, typically multiple genes, involved in the wax biosynthetic pathway. Alternatively or additionally, the transcription factor may be selected for use on the basis of an overexpression or loss-of-function phenotype in a plant (e.g., a plant overexpressing that transcription factor that exhibits a phenotype of increased wax production, or a plant having a dominant repression or loss-of-function mutation of that transcription factor that exhibits a phenotype of decreased wax production). In some embodiments, the transcription factor is an shine (SHN) transcription factor, such as SHN1 (also known as WIN1), SHN2, SHN3, SHN4, SHN5, or MYB 96.

The transcription factors SHN1, SHN2, SHN3, SHN4, SHN5, and MYB96 have been characterized in Arabidopsis and have been shown to regulate wax and/or cutin biosynthesis in Arabidopsis and other plant species. See, e.g., Shi et al., PLoS Genet. 7, e1001388 (2011); Seo et al., Plant Cell 23:1138-1152 (2011); Kannangara et al., Plant Cell 19:1278-1294 (2007); Zhang et al., Plant J. 42:689-707 (2005), Aharoni et al., Plant Cell 16:2463-2480 (2004); Broun et al., Proc. Natl. Acad Sci USA 101:4706-4711 (2004); and Suh et al., Plant Physiol. 139:1649-1665 (2005). Additionally, SHN transcription factor sequences have been identified in a variety of other plants, including, including poplar, Medicago, rice, grasses e.g., Brachypodium, corn, sorghum, barley, spruce, spikemoss, and bryophtyes. Similarly, Myb96 transcription factor sequences have been identified in various other plants including Thellungiella, Medicago, poplar, grape vine, citrus, brachypodium, wheat, barley, rice, and sorghum. Furthermore, the general mechanism of wax/cutin biosynthesis is conserved not only between monocots and dicots, but also within these groups.

In some embodiments, the polynucleotide encoding a transcription factor that regulates the production of wax/cutin a encodes a SHN transcription factor. In some embodiments, the polynucleotides encodes an SHN transcription factor of any one of SEQ ID NOs:37-59, or a variant thereof. Thus, in some embodiments, the polynucleotide encoding a transcription factor that regulates the production of wax/cutin synthesis encodes a protein that is substantially identical to any one of SEQ ID NOS:37-59.

In some embodiments, the polynucleotide encoding a transcription factor that regulates the production of wax cutin synthesis comprises a polynucleotide sequence encodes an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any of SEQ ID NOs:37-59.

In some embodiments, the polynucleotide encoding a transcription factor that regulates the production of wax/cutin a encodes a Myb96 transcription factor. In some embodiments, the polynucleotides encodes a Myb96 transcription factor of any one of SEQ ID NOS:80-93, or a variant thereof. Thus, in some embodiments, the polynucleotide encoding a transcription factor that regulates the production of wax/cutin synthesis encodes a protein that is substantially identical to any one of SEQ ID NOS:80-93.

In some embodiments, the polynucleotide encoding a transcription factor that regulates the production of wax cutin synthesis comprises a polynucleotide sequence encodes an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any of SEQ ID NOS:80-93.

Illustrative protein sequences and/or accession numbers for SHN1, SHN2, SHN3, SHN4, SHN5, or MYB 96 are provided herein. Additionally, amino acid sequence alignments for the transcription factors, showing the amino acid sequences for each of these proteins from multiple plant species, are shown in FIGS. 25 and 26. Gene and protein sequences for these proteins, and methods for obtaining the genes or proteins, are also known and described in the art (see, e.g., references cited hereinabove). One of skill in the art will recognize that these gene or protein sequences known in the art and/or as described herein can be modified to make variant transcription factors, e.g., by making conservative substitutions at one or more amino acid residues. One of skill will also recognize that the known sequences (e.g., the alignments provided herein) provide guidance as to which amino acids may be varied to make a substantially identical transcription factor. For example, using the alignment provided in FIGS. 25 and 26, one of skill will recognize which amino acid residues are not highly conserved and thus can likely be changed without resulting in a significant effect on the function of the transcription factor. Similarly, one of skill can identify highly conserved domain that are conserved in all or almost all of the transcription factors and use this information in identifying variants for use in the invention.

2. Promoters from Downstream Targets of the Transcription Factors that Regulate Wax and/or Cutin Production

In some embodiments, the polynucleotide encoding the transcription factor that regulates wax and/or cutin production is operably linked to a promoter that is a downstream target of the transcription factor. The promoter is heterologous to the polynucleotide encoding the transcription factor that regulates wax and/or cutin production (i.e., is not the native promoter associated with the transcription factor). A promoter is suitable for use with the transcription factor if expression of the promoter is induced, directly or indirectly, by the transcription factor to be expressed, and if the promoter is expressed in the plant at the desired location, e.g., in the leaf of the plant.

In some embodiments, the promoter is substantially identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical) to the native promoter of a gene that is a downstream target of the transcription factor. In some embodiments, the promoter is a CER1, CER2, CER3, CER4, CER5, CER6, CER10, WSD1, Mah1, WBC11, KCS1, KCS2, FATB, LACS1, LACS2, CYP864A, CYP86A7, CYP86A5, KCS10, or KCS5 promoter, or a variant thereof that is substantially identical to a native promoter. In some embodiments, the transcription factor is selected from SHN1, SHN2, SHN3, SHN4, SHN5, or MYB 96 and the promoter is substantially identical to a native promoter selected from CER1, CER2, CER3, CER4, CER5, CER6, CER10, WSD1, Mah1, WBC11, KCS1, KCS2, FATB, LACS1, LACS2, CYP864A, CYP86A7, CYP86A5, KCS10, or KCS5. Alternative promoters may also be used. For example, alternative promoters can be identified by coexpression analysis, e.g., using Atted II database and known promoters as bait; or by identifying functional motifs of interest in the promoters of candidate genes. Promoters from other genes that are induced by the transcription factor may also be used.

In some embodiments, the promoter comprises a subsequence of any one of SEQ ID NOs:60-79, e.g., the sequence form WBC11 or CER1, or a variant thereof. In some embodiments, the promoter comprises a subsequence comprising about 50 to about 1000 or more contiguous nucleotides of any one of SEQ ID NOs:60-79. In some embodiments, the promoter comprises a subsequence of any one of SEQ ID NOs:60-79 comprising 50 to 1000, 50 to 900, 50 to 800, 50 to 700, 50 to 600, 50 to 500, 50 to 400, 50 to 300, 50 to 200, 50 to 100; 75 to 1000, 75 to 900, 75 to 800, 75 to 700, 75 to 600, 75 to 500, 75 to 400, 75 to 300, 75 to 200; 100 to 1000, 100 to 900, 100 to 800, 100 to 700, 100 to 600, 100 to 500, 100 to 400, 100 to 300, or 100 to 200 contiguous nucleotides of the sequence.

Promoters that are downstream targets of the transcription factors described herein are also described in the art. See, for example, review of wax biosynthesis in plants and references cited therein (Schreiber, Trends Plant Sci., 2010; Kunst & Samuels, Curr. Opinion Plant Biol. 12:721-727, 2009; Samuels et al., Annu. Rev. Plant Biol. 59:683-707, 2008; Nawrath, 19:281-287, 2006; Kunst & Samuels, Progress in Lipid Res. 42:51-80, 2003; Lemieux, Trends in Plant Sci. 1:312, 1996). References describing wax mutants analyzed in Arabidopsis include Bourdenx et al., Plant Physiol 156, 29-45 (2011); Panikashvili et al. Mol Plant 3, 563-575 (2010); Weng, et al., Planta 231, 1089-1100 (2010); Lee et al. Plant J 60, 462-475 (2009); Li et al., Plant Physiol 148, 97-107 (2008); Greer et al., Plant Physiol 145, 653-667 (2007); Rowland et al., FEBS Lett 581, 3538-3544 (2007); Rowland et al., Plant Physiol 142, 866-877 (2006); Costaglioli et al., Biochim Biophys Acta 1734, 247-258 (2005); Sturaro et al., Plant Physiol 138, 478-489 (2005); Schnurr et al., Plant Cell 16, 629-642 (2004); Pighin et al., Science 306, 702-704 (2004); Bonaventure et al., Plant Cell 15, 1020-1033 (2003); Chen et al., Plant Cell 15, 1170-1185 (2003); Fiebig et al., Plant Cell 12, 2001-2008 (2000); and Millar et al., Plant Cell 11, 825-838 (1999). Wax biosynthetic pathways are also conserved among plants species (see, e.g., Wang et al., Plant Mol Biol 78, 275-288 (2011); Mao et al., Planta 235, 39-52 (2012); Yu et al., Planta 228, 675-685 (2008); Tacke et al., Plant J 8, 907-917 (1995); Islam et al., Plant Mol Biol 70, 443-456 (2009); Post-Beittenmiller Plant Physiol Bioch 36, 157-166 (1998); and Park et al., Plant Mol Biol 74, 91-103 (2010)).

D. Artificial Positive Feedback Loops

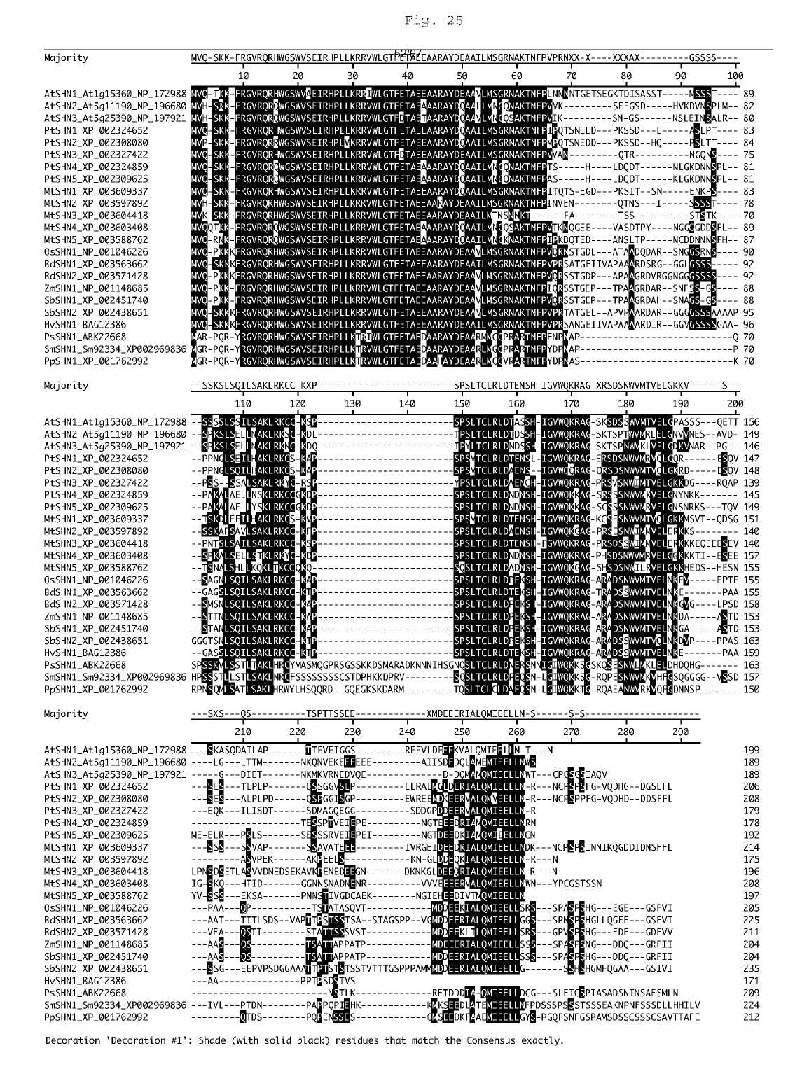

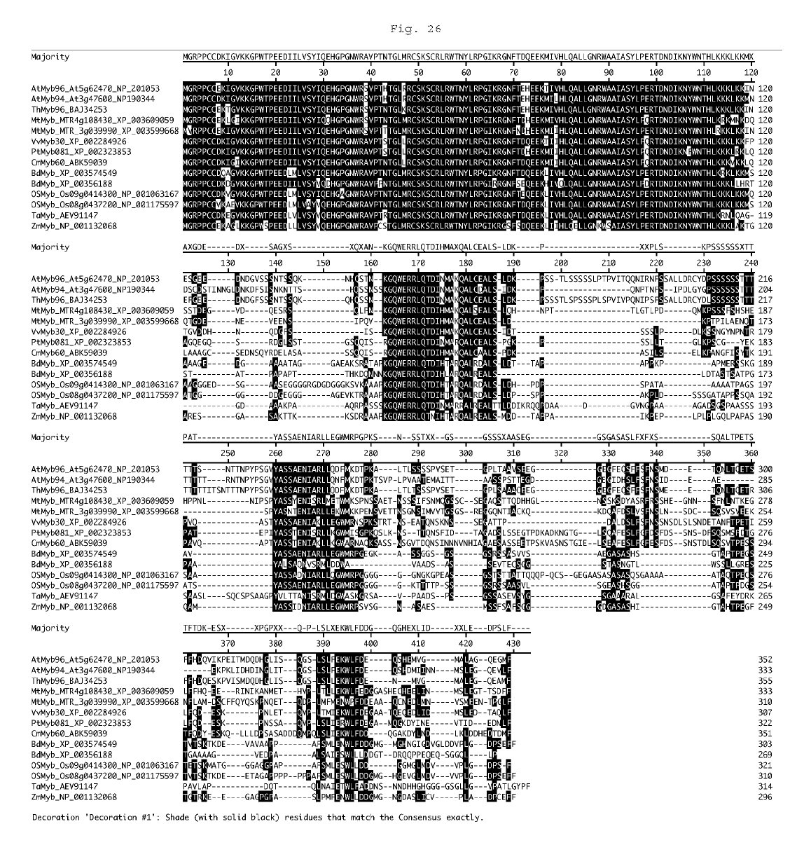

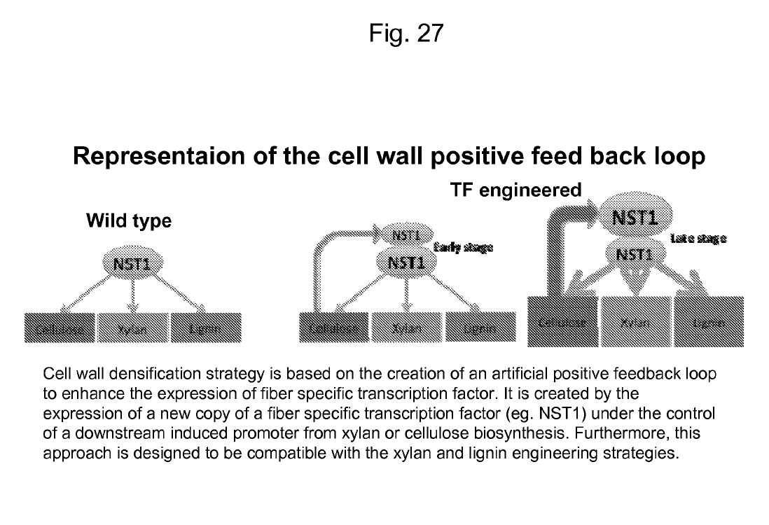

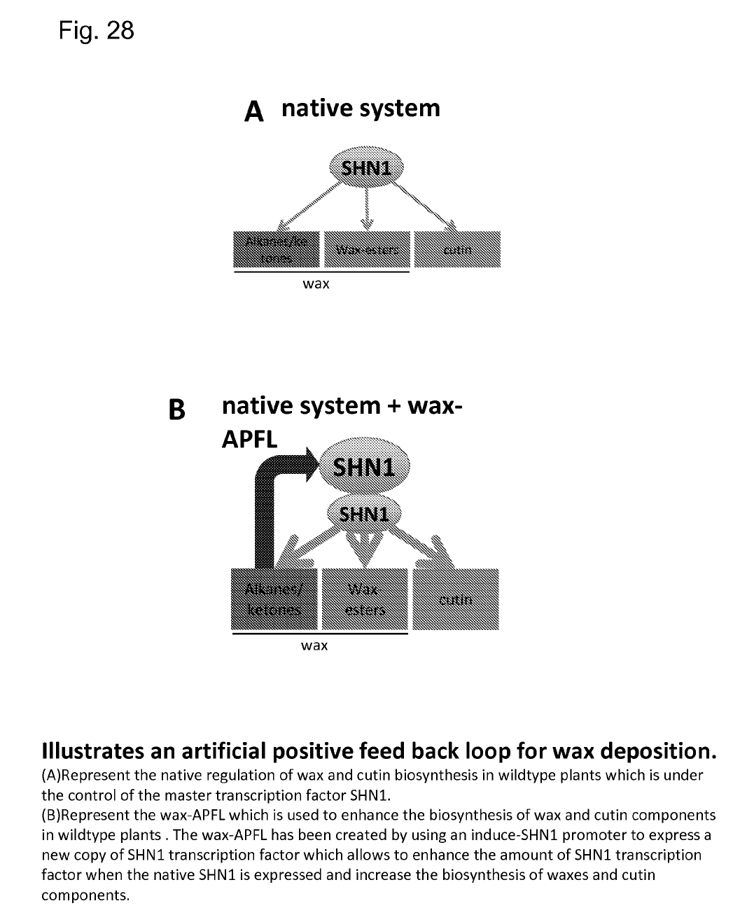

In a further aspect, the invention provides artificial positive feedback loops for regulating gene expression in plants. An APFL over-induces or increases lifetime expression of a particular transcription factor and its downstream pathway. Examples of such systems are described above for secondary wall deposition in fiber stems and for wax deposition. Illustrative examples for cell wall densification and wax deposition of the principle underlying this strategy are shown in FIGS. 27 and 28. A transcription factor suitable for use in an APFL typically plays a role in controlling expression of multiple components of a pathway of interest. A cell type-specific promoter where expression is driven by the transcription factor is used as the promoter in the APFL construct. The APFL is created by introducing an expression construct into a plant cell where the construct comprises a polynucleotide encoding a transcription factor of interest operably linked to the desired promoter. Upon expression of the native transcription factor, expression of downstream gene is induced along with expression of the introduced transcription factor encoded by the APFL construct.

Additional examples of biosynthetic pathways that can employ an APFL include lipid biosynthetic pathways. For example, it is known that lipid biosynthesis and accumulation in seeds and other tissues occurs in specific cell types and is regulated by transcription factors such as WRL1 (WRINKLED; At3g54320), LEC1 (At1g21970), or LEC2 (At1g28300). These transcription factors can thus be used to create an AFPL to increase the accumulation of lipids in a desired tissue such as seed. Other transcription factors and appropriate promoters for use in an APFL can also be identified for other biosynthetic pathways. Lipid biosynthesis pathways are discussed, e.g., in Ohlrogge & Browse, Plant Cell 7:957, 1995; Hildebrand, et al., Plant Lipids: Biology, Utilisation and Manipulation, 67-102 (2005); and Dyer & Mullen, Seed Sci. Res. 15:255-267 (2005).

Other biosynthetic pathways that may be engineered to create an APFL include the terpenoid pathway. For example, an APFL may be created to increase terpenoid indole alkaloid biosynthesis. Transcription factors that may be used for such an APFL include CrMYC2, ORCA2 or ORCA3. A nucleic acid encoding the transcription factor may be operably linked to an induced promoter such as pSTR, which controls the expression of the strictosidine synthase from catharanthus roseus. The terpenoid indole alkaloid pathway is known (see, e.g., Peebles, et al., Metab Eng 11: 76-86 (2009); Liu, et al., J Integr Plant Biol 49:961-974 (2007); Menke, et al., EMBO J 18:4455-4463 (1999), each of which references is incorporated by reference).

A further example of an APFL is one that is employed to increase artemisinin biosynthesis (sesquiterpene). An illustrative transcription factor that may be used for such an APFL is AaWRK1 (from Artemisia annua). A nucleic acid encoding the transcription factor may be operably linked to an induced promoter such as pADS, which controls the expression of the amorpha-4,11-diene synthase from Artemisia annua. This biosynthetic pathway is known (see, e.g., Ma, et al., Plant Cell Physiol 50:2146-2161 (2009), which is incorporated by reference).

Yet another example of an APFL is one that is employed to increase berberine (an alkaloid) biosynthesis. An illustrative transcription factor that may be used for such an APFL is CjWRK1 (from Coptis japonica). A nucleic acid encoding the transcription factor may be operably linked to an induced promoter such as pCYP719A1, which controls the expression of the canadine synthase from Coptis japonica. This biosynthetic pathway is known (see, e.g., Kato, et al., Plant Cell Physiol 488-18 (2007), which is incorporated by reference).

E. Genetic Background of Plants in which an Artificial Feedback Loop is Introduced

In some embodiments, the plant in which the polynucleotide encoding a transcription factor, linked to a promoter from a downstream gene where expression is driven by the transcription factor, as described herein is expressed is a wild-type (i.e., naturally occurring) plant. In some embodiments, the plant in which the polynucleotide encoding a transcription factor as described herein is expressed is a mutant plant. As used herein, a “mutant plant” includes a plant having any loss-of-function or gain-of-function mutation of any gene or genes of interest as well as a plant in which endogenous expression of any gene or genes of interest is suppressed or decreased using known methodology (e.g., by antisense, siRNA, microRNA, dsRNA, or sense suppression). For example, in some embodiments, levels of a gene expression product of a gene or gene of interest can be reduced using known technologies such as riboswitch techniques (see, e.g., U.S. Patent Application Publication Nos. US20100286082, and US20110245326.)

In some embodiments, the plant in which the polynucleotide encoding a transcription factor as described herein is expressed is a plant having spatially modified gene expression of a lignin biosynthesis enzyme and/or a xylan biosynthesis enzyme, as described above. In some embodiments, the plant has been modified to have a reduced level of expression of a lignin biosynthesis enzyme and/or a xylan biosynthesis enzyme at least in tissues other than xylem tissue, and further comprises an expression cassette comprising a polynucleotide encoding the lignin biosynthesis enzyme (e.g., PAL, C4H, 4CL, HCT, C3′H, or CCR1) and/or a xylan biosynthesis enzyme (e.g., IRX8, IRX14, IRX9, IRX7, IRX10, F8H, PARVUS, RWA1, RWA2, RWA3 or RWA4) operably linked to a heterologous vessel-specific promoter (e.g., pVND1, pVND2, pVND3, pVND4, pVND5, pVND6, pVND7, pVNI2, pREF4, or pRFR1).

F. Preparation of Recombinant Expression Vectors

Once the promoter sequence and the coding sequence for the gene of interest (e.g., lignin biosynthesis enzyme, xylan biosynthesis enzyme, or transcription factor regulating the production of secondary cell wall) are obtained, the sequences can be used to prepare an expression cassette for expressing the gene of interest in a transgenic plant. Typically, plant transformation vectors include one or more cloned plant coding sequences (genomic or cDNA) encoding a protein of interest, such as a transcription factor, under the transcriptional control of 5′ and 3′ regulatory sequences. Vectors also typically comprise a dominant selectable marker. In typical embodiments, such plant transformation vectors also contain a promoter of interest (e.g., a vessel-specific promoter as described herein or a promoter whose expression is regulated by a transcription factor regulating the production of secondary cell wall), a transcription initiation start site, an RNA processing signal (such as intron splice sites), a transcription termination site, and/or a polyadenylation signal.

The plant expression vectors may include RNA processing signals that may be positioned within, upstream or downstream of the coding sequence. In addition, the expression vectors may include regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Plant expression vectors routinely also include dominant selectable marker genes to allow for the ready selection of transformants. Such genes include those encoding antibiotic resistance genes (e.g., resistance to hygromycin, kanamycin, bleomycin, G418, streptomycin or spectinomycin), herbicide resistance genes (e.g., phosphinothricin acetyltransferase), and genes encoding positive selection enzymes (e.g. mannose isomerase).

Once an expression cassette comprising a polynucleotide encoding the lignin biosynthesis enzyme, xylan biosynthesis enzyme, or transcription factor regulating the production of secondary cell wall and operably linked to a promoter as described herein has been constructed, standard techniques may be used to introduce the polynucleotide into a plant in order to modify gene expression. See, e.g., protocols described in Ammirato et al. (1984) Handbook of Plant Cell Culture—Crop Species. Macmillan Publ. Co. Shimamoto et al. (1989) Nature 338:274-276; Fromm et al. (1990) Bio/Technology 8:833-839; and Vasil et al. (1990) Bio/Technology 8:429-434.

Transformation and regeneration of plants is known in the art, and the selection of the most appropriate transformation technique will be determined by the practitioner. Suitable methods may include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence in a plant in a manner to cause stable or transient expression of the sequence. Examples of these methods in various plants include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants can be selected using a dominant selectable marker incorporated into the transformation vector. Typically, such a marker will confer antibiotic or herbicide resistance on the transformed plants or the ability to grow on a specific substrate, and selection of transformants can be accomplished by exposing the plants to appropriate concentrations of the antibiotic, herbicide, or substrate.

The polynucleotides coding for a lignin biosynthesis enzyme, xylan biosynthesis enzyme, or transcription factor regulating the production of secondary cell wall, as well as the polynucleotides comprising promoter sequences for vessel-specific promoters or promoters from downstream targets of a transcription factor regulating the production of secondary cell wall, can be obtained according to any method known in the art. Such methods can involve amplification reactions such as PCR and other hybridization-based reactions or can be directly synthesized.

G. Plants in which Gene Expression can be Modified

An expression cassette comprising a polynucleotide encoding the lignin biosynthesis enzyme, xylan biosynthesis enzyme, or transcription factor regulating the production of secondary cell wall and operably linked to a promoter, as described herein, can be expressed in various kinds of plants. The plant may be a monocotyledonous plant or a dicotyledonous plant. In some embodiments of the invention, the plant is a green field plant. In some embodiments, the plant is a gymnosperm or conifer.

In some embodiments, the plant is a plant that is suitable for generating biomass. Examples of suitable plants include, but are not limited to, Arabidopsis, poplar, eucalyptus, rice, corn, switchgrass, sorghum, millet, miscanthus, sugarcane, pine, alfalfa, wheat, soy, barley, turfgrass, tobacco, hemp, bamboo, rape, sunflower, willow, Jatropha, and Brachypodium.

In some embodiments, the plant into which the expression cassette is introduced is the same species of plant as the promoter and/or as the polynucleotide encoding lignin biosynthesis enzyme, xylan biosynthesis enzyme, or transcription factor (e.g., a vessel-specific promoter, lignin biosynthesis enzyme, xylan biosynthesis enzyme, and/or transcription factor from Arabidopsis is expressed in an Arabidopsis plant). In some embodiments, the plant into which the expression cassette is introduced is a different species of plant than the promoter and/or than the polynucleotide encoding lignin biosynthesis enzyme, xylan biosynthesis enzyme, or transcription factor (e.g., a vessel-specific promoter, lignin biosynthesis enzyme, xylan biosynthesis enzyme, and/or transcription factor from Arabidopsis is expressed in a poplar plant). See, e.g., McCarthy et al., Plant Cell Physiol. 51:1084-90 (2010); and Zhong et al., Plant Physiol. 152:1044-55 (2010).

H. Screening for Plants Having Modified Gene Expression

After transformed plants are selected, the plants or parts of the plants may be evaluated to determine whether the expression patterns of the gene or genes of interest have been modified, e.g., by evaluating the level of RNA or protein, by evaluating the lignin content, xylan content, and/or amount of secondary cell wall deposition in the plant or part of the plant, or by determining the amounts of soluble sugars that can be extracted from the plants. These analyses can be performed using any number of methods known in the art.

In some embodiments, plants are screened by evaluating the level of RNA or protein. Methods of measuring RNA expression are known in the art and include, for example, PCR, northern analysis, reverse-transcriptase polymerase chain reaction (RT-PCR), and microarrays. Methods of measuring protein levels are also known in the art and include, for example, mass spectroscopy or antibody-based techniques such as ELISA, Western blotting, flow cytometry, immunofluorescence, and immunohistochemistry.

In some embodiments, plants are screened by evaluating lignin content, xylan content, and/or amount of secondary cell wall deposition. Lignin content can be assessed, for example, by spectrophotometry, microscopy, klason lignin assays, acetyl-bromide reagent or by histochemical staining (e.g., with phloroglucinol). Xylan content can be assessed, for example, by immunohistochemistry (e.g., with LM10 monoclonal antibody). The amount of secondary cell wall deposition can be assessed, for example, by histochemical staining (e.g., phloroglucinol or Maule reagent) or enzymatic or chemical reaction (e.g., polysaccharide hydrolysis or TFA hydrolysis).

### IV. Methods of Using Plants Having Spatially Modified Gene Expression

Plants, parts of plants, or plant biomass material from plants having spatially modified gene expression of one of more of a lignin biosynthesis enzyme, xylan biosynthesis enzyme, and/or transcription factor that regulates secondary cell wall production can be used for a variety of methods. In some embodiments, the plants, parts of plants, or plant biomass material are used in a conversion reaction to generate an increased amount of bioenergy as compared to wild-type plants. For example, the plants, parts of plants, or plant biomass material can be used in a combustion reaction, gasification, pyrolysis, or polysaccharide hydrolysis (enzymatic or chemical). In some embodiments, the plants, parts of plants, or plant biomass material are used in a saccharification reaction, e.g., enzymatic saccharification, to generate an increased amount of soluble sugars as compared to wild-type plants. In some embodiments, the plants, parts of plants, or plant biomass material are used to increase biomass yield or simplify downstream processing for wood industries (such as paper, pulping, and construction) as compared to wild-type plants. In some embodiments, the plants, parts of plants, or plant biomass material are used to increase the quality of wood for construction purposes.

In some embodiments, the modification of cell wall (composition or content) are used to increase stem/stalk strength to reduce lodging of cereals (wheat, barley, corn . . . ) and seed loss.

Methods of conversion, for example biomass gasification, are known in the art. Briefly, in gasification plants or plant biomass material (e.g., leaves and stems) are ground into small particles and enter the gasifier along with a controlled amount of air or oxygen and steam. The heat and pressure of the reaction break apart the chemical bonds of the biomass, forming syngas, which is subsequently cleaned to remove impurities such as sulfur, mercury, particulates, and trace materials. Syngas can then be converted to products such as ethanol or other biofuels.

Methods of enzymatic saccharification are also known in the art. Briefly, plants or plant biomass material (e.g., leaves and stems) are optionally pre-treated with hot water or dilute acid, followed by enzymatic saccharification using a mixture of cellulose and beta-glucosidase in buffer and incubation of the plants or plant biomass material with the enzymatic mixture. Following incubation, the yield of the saccharification reaction can be readily determined by measuring the amount of reducing sugar released, using a standard method for sugar detection, e.g. the dinitrosalicylic acid method well known to those skilled in the art. Plants engineered in accordance with the invention provide a higher sugar yield as compared to wild-type plants.

### EXAMPLES

The following examples are provided to illustrate, but not limit the claimed invention.

### Example 1

### Reengineering Secondary Cell Wall Deposition in Plants

This study pooled two approaches for overcoming cell wall recalcitrance and filling up fiber cells with cell wall polymers without altering plant development. The first approach allowed the reduction of lignin except in the vessels, while the second approach increased cell wall deposition specifically in woody tissues. This strategy of combining approaches uses synthetic biology to fine-tune lignin biosynthesis and to create new feedback loops to reengineer the control of secondary cell wall deposition.

### Materials and Methods

### Construction of Plasmids

The protein-coding regions of the C4H (ref3) gene (AT2G30490), F5H (At4g36220), and CADc gene (AT3G19450) were amplified from Arabidopsis thaliana cDNA, and the 5′ upstream region of 2756 bp, which is from the initial site of translation for VND6 gene (At5g62380), was amplified as pVND6 from genomic DNA with appropriate primers (see Table 1).

TABLE 1
Primers used for plasmid construction and genotyping

| | |
|---|---|
| pVND6-F3- | 5′-cccgggtaccTCCTTTACGATGTTGTTATGGGTTA-3′ |
| KpnI | |
| pVND6-R3- | 5′-cccgactagtGTGTGCGAGACTTTGGATTTGATCTTTTTAATTTTA-3′ |
| SpeI | |
| FY100908- | 5′-ggggacaagtttgtacaaaaaagcaggcttcATGGACCTCCTCTTGCTGGA-3′ |
| C4h-GW-F | |
| FY100908- | 5′-ggggaccactttgtacaagaaagctgggtcACAGTTCCTTGGTTTCATAACG-3′ |
| C4h-GW-R | |
| DL-F5G3- | 5′- |
| At3g19450- | ggggacaagtttgtacaaaaaagcaggcttcATGGGAAGTGTAGAAGCAGGAGAA- |
| GW | 3′ |
| DL-R5G3- | 5′-ggggaccactttgtacaagaaagctgggtcGTTTGTAGTTGTTGCAGCCTCCTC-3′ |
| At3g19450- | |
| GW | |
| FY081508- | 5′-ggggacaagtttgtacaaaaaagcaggcttcATGGAGTCTT CTATATCACA A-3′ |
| F5h-l-GW-F | |
| FY081508- | 5′-ggggaccactttgtacaagaaagctgggtcAAGAGCACAGATGAGGCGCGT -3′ |
| F5h-l-GW-R | |
| ref3-2F1 | 5′-TTC CGT ATC ATG TTC GAT AG-3′ |
| ref3-2R1 | 5′-AAT GTC AAT TTC CCA AAA TC-3′ |
| per-pVND6F1 | 5′-CAAATTGCCACATTGCAGAA-3′ |
| per-REF3-R1 | 5′-CGACGAGATTACGGTGGTTGA-3′ |

The gateway fragment (Invitrogen) was introduced into pCAMBIA1390 and the VND6 promoter was cloned using KpnI-SpeI/AvrII sites, then the C4H and CADc genes were introduced into the expression vector through a gateway system to get the final expression vectors pCAMBIA1390-pVND6:C4H, pCAMBIA1390-pVND6:F5H, and pCAMBIA1390-pVND6:CADc.

### Plant Growth and Transformation

Arabidopsis plants were grown in soil at 22° C. with 8 hr of light daily (short-day condition) for 4-5 weeks and 16 hr of light daily (long-day condition) for 4-5 weeks. The expression vector pCAMBIA1390-pVND6:C4H, pCAMBIA1390-pVND6:F5H, or pCAMBIA1390-pVND6:CADc was introduced into Agrobacterium tumefaciens strain GV3101 by electroporation, and was used to transfer Arabidopsis f5h, cadc/d homozygote ref3-2 (c4h mutant) heterozygote, f5h homozygote and cadc/d homozygote mutant plants, respectively, using the floral dip method (Clough and Bent, 1998).

### Analysis of Genotype of Arabidopsis Plants

Seeds of ref3-2 heterozygote mutants were sowed, genomic DNA of the plants was extracted through the CTAB method, and genotypes were analyzed through PCR with primers ref3-2F1 and ref3-2R1 (see Table 1). PCR products were digested with HinfI. The expected PCR products are 188 bp and 106 bp fragments for wild type plants, and a 294 bp fragment for the ref3-2 homozygotes.

Transformants of pVND6:C4H were identified through PCR with primers per-pVND6F1 and per-REF3-R1. The PCR product is 238 bp for the transformants. The PCR reactions above were carried out by using DyNAzyme DNA polymerase (Finnzymes, USA).

RNA Isolation and cDNA Synthesis

Total RNA was isolated using an RNeasy Plant Mini Kit (Qiagen, Valencia, Calif.) from the leaves of Arabidopsis plants under short day condition for 4 weeks. cDNA was synthesized using the Transcriptor High Fidelity cDNA Synthesis Kit (Roche Applied Science, Indianapolis, Ind.).

### Microscopy Analysis

To investigate the lignin content and anatomy of the cells of the stem, transverse sections were prepared from the base of the stems of mutant, wild-type and transgenic lines (when the plants were 30-35 cm high for healthy plants, 15-20 cm for mutant plants). The stem base of mature plants was embedded in 7% agarose before sectioning to a thickness of 100 μm using a vibratome (Leica VT1000S). Sections were mounted in water and examined under bright field. Lignified cell walls were also visualized under UV illumination. Lignin is a UV absorber so lignified cell walls emitted blue autofluorescence under UV illumination. A 2% (w/v) solution of Phloroglucinol dissolved in a 2:1 mixture of ethanol and concentrated HCl was applied to the stem sections directly to detect all lignin (Adler, 1977). Stem sections were also stained with calcofluor, a specific dye for β-glucans such as cellulose to determine the general antomy of the cells (Mori, 1996). Fresh sections were immersed in 0.5% calcofluor for 5 minutes, followed by 2 water washes of 5 minutes each to remove any excess of unbound calcofluor. Sections were immediately observed using a fluorescent microscope (Leica DM4000B). Images were registered using a Leica DC500 camera.

### Preparation of Alcohol Insoluble Residue (AIR)

Stems of plants were collected, dried and grinded into powder, then alcohol insoluble residue (AIR) was prepared according to Goubet et al. (2009). Grinded powder of stem was mixed with 1 mL 95% Ethanol and incubated at 100° C. for 30 min. After centrifugation, the supernatant was removed and the pellets were washed with 1 mL 70% Ethanol for 2-3 times and dried completely.

### Lignin Measurement

5 mg of AIR samples were analyzed for lignin assay through acetyl bromide methods (Fukushima, 2004). The AIR samples were mixed with 200 uL acetone bromide solution (25% v/v acetyl bromide in glacial acetic acid) in 2 mL Eppendorf tube with screw lids, shaking at 600 rpm in 50° C. for 2 hrs, then diluted to total volume of 1 mL with acetic acid. After centrifugation, 100 uL of supernatant was transferred to a new tube and mixed with 500 uL acetic acid, 300 uL 0.3M sodium hydroxide, and 100 uL hydroxylamine hydrochloride, respectively, then diluted to total volume of 2 mL with acetic acid. 360 uL of the solution was transferred to UV specific 96-well plates (Greiner, Monroe, N.C.) and absorbance at 280 nm was read. The percentage of acetyl bromide soluble lignin (% ABSL) was calculated based on published extinction coefficients (Fukushima, 2004; Foster, 2010).

### Saccharification and DNS Assay

5 mg of AIR samples was pretreated with 170 uL of water, diluted alkaline (1% NaOH, 30 min at 30° C., 30 min at 100° C.) or diluted acid (1.2% H₂SO₄, 30 min at 30° C., 1 hr at 120° C.). HCl or NaOH was added for neutralization for the last pretreatments, then the samples were added with 8 uL 5 mg/mL tetracycline, 25 uL 1M citrate buffer pH 6.2, 2 uL of diluted enzyme mix (Novozyme enzymes NS50013 (cellulase) and NS50010 (beta-glucosidase), 1:10 and 1:100 dilutions in 0.1M citrate buffer pH 5.0, respectively), and diluted to a final volume of 500 ul with water. The samples were shaken at 850 rpm in 50° C. for 24 hr. After saccharification, sugar amounts were analyzed through DNS assay. Glucose of 0, 0.125, 0.25, 0.5, 0.75, 1 and 2 mg/mL in citrate buffer pH 5.0 were used as standards. DNS reagent was added to samples and standards, incubated in 95° C. for 10 min, then absorbance at 540 nm was read for the assay.

### Analysis of the Hemicellulose Compositions

Approximately 5 mg of AIR was hydrolyzed in 1 ml of 2 M TFA for 1 h at 120 C. TFA was removed by drying under vacuum. Monosaccharide composition was subsequently determined by HPAEC-PAD of hydrolyzed material using a PA20 column (Dionex, Sunnyvale, Calif.) as described previously (Obro, 2004; Christensen, 2010). Monosaccharide standards included L-Fuc, L-Rha, L-Ara, D-Gal, D-Glc, D-Xyl, D-GalA and D-GlcA, and were obtained from Sigma. For verification of the response factors, a standard calibration was performed before analysis of each batch of samples.

### Results

Characterization of Vessel Specific Promoter pVND6

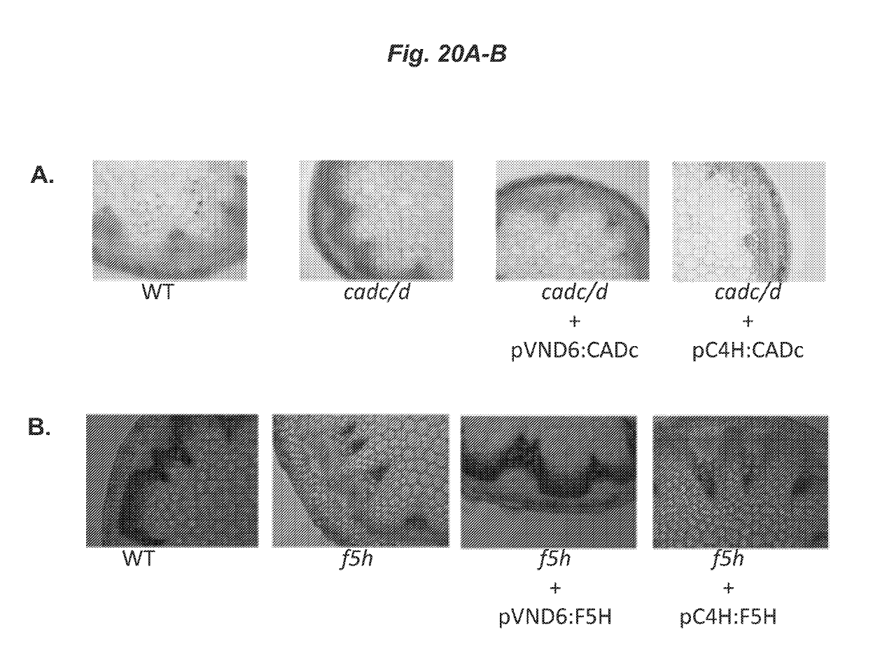

Integrity of the vessels is required for good development of plants due to the importance of the vessel tissue in transporting water and nutrients to photosynthetic organs. The VND-type transcription factors have been characterized as master regulators for vessel formation, suggesting they would have a vessel restricted expression pattern (Kubo et al., 2005). In order to correlate the spatiotemporal expression of these transcription factors with lignin biosynthesis, the promoter pVND6 was used to complement CAD mutants (described in Sibout et al., 2005) (FIG. 20A). The redness disappearance of xylem and the restoration of the vessel integrity were the acceptance criteria to use this promoter.

In order to compare the strength of the promoter pVND6 to promoter pC4H, both promoters were used to complement an f5h mutant (Meyer et al., 1998). The activity of the promoters was compared by measuring the amount of sinapyl alcohol unit incorporated into the lignin using Maule staining as a readout (FIG. 20B). Cross-sections of stems from the lines expressing the F5H gene under the VND6 promoter showed a much lower redness after Maule staining than the one with pC4H. These results indicate that the accumulation of sinapyl alcohol in the lignin in pVND6::F5H lines was due to a lower and more restricted F5H activity as compared to pC4H::F5H lines, a finding which is in agreement with the cadc/d complementation described above (FIG. 20A).

### Restriction of Lignin Biosynthesis

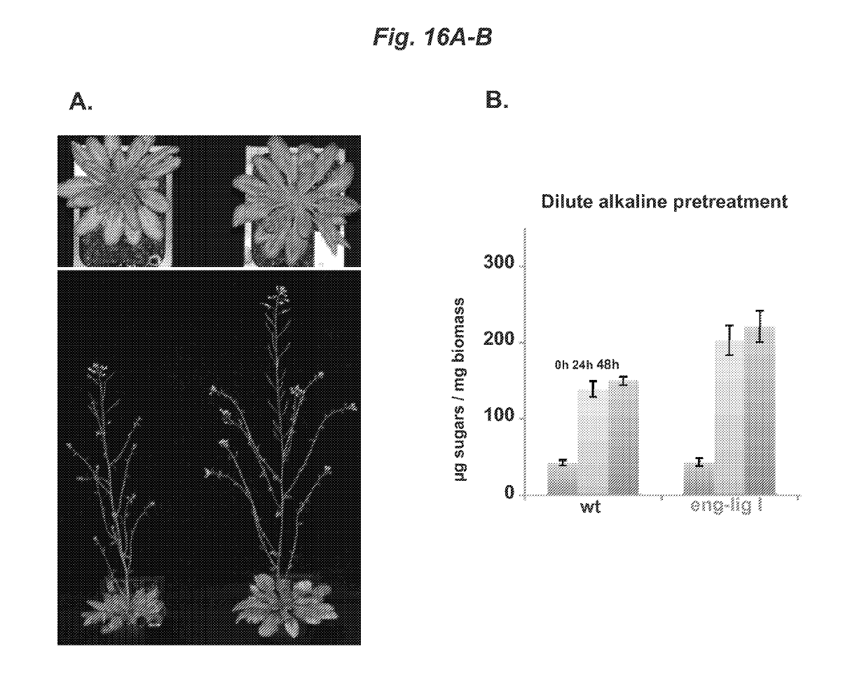

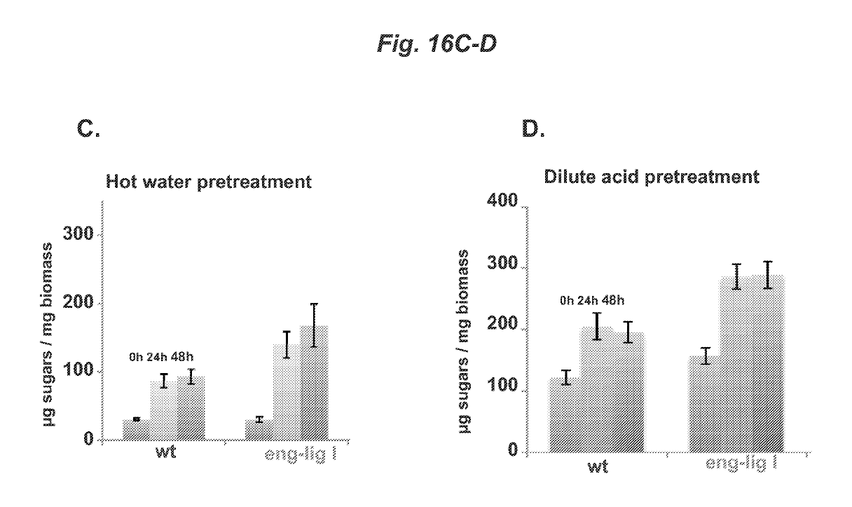

The lignin biosynthetic pathway is well characterized and loss of function of any of several genes of the lignin biosynthesis pathway results to deleterious growth effect and sterility. Therefore, controlling the expression of one of these genes should give the opportunity to control the production of monolignols. We selected the C4H gene, an early gene in the lignin biosynthesis pathway, as a target gene to control the flux of the pathway to produce the monolignols. In order to control the expression of C4H, we used the ref3-2 mutant (Schilmiller et al., 2009) and transformed the heterozygote line, due to the sterility, with a binary vector containing the pVND6::C4H gene construct. Transformants were selected and genotyped for the homozygosity of the ref3-2 allele. Interestingly, ref3-2 homozygotes harboring the pVND6::C4H fragment, called “EngSCW1g” (engineered secondary cell wall 1^{st} generation), did not show a growth difference as compared to Col0 wild-type plants grown at the same time. These transformed plants were able to generate a large rosette and a tall stem and were fertile (FIG. 16A). However, leaves from the transformed plants were purpled due to anthocyanin accumulation only in the vessel, in contrast to wild-type leaves that turned completely purpled under high light. This result demonstrates the restricted activity of the pVND6 promoter as compared to pC4H.

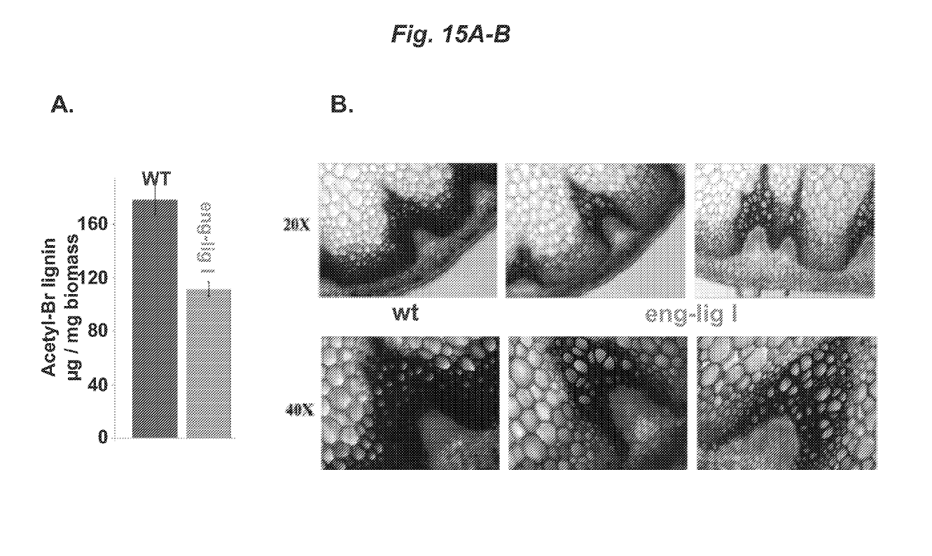

Analysis of lignin content of EngSCW1g plants via an acetyl-bromide method showed that lignin content in senesced stem approached approximately ⅔ of the lignin content of Col0 stem plants grown at the same time under the same conditions. In order to verify the lignin distribution in the stem, cross-sections of approximates 15-20 cm old stems were analyzed using phloroglucinol and Maule staining methods. Cross-sections of the engineered lines showed a reduction of lignin staining in the interfascicular fibers as compared to wild-type plants expressing the C4H gene under the control of its native C4H promoter. In contrast to the homozygote ref3-2 mutant, xylem tissues of the EngCW1g plants exhibited strong phloroglucinol staining and no vessel collapse, similar to the wild-type plants (FIG. 15B and FIG. 21).

### Increase of Cell Wall Deposition

The transcriptional network controlling secondary cell wall deposition in vessels and fibers has already been well investigated. Secondary cell wall deposition is controlled by two independent networks, although these two networks lead to the activation of the same groups of downstream secondary wall biosynthetic genes to regulate the synthesis of cellulose, hemicellulose and lignin. Several groups have showed that overexpressing a secondary cell wall transcription factor with the constitutively active 35S promoter in Arabidopsis generates ectopic secondary cell wall and lignification everywhere, including in elongating cells and photosynthetic tissues, which as a result inhibits plant growth (Zhong et al., 2008; Mitsuda et al., 2005; Goicoechea et al., 2005). Interestingly, even with restrained development, the plants exhibited enhanced secondary cell wall thickness in fiber cells (Zhong et al., 2008), suggesting that increasing the expression of a secondary cell wall transcription factor could be a route to increase cell wall deposition (and therefore increase biomass density).

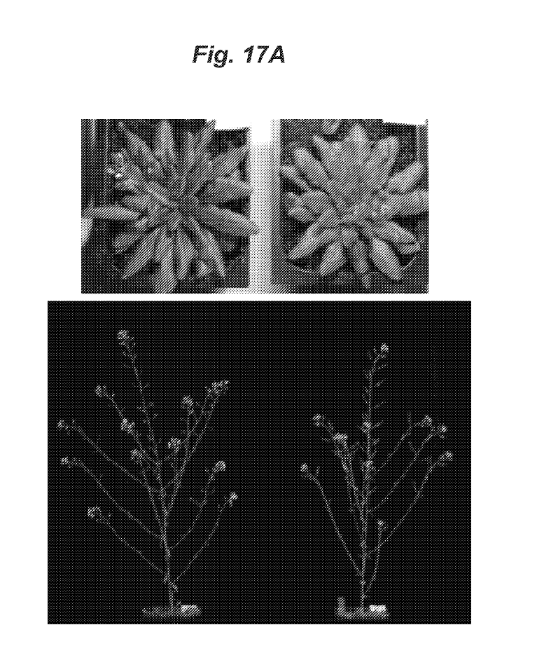

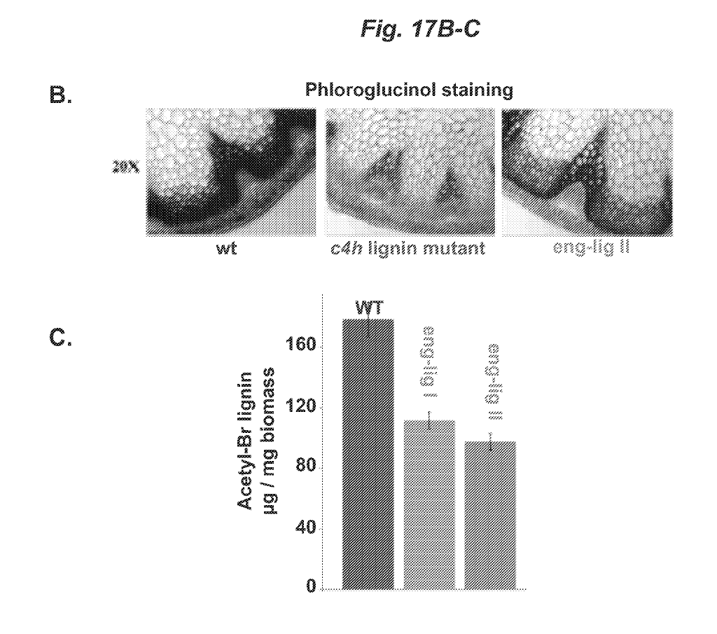

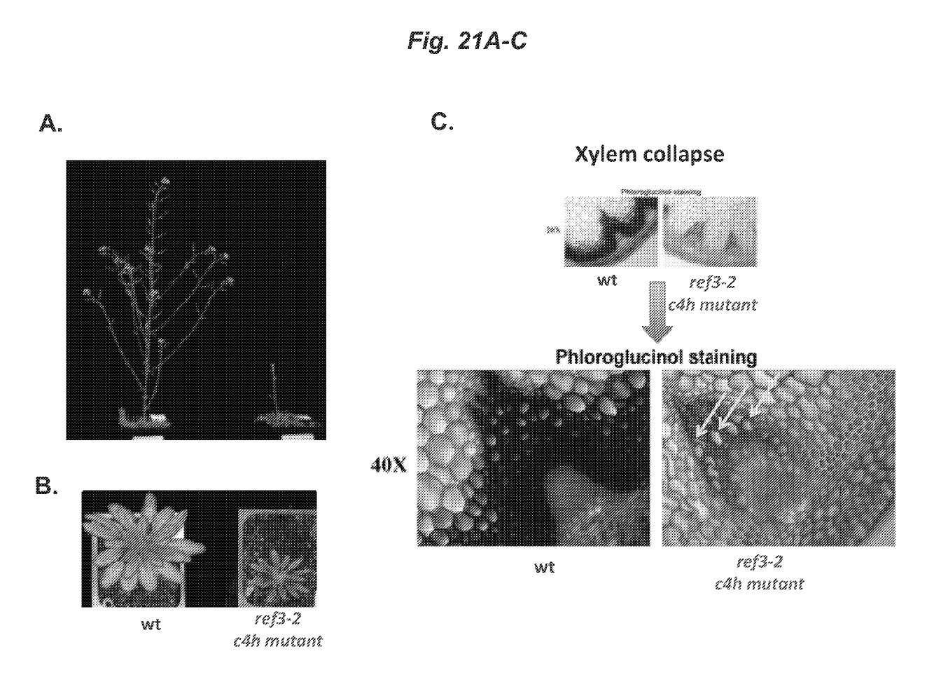

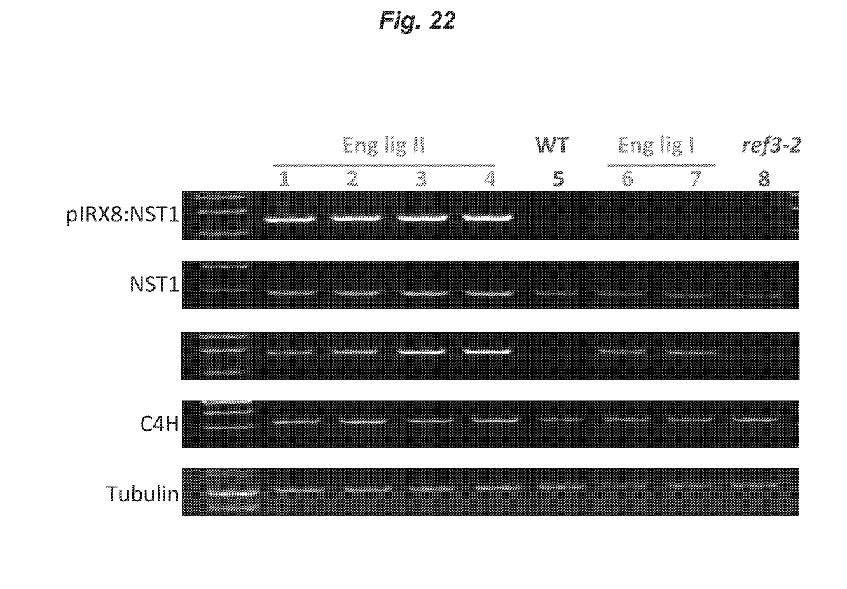

Accordingly, we overexpressed NST1 cDNA in the EngCW1g plant with an IRX8 promoter. Because IRX8 is a gene that is downstream of (i.e., under the control of) the NST1 transcription factor (Mitsuda et al., 2005; Zhong et al., 2010), this pIRX8::NST1 construct creates a positive feedback loop for overexpressing NST1 cDNA only in secondary cell wall tissues. EngCW1g plants were chosen for the transformation because the VND6 promoter is not a downstream target of NST1, and therefore the lignin biosynthesis under the control of pVND6 in EngCW1g should be disconnected from NST1 regulation. The generated plants, which were called “EngSCW2g” (engineered secondary cell wall 2^{nd} generation), did not exhibit a growth difference when compared to Col0 and EngSCW1 g plants grown at the same time. The EngSCW2g plants were able to generate a large rosette and tall stem and were fertile (FIG. 17A). Like EngSCW1g plants, leaves from the EngSCW2g lines were purpled due to anthocyanin accumulation only in the vessel, in contrast to wild-type leaves that turned completely purpled under high light. The verification of the expression of both NST1 genes (native and cDNA) was verified by semi-quantitative PCR and revealed that the native NST1 was expressed at the same level in wild-type, EngSCW1g, and EngSCW2g lines. However, only in the EngSCW2g lines was the expression of the new NST1 copy detected, resulting in a higher general expression level of the NST1 gene (native and cDNA) in the stem (FIG. 22).

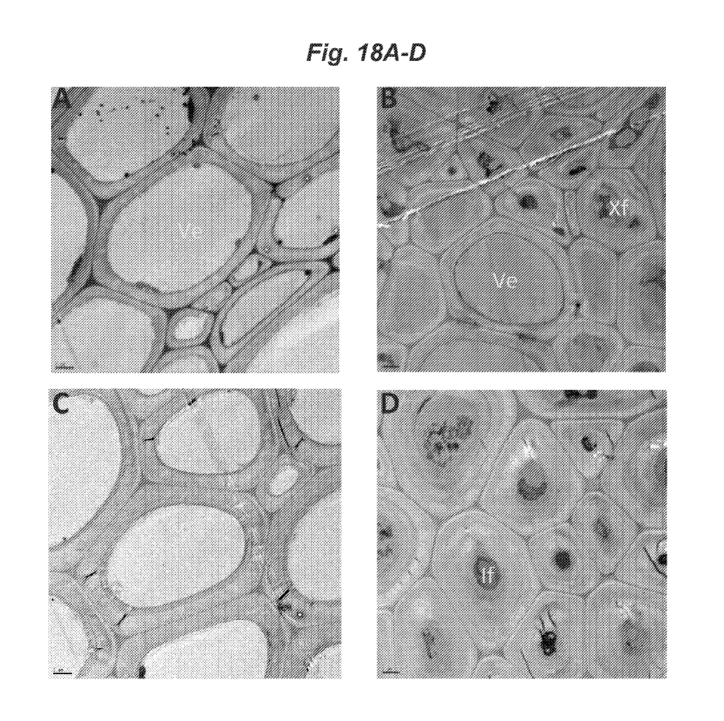

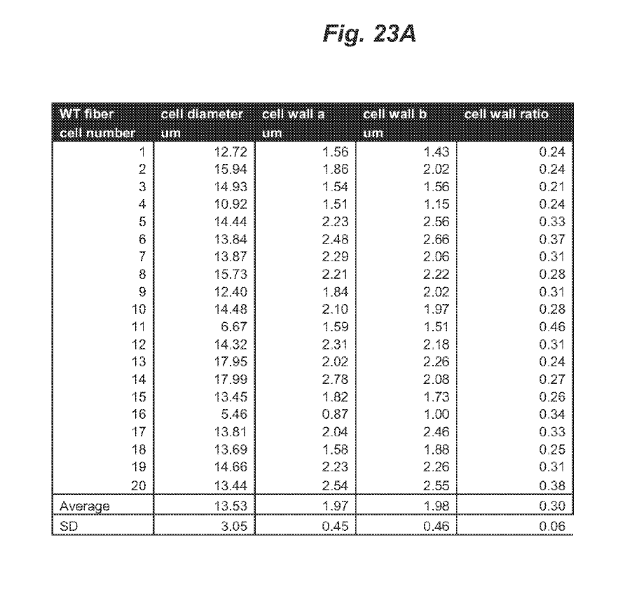

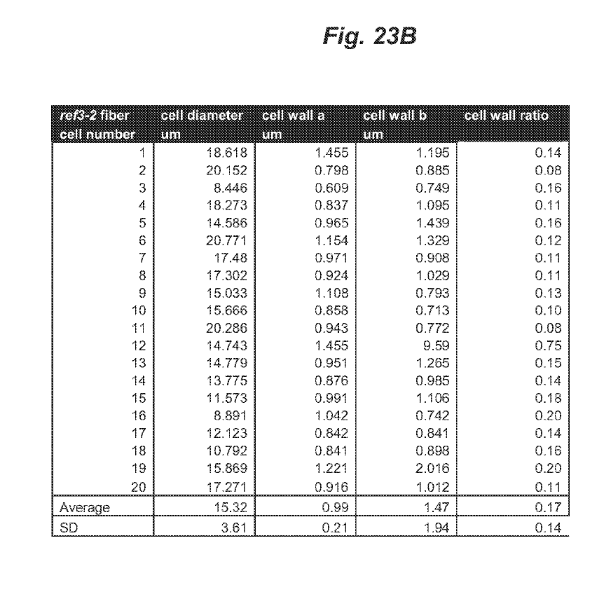

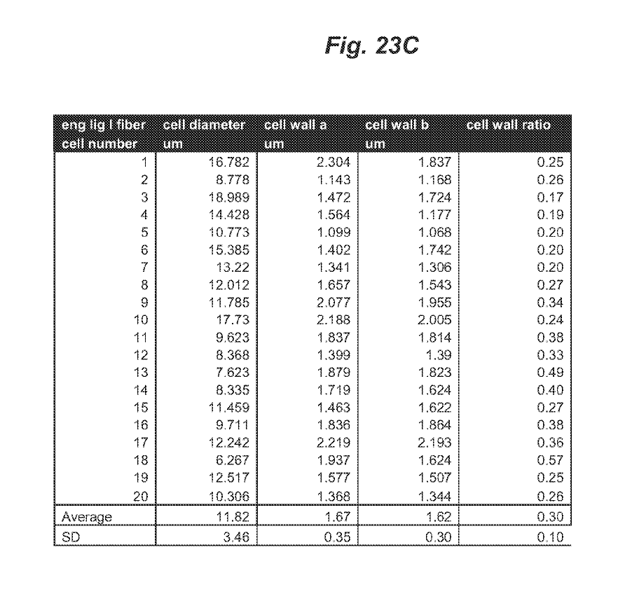

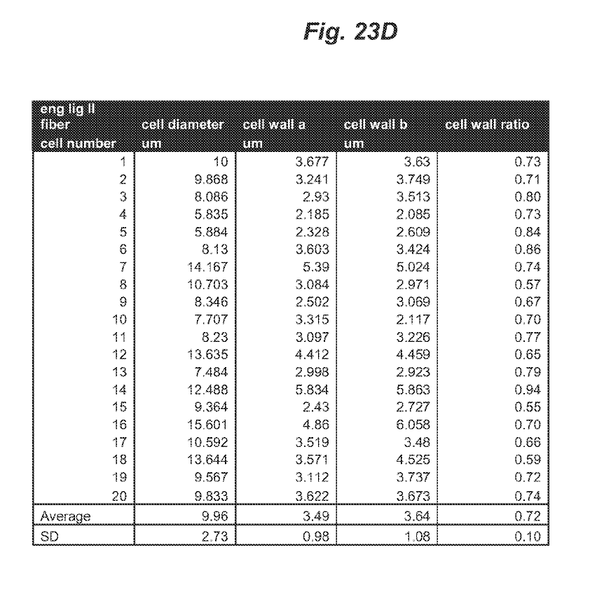

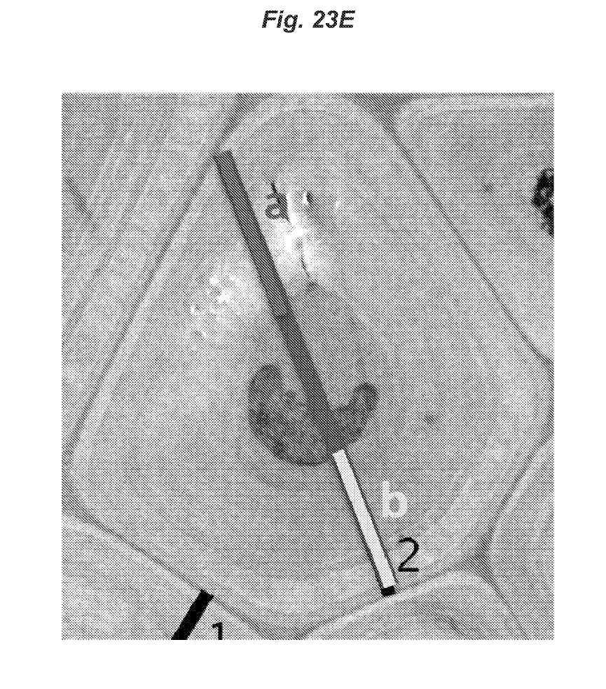

In order to verify the effect of NST1 overexpression on cell wall deposition in stems, lignin distribution in the stem cross-sections of old stems was analyzed using a phloroglucinol staining method. Cross-sections of the EngSCW2g lines still showed a reduction of lignin staining in the interfascicular fibers as compared to wild-type, while xylem tissues exhibited strong phloroglucinol staining and no vessel collapse, similar to wild-type and EngSCW1g lines (FIGS. 15B and 17B). Cell wall thickening was analyzed via transmission electron microscopy (TEM) on cross-sections from the base of xxx cm old stems. Intense thickening of cell wall in EngSCW2g lines compared to wild-type was observed in fiber cells from interfascicular fibers and xylem but not in vessel (FIGS. 18 and 23), which is in agreement with the overexpression of the NST transcription factors (Zhong et al., 2008). In wild-type stem cross-sections, the usual 4 distinct layers (S1, S2 and S3 and the middle lamella) were observed, in contrast to EngSCW2g lines where additional layers with different intensity were observed, which almost fill up the entire cell space.

### Fine-Tuning Secondary Cell Wall Deposition for Bioenergy

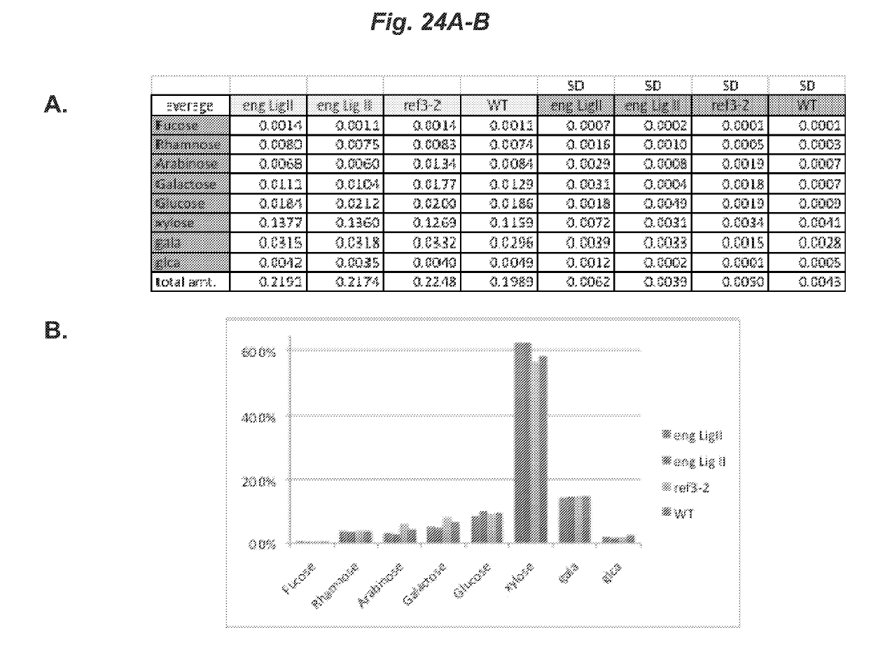

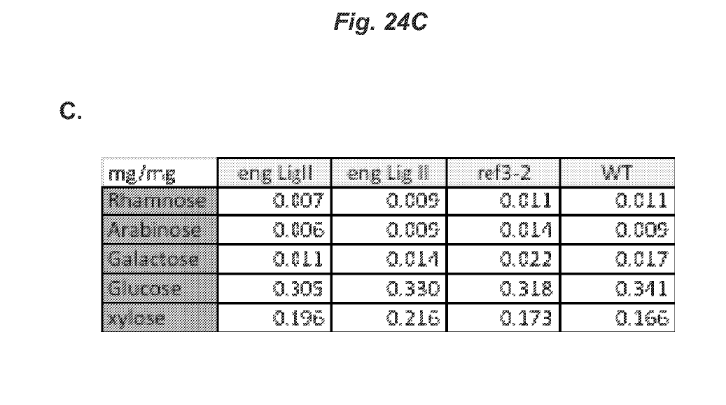

Analyses of cell wall cross-sections from EngSCW2g plants with gold-labeled CBM revealed that the extra cell wall layers contained cellulose, suggesting that the amount of cellulose had been increased. In order to verify an enhancement of cellulose, a complete polysaccharide hydrolysis was performed using H₂SO₄ (Suilter et al 2008, Technical report NREL/TP-510-4218) on senesced stems from EngSCW2g. The amount of glucose and other sugar released from the stem cell wall was similar among wild type, EngSCW1g, and EngSCW2g lines. The amount of xylose and glucuronic acid was also increased, suggesting that hemicellulose deposition was also enhanced in these plants. The composition analysis of hemicellulose by trifluoroacetic acid (TFA) hydrolysis of mature stems from the EngSCW1g and EngSCW2g lines did not exhibit major differences as compared to wild-type plants grown at the same time (FIG. 24).

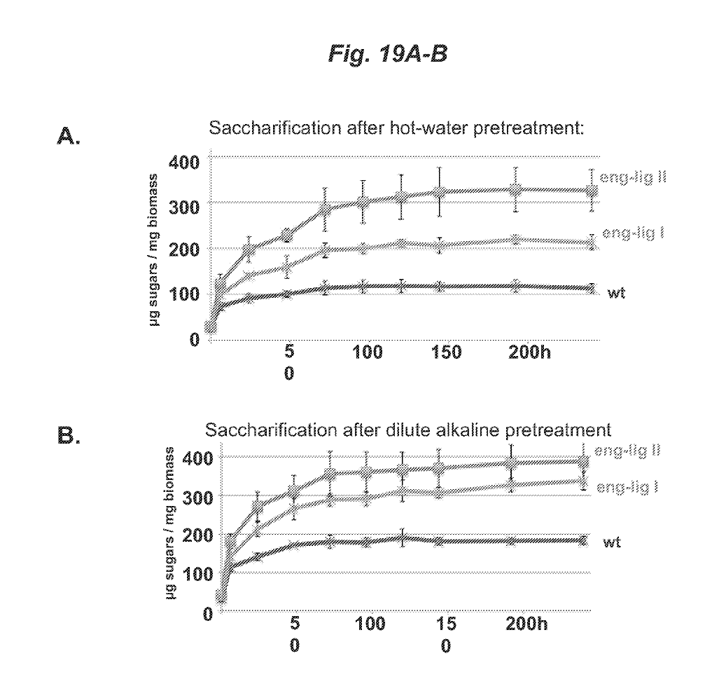

To analyze the saccharification efficiency of the EngSCW2g lines, 5 mg of ball-milled stems from EngSCW2g lines were subjected to two different mild pre-treatments, hot-water and dilute alkaline, followed by a saccharification kinetic. After each of the pre-treatments, glucose was released from the stem in presence of a cellulase cocktail much faster, and 2 to 3 times higher for EngSCW2g plants than for the control plants, when alkaline and hot-water pretreatments respectively were performed prior to a 120 hr saccharification (FIG. 19A-B).

Saccharification improvement was also observed with the EngSCW1g lines; for those plants, sugar hydrolyzed in the presence of the same amount of cellulase after hot water or dilute alkaline pre-treatments was 2.3 and 1.5 fold better than a control plant after hot water or dilute alkaline pre-treatment, respectively. The overexpression of the NST1 transcription factor in EngSCW2g lines increased cell wall deposition but did not reduce saccharification efficiency, which translated into an higher amount of glucose released by this line due to the increased polysaccharide content as compared to the parental EngSCW1g line.

Analysis of Additional Ref3-2 Mutant Plants that are Modified to Express C4H

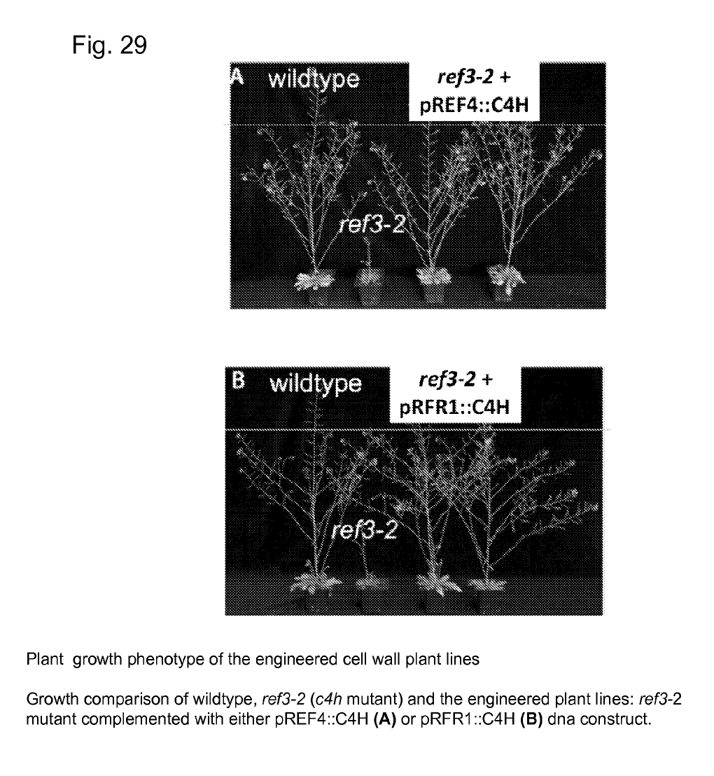

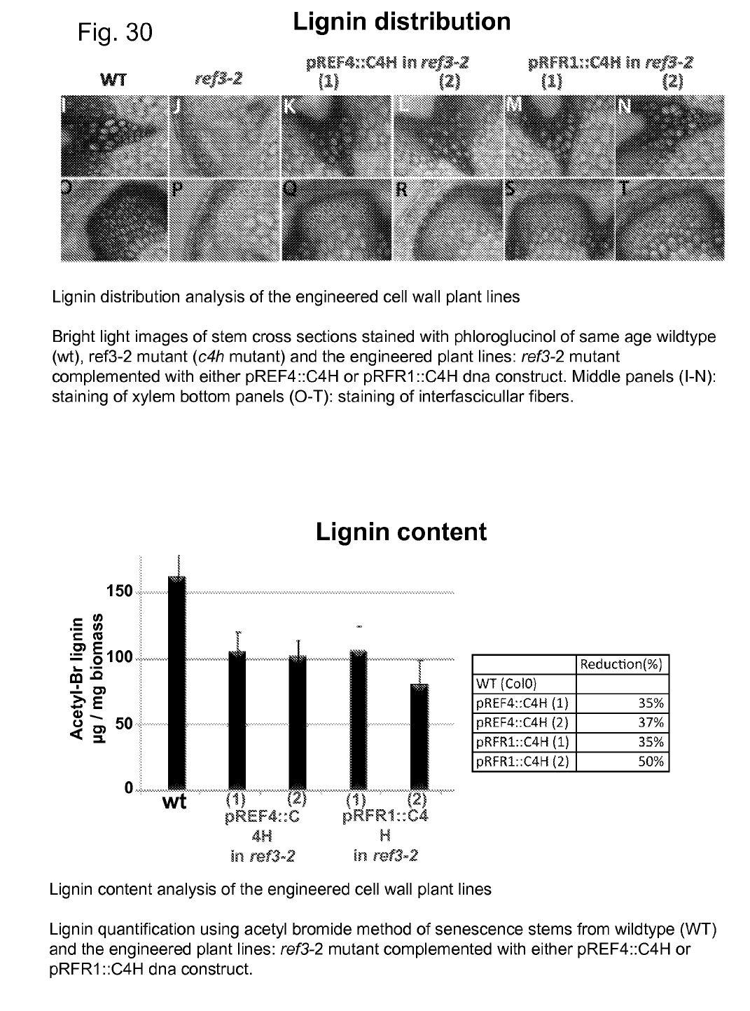

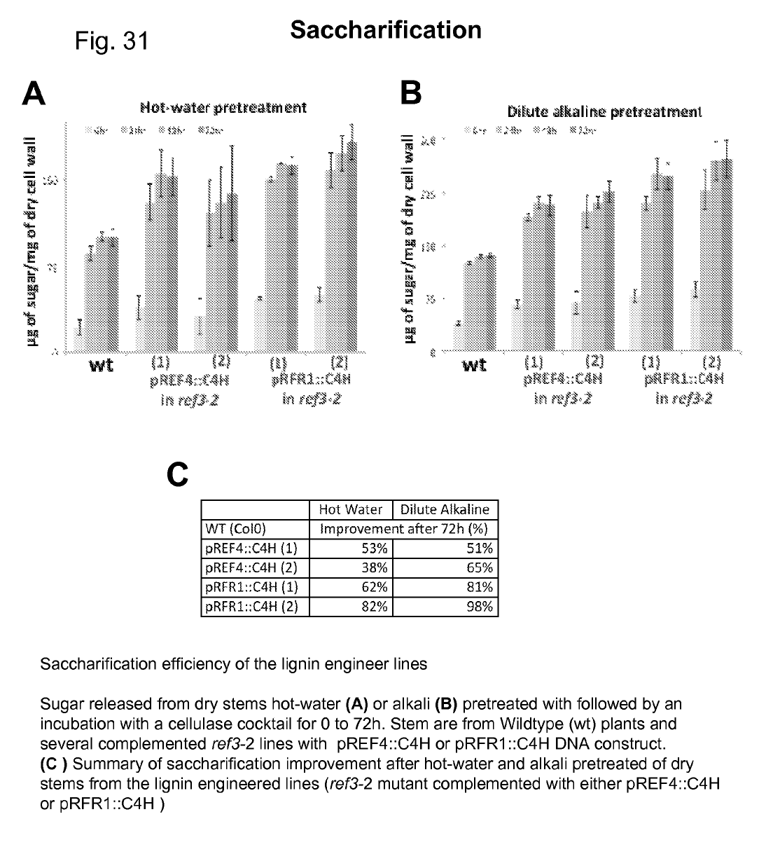

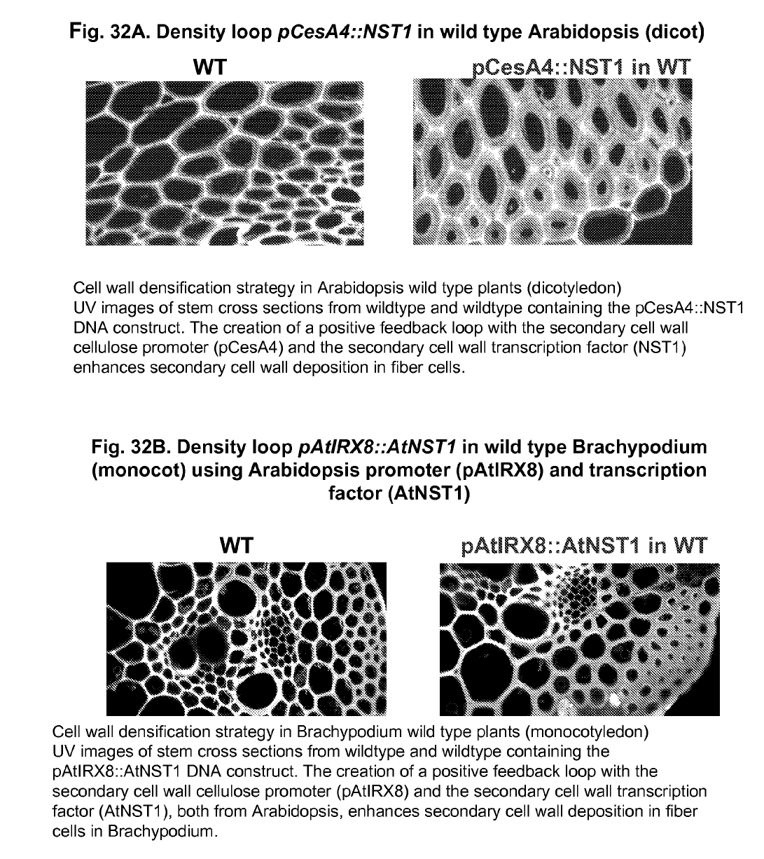

Ref3-2 mutant plants were also engineered to express C4H using either promoter pREF4 or pRFR1. Mutant plants were modified to contain either pREF4::C4H or prFR1::C4H to express C4H. Plant growth and phenotype of engineered cell wall plant lines were analyzed. FIG. 29 shows photographs of the plants. Growth was restored in mutant plants transformed with either construct. Lignin distribution in the plants is shown in FIG. 30. The results show that lignin was produce in the vessels, but reduced in fibers in the engineered plants, which resulted in >35% reduction of the total lignin compared to wild type plants without affecting plant growth. FIG. 31 provides data showing the saccharification efficient of the engineered lines. These results show that the reduction of lignin in fibers greatly improved saccharification efficiency. Therefore, these results demonstrate that both promoters pREF4 and pRFR1 can be used to engineer plants with low lignin similarly to the “EngSCW1g” plants (ref3-2 complemented with pVND6::C4H construct) and be used as genetic background for the secondary cell wall positive feed back loop.

### Example 2

### Positive Feedback Loops Engineered in Arabidopsis (Dicot) and Brachypodium (Monocot)

FIG. 27 illustrates a cell wall deposition positive feed back loop. Cell wall densification is based on the creation of an artificial positive feedback loop to enhance the expression of fiber-specific transcription factor. It is created by the expression of a new copy of a fiber specific transcription factor (e.g., NST1) under the control of a downstream-induced promoter from xylan or cellulose biosynthesis. This approach is compatible with xylan and lignin engineering strategies.

FIG. 31A shows UV images of stem cross-sections from wildtype Arabidopsis (dicotyledon) and wildtype Arabidopsis genetically modified to contain a pCesA4::NST1 expression construct. The creation of a positive feedback loop with the secondary cell wall cellulose promoter (pCesA4) and the secondary cell wall transcription factor (NST1) enhanced secondary cell wall deposition in fiber cells.

FIG. 31B shows UV images of stem cross-sections from wildtype Brachypodium (monocotyledon) and wildtype Brachypodium genetically modified to contain a pAtIRX8::AtNST1 expression construct. The creation of a positive feedback loop with the secondary cell wall cellulose promoter (pAtIRX8) and the secondary cell wall transcription factor (AtNST1) enhanced secondary cell wall deposition in Brachypodium.

This example demonstrated that this pathway is conserved in both monocots and dicots and that positive feedback loop could be generated to enhance secondary cell wall deposition.

### Example 3

### Engineering a Xylan Biosynthesis Enzyme

Arabidopsis mutants irx7-1 (At2g28110, salk_{—}120296), irx8-1 (At5g54690, salk_{—}008642), irx9-1 (At2g37090, salk_{—}058238), irx9-2 (salk_{—}057033C), parvus (At1g19300, CS16279) were obtained from Arabidopsis Biological Resource Center. The wild-type IRX7, IRX8, IRX9, and PARVUS genes were cloned into Gateway entry clones and recombined into Gateway destination vectors with the pVND6 or pVND7 promoters as described above for the lignin biosynthesis genes.

The expression vector pCAMBIA1390-pVND6:IRX7, pCAMBIA1390-pVND7:IRX7, pCAMBIA1390-pVND6:IRX8, pCAMBIA1390-pVND7:IRX8, pCAMBIA1390-pVND6:IRX9, pCAMBIA1390-pVND7:IRX9, pCAMBIA1390-pVND6:PARVUS, pCAMBIA1390-pVND7:PARVUS were introduced into Agrobacterium tumefaciens strain GV3101 by electroporation. Constructs expressing IRX7, IRX8, IRX9, and PARVUS were used to transform Arabidopsis heterozygote mutant plants (irx7-1, irx8-1, irx9-1 and parvus, respectively) using the floral dip method (Clough and Bent, 1998). Constructs expressing IRX9 were also used to transform homozygous mutants of irx9-2.

Seeds of the transformed irx7, irx8, parvus, irx9-1, and irx9-2 plants were planted on growth medium supplemented with hygromycin. Hydromycin resistant plants were recovered and transferred to soil. The plants showed a healthy growth phenotype unlike the untransformed homozygous mutants, which were clearly affected in growth.

Transformed irx7, irx8, irx9-2, parvus, and irx9-1 mutants were selected. The recovered, transformed mutants were characterized by PCR to ensure their homozygous phenotype with respect to the original mutations, and to ensure the presence of the pVND6 or pVND7 driven transgenes. The growth of the plants was compared with that of wild type and homozygous mutants, and their content of xylan determined by sugar composition analysis of inflorescence stems. Lignin was determined by acetyl bromide method. The localization of xylan deposition was determined by immunofluorescence microscopy using LM10 antibody and deposition of lignin by microscopy and determination of autofluorescence under UV illumination and Phloroglucinol staining. Saccharification was determined as described above.

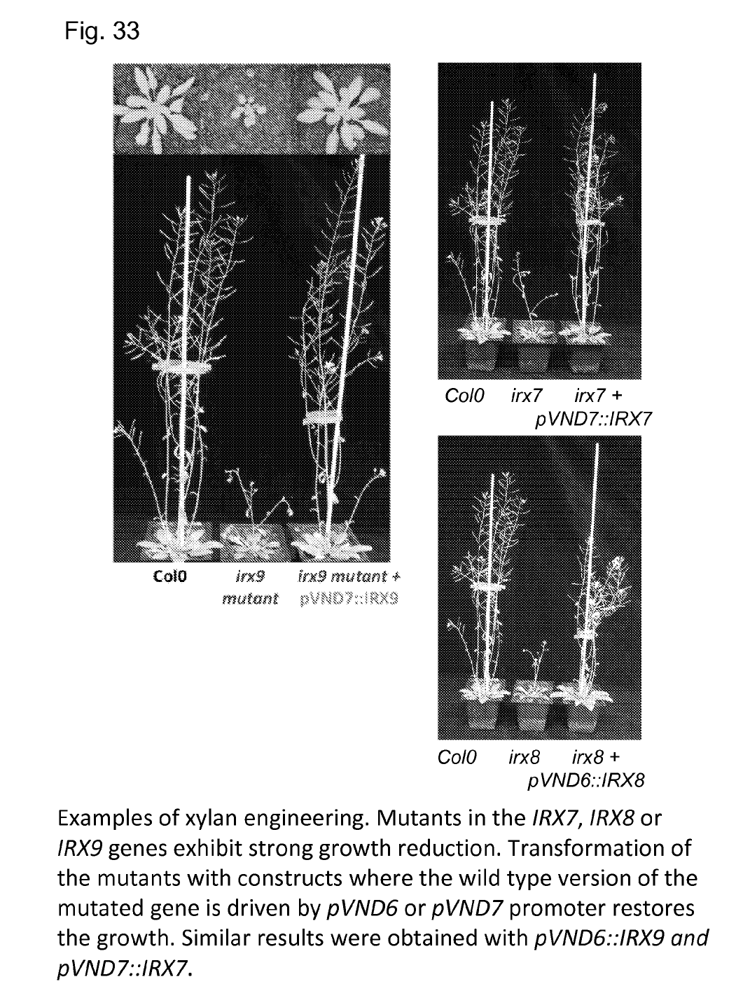

FIG. 33 provides data demonstrating that mutants in the IRX7, IRX8 or IRX9 genes exhibited strong growth reduction. Transformation of the mutants with constructs where the wild type version of the mutated gene was driven by pVND6 or pVND7 promoter restored the growth. Similar results were obtained with pVND6::IRX9 and pVND7::IRX7.

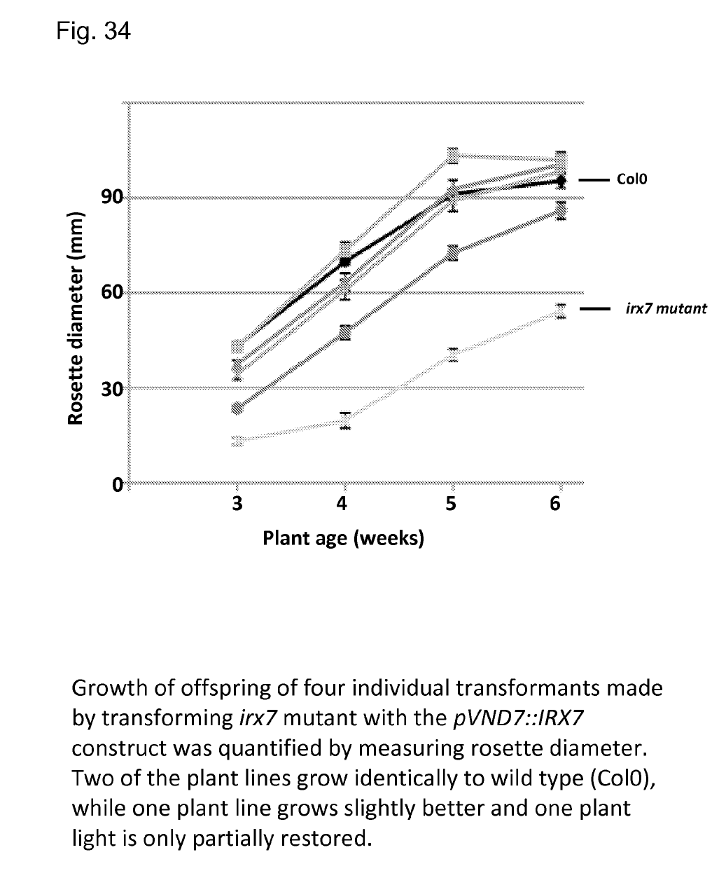

FIG. 34 provides data showing growth of offspring of four individual transformants made by transforming irx7 mutant with the pVND7::IRX7 construct. Growth was quantified by measuring rosette diameter. Two of the plant lines grew identically to wild type (Col0), while one plant line grew slightly better than the wildtype plant and for one plant, growth was only partially restored.

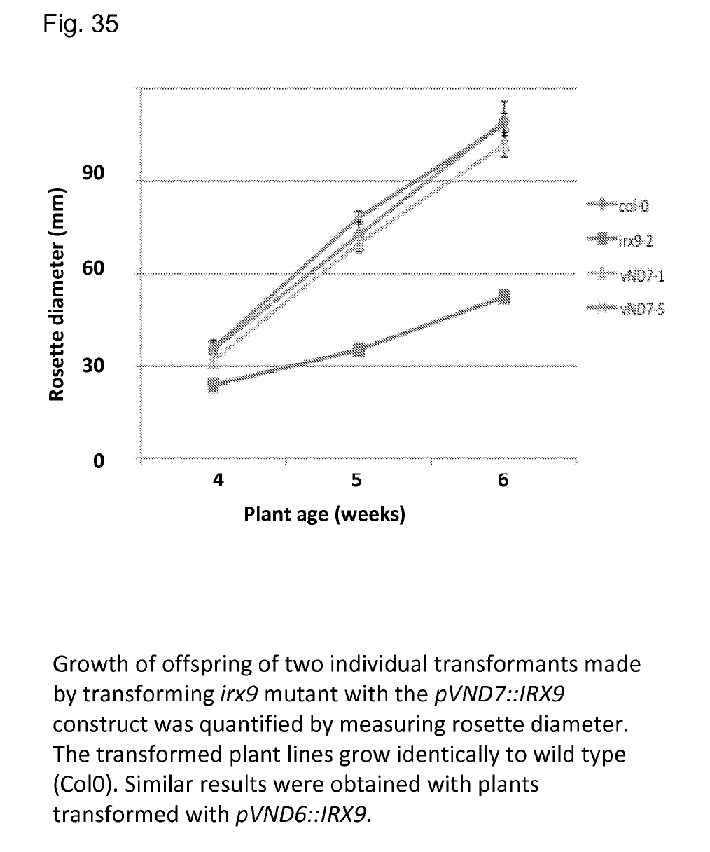

FIG. 35 provides data showing growth of offspring of two individual transformants made by transforming irx9 mutant with the pVND7::IRX9 construct. Growth was quantified by measuring rosette diameter. The transformed plant lines grew identically to wild type (Col0). Similar results were obtained with plants transformed with pVND6::IRX9.

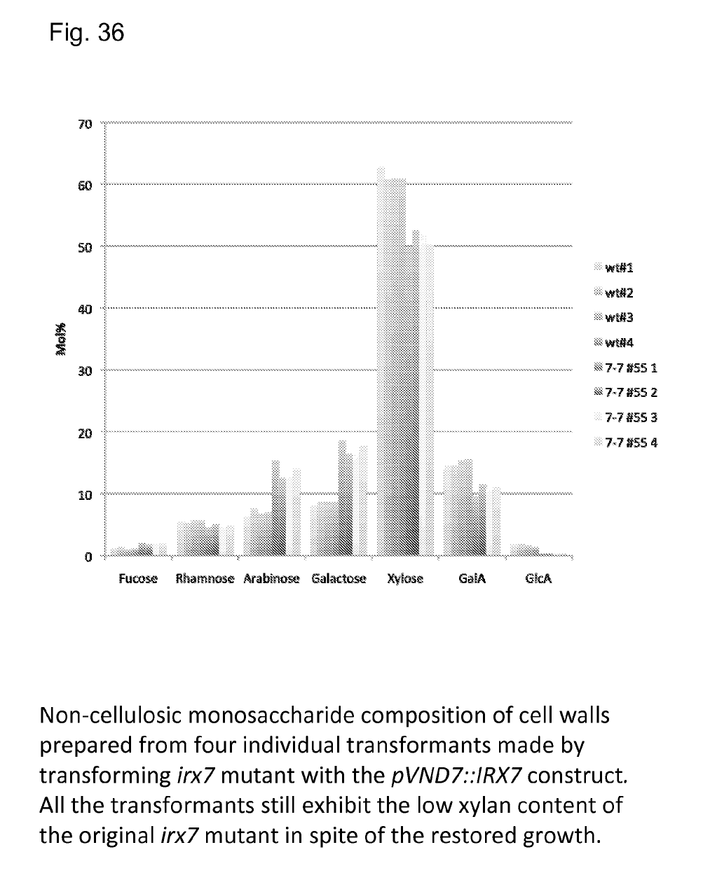

FIG. 36 provides data showing an analysis of non-cellulosic monosaccharide composition of cell walls prepared from four individual transformants made by transforming irx7 mutant with the pVND7::IRX7 construct. All the transformants still exhibited the low xylan content of the original irx7 mutant in spite of the restored growth.

FIG. 37 provides data showing an analysis of non-cellulosic monosaccharide composition of cell walls prepared from offspring of four individual transformants made by transforming irx8 mutant with the pVND6::IRX8 construct. All the transformants still exhibited the low xylan content of the original irx8 mutant in spite of the restored growth.

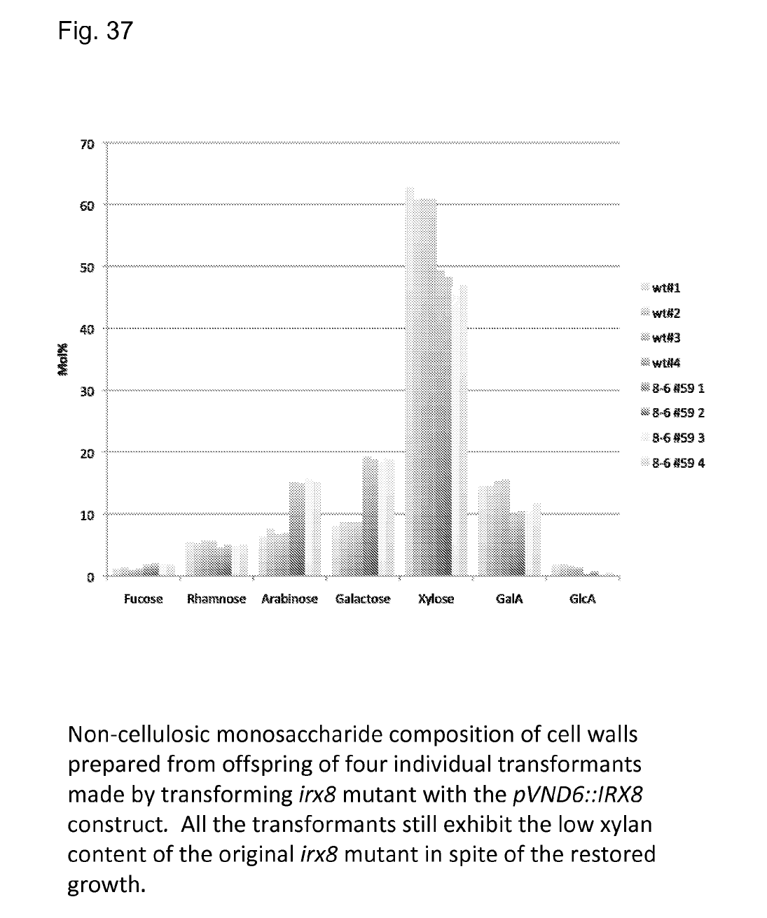

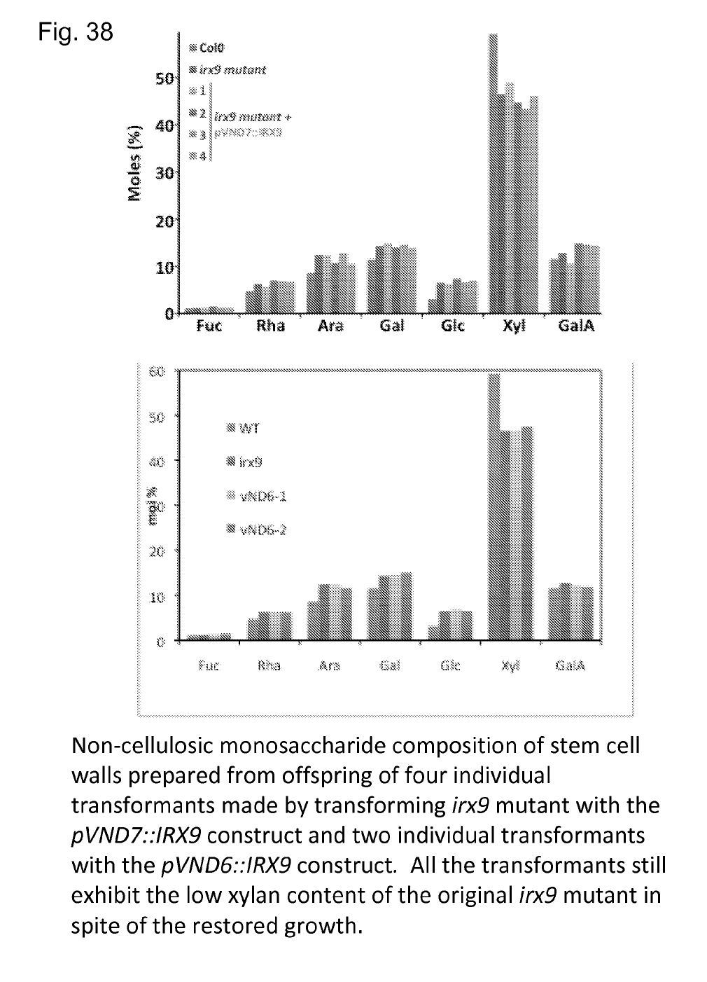

FIG. 38 provides data showing an analysis of non-cellulosic monosaccharide composition of stem cell walls prepared from offspring of four individual transformants made by transforming irx9 mutant with the pVND7::IRX9 construct and two individual transformants with the pVND6::IRX9 construct. All the transformants still exhibited the low xylan content of the original irx9 mutant in spite of the restored growth.

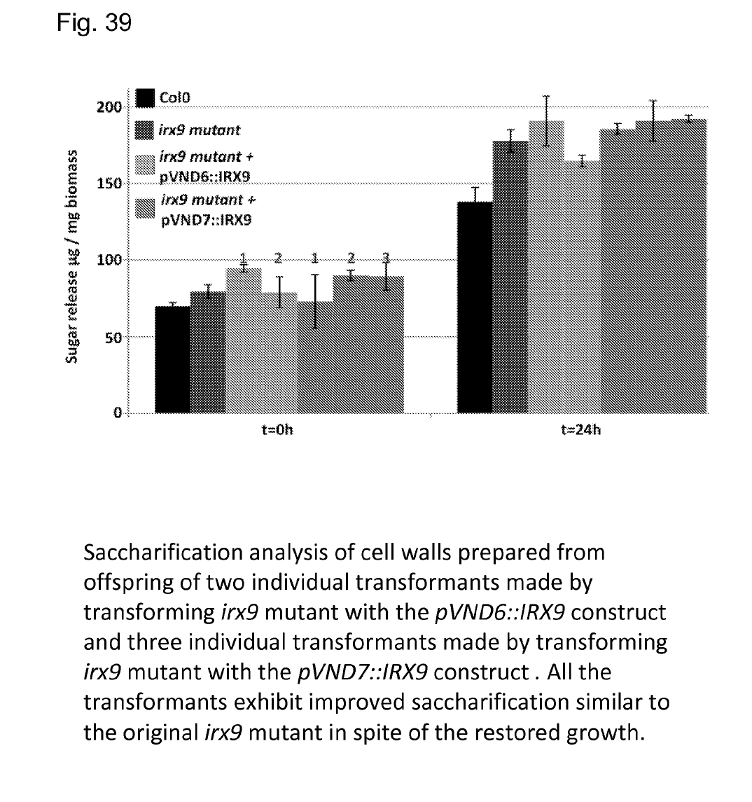

FIG. 39 provides data showing a saccharification analysis of cell walls prepared from offspring of two individual transformants made by transforming irx9 mutant with the pVND6::IRX9 construct and three individual transformants made by transforming irx9 mutant with the pVND7::IRX9 construct. All the transformants exhibited improved saccharification similar to the original irx9 mutant in spite of the restored growth.

### Example 4

### Generation of Wax-APFL in Epidemic Cells and Conservation Across Species

Waxes are highly energetic and contain large amounts of long chain alkanes and fatty acids that have potential fuel applications. Therefore, using the wax-APFL to generate plants capable to produce and accumulate large amount of waxes in non-essential tissues such as pith and fiber in stems offer new opportunities generate bioenergy crops with high energy density that are also water use efficient.

FIG. 28 illustrates an artificial positive feed back loop for wax deposition.

This example employed Arabidopsis as a model plant to develop the wax-APFL to increase wax biosynthesis and accumulation in epidermis cells. Eight DNA constructs were designed to create a wax AFPL in epidermal cells, which produce some wax. These constructs were generated by using pAtCER1 or pAtWBC11 as promoters to express AtSHN1 (NP_{—}172988) from Arabidopsis and selected homologs OsSHN1 (NP_{—}001046226), BdSHN1 (XP_{—}003563662) or SmSHN1 (XP_{—}002969836) from Rice, Brachypodium and Selaginella respectively. All constructs were transferred individually to wildtype Arabidopsis using Agrobacterium transformation. For each wax-APFL, several transgenic plants were recovered.

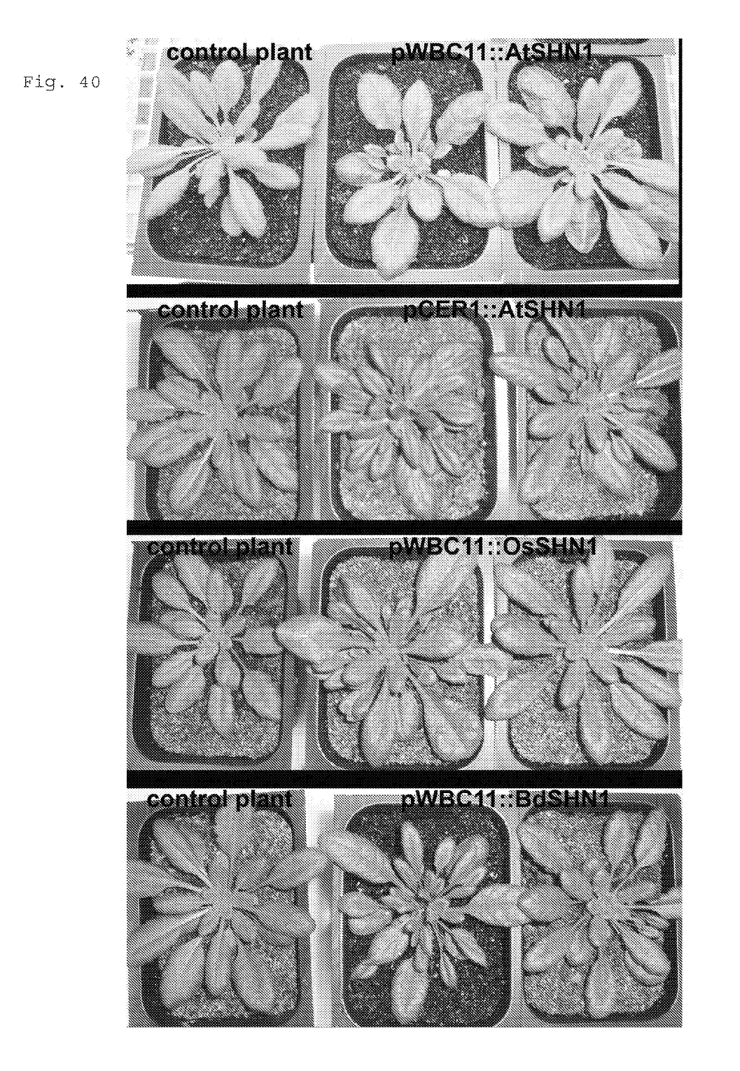

In Arabidopsis, as in many plant species, wax biosynthesis occurs principally in epidermic cells from leaves and stems. It has also been reported by several studies that plants over-expressing SHN genes using constitutive or chemically induced-promoters resulted in shiny phenotype of the leaves or/and stem surfaces, which was attributed to modifications of wax deposition or/and composition (McNevin et al 1993; Broun et al 2004; Kannangara et al 2007; Shi et al. 2011). Visual analysis of the Arabidopsis plant transformed with the different constructs showed increased shininess of the leaves (FIG. 40).

Additional analyses are performed on homozygous lines, including composition analysis of leave and stem epidermal waxes. Plant development, additional assessment of shininess of leaf epidermis, chlorophyll leaching assays, wax accumulation and composition analysis, gene expression analysis and biological impact on drought stress and water losses are the primary criteria used to characterize the wax-APFL in plants. The chlorophyll leaching assays is a general assay to indentify modification of the cuticle permeability to ethanol and is performed by monitoring the chlorophyll extraction on intact leaves in presence of ethanol (Aharoni et al., Plant Cell 2004 supra; Seo et al, Plant Cell 2011, supra). Epicuticular wax accumulation and composition are analyzed after being extracted by short immersing of whole leaf or stem into chloroform containing some n-triacontane as standard. The general composition the extracts are pre-analyzed by TLC plates using hexane:ethyl-ether:acetic-acid at 90:7.5:1 solvent system and derivatized with N,Obis(trimethylsilyl)trifluoroacetamide):trimethylchlorosilane at 99:1 for GC/MS analysis (Aharoni et al. Plant Cell, 2004, supra; Kannangara et al., Plant Cell, 2007, supra). In order to evaluate the impact of enhanced wax deposition on plant water use efficiency, water loss assays are performed on detached leaves by monitoring weight losses. Finally, the impact of wax deposition modification on plant drought stress tolerance are performed by plant survival counts of 5-6 weeks old plants after 7-15 days dehydration period followed by 1 week of watering recovery period.

### Discussion

Modifying lignin content has always been a challenge in crops or trees, since the more severe the reduction is, the more biomass yields are affected. This reduction is also often associated with a loss of integrity of the vessel tissues that are responsible for water and nutrient transport and distribution from the root into the aboveground organs. Lignin is one of the main inhibitory factors for efficient enzymatic hydrolysis of plant cell wall polysaccharides. Therefore, our strategy focused on reducing lignin in most tissues except vessels (in order to maintain vessel integrity) and on the disconnection of lignin biosynthesis from key secondary cell wall transcription factor switches in order to manipulate the expression of the transcription factors without affecting lignin deposition.

Our strategy to reengineer secondary cell wall biosynthesis demonstrated that we can reduce the lignin content and increase cell wall thickening in woody tissues without altering plant growth. Replacing the promoter of a gene controlling an essential step in the lignin biosynthesis by another one with a more restricted spatiotemporal expression profile gives better control of the lignin deposition that silencing approaches alone. This fine-tuning avoids the reduction of lignin deposition in every tissue and allows keeping it in essential tissues such as vessels, in contrast to silencing approaches that affect every tissue and therefore limit the power of such a strategy. The use of the pVND6 promoter to control the activity of C4H allowed a partial disconnection of the lignin biosynthesis from the general transcription factor network controlling secondary cell wall deposition in fiber cells and permitted for the first time to increase polysaccharide deposition without over-lignification. In order to increase secondary cell wall deposition only in woody tissue with a self-induction, we generated an artificial positive feedback loop using the pIRX8 promoter to express a second copy of the master transcription factor NST1. This promoter is specifically active in tissue producing secondary cell wall and is already under the control of the NST1 transcription factor in fiber cells. Therefore, such a chimeric gene allowed the overexpression of NST1 by self-induction, increasing as well the expression of downstream target genes involved in polysaccharide biosynthesis. In addition, using a downstream promoter of NST1 to express a new copy of itself may have increased the time-dependent expression of the NST1 transcription factor, therefore increasing the time of secondary cell wall deposition in fiber cells, which consequently increases cell wall thickness.

To our knowledge, only one artificial negative feedback loop has been generated in plants to regulate a developmental process, and it corresponds to the delay of senescence (Gan and Amasino, Science 1995). This strategy corresponds to the expression of the IPT gene encoding for an isopentenyltransferase at the beginning of the senescencing process using an early senescence induced promoter (pSAG12) in order to produce cytokinin specifically at that stage. This hormone is known to repress senescencing processes and keep the plants photosynthetically active much longer (Gan and Amasino, Science 1995). Due to the conservation of the regulatory mechanism and gene network of the senescence processes across species, and in particular the delay of senescencing processes by the hormone cytokinin, this synthetic construct was transferred into various crops (grasses and dicots) and could improve biomass yields due to an increase life time of the plant (McCabe et al., 2001; Lin et al., Acta Botanica Sinica 2002, 44:1333-1338; Robson et al., 2004; Li et al., 2004; Swartzberg et al., 2006; Calderini et al., 2007; Li et al., Plant Physiology 2010; and Chen et al., Molecular Breeding 2001).

Secondary cell wall biosynthesis falls in the same category of conserved regulatory networks, since this biological process is well conserved within vascular plants (Zhong et al., 2010). For example, transcriptional networks and genes involved in secondary cell wall biosynthesis are well conserved. The conservation of this network allowed us the utilization of the model plant Arabidopsis, allowing rapid testing and robustness of this approach. Because increased polysaccharide content has multiple applications from bioenergy to the paper industry, including forage crops, the transferability of this strategy need to be versatile. The approach described herein should be compatible and rapidly transferable from model species to bioenergy crops (dicots and monocots). It has previously been shown that overexpressing secondary cell wall transcription factors across species results in similar phenotypes and functions, suggesting that promoter regulatory elements are also well conserved. See, e.g., Shen et al., 2009 Bioenerg. Res 2:217-232; Zhong et al., 2010 Plant Physiol 152:1044-1055; Goicoechea et al 2005 Plant J 43:553-567; Franke et al., 2000, Plant J. 22:223-234. Therefore, the genome sequence of the target crop should not be required and the cassette promoter (e.g., pIRX5) and the transcription factor (e.g., NST1) from another species, such as Arabidopsis or a crop-related species, could be used to transform the target plant.

In contrast to yeast, E. coli, physcomitrella, and a few other species, promoter replacement by in vivo recombination in plants still has to be developed; therefore, in order to manipulate tissue specific lignin deposition, mutants are required. Natural loss of function mutants in essential genes in the lignin biosynthetic pathway are poorly available in crops due to the deleterious effects of mutations. In addition, tissue/cell specific gene expression inhibition has not yet been developed in plants. Therefore, general silencing strategies are regularly used to modify gene expression in order to reduce enzymatic activity in crops, which at least requires EST sequences of genes involved in the targeted biosynthesis pathway. One concern with the lignin biosynthesis pathway is that compromises between the gene repression level, plant health, and desired phenotype are often conflicting. For example, the improvement in saccharification by the repression of genes involved in the monolignol biosynthesis very often affects vessel integrity, therefore affecting water and nutrient transport and consequently plant growth. In order to transfer the presented technology to crops, the degeneracy of the genetic code (flexibility of the codon usage) could be used to generate silent resistant lignin genes that would be expressed with a vessel specific promoter from Arabidopsis or related species of the target crop together with a silencing construct to reduce or eliminate the expression of the corresponding native gene. For example, expressing in poplar a different 4CL encoding sequence with a vessel specific promoter such as VND6 would restore the growth and biomass yield of a 4CL antisense lines (Kitin et al., 2010; Voelker et al., 2010) and retain good saccharification efficiency. Alternatively, strategies that could bypass the defective enzymatic steps could be exploited. For example, the SmF5H gene from Selaginella could be expressed with a vessel specific promoter in a C3H RNAi-expressing poplar to restore the integrity of vessel and normal plant growth (Coleman et al., 2008a, 2008b). This SmF5H gene was recently shown in Arabidopsis to be able to restore the growth of HCT and C3H deficient mutants (Li et al., 2010 Plant Cell 22:1620-1632) and lignin mutants lacking the ability to produce p-coumaroyl shikimate and to meta-hydroxylate the p-coumaroyl shikimate respectively, which are essential steps in the lignin biosynthesis (Weng et al 2010). Similarly to this SmF5H strategy, both enzymatic steps converting phenylalanine into p-coumaric acid could be bypassed by using a tyrosine ammonia lyase (TAL) gene that converts tyrosine into p-hydroxycoumaric acid.

In summary, we have demonstrated that two approaches, one to increase cell biomass density and one to restrict lignin biosynthesis into essential tissue containing the vessels, were compatible and allowed the generation of healthy plants with a large amount of non-recalcitrant cell wall, allowing efficient enzymatic conversion into fermentable sugar without severe pre-treatments. These approaches open new doors for crop optimization and should benefit the lignocellulosic biofuel, paper and forage industries.

### REFERENCES

Anders, N., Wilkinson, M. D., Lovegrove, A., Freeman, J., Tryfona, T., Pellny, T. K., Weimar, T., Mortimer, J. C., Stott, K., Baker, J. M., Defoin-Platel, M., Shewry, P. R., Dupree, P., and Mitchell, R. A. (2012). Glycosyl transferases in family 61 mediate arabinofuranosyl transfer onto xylan in grasses. Proc Natl Acad Sci USA 109, 989-993
Brown D, Wightman R, Zhang Z, Gomez Ivan Atanassov L D, Bukowski J P, Tryfona T, McQueen-Mason S J, Dupree P, Turner S (2011) Arabidopsis genes IRREGULAR XYLEM (IRX15) and IRX15L encode DUF579 containing proteins that are essential for normal xylan deposition in the secondary cell wall. Plant J
Brown D M, Zhang, Z. N., Stephens, E., Dupree, P. and Turner, S. R. (2009) Characterization of IRX10 and IRX10-like reveals an essential role in glucuronoxylan biosynthesis in Arabidopsis. Plant J 57: 732-746
Cantarel B L, Coutinho P M, Rancurel C, Bernard T, Lombard V, Henrissat B (2009) The Carbohydrate-Active EnZymes database (CAZy): an expert resource for Glycogenomics. Nucleic Acids Res 37: D233-238
Gille, S., De Souza, A., Xiong, G., Benz, M., Cheng, K., Schultink, A., Reca, I. B., and Pauly, M. (2011). O-Acetylation of Arabidopsis Hemicellulose Xyloglucan Requires AXY4 or AXY4L, Proteins with a TBL and DUF231 Domain. Plant Cell 23, 4041-4053.
Lee C, Teng Q, Huang W, Zhong R, Ye Z H (2009) Down-regulation of PoGT47C expression in poplar results in a reduced glucuronoxylan content and an increased wood digestibility by cellulase. Plant Cell Physiol 50: 1075-1089
Lee C, Teng Q, Huang W, Zhong R, Ye Z H (2009) The F8H glycosyltransferase is a functional paralog of FRA8 involved in glucuronoxylan biosynthesis in Arabidopsis. Plant Cell Physiol 50: 812-827
Liepman A H, Wightman R, Geshi N, Turner S R, Scheller H V (2010) Arabidopsis—a powerful model system for plant cell wall research. Plant J 61: 1107-1121
Manabe Y, Nafisi M, Verhertbruggen Y, Orfila C, Gille S, Rautengarten C, Cherk C, Marcus S, Somerville S, Pauly M, Knox J P, Sakuragi Y, Scheller H V (2011) Loss-of-Function Mutation of REDUCED WALL ACETYLATION 2 in Arabidopsis Leads to Reduced Cell Wall Acetylation and Increased Resistance to Botrytis cinerea. Plant Physiol
Mortimer J C, Miles G P, Brown D M, Zhang Z, Segura M P, Weimar T, Yu X, Seffen K A, Stephens E, Turner S R, Dupree P (2010) Absence of branches from xylan in Arabidopsis gux mutants reveals potential for simplification of lignocellulosic biomass. Proc Natl Acad Sci USA 107: 17409-17414
Oikawa A, Joshi H J, Rennie E A, Ebert B, Manisseri C, Heazlewood J L, Scheller H V (2010) An integrative approach to the identification of Arabidopsis and rice genes involved in xylan and secondary wall development. PLoS One 5: e15481
Pena M J, Zhong R, Zhou G K, Richardson E A, O'Neill M A, Darvill A G, York W S, Ye Z H (2007) Arabidopsis irregular xylem8 and irregular xylem9: implications for the complexity of glucuronoxylan biosynthesis. Plant Cell 19: 549-563
Persson S, Caffall K H, Freshour G, Hilley M T, Bauer S, Poindexter P, Hahn M G, Mohnen D, Somerville C (2007) The Arabidopsis irregular xylem8 mutant is deficient in glucuronoxylan and homogalacturonan, which are essential for secondary cell wall integrity. Plant Cell 19: 237-255
Scheller H V, Manabe Y, Verhertbruggen Y, Gille S, Nafisi M, Rennie E, Oikawa A, Htwe S, Ebert B, Orfila C, Logue D, Knox P, Pauly M, Sakuragi Y (2010) Homologs of Cryptococcus Cas1p are required for Glycan Acetylation in Plants and Function Upstream of the Acetyltransferases. Glycobiology 20: 195
Scheller H V, Ulvskov P (2010) Hemicelluloses. Annu Rev Plant Biol 61: 263-289
Wu A M, Hornblad E, Voxeur A, Gerber L, Rihouey C, Lerouge P, Marchant A (2010) Analysis of the Arabidopsis IRX9/IRX9-L and IRX14/IRX14-L pairs of glycosyltransferase genes reveals critical contributions to biosynthesis of the hemicellulose glucuronoxylan. Plant Physiol 153: 542-554
Zhou G K, Zhong R, Himmelsbach D S, McPhail B T, Ye Z H (2007) Molecular characterization of PoGT8D and PoGT43B, two secondary wall-associated glycosyltransferases in poplar. Plant Cell Physiol 48: 689-699
Zhou G K, Zhong R, Richardson E A, Morrison W H, 3rd, Nairn C J, Wood-Jones A, Ye Z H (2006) The poplar glycosyltransferase GT47C is functionally conserved with Arabidopsis Fragile fiber8. Plant Cell Physiol 47: 1229-1240

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference in their entirety for all purposes.

### Illustrative Genes Involved in Wax/Cutin Biosynthesis: Including Accession Numbers and Synonymous Gene Designations.

AtCER1: At1g02205: Aldehyde decarbonylase
AtCER2: VC2: At4g24510: BAHD-type acyl-transferase
AtCER3: WAX2: At5g57800: Sterol desaturase
AtCER4: FAR3: At4g33790: Fatty acyl-CoA reductase
AtCER5: WBC12: ABCG12: At1g51500: ABC transporter
AtCER6: CUT1: KCS6: At1g68530: Very long chain fatty acid condensing enzyme
AtCER10: ECR: At3g55360: Enoyl-CoA reductase
AtWSD1: At5g37300: Wax ester synthase
AtMAH1: CYP96A15: At1g57750: Mid Chain alkane hydrolase
AtWBC11: ABCG11: DSO: COF1: At1g17840: ABC transporter
AtKCS1: At1g01120: Very long chain fatty acid condensing enzyme
AtKCS2: DAISY: At1g04220: Very long chain fatty acid condensing enzyme
AtFATB: At1g08510: Acyl Carrier
AtLACS1: At2g47240: Long chain acyl-CoA synthase
AtLACS2: At1g49430: Long chain acyl-CoA synthase
AtCYP86A4: At1g01600: Cytochrome P450-dependent fatty acid hydroxylase
AtCYP86A7: At1g63710: Cytochrome P450-dependent fatty acid hydroxylase
AtLCR: CYP86A5: At2g45970: Cytochrome P450-dependent fatty acid hydroxylase
AtKCS10: FDH: At2g26250: Very long chain fatty acid condensing enzyme
AtCER60: KCS5: At1g25450: Very long chain fatty acid condensing enzyme

ILLUSTRATIVE SEQUENCES

SEQ ID NO: 1
Arabidopsis thaliana PAL1 nucleic acid (At2g37040) NM_129260
atggagattaacggggcacacaagagcaacggaggaggagtggacgctatgttatgcggcggagacatcaagacaaagaacatgg
tgatcaacgcggaggatcctctcaactggggagctgcagcggagcaaatgaaaggtagccatttggatgaagtgaagagaatggttg
ctgagtttaggaagccagttgtgaatcttggtggtgagactctgaccattggacaagtggctgcgatctcaactattggtaacagtgtgaa
ggtggagctatcggagacagctagagccggtgtgaatgctagtagtgattgggttatggagagtatgaacaaaggcactgatagttat
ggtgttactactggttttggtgctacttctcatcggagaaccaaaaacggtgtcgcacttcagaaggaacttattagattccttaacgccgg
aatattcggaagcacgaaagaaacaagccacacattgccacactccgccacaagagccgccatgcttgtacgaatcaacactctcctc
caaggattttccggtatccgatttgagattctcgaagcaattaccagtttcctcaacaacaacatcactccatctctccccctccgtggtac
aatcaccgcctccggagatctcgttcctctctcctacatcgccggacttctcaccggtcgtcccaattccaaagctactggtcccaacgg
tgaagctttaacagcagaggaagctttcaaattagcaggaatcagctccggattctttgatctccagcctaaggaaggtctcgcgctagt
caatggcacggcggttggatctggaatggcgtcaatggtgttattcgaaacgaatgttctctctgttttggctgagattttgtcggcggtttt
cgcagaggtgatgagtggtaagcctgagttcaccgatcatctcactcacagacttaaacatcatcccggtcaaatcgaagcggcggcg
ataatggagcatatcctcgacggaagctcgtacatgaaattagctcagaagcttcacgagatggatccgttacagaaacctaaacaaga
tcgttacgctcttcgtacttctcctcaatggttaggtcctcaaatcgaagtgatccgttacgcaacgaaatcgatcgagcgtgagattaact
ccgtcaacgataatccgttgatcgatgtttcgaggaacaaggcgattcacggtggtaacttccaaggaacaccaatcggagtttcaatg
gataacacgagattggcgatagcagcgattggtaaactcatgtttgctcaattctcagagcttgtgaatgatttctacaacaatggtttacc
ctcgaatctaaccgcttcgaggaatccaagtttggattatggattcaagggagctgagattgcaatggcttcttattgttcagagcttcaat
acttagctaatcctgtgactagccatgttcaatcagcagagcaacataaccaagatgtcaactctttgggactaatctcgtctcgcaaaac
ttctgaagctgttgatattctcaagcttatgtcaacaacgttcctcgttgcgatttgtcaagctgtggatttgagacatttggaggagaatttg
agacagactgtgaagaacactgtctctcaagtggcgaagaaagttcttactactggagtcaatggtgagcttcatccttctcgcttctgcg
aaaaggatttactcaaagttgtagaccgtgaacaagtctacacatacgcggatgatccttgtagcgcaacgtacccgttgattcagaagc
tgagacaagttattgttgaccatgattgatcaatggtgagagtgagaagaatgcagtgacttcaatcttccataagattggagctttcgag
gaggagcttaaggcagtgctaccgaaagaagtggaagcagcaagagcagcctacgataacggaacatcggctatcccgaacagga
tcaaggaatgtaggtcgtatccattgtatagattcgtgagggaagagcttggaacagagcttttgaccggagagaaagtgacgtcgcct
ggagaagagttcgacaaggttttcacggcgatttgtgaaggtaaaatcattgatccgatgatggaatgtctcaacgagtggaacggagc
tcccattccaatatgttaa
SEQ ID NO: 2
Arabidopsis thaliana PAL1 protein (At2g37040) NP_181241
MEINGAHKSNGGGVDAMLCGGDIKTKNMVINAEDPLNWGAAAEQMKGSHLDEVK
RMVAEFRKPVVNLGGETLTIGQVAAISTIGNSVKVELSETARAGVNASSDWVMESM
NKGTDSYGVTTGFGATSHRRTKNGVALQKELIRFLNAGIFGSTKETSHTLPHSATRA
AMLVRINTLLQGFSGIRFEILEAITSFLNNNITPSLPLRGTITASGDLVPLSYIAGLLTGR
PNSKATGPNGEALTAEEAFKLAGISSGFFDLQPKEGLALVNGTAVGSGMASMVLFET
NVLSVLAEILSAVFAEVMSGKPEFTDHLTHRLKHHPGQIEAAAIMEHILDGSSYMKL
AQKLHEMDPLQKPKQDRYALRTSPQWLGPQIEVIRYATKSIEREINSVNDNPLIDVSR
NKAIHGGNFQGTPIGVSMDNTRLAIAAIGKLMFAQFSELVNDFYNNGLPSNLTASRN
PSLDYGFKGAEIAMASYCSELQYLANPVTSHVQSAEQHNQDVNSLGLISSRKTSEAV
DILKLMSTTFLVAICQAVDLRHLEENLRQTVKNTVSQVAKKVLTTGVNGELHPSRFC
EKDLLKVVDREQVYTYADDPCSATYPLIQKLRQVIVDHALINGESEKNAVTSIFHKIG
AFEEELKAVLPKEVEAARAAYDNGTSAIPNRIKECRSYPLYRFVREELGTELLTGEKV
TSPGEEFDKVFTAICEGKIIDPMMECLNEWNGAPIPIC
SEQ ID NO: 3
Arabidopsis thaliana C4H nucleic acid (At2g30490) NM_128601
atggacctcctcttgctggagaagtctttaatcgccgtcttcgtggcggtgattctcgccacggtgatttcaaagctccgcggcaagaaat
tgaagctacctccaggtcctataccaattccgatcttcggaaactggcttcaagtcggagatgatctcaaccaccgtaatctcgtcgatta
cgctaagaaattcggcgatctcttcctcctccgtatgggtcagcgaaacctagtcgtcgtctcctcaccggatctaacaaaggaagtgct
cctcactcaaggcgttgagtttggatccagaacgagaaacgtcgtgttcgacattttcaccgggaaaggtcaagatatggtgttcactgtt
tacggcgagcattggaggaagatgagaagaatcatgacggttcctttcttcaccaacaaagttgttcaacagaatcgtgaaggttggga
gtttgaagcagctagtgttgttgaagatgttaagaagaatccagattctgctacgaaaggaatcgtgttgaggaaacgtttgcaattgatg
atgtataacaatatgttccgtatcatgttcgatagaagatttgagagtgaggatgatcctcttttccttaggcttaaggctttgaatggtgaga
gaagtcgattagctcagagctttgagtataactatggagatttcattcctatccttagaccattcctcagaggctatttgaagatttgtcaag
atgtgaaagatcgaagaatcgctcttttcaagaagtactttgttgatgagaggaagcaaattgcgagttctaagcctacaggtagtgaag
gattgaaatgtgccattgatcacatccttgaagctgagcagaagggagaaatcaacgaggacaatgttctttacatcgtcgagaacatc
aatgtcgccgcgattgagacaacattgtggtctatcgagtggggaattgcagagctagtgaaccatcctgaaatccagagtaagctaag
gaacgaactcgacacagttcttggaccgggtgtgcaagtcaccgagcctgatcttcacaaacttccataccttcaagctgtggttaagg
agactcttcgtctgagaatggcgattcctctcctcgtgcctcacatgaacctccatgatgcgaagctcgctggctacgatatcccagcag
aaagcaaaatccttgttaatgcttggtggctagcaaacaaccccaacagctggaagaagcctgaagagtttagaccagagaggttcttt
gaagaagaatcgcacgtggaagctaacggtaatgacttcaggtatgtgccatttggtgttggacgtcgaagctgtcccgggattatattg
gcattgcctattttggggatcaccattggtaggatggtccagaacttcgagcttcttcctcctccaggacagtctaaagtggatactagtg
agaaaggtggacaattcagcttgcacatccttaaccactccataatcgttatgaaaccaaggaactgttaa
SEQ ID NO: 4
Arabidopsis thaliana C4H protein (At2g30490) NP_180607
MDLLLLEKSLIAVFVAVILATVISKLRGKKLKLPPGPIPIPIFGNWLQVGDDLNHRNLV
DYAKKFGDLFLLRMGQRNLVVVSSPDLTKEVLLTQGVEFGSRTRNVVFDIFTGKGQ
DMVFTVYGEHWRKMRRIMTVPFFTNKVVQQNREGWEFEAASVVEDVKKNPDSAT
KGIVLRKRLQLMMYNNMFRIMFDRRFESEDDPLFLRLKALNGERSRLAQSFEYNYG
DFIPILRPFLRGYLKICQDVKDRRIALFKKYFVDERKQIASSKPTGSEGLKCAIDHILEA
EQKGEINEDNVLYIVENINVAAIETTLWSIEWGIAELVNHPEIQSKLRNELDTVLGPGV
QVTEPDLHKLPYLQAVVKETLRLRMAIPLLVPHMNLHDAKLAGYDIPAESKILVNA
WWLANNPNSWKKPEEFRPERFFEEESHVEANGNDFRYVPFGVGRRSCPGIILALPILG
ITIGRMVQNFELLPPPGQSKVDTSEKGGQFSLHILNHSIIVMKPRNC
SEQ ID NO: 5
Arabidopsis thaliana 4CL2 nucleic acid (At3g21240) NM_113019
atgacgacacaagatgtgatagtcaatgatcagaatgatcagaaacagtgtagtaatgacgtcattttccgatcgagattgcctgatatat
acatccctaaccacctcccactccacgactacatcttcgaaaatatctcagagttcgccgctaagccatgcttgatcaacggtcccaccg
gcgaagtatacacctacgccgatgtccacgtaacatctcggaaactcgccgccggtcttcataacctcggcgtgaagcaacacgacgt
tgtaatgatcctcctcccgaactctcctgaagtagtcctcactttccttgccgcctccttcatcggcgcaatcaccacctccgcgaacccg
ttcttcactccggcggagatttctaaacaagccaaagcctccgcggcgaaactcatcgtcactcaatcccgttacgtcgataaaatcaag
aacctccaaaacgacggcgttttgatcgtcaccaccgactccgacgccatccccgaaaactgcctccgtttctccgagttaactcagtc
cgaagaaccacgagtggactcaataccggagaagatttcgccagaagacgtcgtggcgcttcctttctcatccggcacgacgggtctc
cccaaaggagtgatgctaacacacaaaggtctagtcacgagcgtggcgcagcaagtcgacggcgagaatccgaatctttacttcaac
agagacgacgtgatcctctgtgtcttgcctatgttccatatatacgctctcaactccatcatgctctgtagtctcagagttggtgccacgatc
ttgataatgcctaagttcgaaatcactctcttgttagagcagatacaaaggtgtaaagtcacggtggctatggtcgtgccaccgatcgtttt
agctatcgcgaagtcgccggagacggagaagtatgatctgagctcggttaggatggttaagtctggagcagctcctcttggtaaggag
cttgaagatgctattagtgctaagtttcctaacgccaagcttggtcagggctatgggatgacagaagcaggtccggtgctagcaatgtc
gttagggtttgctaaagagccgtttccagtgaagtcaggagcatgtggtacggtggtgaggaacgccgagatgaagatacttgatcca
gacacaggagattctttgcctaggaacaaacccggcgaaatatgcatccgtggcaaccaaatcatgaaaggctatctcaatgacccctt
ggccacggcatcgacgatcgataaagatggttggcttcacactggagacgtcggatttatcgatgatgacgacgagcttttcattgtgg
atagattgaaagaactcatcaagtacaaaggatttcaagtggctccagctgagctagagtctctcctcataggtcatccagaaatcaatg
atgttgctgtcgtcgccatgaaggaagaagatgctggtgaggttcctgttgcgtttgtggtgagatcgaaagattcaaatatatccgaag
atgaaatcaagcaattcgtgtcaaaacaggttgtgttttataagagaatcaacaaagtgttcttcactgactctattcctaaagctccatcag
ggaagatattgaggaaggatctaagagcaagactagcaaatggattaatgaactag
SEQ ID NO: 6
Arabidopsis thaliana 4CL2 protein (At3g21240) NP_188761
MTTQDVIVNDQNDQKQCSNDVIFRSRLPDIYIPNHLPLHDYIFENISEFAAKPCLINGP
TGEVYTYADVHVTSRKLAAGLHNLGVKQHDVVMILLPNSPEVVLTFLAASFIGAITT
SANPFFTPAEISKQAKASAAKLIVTQSRYVDKIKNLQNDGVLIVTTDSDAIPENCLRFS
ELTQSEEPRVDSIPEKISPEDVVALPFSSGTTGLPKGVMLTHKGLVTSVAQQVDGENP
NLYFNRDDVILCVLPMFHIYALNSIMLCSLRVGATILIMPKFEITLLLEQIQRCKVTVA
MVVPPIVLAIAKSPETEKYDLSSVRMVKSGAAPLGKELEDAISAKFPNAKLGQGYGM
TEAGPVLAMSLGFAKEPFPVKSGACGTVVRNAEMKILDPDTGDSLPRNKPGEICIRG
NQIMKGYLNDPLATASTIDKDGWLHTGDVGFIDDDDELFIVDRLKELIKYKGFQVAP
AELESLLIGHPEINDVAVVAMKEEDAGEVPVAFVVRSKDSNISEDEIKQFVSKQVVFY
KRINKVFFTDSIPKAPSGKILRKDLRARLANGLMN
SEQ ID NO: 7
Arabidopsis thaliana HCT nucleic acid (At5g48930) NM_124270
atgaaaattaacatcagagattccaccatggtccggcctgccaccgagacaccaatcactaatctttggaactccaacgtcgaccttgtc
atccccagattccatacccctagtgtctacttctacagacccaccggcgcttccaatttctttgaccctcaggtcatgaaggaagctctttc
caaagcccttgtccctttttaccctatggctggtcgcttgaagagagacgatgatggtcgtattgagatcgattgtaacggtgctggtgttc
tcttcgttgtggctgatactccttctgttatcgatgattttggtgattttgctcctacccttaatctccgtcagcttattcccgaagttgatcactc
cgctggcattcactctttcccgcttctcgttttgcaggtgactttctttaaatgtgggggagcttcacttggggttgggatgcaacatcacgc
ggcagatggtttctctggtcttcattttatcaacacatggtctgatatggctcgtggtcttgacctaaccattccacctttcattgatcgaaca
ctcctccgagctagggacccgccacagcctgcttttcatcatgttgaatatcagcctgcaccaagtatgaagatacctcttgatccgtcta
aatcaggacctgagaataccactgtctctatattcaaattaacacgagaccagcttgttgctcttaaggcgaaatccaaggaggatggga
acactgtcagctacagctcatacgagatgttggcagggcatgtgtggagatcagtgggaaaggcgcgagggcttccaaacgaccaa
gagacgaaactgtacattgcaactgatggaaggtctagactacgtccgcagctgcctcctggttactttgggaatgtgatattcactgca
acaccattggctgttgcaggggatttgttatctaagccaacatggtatgctgcaggacagattcatgatttcttggttcgtatggatgataa
ctatctgaggtcagctcttgactacctggagatgcagcctgatctgtcagcccttgtccgcggtgcacatacctacaagtgcccaaatttg
ggaatcacaagctgggttagattacctatttatgatgcagactttggttggggtcgtcctatctttatgggacctggtggaattccatacga
gggtttgtcttttgtgctaccaagtcctactaatgatggcagcttatccgttgccattgccctccaatctgaacacatgaaactgtttgagaa
gtttttgtttgagatatga
SEQ ID NO: 8
Arabidopsis thaliana HCT protein (At5g48930) NP_199704
MKINIRDSTMVRPATETPITNLWNSNVDLVIPRFHTPSVYFYRPTGASNFFDPQVMKE
ALSKALVPFYPMAGRLKRDDDGRIEIDCNGAGVLFVVADTPSVIDDFGDFAPTLNLR
QLIPEVDHSAGIHSFPLLVLQVTFFKCGGASLGVGMQHHAADGFSGLHFINTWSDMA
RGLDLTIPPFIDRTLLRARDPPQPAFHHVEYQPAPSMKIPLDPSKSGPENTTVSIFKLTR
DQLVALKAKSKEDGNTVSYSSYEMLAGHVWRSVGKARGLPNDQETKLYIATDGRS
RLRPQLPPGYFGNVIFTATPLAVAGDLLSKPTWYAAGQIHDFLVRMDDNYLRSALD
YLEMQPDLSALVRGAHTYKCPNLGITSWVRLPIYDADFGWGRPIFMGPGGIPYEGLS
FVLPSPTNDGSLSVAIALQSEHMKLFEKFLFEI
SEQ ID NO: 9
Arabidopsis thaliana C3H nucleic acid (At4g34050) NM_119566
atggcgacgacaacaacagaagcaacgaagacatcatcgaccaatggagaagatcagaagcagtctcagaatcttcgacatcaaga
agttggtcacaagagtctcttacagagcgatgatctctaccagtatatactggagacaagtgtgtatcctagagaaccagaatcaatgaa
ggaactcagggaagtgacagcaaaacatccatggaacataatgaccacatcagctgatgaaggacagttcttaaacatgcttatcaag
ctcgttaacgccaagaacacaatggagatcggagtttacactggctactctcttctcgccaccgctcttgctctccctgaagacggcaaa
attctggctatggatgtcaacagagagaattacgaattgggtttaccgatcattgagaaagccggcgttgctcacaagatcgacttcagg
gaaggccctgctcttcccgttcttgatgaaatcgttgctgacgagaagaaccatggaacatatgactttatattcgttgatgctgacaaag
acaactacatcaactaccacaagcgtttgatcgatcttgtgaaaattggaggagtgattggctacgacaacactctgtggaatggttctgt
cgtggctcctcctgatgcaccaatgaggaagtacgttcgttactacagagactttgttcttgagcttaacaaggctcttgctgctgaccctc
ggatcgagatctgtatgctccctgttggtgatggaatcactatctgccgtcggatcagttga
SEQ ID NO: 10
Arabidopsis thaliana C3H protein (At4g34050) NP_850337
MSWFLIAVATIAAVVSYKLIQRLRYKFPPGPSPKPIVGNLYDIKPVRFRCYYEWAQSY
GPIISVWIGSILNVVVSSAELAKEVLKEHDQKLADRHRNRSTEAFSRNGQDLIWADY
GPHYVKVRKVCTLELFTPKRLESLRPIREDEVTAMVESVFRDCNLPENRAKGLQLRK
YLGAVAFNNITRLAFGKRFMNAEGVVDEQGLEFKAIVSNGLKLGASLSIAEHIPWLR
WMFPADEKAFAEHGARRDRLTRAIMEEHTLARQKSSGAKQHFVDALLTLKDQYDL
SEDTIIGLLWDMITAGMDTTAITAEWAMAEMIKNPRVQQKVQEEFDRVVGLDRILTE
ADFSRLPYLQCVVKESFRLHPPTPLMLPHRSNADVKIGGYDIPKGSNVHVNVWAVA
RDPAVWKNPFEFRPERFLEEDVDMKGHDFRLLPFGAGRRVCPGAQLGINLVTSMMS
HLLHHFVWTPPQGTKPEEIDMSENPGLVTYMRTPVQAVATPRLPSDLYKRVPYDM
SEQ ID NO: 11
Arabidopsis thaliana CCR1 nucleic acid (At1g15950) NM_101463
atgccagtcgacgtagcctcaccggccggaaaaaccgtctgcgtcaccggagctggtggatacatcgcttcttggattgttaagatact
tctcgagagaggttacacagtcaaaggaaccgtacggaatccagatgatccgaagaacacacatttgagagaactagaaggaggaa
aggagagactgattctgtgcaaagcagatcttcaggactacgaggctcttaaggcggcgattgatggttgcgacggcgtctttcacacg
gcttctcctgtcaccgacgatccggaacaaatggtggagccggccgtgaatggagccaagtttgtaattaatgctgcggctgaggcca
aggtcaagcgcgtggtcatcacctcctccattggtgccgtctacatggacccgaaccgtgaccctgaggctgtcgttgacgaaagttgt
tggagtgatcttgacttctgcaaaaacaccaagaattggtattgttacggcaagatggtggcggaacaagcggcgtgggagacagca
aaggagaaaggtgttgacttggtggtgttgaatccggtgctggttcttggaccgccgttacagccgacgatcaacgccagtctttacca
cgtcctcaaatatctaaccggctcggctaagacttatgctaatttgactcaagcttatgtggatgttcgcgatgtcgcgctggctcatgttct
ggtctatgaggcaccctcggcctccggacgttatctcctagccgagagtgctcgccaccgcggggaagttgttgagattctggctaag
ctattcccggagtatcctcttccgaccaagtgcaaggacgagaagaaccctagagccaagccatacaaattcactaaccagaagatta
aggacttaggcttagagttcacttccaccaagcaaagcctctacgacacagtcaagagcttacaagagaaaggccatcttgctcctcct
cctcctcctccttcagcatcgcaagaatccgtggaaaatggcattaagatcgggtcttga
SEQ ID NO: 12
Arabidopsis thaliana CCR1 protein (At1g15950) NP_173047
MPVDVASPAGKTVCVTGAGGYIASWIVKILLERGYTVKGTVRNPDDPKNTHLRELE
GGKERLILCKADLQDYEALKAAIDGCDGVFHTASPVTDDPEQMVEPAVNGAKFVIN
AAAEAKVKRVVITSSIGAVYMDPNRDPEAVVDESCWSDLDFCKNTKNWYCYGKMV
AEQAAWETAKEKGVDLVVLNPVLVLGPPLQPTINASLYHVLKYLTGSAKTYANLTQ
AYVDVRDVALAHVLVYEAPSASGRYLLAESARHRGEVVEILAKLFPEYPLPTKCKDE
KNPRAKPYKFTNQKIKDLGLEFTSTKQSLYDTVKSLQEKGHLAPPPPPPSASQESVEN
GIKIGS
SEQ ID NO: 13
Arabidopsis thaliana NST1 (At2g46770) nucleic acid NM_130243
atgatgtcaaaatctatgagcatatcagtgaacggacaatctcaagtgcctcctgggtttaggtttcatccgaccgaggaagagctgttg
cagtattatctccggaagaaagttaatagcatcgagatcgatcttgatgtcattcgcgacgttgatctcaacaagctcgagccttgggaca
ttcaagagatgtgtaaaataggaacaacgccacaaaacgactggtatttctttagccacaaggacaaaaaatatccgacgggaacgag
aactaacagagccactgcggctggattttggaaagcaactggccgcgacaagatcatatatagcaatggccgtagaattgggatgag
aaagactcttgttttctacaaaggccgagctcctcacggccaaaaatctgattggatcatgcatgaatatagactcgatgacaacattattt
cccccgaggatgtcaccgttcatgaggtcgtgagtattataggggaagcatcacaagacgaaggatgggtggtgtgtcgtattttcaag
aagaagaatcttcacaaaaccctaaacagtcccgtcggaggagcttccctgagcggcggcggagatacgccgaagacgacatcatc
tcagatcttcaacgaggatactctcgaccaatttcttgaacttatggggagatcttgtaaagaagagctaaatcttgaccctttcatgaaact
cccaaacctcgaaagccctaacagtcaggcaatcaacaactgccacgtaagctctcccgacactaatcataatatccacgtcagcaac
gtggtcgacactagctttgttactagctgggcggctttagaccgcctcgtggcctcgcagcttaacggacccacatcatattcaattaca
gccgtcaatgagagccacgtgggccatgatcatctcgctttgccttccgtccgatctccgtaccccagcctaaaccggtccgcttcgtac
cacgccggtttaacacaggaatatacaccggagatggagctatggaatacgacgacgtcgtctctatcgtcatcgcctggcccattttgt
cacgtgtcgaatggtagtggataa
SEQ ID NO: 14
Arabidopsis thaliana NST1 (At2g46770) protein NP_182200
MMSKSMSISVNGQSQVPPGFRFHPTEEELLQYYLRKKVNSIEIDLDVIRDVDLNKLEP
WDIQEMCKIGTTPQNDWYFFSHKDKKYPTGTRTNRATAAGFWKATGRDKIIYSNGR
RIGMRKTLVFYKGRAPHGQKSDWIMHEYRLDDNIISPEDVTVHEVVSIIGEASQDEG
WVVCRIFKKKNLHKTLNSPVGGASLSGGGDTPKTTSSQIFNEDTLDQFLELMGRSCK
EELNLDPFMKLPNLESPNSQAINNCHVSSPDTNHNIHVSNVVDTSFVTSWAALDRLV
ASQLNGPTSYSITAVNESHVGHDHLALPSVRSPYPSLNRSASYHAGLTQEYTPEMEL
WNTTTSSLSSSPGPFCHVSNGSG
SEQ ID NO: 15
Arabidopsis thaliana NST2 (At3g61910) nucleic acid NM_116056
atgaacatatcagtaaacggacagtcacaagtacctcctggctttaggtttcacccaaccgaggaagagctcttgaagtattacctccgc
aagaaaatctctaacatcaagatcgatctcgatgttattcctgacattgatctcaacaagctcgagccttgggatattcaagagatgtgtaa
gattggaacgacgccgcaaaacgattggtacttttatagccataaggacaagaagtatcccaccgggactagaaccaacagagccac
cacggtcggattttggaaagcgacgggacgtgacaagaccatatataccaatggtgatagaatcgggatgcgaaagacgcttgtcttc
tacaaaggtcgagcccctcatggtcagaaatccgattggatcatgcacgaatatagactcgacgagagtgtattaatctcctcgtgtggc
gatcatgacgtcaacgtagaaacgtgtgatgtcataggaagtgacgaaggatgggtggtgtgtcgtgttttcaagaaaaataacctttgc
aaaaacatgattagtagtagcccggcgagttcggtgaaaacgccgtcgttcaatgaggagactatcgagcaacttctcgaagttatggg
gcaatcttgtaaaggagagatagttttagaccctttcttaaaactccctaacctcgaatgccataacaacaccaccatcacgagttatcagt
ggttaatcgacgaccaagtcaacaactgccacgtcagcaaagttatggatcccagcttcatcactagctgggccgctttggatcggctc
gttgcctcacagttaaatgggcccaactcgtattcaataccagccgttaatgagacttcacaatcaccgtatcatggactgaaccggtcc
ggttgtaataccggtttaacaccagattactatataccggagattgatttatggaacgaggcagatttcgcgagaacgacatgccacttgt
tgaacggtagtggataa
SEQ ID NO: 16
Arabidopsis thaliana NST2 (At3g61910) protein NP _191750
MNISVNGQSQVPPGFRFHPTEEELLKYYLRKKISNIKIDLDVIPDIDLNKLEPWDIQEM
CKIGTTPQNDWYFYSHKDKKYPTGTRTNRATTVGFWKATGRDKTIYTNGDRIGMRK
TLVFYKGRAPHGQKSDWIMHEYRLDESVLISSCGDHDVNVETCDVIGSDEGWVVCR
VFKKNNLCKNMISSSPASSVKTPSFNEETIEQLLEVMGQSCKGEIVLDPFLKLPNLECH
NNTTITSYQWLIDDQVNNCHVSKVMDPSFITSWAALDRLVASQLNGPNSYSIPAVNE
TSQSPYHGLNRSGCNTGLTPDYYIPEIDLWNEADFARTTCHLLNGSG
SEQ ID NO: 17
Arabidopsis thaliana NST3/SND1 (At1g32770) nucleic acid NM_103011
atggctgataataaggtcaatctttcgattaatggacaatcaaaagtgcctccaggtttcagattccatcccaccgaagaagaacttctcc
attactatctccgtaagaaagttaactctcaaaagatcgatcttgatgtcattcgtgaagttgatctaaacaagcttgagccttgggatattc
aagaggaatgtagaatcggttcaacgccacaaaacgactggtacttcttcagccacaaggacaagaagtatccaaccgggaccagg
acgaaccgggcaacagtcgctggattctggaaagctaccggacgtgacaaaatcatctgcagttgtgtccggagaattggactgagg
aagacactcgtgttctacaaaggaagagctcctcacggtcagaaatccgactggatcatgcatgagtatcgcctcgacgatactccaat
gtctaatggctatgctgatgttgttacagaagatccaatgagctataacgaagaaggttgggtggtatgtcgagtgttcaggaagaagaa
ctatcaaaagattgacgattgtcctaaaatcactctatcttctttacctgatgacacggaggaagagaaggggcccacctttcacaacact
caaaacgttaccggtttagaccatgttcttctctacatggaccgtaccggttctaacatttgcatgcccgagagccaaacaacgactcaa
catcaagatgatgtcttattcatgcaactcccaagtcttgagacacctaaatccgagagcccggtcgaccaaagtttcctgactccaagc
aaactcgatttctctcccgttcaagagaagataaccgaaagaccggtttgcagcaactgggctagtcttgaccggctcgtagcttggca
attgaacaatggtcatcataatccgtgtcatcgtaagagttttgatgaagaagaagaaaatggtgatactatgatgcagcgatgggatctt
cattggaataatgatgataatgttgatctttggagtagtttcactgagtcttcttcgtctttagacccacttcttcatttatctgtatga
SEQ ID NO: 18
Arabidopsis thaliana NST3/SND1 (At1g32770) protein NP_174554
MADNKVNLSINGQSKVPPGFRFHPTEEELLHYYLRKKVNSQKIDLDVIREVDLNKLE
PWDIQEECRIGSTPQNDWYFFSHKDKKYPTGTRTNRATVAGFWKATGRDKIICSCVR
RIGLRKTLVFYKGRAPHGQKSDWIMHEYRLDDTPMSNGYADVVTEDPMSYNEEGW
VVCRVFRKKNYQKIDDCPKITLSSLPDDTEEEKGPTFHNTQNVTGLDHVLLYMDRTG
SNICMPESQTTTQHQDDVLFMQLPSLETPKSESPVDQSFLTPSKLDFSPVQEKITERPV
CSNWASLDRLVAWQLNNGHHNPCHRKSFDEEEENGDTMMQRWDLHWNNDDNVD
LWSSFTESSSSLDPLLHLSV
SEQ ID NO: 19
Arabidopsis thaliana SND2 (At4g28500) nucleic acid NM_118992
atgacttggtgcaatgaccgtagcgatgttcagaccgttgaaagaatcattccctccccgggggcggctgagtcccccgtagcctcact
tccggtctcttgtcacaaaacttgcccttcttgtggccataacttcaagtttcacgaacaggctgggatccatgacttgccgggacttcctg
ctggagtaaaatttgatccgacggatcaagaggtcttggagcatcttgaaggcaaggtaagagatgacgcaaaaaagcttcatcctctc
attgatgagtttatccgtaccatcgatggtgaaaacggcatttgttatacccatcctgaaaaattgccaggagtgaacaaggacgggacg
gtgcgtcatttcttccaccgaccgtcgaaggcatacacgacgggaacaagaaagcgacgtaaagtccacactgattctgacgtcggtg
gagagacacggtggcacaagacaggaaaaacacggccagttcttgctggaggaagagtgagaggctacaagaaaatcctagtgct
ctacacaaactacggcaaacaaaaaaaacccgagaagactaattgggtaatgcatcaatatcatcttggcactagcgaggaagagaa
agaaggtgagctcgtcgtctccaaagtcttttaccagactcaaccacgtcaatgcggtggctccgttgctgctgcagccaccgctaagg
accgaccttacctccacggcctcggtggaggtggtggccgccaccttcattaccatcttcatcataacaacggtaacggtaagagcaa
cggcagtgggggaaccgccggagccggtgagtattatcacaatattccggctattatctcgttcaatcagaccgggatacagaaccac
ttggttcatgactctcaaccttttatcccttaa
SEQ ID NO: 20
Arabidopsis thaliana SND2 (At4g28500) protein NP_194579
MTWCNDRSDVQTVERIIPSPGAAESPVASLPVSCHKTCPSCGHNFKFHEQAGIHDLPG
LPAGVKFDPTDQEVLEHLEGKVRDDAKKLHPLIDEFIRTIDGENGICYTHPEKLPGVN
KDGTVRHFFHRPSKAYTTGTRKRRKVHTDSDVGGETRWHKTGKTRPVLAGGRVRG
YKKILVLYTNYGKQKKPEKTNWVMHQYHLGTSEEEKEGELVVSKVFYQTQPRQCG
GSVAAAATAKDRPYLHGLGGGGGRHLHYHLHHNNGNGKSNGSGGTAGAGEYYHN
IPAIISFNQTGIQNHLVHDSQPFIP
SEQ ID NO: 21
Arabidopsis thaliana SND3 (At1g28470) nucleic acid NM_102615
atgagttggtgtgatggttcagatgataactacgatcttaatcttgaaagagtatcgaacactgatcatccatcggttcaactcaaagacca
atctcaatcatgtgtaacgagccgtccagattccaagattagcgctgaaactcccatcacgacttgtccttcttgcggacacaagctccat
catcaccaagacgaccaggttggtagcatcaaagatttaccaagcttaccggcaggagtcaaattcgatccgtcggataaagagatcc
ttatgcatttggaggcgaaggtatcatccgataagcgaaaacttcatccgttgattgatgaatttatacctacgcttgaaggagagaatgg
aatttgttatacgcatcctgagaaacttcctggagtaagcaaggacgggcaagtacggcacttcttccaccggccatcaaaggcttatac
gaccggaacacgaaaacgaagaaaagtgagcacagatgaggaaggccatgaaacaaggtggcacaaaacaggcaagactcgac
ctgttttgtctcaatcaggagaaaccggtttcaagaagatcctagtgctctacaccaactatggtcgccagaagaagcctgagaagacg
aattgggtgatgcatcagtatcatttaggtagcagcgaggacgaaaaagacggtgaaccagtcctctctaaagtcttctaccaaacaca
gcctaggcaatgcggttcgatggaacctaaaccgaaaaatctcgtaaacctaaaccggtttagttatgaaaatattcaggccggtttcgg
gtatgagcatggtggtaaaagtgaagagacgacgcaggtgattcgagagttggtagttcgtgaaggcgatgggtcatgttcgtttctta
gttttacttgtgatgcaagtaagggtaaagaaagcttcatgaagaatcaatag
SEQ ID NO: 22
Arabidopsis thaliana SND3 (At1g28470) protein NP_564309
MSWCDGSDDNYDLNLERVSNTDHPSVQLKDQSQSCVTSRPDSKISAETPITTCPSCG
HKLHHHQDDQVGSIKDLPSLPAGVKFDPSDKEILMHLEAKVSSDKRKLHPLIDEFIPT
LEGENGICYTHPEKLPGVSKDGQVRHFFHRPSKAYTTGTRKRRKVSTDEEGHETRW
HKTGKTRPVLSQSGETGFKKILVLYTNYGRQKKPEKTNWVMHQYHLGSSEDEKDGE
PVLSKVFYQTQPRQCGSMEPKPKNLVNLNRFSYENIQAGFGYEHGGKSEETTQVIRE
LVVREGDGSCSFLSFTCDASKGKESFMKNQ
SEQ ID NO: 23
Arabidopsis thaliana MYB103 (At1g63910) nucleic acid NM_105065
atgggtcatcactcatgctgcaaccagcaaaaggtgaagagagggctttggtcaccggaagaagatgagaagcttattagatatatca
caactcatggctatggatgttggagtgaagtccctgaaaaagcagggcttcaaagatgtggaaaaagttgtagattgcgatggataaac
tatcttcgacctgatatcaggagaggaaggttctctccagaagaagagaaattgatcataagccttcatggagttgtgggaaacaggtg
ggctcatatagctagtcatttaccgggaagaacagataacgagattaaaaactattggaattcatggattaagaaaaagatacgaaaac
cgcaccatcattacagtcgtcatcaaccgtcagtaactactgtgacattgaatgcggacactacatcgattgccactaccatcgaggcct
ctaccaccacaacatcgactatcgataacttacattttgacggtttcactgattctcctaaccaattaaatttcaccaatgatcaagaaacta
atataaagattcaagaaacttttttctcccataaacctcctctcttcatggtagacacaacacttcctatcctagaaggaatgttctctgaaaa
catcatcacaaacaataacaagaacaatgatcatgatgacacgcaaagaggaggaagagaaaatgtttgtgaacaagcatttctaaca
actaacacggaagaatgggatatgaatcttcgtcagcaagagccgtttcaagttcctacactggcgtcacatgtgttcaacaactcttcca
attcaaatattgacacggttataagttataatctaccggcgctaatagagggaaatgtcgataacatcgtccataatgaaaacagcaatgt
ccaagatggagaaatggcgtccacattcgaatgtttaaagaggcaagaactaagctatgatcaatgggacgattcacaacaatgctcta
actttttcttttgggacaaccttaatataaacgtggaaggttcatctcttgttggaaaccaagacccatcaatgaatttgggatcatctgcctt
atcttcttctttcccttcttcgttttaa
SEQ ID NO: 24
Arabidopsis thaliana MYB103 (At1g63910) protein NP_176575
MGHHSCCNQQKVKRGLWSPEEDEKLIRYITTHGYGCWSEVPEKAGLQRCGKSCRLR
WINYLRPDIRRGRFSPEEEKLIISLHGVVGNRWAHIASHLPGRTDNEIKNYWNSWIKK
KIRKPHHHYSRHQPSVTTVTLNADTTSIATTIEASTTTTSTIDNLHFDGFTDSPNQLNF
TNDQETNIKIQETFFSHKPPLFMVDTTLPILEGMFSENIITNNNKNNDHDDTQRGGRE
NVCEQAFLTTNTEEWDMNLRQQEPFQVPTLASHVFNNSSNSNIDTVISYNLPALIEGN
VDNIVHNENSNVQDGEMASTFECLKRQELSYDQWDDSQQCSNFFFWDNLNINVEGS
SLVGNQDPSMNLGSSALSSSFPSSF
SEQ ID NO: 25
Arabidopsis thaliana MYB85 (At4g22680) nucleic acid NM_118394
atggggagacagccatgctgtgacaagctaggggtgaagaaagggccgtggacggtggaggaagataagaagcttataaacttcat
actaaccaatggccattgttgctggcgtgctttgccgaagctggccggtctccgtcgctgtggaaagagctgccgcctccggtggacta
actatctccggcctgacttaaaacgaggccttctctcgcatgatgaagaacaacttgtcatagatcttcatgctaatctcggcaataagtg
gtctaagatagcttcaagattacctggaagaacagataacgaaataaaaaaccattggaatactcatatcaagaagaaacttcttaagat
gggaatcgatcctatgacccatcaacccctaaatcaagaaccttctaatatcgataattccaaaaccattccgtccaatccagacgatgtc
tcagtggaaccaaagacaactaacacgaaatacgtggagataagtgtcacgacaacagaagaagaaagtagtagcacggttactgat
caaaacagttcgatggataatgaaaatcatctaattgacaacatttatgatgatgatgaattgtttagttacttatggtccgacgaaactact
aaagatgaggcctcttggagtgatagtaactttggtgttggtggaacattatatgaccacaatatctccggcgccgatgcagattttccga
tatggtcaccggaaagaatcaatgacgagaagatgtttttggattattgtcaagactttggtgttcatgattttgggttttga
SEQ ID NO: 26
Arabidopsis thaliana MYB85 (At4g22680) protein NP_567664
MGRQPCCDKLGVKKGPWTVEEDKKLINFILTNGHCCWRALPKLAGLRRCGKSCRLR
WTNYLRPDLKRGLLSHDEEQLVIDLHANLGNKWSKIASRLPGRTDNEIKNHWNTHI
KKKLLKMGIDPMTHQPLNQEPSNIDNSKTIPSNPDDVSVEPKTTNTKYVEISVTTTEE
ESSSTVTDQNSSMDNENHLIDNIYDDDELFSYLWSDETTKDEASWSDSNFGVGGTLY
DHNISGADADFPIWSPERINDEKMFLDYCQDFGVHDFGF
SEQ ID NO: 27
Arabidopsis thaliana MYB46 (At5g12870) nucleic acid NM_121290
atgaggaagccagaggtagccattgcagctagtactcaccaagtaaagaagatgaagaagggactttggtctcctgaggaagactca
aagctgatgcaatacatgttaagcaatggacaaggatgttggagtgatgttgcgaaaaacgcaggacttcaaagatgtggcaaaagct
gccgtcttcgttggatcaactatcttcgtcctgacctcaagcgtggcgctttctctcctcaagaagaggatctcatcattcgctttcattccat
cctcggcaacaggtggtctcagattgcagcacgattgcctggtcggaccgataacgagatcaagaatttctggaactcaacaataaag
aaaaggctaaagaagatgtccgatacctccaacttaatcaacaactcatcctcatcacccaacacagcaagcgattcctcttctaattcc
gcatcttctttggatattaaagacattataggaagcttcatgtccttacaagaacaaggcttcgtcaacccttccttgacccacatacaaac
caacaatccatttccaacgggaaacatgatcagccacccgtgcaatgacgattttaccccttatgtagatggtatctatggagtaaacgc
aggggtacaaggggaactctacttcccacctttggaatgtgaagaaggtgattggtacaatgcaaatataaacaaccacttagacgagt
tgaacactaatggatccggaaacgcacctgagggtatgagaccagtggaagaattttgggaccttgaccagttgatgaacactgaggt
tccttcgttttacttcaacttcaaacaaagcatatga
SEQ ID NO: 28
Arabidopsis thaliana MYB46 (At5g12870) protein NP_196791
MRKPEVAIAASTHQVKKMKKGLWSPEEDSKLMQYMLSNGQGCWSDVAKNAGLQR
CGKSCRLRWINYLRPDLKRGAFSPQEEDLIIRFHSILGNRWSQIAARLPGRTDNEIKNF
WNSTIKKRLKKMSDTSNLINNSSSSPNTASDSSSNSASSLDIKDIIGSFMSLQEQGFVN
PSLTHIQTNNPFPTGNMISHPCNDDFTPYVDGIYGVNAGVQGELYFPPLECEEGDWY
NANINNHLDELNTNGSGNAPEGMRPVEEFWDLDQLMNTEVPSFYFNFKQSI
SEQ ID NO: 29
Arabidopsis thaliana MYB83 (At3g08500) nucleic acid NM_111685
atgatgatgaggaaaccggacattactacgatcagagacaaaggcaagccaaatcatgcatgtggtggtaataacaacaaaccgaag
ctaagaaaaggactttggtcgcctgatgaagatgagaagctgataagatacatgttgactaatggacaaggatgttggagtgacatcgc
tagaaatgctggtcttttacgttgtggtaaaagttgtcgccttcgctggatcaattacttgaggcctgatcttaaacgtggatccttctctcct
caggaggaggatctcatcttccatttgcattccattcttggtaacaggtggtctcaaatagctactcggcttccaggtagaacagacaacg
agatcaaaaacttttggaactcgacattgaagaagcggcttaagaacaacagcaacaacaatacttcatcaggatcatcacctaacaat
agtaatagtaattccttggacccaagagatcaacatgtggatatgggaggcaactcaacttcattgatggatgactatcatcatgatgaaa
acatgatgacagtggggaacaccatgcgcatggactcttcctccccattcaatgttggaccaatggttaatagtgtgggcttaaaccaac
tttatgatcccttgatgatatcagtgccggataacggatatcaccaaatgggaaacacagtgaatgtgttcagcgttaatggtttaggaga
ttatggaaacacaattcttgatccaattagcaagagagtatcagtagaaggtgatgattggttcattcccccctcggagaataccaacgtc
attgcttgtagtacaagcaacaacctaaacttacaggcccttgatccttgcttcaatagcaaaaatctttgtcattcagaaagcttcaaggta
gggaatgtgttggggatagagaatggttcttgggaaatagaaaaccctaaaatcggagattgggatttggatggtctcatcgataacaa
ctcttcttttcccttccttgatttccaagtcgattga
SEQ ID NO: 30
Arabidopsis thaliana MYB83 (At3g08500) protein NP_187463
MMMRKPDITTIRDKGKPNHACGGNNNKPKLRKGLWSPDEDEKLIRYMLTNGQGCW
SDIARNAGLLRCGKSCRLRWINYLRPDLKRGSFSPQEEDLIFHLHSILGNRWSQIATRL
PGRTDNEIKNFWNSTLKKRLKNNSNNNTSSGSSPNNSNSNSLDPRDQHVDMGGNSTS
LMDDYHHDENMMTVGNTMRMDSSSPFNVGPMVNSVGLNQLYDPLMISVPDNGYH
QMGNTVNVFSVNGLGDYGNTILDPISKRVSVEGDDWFIPPSENTNVIACSTSNNLNL
QALDPCFNSKNLCHSESFKVGNVLGIENGSWEIENPKIGDWDLDGLIDNNSSFPFLDF
QVD
SEQ ID NO: 31
Arabidopsis thaliana MYB58 (At1g16490) nucleic acid NM_101514
atgggcaaaggaagagcaccatgttgtgacaaaaccaaagtgaagagaggaccatggagccatgatgaagacttgaaactcatctct
ttcattcacaagaatggtcatgagaattggagatctctcccaaagcaagctggattgttgaggtgtggcaagagttgtcgtctgcgatgg
attaattacctcagacctgatgtgaaacgtggcaatttcagtgcagaggaagaagacaccatcatcaaacttcaccagagctttggtaac
aagtggtcgaagattgcttctaagctgcctggaagaacagacaatgagatcaagaatgtgtggcatacacatctcaagaaaagattga
gctcggaaactaaccttaatgccgatgaagcgggttcaaaaggttctttgaatgaagaagagaactctcaagagtcatctccaaatgctt
caatgtcttttgctggttccaacatttcaagcaaagacgatgatgcacagataagtcaaatgtttgagcacattctaacttatagcgagttta
cggggatgttacaagaggtagacaaaccagagctgctggagatgccttttgatttagatcctgacatttggagtttcatagatggttcaga
ctcattccaacaaccagagaacagagctcttcaagagtctgaagaagatgaagttgataaatggtttaagcacctggaaagcgaactc
gggttagaagaaaacgataaccaacaacaacaacaacagcataaacagggaacagaagatgaacattcatcatcactcttggagagt
tacgagctcctcatacattaa
SEQ ID NO: 32
Arabidopsis thaliana MYB58 (At1g16490) protein NP_173098
MGKGRAPCCDKTKVKRGPWSHDEDLKLISFIHKNGHENWRSLPKQAGLLRCGKSCR
LRWINYLRPDVKRGNFSAEEEDTIIKLHQSFGNKWSKIASKLPGRTDNEIKNVWHTH
LKKRLSSETNLNADEAGSKGSLNEEENSQESSPNASMSFAGSNISSKDDDAQISQMFE
HILTYSEFTGMLQEVDKPELLEMPFDLDPDIWSFIDGSDSFQQPENRALQESEEDEVD
KWFKHLESELGLEENDNQQQQQQHKQGTEDEHSSSLLESYELLIH
SEQ ID NO: 33
Arabidopsis thaliana MYB63 (At1g79180) nucleic acid NM_106569
atggggaagggaagagcaccttgttgtgacaagaccaaagtgaagagaggtccatggagcccagaagaagacattaaactcatctct
ttcattcaaaagtttggtcatgagaactggagatctctccccaaacaatctgggctattgaggtgtgggaagagttgtcgtctaaggtgga
ttaactatcttaggccagatctgaagcgtggcaacttcacttcagaggaggaagaaacaatcattaagcttcaccacaactatgggaac
aagtggtcgaaaatcgcttctcaacttccaggtagaacagataacgagatcaagaatgtgtggcacactcatctaaagaaaagactgg
ctcagagctcaggaactgcagatgaaccggcctcgccttgttcgagtgattctgtttctcgtgggaaagatgataagtcatctcacgtag
aagattctttgaacagagagactaatcataggaatgagttgtctacatctatgtcttctgggggttccaaccaacaagatgatccaaagat
agacgaactcaggtttgagtatatagaagaagcttatagcgagtttaacgacattattattcaagaggtagacaaacccgatctgctgga
gataccatttgattcagatcctgacatttggagtttcttagatacttcaaactcatttcaacaatccactgcaaatgagaacagctcaggctc
aagagcaacaacagaagaagagtctgatgaggatgaggttaagaaatggttcaagcacctagaaagcgaactcgggttagaagaag
acgataatcaacaacaatacaaagaagaagaatcatcatcatcatcactcttgaagaactacgagctcatgatacattga
SEQ ID NO: 34
Arabidopsis thaliana MYB63 (At1g79180) protein NP_178039
MGKGRAPCCDKTKVKRGPWSPEEDIKLISFIQKFGHENWRSLPKQSGLLRCGKSCRL
RWINYLRPDLKRGNFTSEEEETIIKLHHNYGNKWSKIASQLPGRTDNEIKNVWHTHL
KKRLAQSSGTADEPASPCSSDSVSRGKDDKSSHVEDSLNRETNHRNELSTSMSSGGS
NQQDDPKIDELRFEYIEEAYSEFNDIIIQEVDKPDLLEIPFDSDPDIWSFLDTSNSFQQST
ANENSSGSRATTEEESDEDEVKKWFKHLESELGLEEDDNQQQYKEEESSSSSLLKNY
ELMIH
SEQ ID NO: 35
Promoter pIRX8
ACGAGCTGACTTGTACCGATGAGCTGGCTCTTCTGGGCGAGCTGGCTGATCTTGA
CGAGCAGACTTCTCCCGACGAGCTGACTTGTGTCGATGAGCTGGCTCTTCTGGGC
GAGTTGGCTGATCTTGACGAGCAGACTTCTCCCGACGAGCTGACTTGTGTCGATG
AGCTGGCTCTTCTGGGCGAACTGGCTGATCTTGACGAGCAGACTTCTCCCGACGA
GCTGACTTGTGCTATCCTTTCTCCAGGTCTCGAAAAAGTCCCCTTTCCCGAGACTT
TCTATTCCTTATTTATACCCGTCCGTATAGTAGGGTACGCAAGGTGAATTCTCGA
GAGTGCCCCTTTTCTACGCAGCCGAACTCACATCCTGACCAGGCCGGGCTTCGGC
CTGGTGGGCCGGCTCGAGTTCTAAAGTGATGGTCGGGGCTGGGTCGTTATTCCTT
GAAATGGGCCGGTTGATCACTGAGGCCCAATTGATGTATCAACATGTGGTTTTTA
TAAAAAGAGTCGTGAGAAGAGTTTTCTCTAAAAATCCCTTGTGTTTGGTAATCAA
ACTTCATTCAACCAACGAATTCCAAAAAAACAACTAAATTGTTCGGGTATATAAA
ATGATTGGTAATGATATATCCCATAGAGGCCGTAGACATAGGCCCAAAAAGTTTC
CATAACTAGCAGAAATTGAAACTTGCAAGTTGCAAATATTATTACACTGGAAAG
GCAACAAGTCTTGAAGTACAAACTACAAAGACTTCTTGTTTGGATGGGGACGACT
GACGAGTTTGAATAACTTAAGAGAAAAGGGTCGCAATCGAAATTAGACAAGAAA
TTAGTCCTCAAAAAGTAAATTCTGAAGTTGAAGCTCCAATGTCTTTGTTCAAAGA
CTTTATTTAGATGTAAAGTTATGTCTTGTAACCACCAAACAGCTCCTTTTCATCTA
CACTCCCAATTTTTTTAACATCTATGTTTTGCATTGCCTTTGACTTGTCTTTCTCTC
TCCAACTTCTCTCCTTCAACATAAAGCCAAATCCTAAATCCAAATCCCTTAAACC
GAACCGAATTAAACCGAAGCTGTTGAACTATCGCAAAATTTCAGATCTTACTAAT
CATAAACATGTGACGTTTAATTCATTTTAAGAGTTTCATGATTTGCACTGAATGGT
ATTCCGAGTCCACCGGAAAAAAACTTTTCCTACAAGTAGAAAAAGGATAACCCC
ATAAATCCAAATAACCTAACCGATCAAACATATACCAATATAAACCAAAACAAG
ATTCAGATTCATCGGTTTAGTAATCGAAGTAATGTACTAATGTGTAATATTGATT
CCACCACCAGCTTAGAGATTCGAACCAAAAACCGAATAGCGCATAACCGAGAAA
ACCCAAAGCTTCCTAACAAATACATAAAACCGTGGTGTTTCTAATTCTAACCAAC
ACACGTTTCCTTTTTATTCACAAGAAACATCAGAGTTATGATCTGCCATTAATAA
CCTAAACACAAAGCAAGGTTAGGTAAATGATATGGACCCCTAATGAATAATCAT
ACAATACATAACAACGTAAGATCCAGTTTCCCTCTTCG
SEQ ID NO: 36
Promoter pVND6
CGCCAAAAAGATGTTATGATGTGATGCATTCTTTAATATAGATTAAACTATTGGT
GATTTGTTTTTCTATAGTTAATCACTAGCAAGAACATTTTTCTTTTATGTTTACAG
TTTTTAGATTATAAAAAAATGGTGTAATAAGAACATGTCAAATCAAATGTATTTA
ATTTGTTAATATAGATTTGTGTATAAACAATTGGTAGTTTTGATAATTCAATTTTT
CAGCAATCATCATAATAATACTTTACAAGAAAAGCTAAATCATCTGAAAATTTTA
TATGAATACGTACGGTTTAATTCCCAATCTACAACTTTTTTAGTTGGATTTATTAT
AACCGTTTTTCTAAAACAAAACACATTAAAATTTATAAGTGAAGATCCAATGGGT
TCAAACCTTTTAATTCTCAATAAATATACATAGATTCTCGAAGATATCCTATCAA
CTTGTAAAAGTTGTTTAATCAATCTTTTGTTTGATGAAATCTTGTTCAACTGTTGA
TTTGGTTAAGTTTTATAGCTGAAATGTGTATAAGTGTTTCTGAACCTTTTTAATTA
CTGCTAAATCAATTTATGTCTTACAAACTTGCCGATGTATCATGTATGTTCATTAG
GGGTGTCAAAATGAGCCAGCTCGCTCAGCTCAGCTCATGATGAACTTAAATCTTT
TATGAGCCAGCTCAGCTCAACTCATTTATTATATGAGCTTCAAAATACAAACTCG
GACTCAGATCATCTAGATCACGAGCTAAATGAGGTAGTTCGCGAGCTAATGCAA
ATAAATGAAAAAATTTAAATTTTCTAAAATTTTTATGTAAATTATATATTTTTAAA
AATATATTTCTATTTTTATAGATTATATAAATATTTATGTTTTGATTGTTGACTTTT
TGTGTTTATTACTAATATTTATTTTCAGAAATTATTATGTAACTTATATATTTTCTA
AAATATATTTCTATTATTTATAGAAAAATATATATATACTTTGATTGTTAGTTTTT
TGTATATATTACTTCAAAAAAGCCAAACTCATGAGCTAGCTCATGTTCATTAAAG
CTCGCTCATATAACTCGTGAGCTAAATAAAGTTCGATCACTAAACTCATTTATTA
AATGATCCTAAAAAATAGAATTCGCGCTCATGAATAACTGAGTCGAGTTGAGCC
AGCTCATGAGCTATCAGCTCATTTTGACACCATTAATGTTCATATAATAATCGTA
ATCCATCATGACCAATTAGGCAATTAAGACATACTATAACAAAACATTTTTTTTT
TTTTCGTCAAACATTGTTTTGTTAAAGGTTTCAAAAAGAACATGCGTATCAATTTC
ACCAAACGAATCTAATAAATAGCACGACCATCGGATATTATATTGCTATTTGACG
ATCAACGTACGTTCGATTAAAAAAAGTACGGGGATTGTTTAGTTTAATCCAAGTG
TAGTGTTATTTAGACTTCGAGTAACATCAACGCGGGAGAAGAAACGCATGGGAT
GAATGTGTAAAGTGGTTAACTTTCAACAATGTTTCTTAATTGCTAAGATGTTTAA
AACGAGTTTACATGAATTCTTGTGTTACTTATCGAATTATCTTTTATGAGATAATT
AGTACACTACCTTATTAGGGCTCCATTTCTTTTTCTATCTAGGCCTAGGTCGATCA
GTACTGTGTATGTTACACATATGATATTAAAAAAAAAATTGATTCTATAATAATC
TTCATAACTAAAGGCAAGTATAGTTTATTTGAAAACGTCTCATTCAATAGTTGGT
AGAGATAGTTCTAAAAGATCACATTACTTCTCTATCACACAGAGTGTAGAAAAGT
GAAAACTTATGCAACACTTCAGGTAGAAAGAGAAAAGACAGTGATAGCTTAGTT
ATAATTAAAGACCCCCAAAATCCAAATAGAATCTTCTCTTAAATAAACTATTGAA
AAAATATTCACAAAAAAATAAAAAGCACATTTCCTTTTGCTTGCATCACGAGAGC
TTTTGTCTCTCTTTACATTTATGTGCCTATATATAAGCCTAGAGAATCCACCATCA
TCGGAGTACTCGTTGTCTTTCATGATCTCAACATAAACAAGACAAAACGCTTTTG
TCTCTTGTCTATCTAAATTAAATCTACGAGGAAACGAAGAGAAGCAACAAAATTC
GATATTTTAGAAGTCTTGCATAAATAAGAAGAAGGTTTCAAGTAAGTTTCTTTCA
GTACATAGAGAAACCATGTATATGATTTATGATTGTCAAGATGTTACACAGTCGT
ATATATAATTATAAGTATTTCATAAAAGTAATTTATGAAACCATGCACATAACTA
ACTTTTGTTTTTGATTTTGTAAATTGAGTAGTTTTTGTTTTTATTATCATTTTTATG
TGTTTATAGTTGGTTCAGCCGAGATATTATGAGTAACCAAACGTAACCTTTTTCAT
AATGAAACGGATCAAATATACTTTAATCTTTTTCCTACATATGCTTAGTTACTTGA
AAACTTGATTTCACATTACTTCTATGCATATCTTTTCTATGTACCGCGCGATGATA
AAGTATGTGTTACAAATTGCCACATTGCAGAAAATATAAAATTAAAAAGATCAA
ATGGAAAGTCTCGCACAC

Illustrative SHN1 Protein Seuqences and accession numbers

SEQ ID NO: 37 AtSHN1_At1g15360_NP_172988
MVQTKKFRGVRQRHWGSWVAEIRHPLLKRRIWLGTFETAEEAARAYDEAAVLMSG
RNAKTNFPLNNNNTGETSEGKTDISASSTMSSSTSSSSLSSILSAKLRKCCKSPSPSLTC
LRLDTASSHIGVWQKRAGSKSDSSWVMTVELGPASSSQETTSKASQDAILAPTTEVEI
GGSREEVLDEEEKVALQMIEELLNTN
SEQ ID NO: 38 AtSHN2_At5g11190_NP_196680
MVHSRKFRGVRQRQWGSWVSEIRHPLLKRRVWLGTFETAEAAARAYDQAALLMN
GQNAKTNFPVVKSEEGSDHVKDVNSPLMSPKSLSELLNAKLRKSCKDLTPSLTCLRL
DTDSSHIGVWQKRAGSKTSPTWVMRLELGNVVNESAVDLGLTTMNKQNVEKEEEE
EEAIISDEDQLAMEMIEELLNWS
SEQ ID NO: 39 AtSHN3_At5g25390_NP_197921
MVHSKKFRGVRQRQWGSWVSEIRHPLLKRRVWLGTFDTAETAARAYDQAAVLMN
GQSAKTNFPVIKSNGSNSLEINSALRSPKSLSELLNAKLRKNCKDQTPYLTCLRLDND
SSHIGVWQKRAGSKTSPNWVKLVELGDKVNARPGGDIETNKMKVRNEDVQEDDQ
MAMQMIEELLNWTCPGSGSIAQV
SEQ ID NO: 40 PtSHN1_XP_002324652
MVQSKKFRGVRQRHWGSWVSEIRHPLLKRRVWLGTFETAEEAARAYDQAAILMSG
RNAKTNFPIPQTSNEEDPKSSDEASLPTPPNGLSEILHAKLRKCSKAPSPSMTCLRLDT
ENSLIGVWQKRAGERSDSNWVMRVQLGQRESQVSESTLPLPQSSGGVSEPELRAEM
GEDERIALQMIEELLNRNCPSPSFGVQDHGDGSLFL
SEQ ID NO: 41 PtSHN2_XP_002308080
MVPSKKFRGVRQRRWGSWVSEIRHPLVKRRVWLGTFETAEEAARAYDQAAILMSG
RNAKTNFPMPQTSNEDDPKSSDHQPSLTTPPNGLSQILHAKLRKCSKAPSPSMTCLRL
DAENSIGVWQQRAGQRSDSNWVMTVQLGKRDESQVSESALPLPDQSPGGISGPEWR
EEMDKEERVALQMVEELLNRNCPSPPFGVQDHDDDSFFL
SEQ ID NO: 42 PtSHN3_XP_002327422
MVQSKKFRGVRQRHWGSWVSEIRHPLLKRRVWLGTFDTAEEAARAYDEAAILMSG
RNAKTNFPVVANQTRNGQNSPSSSSALSAKLRKYCRSPYPSLTCLRLDAENCHIGVW
QKRAGPRSVSNWIMTVELGKKDGRQAPEQKILISDTSDMAGQEGGSDDGPDDEERV
ALQMIEELLNR
SEQ ID NO: 43 PtSHN4_XP_002324859
MVQSKKFRGVRQRQWGSWVSEIRHPLLKRRVWLGTFETAEAAARAYDQAAILMNG
QNAKTNFPTSHLDQDTNLGKDNNSPLPAKALAELLNSKLRKCCGKDPSPSLTCLRLD
NDNSHIGVWQKKAGSRSSSNWVMKVELGNYNKKTESSPTVEIEPENGTEEEDRIAM
QMIEELLNRN
SEQ ID NO: 44 PtSHN5_XP_002309625
MVQSKKFRGVRQRQWGSWVSEIRHPLLKRRVWLGTFETAEAAARAYDQAAILMNG
QNAKTNFPASHLDQDTKLGKDNNSPLPAKALAELLYSKLRKCCGKDPSPSLTCLRLD
NDNSHIGVWQKKAGSCSSSNWVMRVELGNSNRKSTQVMEELRPSLSSESSSRVEIEP
EINGTDEEDKIAMQMIDELLNCN
SEQ ID NO: 45 MtSHN1_XP_003609337
MVQSKKFRGVRQRHWGSWVSEIRHPLLKRRVWLGTFETAEEAARAYDQAAILMSG
RNAKTNFPITQTSEGDPKSITSNENKPSTSKDLEEILHAKLRKCSKVPSPSMTCLRLDT
ENSHIGVWQKRAGKCSESNWVMTVQLGKKMSVTQDSGSSSSSVAPSSAVATEEEIV
RGEIDEEDRIALQMIEELLNDKNCPSPSINNIKQGDDIDNSFFL
SEQ ID NO: 46 MtSHN2_XP_003597892
MVHSKKFRGVRQRHWGSWVSEIRHPLLKRRVWLGTFETAEEAAKAYDEAAILMSG
RNAKTNFPINVENQTNSISSSSTSSKAFSAVLSAKLRKCCKFPSPSLTCLRLDAENSHIG
VWQKGAGPRSESNWIMMVELERKKSASVPEKAKPEELSKNGLDDEQKIALQMIEEL
LNRN
SEQ ID NO: 47 MtSHN3_XP_003604418
MVKSKKFRGVRQRHWGSWVSEIRHPLLKRRVWLGTFETAEEAARAYDEAAILMTN
SNNKTFATSSSTSTKPNTSLSAILSAKLRKCCKSPSPSLTCLRLDTENSHFGVWQKRA
GPRSDSSWIMMVELERKKKEQEEESEVLPNSDSETLASVVDNEDSEKAVKPENEDEE
GNDKNKGLDEEQRIALQMIEELLNRN
SEQ ID NO: 48 MtSHN4_XP_003603408
MVQQTKKFRGVRQRQWGSWVSEIRHPLLKRRVWLGTFETAEAAARAYDQAAILMN
GQSAKTNFPVTKNQGEEVASDTPYNGGGGDDSFLSPKALSELLSTKLRKYCKDPSPS
LTCLRLDNDNSHIGVWQKRAGPHSDSNWVMRVELGGKKKTIESEEIGSKQHTIDGG
NNSNADNENRVVVEEEERVALQMIEELLNWNYPCGSTSSN
SEQ ID NO: 49 MtSHN5_XP_003588762
MVQRNKFRGVRQRQWGSWVSEIRHPLLKRRVWLGTFETAEAAARAYDQAAILMN
GKNAKTNFPIPKDQTEDANSLTPNCDDNNNSFHTSNALSHLLKQKLTKCCQKQSQSL
TCLRLDADNSHIGVWQKGAGSHSDSNWILRVELGKKHEDSHESNYVSSSEKSAPNN
STIVGDCAEKNGIEHEEDIVTMQMIEELLN
SEQ ID NO: 50 OsSHN1_NP_001046226
MVQPKKKFRGVRQRHWGSWVSEIRHPLLKRRVWLGTFETAEEAARAYDEAAVLMS
GRNAKTNFPVQRNSTGDLATAADQDARSNGGSRNSSAGNLSQILSAKLRKCCKAPSP
SLTCLRLDPEKSHIGVWQKRAGARADSNWVMTVELNKEVEPTEPAAQPTSTATASQ
VTMDDEEKIALQMIEELLSRSSPASPSHGEGEGSFVI
SEQ ID NO: 51 BdSHN1_XP_003563662
MVQSKKKFRGVRQRHWGSWVSEIRHPLLKRRVWLGTFETAEEAARAYDEAAILMS
GRNAKTNFPVPRSATGEIIVAPAAARDSRGGGLGSSSGAGSLSQILSAKLRKCCKTPS
PSLTCLRLDTEKSHIGVWQKRAGTRADSSWVMTVELNKEPAAAATTTTLSDSVAPT
TPSTSSTSASTAGSPPVGMDDEERIALQMIEELLGGSSPNSPSHGLLQGEEGSFVI
SEQ ID NO: 52 BdSHN2_XP_003571428
MVQPKKKFRGVRQRHWGSWVSEIRHPLLKRRVWLGTFETAEEAARAYDEAAVLMS
GRNAKTNFPVQRSSTGDPAPAAGRDVRGGNGGGSSSSSMSNLSQILSAKLRKCCKAP
SPSLTCLRLDPEKSHIGVWQKRAGARADSNWVMTVELNKGVGLPSDVEAQSTISTA
TTSSSVSTMDDEEKLTLQMIEELLSRSGPVSPSHGEDEGDFVV
SEQ ID NO: 53 ZmSHN1_NP_001148685
MVQPKKFRGVRQRHWGSWVSEIRHPLLKRRVWLGTFETAEEAARAYDEAAVLMSG
RNAKTNFPIQRSSTGEPTPAAGRDARSNFSSGSSTTNLSQILSAKLRKCCKAPSPSLTC
LRLDPEKSHIGVWQKRAGARADSNWVMTVELNKDAASTDAASQSTSATTAPPATP
MDEEERIALQMIEELLSSSSPASPSNGDDQGRFII
SEQ ID NO: 54 SbSHN1_XP_002451740
MVQPKKFRGVRQRHWGSWVSEIRHPLLKRRVWLGTFETAEEAARAYDEAAVLMSG
RNAKTNFPVQRSSTGEPTPAAGRDAHSNAGSGSSTANLSQILSAKLRKCCKAPSPSLT
CLRLDPEKSHIGVWQKRAGARADSNWVMTVELNKGAASTDAASQSTSATTAPPATP
MDDEERIALQMIEELLSSSSPASPSHGDDQGRFII
SEQ ID NO: 55 SbSHN2_XP_002438651
MVQSKKKFRGVRQRHWGSWVSEIRHPLLKRRVWLGTFETAEEAARAYDEAAVLMS
GRNAKTNFPVPRTATGELAPVPAARDARGGGGSSSAAAAPGGGTSNLSQILSAKLRK
CCKTPSPSLTCLRLDPEKSHIGVWQKRAGARADSSWVMTVQLNKDVPPPASSSGEEP
VPSDGGAAATTPTSTSTSSTVTTTGSPPPAMMMDDEERIALQMIEELLGSSHSHGMFQ
GAAGSIVI
SEQ ID NO: 56 HvSHN1_BAG12386
MVQSKKKFRGVRQRHWGSWVSEIRHPLLKRRVWLGTFETAEEAARAYDEAAILMS
GRNAKTNFPVPRSANGEIIVAPAAAARDIRGGVGSSSSGAAGASSLSQILSAKLRKCC
KTPSPSLTCLRLDTEKSHIGVWQKRAGARADSSWVMTVELNKEPAAAAPPTPSDSTV
S
SEQ ID NOP: 57 PsSHN1_ABK22668
MARPQRYRGVRQRHWGSWVSEIRHPLLKTRIWLGTFETAEDAARAYDEAARMMCG
PRARTNFPFNPNAPQSPSSKVLSSTLTAKLHRCYMASMQGPRSGSSKKDSMARADK
NNNIHSGNQSLTCLRLDNERSNNIGIWQKKSGSKQSESNWLMKLELDHDQHGNSTL
KRETDDDIAQMIEELLDCGSLEICSPIASADSNINSAESMLN
SEQ ID NO: 58 SmSHN1_Sm92334_XP002969836
MGRPQRYRGVRQRHWGSWVSEIRHPLLKTRVWLGTFETAEDAARAYDEAARLMG
GPRARTNFPYDPNAPPHPSSSTLLSTLSAKLNRCFSSSSSSSSCSTDPHKKDPRVSQSLT
CLRLDPEQSNLGIWQKKSGRQPESNWVMKVHFGSQGGGGVSSDIVLPTDNPAPPQPI
EHKKMKSEEDLATEMIEELLNFPDSSSPSSSTSSSEAKNPNFSSSDLLHHILV
SEQ ID NO: 59 PpSHN1_XP_001762992
MGRPQRYRGVRQRHWGSWVSEIRHPLLKTRVWLGTFETAEDAAHAYDEAARLMC
GVRARTNFPYDPNASKRPNSQMLSATLSAKLHRWYLHSQQRDGQEGKSKDARMTQ
SLTCLCLDAEQSNLGIWQKKTGRQAEANWVRKVQFGDNNSPQTDSPQPENSSESCM
SEEDKFAAEMIEELLGYSPGQFSNFGSPAMSDSSCSSSCSAVTTAFE
Legend: At: Arabidopsis thaliana, Pt: Populus trichocarpa, Mt: Medicago truncatula, Os: Oryza sativa, Bd: Brachypodium distachyon, Zm: Zea mays, Sb: Sorghum bicolor, Hv: Hordeum vulgare, Ps: Picea sitchensis, Sm: Selaginella moellendorffi, Pp: Physcomitrella patens

Illustrative promoter sequences of wax cutin genes that can be used to drive
transcription factor expression in wax/cutin APFL:

SEQ ID NO: 60 pAtCER1_At1g02205
TGGGATCCTCTCCATCGTTTCCATCATGGGATACATTACTTACATTGACTTCATGAACAACATGGGACATTGCAA
CTTCGAGCTTTTCCCTAAGCGTCTCTTCCACCTCTTCCCTCCCCTCAAGTTCCTCTGTTACACCCCCTCGTACGT
CGTTTGTAAAAACACTACTATTTATTTGGATTTGAATATATGCATGCATTTATAACATGGCATCAAATATGTTGA
AGGTTTCACTCGCTCCACCACACGCAGTTCCGGACCAACTACTCTCTGTTCATGCCAATATACGACTTCATCTAC
GGGACGACTGATAATTTGACCGACTCACTATACGAGAGGTCGTTGGAGATAGAGGAAGAATCACCCGACGTCATC
CACCTCACTCACCTCACCACACACAACTCCATCTACCAAATGCGCCTTGGTTTCCCGTCCCTCTCCTCTTGCCCC
TTGTGGTCTCGACCCCCATGGTACCTCACATGCTTCATGTGGCCCTTCACTCTCCTCTGCTCATTTGCCCTCACT
TCAGCTATCCCTTTGCGCACCTTTGTCTTTGAGCGAAACCGTCTCCGTGACCTCACCGTTCACTCTCACCTCCTT
CCCAAGTTTTCCTTTCACGTAAGCATTTTACACGCATGCATATGCACTTTCCCTCTCTCTCGCTCTCTTGACACA
AACGGGTTTACTTATTATGTTGTACTTCACACCACTATTTTCCAAACTTTCACACACAAAAAAAACATTGATTCT
CACTTAACTTGTACGTACTACGTAGGAAGGACATAACCTTTTATACAAAAAGAAAATTAAAACAACCTAAAAATG
AACAATGAACATTTTCTTGTGGGTATACCTTTTGTAAGTTGCATCTAAAGTTTATTAATAATTTTGCCTCATAAA
CATGCGGCGGTAGTATAAATCACAGCGCCACCATGAGTCCATCAACACTATCATAGAGGAAGCTATCCTTGAAGC
AGATGAAAAGGGTGTGAAAGTAATGAGTCTTGGCCTGATGAACAATGTAAGTTTATTTACTTCAAATCCCCCTAA
ATATATATGAATCACTATTGGATAAGTACTGTAACTAATTACACACGCTGGCAAAAGAAAAAAGTAACTAATCAA
CACACAATTAATTAATGAGAGGGAGGAACTAAACGGGTCTGGAGAAATGTACGTGCAAAAGTATCCGAAGTTGAA
GATAAGACTAGTGGACGGGAGCAGCATGGCAGCTACGGTGGTTATCAACAATATTCCAAAGGAAGCCACGGAAAT
TGTCTTTAGAGGAAATCTCACAAAGGTGGCTTCGGCTGTTGTCTTTGCTCTGTGCCAAAAGGGCGTCAAGGTTTG
TGAATATATGAGGGAATTGGAATATATATACTCAGCTTCTCATATCAAACAAAAAATAATGTAGTAATGTGTATA
TATAGGTGGTCGTGTTACGCGAGGAGGAACACAGCAAACTCATCAAATCTGGGGTTGACAAGAATCTGGTACTGT
CTACAAGCAATAGTTATTACTCCCCAAAGGTGTGGTTGGTGGGGGATGGAATAGAGAACGAAGAGCAGATGAAAG
CAAAAGAAGGAACCCTCTTTGTTCCCTTTTCTCACTTTCCGCCCAACAAACTCCGCAAGGACTGTTTCTACCAGT
CCACTCCAGCTATGCGTGTTCCCAAGTCTGCCCAAAACATCGACTCCTGTGAGGTACATCTTTGAATTCTTATAG
ATATATCTGTAACTTTTATATTATATAAGCTGATAGATGTGTTCATCTATAATGAATGAATGGTTGTTATATATA
TATAGAACTGGCTGGGGAGGAGGGTGATGAGTGCATGGAAAATAGGAGGTATAGTGCATGCACTTGAGGGTTGGG
AGGAGCATGACTGCGGCAACACTTGCAACGTCCTCCGTCTCCACGCCATATGGGAAGCTGCTCTTCGCCATGATT
TCCAACCTCTCCCACCATCTCCTCTATGAGCTTTTTTCATATTCATACATCTATGTCCCCTTTCTTGATTATATC
TACTTCCCTTCCATCATTGTTTGCTGTTTACTATGTTTTTCTATCGACAATATATAAGTACCCTTGTTACCCTTG
GTGCCACGTGCTTCATATATGTTAGAAAGGGCAAAAAAATTCGTCGTATGATATGCTTAGTTAAATTTTATAAAA
CTCAATAAAAATCTTCAGAAACAGTGCTATGATCATTACATCTTAACTAAGTGATATATATCTGCGTGCCTATTT
AACAAAATAAACAAAAAAACAAAACAAAATATATTTGGGTGCATCATCAAATCAAAGTAGTTGCAAAAACTGGAC
GAGGTTTTTACTTAAATGGTCCTTACCCCGCATGGTCCACTTGCTACCTAATTAAGGATTGGTAGGGTGCGTATA
CGTATATAAATTGTGGCGGTGGGAGATGGAGTTACTAAAAACGAAACGTACAAGTATTATTCATAGCTCTCGTAT
AAGGGGTTAGTCCTTAGATCTAGATATTTTCACTTTTCTTTCATTTATGTCGGAGCAACAGACACTAGCTGGCGC
TTCAACGTGCATGATCTTGATTGGCTAGTAAATTCCAAGCATCAATACCTAACACATGCCCAACTTGGTTCATTA
GTATTCTTTCATTGGTAAAATACCCTTACCTTTCAATAATATCCAGAAATAAATATATGAAGCCATCCATCAACC
GGTGCATTTCCTCAAGGCATGGATATGATATCAGAACATCGATGAAGGTGGGAGGGGGTAATTAGCTGAGTGTCA
TAAATGAGGATCCATGTGGAGATCATCGAATGGTAGTAGTACATGTTTGGTCTTAGCTGGCCCCACCACAAGGAA
TTGGACTGGTGGGAAGATAGGGGTGGTTACGTCATTCCACATATCTACCAATTAAGGAGTTTAATATAAACCTTG
CTATATAATGTACCTTGGCTCACAAGAGTTGAAGAGACACAGTGACGACACAAACATATTACATTCGACGGTATA
SEQ ID NO: 61 pAtCER2_VC2_At4g24510
ATCGTACTAAAAATCTGGGAAGTACACTGATCCGATGGAAATGAAGGAGAAGATTGTACATTTTATCGTGATGAT
TCGTCATAAACTGAGACTGGAGAGAGCGAAGCTGAACAGCTTTAGCAGCTGAAGCTGCTTCCTCTTCTGGCTTAG
AAGCATTCCTTGGACGACCGTGCATTCTTCTCCGACGACTGTGAGAGATGCGACAGTGTGATTCAACTTTTCCGG
TTAAATCTGATCAGAAATTACGAAAACAGTTAAAACAAACACTAGATCGAGGAAAGAAAAAACGTGATCCAAATC
TAGAAACAACAGAGATGACGAAATGGAGAATAAGAAGCAGATCACCTGAAAGGAGCACGTTGCGGCGTCGATCGA
GAGAGAGAGACTTTTGTTTTCGGGCAATTGAGCGACTGAGAGAAGGTTCTATCGGAAAATTTAATGGGCCGGTTT
AATGATTTCAATCGCTTAAACCAGTAAACCGGGTTTAATTGAAAAATACGGTCGAACACTAACCCGGTTTTGGAA
GTCTTCGTTTATGTAGTGAAAGGCTTCGTCATCATCGTTGTTTCACAGTTGAAGGTTTTCACGAGAAAGTGAAGA
TTTTTGGAGAAACCATACTTTTGTGAAGGTTCGTTTCACCTTCAATTTCGTTTTTCCAATTTGGGTATCCGATAA
TTGTAAGAATTTGGGGTTTTAAGGTCAGATTGATTATGTATCTAATGCTTTTACATTGTAGTAGCTCGTGTTTTG
GATTTTGAAGGATTGATTTCTAAATTTCCTTTTGATCTTGTAGTTGAATCACAATGGAAGATTCAGAGAAAAGAA
AACAGATGTTAAAAGCAATGCGAATGGAAGCTGCAGCGCAGAATGATGATGATGCTACTACAGGTACTGAAACAT
CTATGAGCACAGGTCACCTCTCCAATCCATTGGCAGAGACATCCAATCACCAGCAGGATTCATTTGAAACGCAAA
GGTTTGATTATTATACCGATCCCATGGCTGCTTATTCTAGTTTCAAGAAAAACAAGACCCCTAAGCAACAATACA
TCTCATCTCCTAGTCATCAAGGAAGCTCTCCTGTACCACCTCAGTTTCCACCATCAGTTCCTCCAGGTGCTTGTT
TCTGCCTTGATAGTCAATGACTCTGTCTTAATTTTTGTTTTGTCTATATTCTTGTGAGGCCAAAGAAAGTAGTCC
ACTGATAAAGAAGATTGCCTAATATCATGAGAGTTTAACTTACAGTATGTTAGTTGGTCCTTGGTGAGTATAGGT
ATAGGATTGAACAGAATCAGGAATTCACTTTGGTATTTGGTTGTTGGGCTTTCGTAGAGTTAGGTGATGATTCTC
TTCTACAAATCTATATGAAATTTTTGTTCTTTGTTTGGGTTTAGTCATGTGTTCTTGATTATAATCTACAAATCA
AGATCAATGGAGGAAGAAATTTGTCTCTCAGTCTCAGGCTTCAAACTTATTAGCTTTAGCTACTTTAATTTGATA
TATCTCTTTAAGTTTCACTTTCTAGGAATTGACAGTATGAAAATCACATCAATTATGGAATTCAGTTAGGTTTGT
GTCCTGTGCCTTGCAATGAACAGTAATCCAGTTCTGATCCTTGCATTATAACTTTGAACAGGATCATTATGTAGT
GAGTATCAGGCACAGACCAATCATGGTGGCTTTCATGCAGCTCATTATGAACCAAGAGGGATGGCACATCTTTCA
CCCTCACACAGAGGTCCACCCGCCGGTTGGAATAACAACTTTAGGCCTCCACCAGTTAACCATTCAGGTCCACCT
CAATGGGTACCTCGCCCTTTTCCATTCTCTCAAGAAATGCCGAACATGGGGAATAATAGATTTGGTGGTCGAGGC
AGCTACAACAATACTCCTCCACAGTTTTCCAATTATGGACGACAAAATGCAAACTGGGGTGGAAACACGTATCCT
AACTCAGGAAGAGGCAGAAGTCGAGGACGCGGTATGAACACAAGCTTTGGGAGAGATGGAGGAAGAAGACCCATG
GAACCAGGGGCAGAACGATTTTACTCCAACTCTATGGCTGAAGATCCATGGAAGCATCTTAAGCCAGTCTTATGG
AAGAATTGCTCAGATGCTTCGAGCAGCAGCTCAACAGGTCAAGCCTGGCTTCCCAAGTCTATAGCACCAAAAAAA
TCTGTGACCTCAGAAGCTACCCACAAAACTAGCAGTAATCAGCAGAGCCTTGCTGAGTACCTTGCTGCTTCTCTA
GATGGTGCTACATGCGATGAGTCAAGCAATTAATCAGGACAACTGGCCTAGTGAAAATACACCAAGTCCAAGCAT
CTGAGGTACCTGGAAAGATCACCGTTGACTCTTTCGAACTAGGTATTGTTCAGTGACTTGTGTTTATTTTCTTAA
CAAGACACAACAGCGAATGATGAACATCTCTGAGGGCGCAATTAGGAGTAGATTGGTTGGCAATAGGGATGTTCT
CTACCAAAAATTTTACTGTTTTTTCGCAAGATTTAGTTATCGTACAATTATGTAAAATCATTATCAGGAAATTTG
TTGCATGATTGTGTTTGAGGTGGAAATGAACCGCATCCGTATTAAGATCATTTTTGCTGGTGGAAACAATGTTAC
CAGGAAACTGAACTTGGTTTTTTATAGATTAATGTGACTTGTTAGGTACCGTAATATAATACTAGTTGGCTACGA
CACGTACATGTGCGTTTATTGCTTGAAGCCAATAAGGACAAGGTGGACGTAATAAAGTGTGCTTGTTGTTGGATG
GATCTGAATATGATGACTCAACTGTCCAACTCTAATGTTGTTGCTAAAGACCCAAATCCCACCCACATTTAATGT
TGCCGTCACGGAAACAGTTTTCCCAACTGTCCTAAATCAGTGATACCCATGCCTATTCTGAACTCAACTCTCTTT
CGAAACTCAATCCTTATATAACACATCCCATTTAAGCCTATAAGCTACACATATCAGCTCTCTCACAAAAATAAA
SEQ ID NO: 62 pAtCER3_WAX2_At5g57800
ATTTTAAGAAATTAAATCTTTTTTTTTGGTATCTGTGTGTTACTTTTTATAAATGTATTTTGAAAAGTATTATTT
CAATTTATAGATAAATATAATTATGACTCTCAAGTGGATTTTTTTTTCACGGTCAATTGGAGTTTGGCTGTGAAT
CTTGTGAATAATATCAACTAATTAAGAGTCGGCCGATACTCCGTATCTTTGATCACTTTCACCCATATCTTGAAA
TTTACCAAATGTTTTCTTTCATTGGGGTTTCTATACCTTTTTTTTTTGGTTCTTTCTGTAATTTGGGTTTTAAAG
AAAAAACCTATAATTACAAAAAAGGTTAAAAGTTAAAACTAAGCACGTTCAAATATGTTCTTTCGAGATCCCACC
TTCCTGTCTATCTTATAAGAAGTCGAACACGTGTGCTCGTTGTGTGAATTAGAGTGGTTATTTCAACATATATAA
TTAAACTATGTATGTATCATAAATACACTGAATATGTTGGAGTTGGGTCTTGTGGTAGGCTAACTTGTTCGCTTC
ACTTTGGTTTAGTTTAGCCTTGTAATCGGTGGTTTCATATACTGAAATTTTTAATTTTCTAATATAATCCAGATG
ACAAAAAAAAATCATAAATACACTGCAATGGCCATTTTTAAAAAAACTTTAAACGAAATATATATACATCACCAA
GTATCAAGTAAGTAATTTGGTTGTTTTGATTTTTCAAAAAAACATTTAGGTGATTCTTTTAACAACCAATCATAT
ATGTAGCAAAGATTAATTATGTCAATCTTTTAACGTCACTTATATAGTTTAGCTGTAGTATTATTGTAAGCCAAT
TGTACGGACTTCGTAACCGTTTCGACAAAGATACAAACTAATCAATGTTTAAGATGGCATAATATTCCGACTTCC
CCTGCCACTCGGTAGTACTGCTGCGCCATCCTACAAGAAGCACTCGTAAATGAGTGGTTAGACGGCCAAAAATTA
GCAGGATTCAATCCTTGATTAAGAGATGGTTCAGATAAGCCCTCTAGGCCTCTACTCGTCACAATTCATATATTA
ATTGATCACACTGCATGAAATGATAAGATCAAGTCAATTACCTCAAAATTAATTGAAAGATAAAATGATAAAGCA
ACAAGTAATGATCATATATGGATGTAACAACCTTCTTCATCCATACGAATATGATAATTCTTTTGTCGATCATTT
GCCACATCTATTTAACTAAGTATTTTGTACATTAAGATCATTTTGCGAGTATTCCCGCGTTCCCAACAAAAACGA
ATTGAAAAACTACATAGGGTGAATTTTGCTCTCGTGGCATTAAAGAGAGAGGCTGACATTATAGATTATGATATA
TTTATTATTTTTCTTAGTAGATTTCTTGACCAGCCTTTAGATATGCTTGCTCTGTCATGGCATAAAGCCTTAAAC
CCTAAAGCCCTTTACATTTGTACAGTACCAACAATTGTACCCAATGTTAAATGAATGCGGATTAATTTGTGATAT
GAGACCATCAAAATAAAATACTTATAAGATAGTAGATATATAGAACCGTAACAAAAAGATGAGAAGAAAAAGACA
AATTGAATAGAAACTGGACCAAAAGATAAAAGAGTATATGAAGAAGCAAGCCACCAAGAAGTGTAACCATCGTTG
CTTCAACTGTAAACCGCACATTAAATGTGTGTGAGAGAAAGAGAGATAGACTCCAGCTGTGAGAGCTACGTCCAT
TGCATCAATTGGGTTATATGTCGTTAACAATTTTTTAAAAATACATAAATAAATCAATAAAATATTTAGAAAAAA
TCAATATTATAATGATATTTTGAAGAAGTTAGGTCCATAATGGAAATATAGAGACTTAGTGTCTATTTATCAAGA
AATAGTGTCTATTTATCAAGAAATAATGTCTATTTGGTCCACAATTATCATATTTTTTGTGGCCACTCAAACTAC
GAAAATAATGAATATTGTTAATTTTTGTCTTATTTAAGTCTATAACTAACTTACTAATTCGGTTGAAATTGCTAA
TATGCATTTTCCTTGGTCCACTATTACAGTGCTACTGCTGTCTTATTCTTGAACACAAAATAAAAAAGATTTCAA
ACACAAAATATGCATTTTTTTGTTCCACCATTACAAACCTTTATTTGGAGAAGTATACAGTTGGATATTAAATAT
TTTAATTAAAAAATTGAAAATATTGAAGTTCTTTTTCTCCAGTATTTTGGCTGGAGCAAAACTAAATAAATTCCA
AATTAAAAGCTCGAACGGAAAACATTTACTATGCACCAAAATAAATGTTGATATGAAATTTGCATCAGCATATAC
AGTATGTATGTAGTACAATTGAACAGAGTCAAAATTAATTGTCACTGAAACAGTCAGATTTTATTTCTGTTAAAA
AATATTTTCAGACAAAAAATGATTTTTTAACGTTTTTTTATATAATTTTATTAAACCTCAAAACATTCCTCAGCA
GATTGTTTTATAATGTTATTATTATTTTTTCTTAAATAATTTTATTTGAATTTCGTTATAGAGAGATAAATAAAA
GATTTTTGGGGTCTAGTTGGCTCTCATAGTTTGAATGCTTATATAAGGAAACAGAGCTCGAGGCTCTCAATAATG
CCACATAAAACACAACACCAAAGCCTTTATAACTCTTTCTGGGGTTTTTAATTCTCTCAACGAACAAAAAAAAAA
ACTCAACTAATATTCTCCATCCTCTTTCTTCAGATTTAAGAGAAACCTTATCGTTATATTCTCTCTTCTCTTTTC
TTTTTGACTCCCACAAGAAGAAAGCAAATTTCTTTTTCTCAGGATTGCTTGAAAACTGTAGAAATCTTTAAGGTT
TTTTTACACCAATAAGAGTCAGAGAGAGAGAGAGAGAGAGAAAGAGAGATCTATTCATAAAAAAGACAGGTAATC
TCAACTCCGAGGAGATAACTGATATAACAATTTGAGAGAATAAGAAGAAGAAGAAGAAGAAGACCAAGCTAAAGA
SEQ ID NOP63 pAtCER4_FAR3_At4g33790
TTCTTCAAGGAACAAAGATCTATCTTTTGGTTGCGTTTACATAGTGTTTACGTTCCATGTTATAATTATTAGCTC
ATATTAGAAACCCAGACGTAAACACAAACAAATAATCATGTCACTATACAGAATTTACCTAGTATAGAAGTCCAT
GATTTTTGTTCTTTTTACCTTTTAATAGAAAAGTTTTGTTCTTTAGTTTTTTGGGATTTAACATTCGTTTTAATA
TATCTTGTAGATGTTGCAGAAAAGTGTTGGTGGTAATAAGATAGAGCAAACCTTTCCAGAAAGTACATTGGATTA
CTCACAAGGCTTCTCGGCTACTCAGTTTCAGGTTTCTCTCTCTTCACTATCTCTCTGTTAGGTTCTGTTGAGTAC
ACGTGACTTGTCTCAAGAATCTCACTATATATGTCTTTGGGTGTTGAAACAAGCAGGACAAAACAGCATTCCAAG
AGCAGTGTGGTTATTTGCATATGGAAACCAGATTCTAAATAACCAGTTCAGTAATTTTTTATTATTATTTCTGTA
GATGTTCAACTACTTTGTTTATTCTTACTTTATAAAGAATATATCAAATCTGACTAAAATTAACTTTGAAATTCG
CTGGTGAAGATCAAACTGATGAAGAGCAGCAGCTATTTTGGACATTTGTGAAGCTTTTATGGATTCAACTTGAAT
CATTTCAAGCAGTGATTAAAGTGAAACTTTAACGTCCCAAATATGAAAAATAGGAAGGAGTTTTAAGCAAAAGAC
TGATATAGAACGAAAAAAAAAAAGGGTTCTCAAATCTCAACTGGACTGATTCTTTTCTCTCTTTTGCTTTGTAAT
TTCGTCACAAACAAGTTTTCCTTGTAGCCGCCTTTATGTAACTCGTGGTTTACAAATTACAACACTTCACTTCCA
TTGCAATTTTCTTTCATAGTATAACAAATTTCAATGCCTGAATAAGCTTTTTTTTTGTGTGGGGAAAAAAACTTC
CAAGAAATATCTAGCAAAACAAATGATCTTAATGGTTCTTTTCAGAGCTGCTAAATTCTCAAAAGAATAATTCGA
AGTAGATGACAAAGAGGGCATATTCAAAACTTATAAAGAAACTAATAAAACGTATTACTTTAAATAGAGCACGAA
AACCTATATATAACCAAAATGCTAACCCTAAAAGTAACACTTGAAGAATGCGTAACACTAACGCAATAAAAGGGG
AAAATAGAAACTTATCAAAATCTGGTTATCGGCTTGTTTTTATAGTCACTCTTCATATGCATCAGTGTTTGAGAT
TCTGCAGATTGTTGTAGCAGATCATGTTTAAAGTCCGTTATTTGTAGCTCTTTTGAGTTACTAGCGATTTTGGAG
GTGTTAACCGGCCCAAGCATGAATAGGCCTGATGGTATTCTGGTCCATCTGAGCTTGGCCGAAGTTAATTGCAAA
ATTAAAATTAAGTTTATATTATTATGCTTTTGTCTATCTCTTGACTTTAATTAATCTTTTAAGGTTAATTGGTTA
TTGGTAGTTGAGGGTGTTTCAGTTAACCTTAAATAAAAAACTTAATTGACTTAAATTATAGTAAAAACCAAACCA
AACCAAATAGAGATCCAAATTTAAATGTACATAATCTACCACTTTCATAATGTAAAAATCCAAGTGTTATCAGCT
GCCGCATTTGTTCTGTCTTCTATATGTCGAATACGAGTACATAAACTACAACTTTGATAATCTAATTTTAGGATT
TTAATATGCTAGGCTGTTTTGCGTTTGTCATGAGTAGGGTCGAATCATGCCTTAGCCAGTCGTACTAGTATAGAT
GGTTGTAAGTTGGATTTGATGACAACTGCTTTGGATTTGATGACAAGTAACTACTTAAAAGCAATGGTTAGGCAG
ACATTAAATAGGTGGTGATCTAATGTCTAACATCTAAAACGTTTTAATTGGAATTTAAACGAGTTTAGATGGTCT
AATTAAGCGTTTATTATGTACATAATAGAACGTATAAAATATCATGATTCACAGAAGTAAATTTAAAATGTTTTA
ATTTTAGAAGTAAATTTAAAATTCAAACTACATCAAAAATTCAATAGAAATCAACTTAAAGATAACCATAAAATC
AATTCAATTGACATCAAATAATAGAAGCCATCTACTTTTTATGTTATAAAACTTATAATGAATTTTATGTCATTA
TAAATCGAAAAACTACACTGAAATGAACAAAAAGTCAAATGACAACAGATTAATGATTATGTAAACTGTATAATG
TCGATTAAAACCACATAATGCCATCAATTATTTAGCACCTATCAAATAATTGTCACGGTTGTATATATGGCGCGC
CTAGACCGCCACTTAAAGCGCTATTTCAAGTATTGTTACAAACGAAGAAGGTAGGCGGTCTAATTTTTAAGTCTA
AAATTAGATATCTTTGGACGCCTCATATTACTTATTGTTTTACTCTGTCGGATCCTATCTCACCCAACCATACAT
TTGTGGCAACAGAAATTGCCTCCAAATGCAAAAAAAAAAATCATTCTTTCTACTGTTAATACTTCGAAATATACA
ATTTTTAAAGTTAATATGTGTTTAAATACCATTTTGATCTAAATTTTGTGTTTTGTGTTTCTTTCTATATATATA
TGTACGTTTTCCATTATATCAGAATCGAATCATATCTAACCATATATGAATTAGTCTCGGTAGTTGTAATTTGGA
ATATTTATTTCGCTTTTCAACGTTGGTAGGTTCAAAAATACTTAAGTTAAGCCACGTTTAATTTAAATACGTTCA
TATTTAGAATATGTAGAGTTGAAATTTATAACTTAAGTATATGTTATGGAATTTGTTGACAGCTATTTTAAAGTA
GTTGGGACAAAACAAAAATGTAGGTAGGTGCTCACATTAATTTCACCTTGTAAAGCCTAAAACTATAAATCTTAG
TAACACATATGTGATATGTCAGTTGTACTCTGTTGCATTATTTCTCTCACTCAAACGTATATACAAACATAAATT
SEQ ID NO: 64 pAtCER5_WBC12_ABCG12_At1g51500
TATTGTGAAGCTAATTTTTTTGCTTAGTTATGTTGATATCATTTTGCCTTTTGGTTTGTTATAGTGAAGCTTGAT
TTGTTGGATTGTTGTCATGATGTAATTTAGAGATGAATGCAATACTGTTTTGTCGTAGATGTTGACTTTACTCTT
TGACATGCTTTGTGTTAATCAATGTAATACTGAATATCTGAAATTGAGAGCCAAAAATGATAAATTTATCTTTGC
AATGTTACATTGCTTTTAAGAGGGAGATGTAATATTCTGTAAAGTGCAATGCTGTTCCCTAAGGACACTTCGTTT
AAGTTACTTCAAAGTGTCAGAAATGACGGGTCGAATAAGATAAGCTGTCTTCTAGATGCGAGTTTAGGAATCAAA
TATTATTTTTCTTTAGTTTGCTTGAGTTCTCATGGAAGTGGAAGTAACACTTTGCTTTTGTTTTTTTTGATAAGG
GATATGCTTTGATAGAATATGAGAAGAAGGAAGAAGCACAGAGCGCTATTTCAGCAATGAATGGTGCTGAGCTTC
TAACACAGAATGTTAGCGTTGACTGGGCCTTCAGCAGCGGTCCCAGTGGTGGTGAATCTTACAGGAGAAAGAACT
CGAGGTTTGTATCCTAATCTCTTTTCTGTTCCGCTACGACTTGTCTGATCTTGTGCCGATATGAAGTCAAGAGTG
TCAAAAAGTATCAATTAAGATATAGACTTAGAAGTTTTAACAGTTGACAAGGTAGTATATTGTACTAAGCACCTA
CCAAACTTTACTGTCTTTATGCTAATGAATCGAAATCACTTGTGACTTGTCAAGTAGTTATACCTTTCTTTAAAT
CATCATCCATTGATCATTGGGGTTTTACAGGTATGGGAGGTCGCAACGTTCAAGAAGTCCGAGAAGACGTTACTG
ATTGTATGATTTGGTGGAGAGTTCTCCCCATATCTCTCTGTATTAGGGATTCCCTCTCTTATCTGTAAGGTACTG
ATGCAAGACGCTGAGTGTTGAACAAATTTTTTTCTGGGGTTTTTGTAGGGTTTGGCTGCTTTTGCATTGGTTTAT
CTCTTTATGCATTTGCATGGGATTGTTGCGTTTAAACACCATGAGATCTGTTTTAATGCTTTGTATTGGTAAAAG
AAGAGAAACATTGGATGAGGAAGAATGTTTTAATTTTCATTCTCTTATTGATTCATATTTAGGTCAGACAGAATT
TGAAAATCAAAGACCTTCCATTACACTAGAAAACCAAATATGATTATATAGAAAAAATCAAAGAATTTTCAACCA
ATGGCATTATTTCACACTGGTTTATTATTCTAGGCGTAACTATTGGTAACCTAAATATGTTAACAGAAATCATCA
AATGACTATTAAATATTTTGGCTGAAATTTTCCAATCAATGGCAATATTCACATCGTTTGTTTTTATAGGCGTAA
CTATTCGTATCCTAAATATGTTAAAAGAAATCATCAAAGGTTTGATGATTTGAGATAGAGCATGTACTTTCTTGC
TACTCACAGGTTGCATGATTTCATTTGTACGAATCAATCATCTATACTTTGTGCATTGAGCCAGTCCCACATTAT
GTTTATGTATCCATCTATTTTAAGGGCTCTCATATACTAACTTACAGCTGAAAAGTATAAATATGAAACAGTTGG
AACAGTTATATAAGTTCTGCTTTTGTTGTCACCAACAAAAAAAATGATATGCTTTTACATGATTCCAACAAAAAA
AAAACAGTTTTGTATTCTTGTTAATGATGGTGCCAAAGGCTTATAAGACCATACCTTAGATTCTGTTGAATTTAG
AATCTGGGTTTCTATCTCTTTTCTGGAAATCTTGTTTCTTTTTGTTTTCTTTTTATACCAAAACTTTGTGAAATC
TTAGGTACTTTTGTTTAGTATCAGTTTACTCGTTTAAAAAGAACCAATAAACCTACTAGATATAAATAGTAAGAA
GTCTATAACTTAGGGTTGTGTTAAATACTAAACAATGCTTAAAAATGTGGTTAGACATAGCGCTATACTAAAGCT
AAAAAGCTAAAGATAACCATAAAATCATGAAGATAATAAATGGACGGATAGACCAAAAAGAAATAATGAAGCATA
ATACAAAAAAAAAACTATATCGGTTCCAACTACCACACATTTAATTCCTTTCCCTTTTCATTTAACACCTGTGAA
GACTCGTTGCGAGGGTTCTCTACCTCTTTATGTCATTCAAACTTATCAATTATCAAGTCTCTTGATTTTACATGT
TCAACCAAACACGATTTTTCAAACTTAATTTTATATATCTTCTTCATTAATTATCGAATATCACTTCTCTTGCTT
ATAGTTACAAAAACTAATACAATGATAATTATATAATTCTATGTGTGAACAAAACGATAAATTTGTCAAAGTATC
ACTATCAATCCCTTGGAATTAGTAGACAACCAAATATGGATCCTTTAATTGATTTGTGATGTTTCTATTCATTTC
GAACTAAATGAAAACATCTCATCGTAAATAATCACACTGTTAAATTTCATTTCACAAAAATATCATTTGATTTCT
GTGTGGACGAAATTTCTTGTTAGTTGGCAAGAACCGACATAAAAAATATATTTCATACGTATAATAGTATGTCAA
TAAGTAGGTGGTTGCTTACTAGTTACTACTACACATGGACGATTGTATAAATATTAGACCTCATTCACACAACTC
TCACTCTCTTACATTTAATGTGCAAGAAAATAAAAAAAACTATATACTATAGACGTGTATCTATATATGGTTATG
ATATTAAAGCCATCCATTAAATGTAATTAGTTGTTTGTTGATTGTTGCTCTCAAGTCTCACCTAAAGAAAACCTT
AGGCCTACAAAAATTAATCACTATCTTTATAATTCCATCTCCAACCAATATAATAAAAATAACCTAGACGAAGAA
CAAACAAAACTCTCTCTTTCTCTCTCTCTCTCTAATAAGTAAGGTTTTGATCTTTTTCAAGATCAAACAAAAACA
SEQ ID NO: 65 pAtCER6_CUT1_KCS6_At1g68530
ACTAAAAATTAAGGGAACTTGTCTTTGATATTTTATTTACTAGTTATATATAGTCCTTACCGAACCTCGTCGGCA
ACATCATCAGTGCGATCAAGAATCGATCACCATTAAAATGCTTTATTGAGCACGATCAGTGTTCACCATGTAAGA
AGATTATTGTCTCACAATTCAAATCAAACCTAGCCTAGGCAACAAAAATTAAATTTCTTTAAACAATAGAACAAA
ACTCGTCCGCCCCAACCATCCTCTTCACAACATTCCTTAATCTTACAATATCTATATAACTAACGCATCATTTGG
ATTTCACTATGTAACTAAGTCGAACTGGTGTGAAATAATTATAAATTTAAATAAATAAAAAGGTGAAAATATGGT
TGTACACGAAGGTATGTGAAATAATTATGAATAACTAAAATGTACTGAATTATTTATTTTATTTTAATAGCAATG
GAATCTAAATTAAATCAGGATATAATGAAAATTTTGTAAAATTGTGATTAAATTGTCATTCCAAAGAAATCAGAG
AACTGTCTTTAAAAATTTGGAAGCGAGTTTAATGATATAGCCTATATGAATGGTACAGTGTGAATGTCTCTGAGA
AAAATTACAATTCCACATTTTTATTACAGACTAGATGAGAAAAAAACATTGATGAGTAGGTCAAATTAAAAAACT
ATATTCACGTTTTCGGTGATACATTGGAAAACCATATAAAACGATCATTTTTCAAAGGAAGAATAGAAACTTTAT
TTACATATTTAATTATTATGGAAGATGAAGGAAGAACTTTATCATAGACTACGAAATCGGTGTGGCTAATGAAAT
ACAGACACCTTATATATTACATATTAAATGTACATTTCAATCTGGTGGATAGTTCCAAACTAATCTTCTTCATCA
AATATTGTTGGCATTAATGATCTCACTATGGAACACGAAAATTCAGCAATGTTCATTTTTAGTTAAGAAACAAAT
CCTCTTACGAACTCTGTAAGATCTTGAAGGATCAAAAGCCTTTGATCTCAATTTCTGTATAGTTTTTATTAGCCT
AGTGCTTTATATATGTTTGATACTTCTGTTTGGCAATATCAATCATAGTAGAAAAGATATGGACTTCATTTGAGG
TTTTTGGTGGATTGTGTCTATATGTGAAATCATGGGATCTCAAGATTTGTCTGCATTCAGTTTCCAAGTCAAACA
TCGTAACTACTGTTTGATTTTCCCTCATGCTTGCAGTTTTCATGGATATCTCAAGATTTGTCTTCTTGCACTTTC
CAAGTCAAACATAAAGTAACTACTGATTGATATTCCCTCGTGTATTACCCTCTTTCAAATGACACAATTGGGCCC
AAGTAGAGGAATTTCATAGTGAATTCAAAAGATTAACTGTATTCCACCGTCGTATTTTGATAACATTTAGTTATT
CCTTTTCTTTTTTTTCTTCTGCAACAGTTTTTTTTTAATACATTTAGTGTTGGTTTGGTTCAATGAAATATTATA
TGTTACTTCTTTTTTTGGAAATAAATTATTCATTCTTTCTACTATAAAAGGAATTGTTCATGCTTTTTTGATACA
ATAGTATACCATTTCAAAAGATACCATAGACCAGTTATTACATGAATCGCCAAAACAACACTAAAATCAGAAAAT
CAGTATATTTTGGTATAGTCTCCAACATACAATCATAAAACCTCTGTGAAATTTAAAATCTATATTTGACATTTC
AAAGTTTAACAACATAGTTCTAAATAATTACCTAAATTTTAAGTCAAATGTGAATTATATTTTACTCTTCGATAT
CGGTTGTTGACGATTAACCATGCAAAAAAGAAACATTAATTGCGAATGTAAATAACAAAACATGTAACTCTTGTA
GATATACATGTATCGACATTTAAACCCGAATATATATGTATACCTATAATTTCTCTGATTTTCACGCTACCTGCC
ACGTACATGGGTGATAGGTCCAAACTCACAAGTAAAAGTTTACGTACAGTGAATTCGTCTTTTTGGGTATAAACG
TACATTTAATTTACACGTAAGAAAGGATTACCAATTCTTTCATTTATGGTACCAGACAGAGTTAAGGCAAACAAG
AGAAACATATAGAGTTTTGATATGTTTTCTTGGATAAATATTAAATTGATGCAATATTTAGGGATGGACACAAGG
TAATATATGCCTTTTAAGGTATATGTGCTATATGAATCGTTTCGCATGGGTACTAAAATTATTTGTCCTTACTTT
ATATAAACAAATTCCAACAAAATCAAGTTTTTGCTAAAACTAGTTTATTTGCGGGTTATTTAATTACCTATCATA
TTACTTGTAATATCATTCGTATGTTAACGGGTAAACCAAACCAAACCGGATATTGAACTATTAAAAATCTTGTAA
ATTTGACACAAACTAATGAATATCTAAATTATGTTACTGCTATGATAACGACCATTTTTGTTTTTGAGAACCATA
ATATAAATTACAGGTACGTGACAAGTACTAAGTATTTATATCCACCTTTAGTCACAGTACCAATATTGCGCCTAC
CGGGCAACGTGAACGTGATCATCAAATCAAAGTAGTTACCAAACGCTTTGATCTCGATAAAACTAAAAGCTGACA
CGTCTTGCTGTTTCTTAATTTATTTCTCTTACAACGACAATTTTGAGAAATATGAAATTTTTATATCGAAAGGGA
ACAGTCCTTATCATTTGCTCCCATCACTTGCTTTTGTCTAGTTACAACTGGAAATCGAAGAGAAGTATTACAAAA
ACATTTTTCTCGTCATTTATAAAAAAATGACAAAAAATTAAATAGAGAGCAAAGCAAGAGCGTTGGGTGACGTTG
GTCTCTTCATTAACTCCTCTCATCTACCCCTTCCTCTGTTCGCCTTTATATCCTTCACCTTCCCTCTCTCATCTT
CATTAACTCATCTTCAAAAATACCCTAATCACATTTTGTAACAATAATACAATTATACATTAAAACTCTCCGACG
SEQ ID NO: 66 pAtCER10_ECR_At3g55360
CCTTTGCATGGCGAGATTTTTTATCCCGAAAGTTCAGTCTAAGAATGATGTAAAATTCCTCTGTTAAGAAAAAAA
TGCCTTTAACAAAATAAAGAATTCGATGTTGAAGAAGAAGAATATATATATTTTGGTGCTAATTTGCAGCACAAG
ATATAAAAATTAGAAAGTATAAGCCAAAAAATCAAATCATGATTCGACAAGGTTGTTTTTAATTCTTTCGTGATT
CTTGATGTTATACCCCTAATATAACTTACCGATAACATTGAATTAGTGAAAAATATTTCGAATTATGATCACCAG
TCTCTATATTATATTTGATGATTTTATGGTTACGAATGTGAGGGGTACGTAAGATTAGTAAAGACGGACATGTTG
TAGGAAGGTGTCCCAGTCCGCGATTTCTAAAAGAAAGATTTGTGGTCCAAACGTAAAATTTAATCAGACCTTCTT
TTTAATGGACCTCACTATGTCGACACACAACATCAAACATATCCCTAACTATATAGTTCATAATCAAGATGTTTT
TTACCAGAAAAATATAACATCAAACATGTCTCTCTCTAATCATTATATTTATAAGTTTTTCGTATCAGACTAAGA
AAGTATATATCAACCTGAGACAAAATGATCCGGTAAGAAGAACTTCAATTTAGATGACAAAACTTCCAATTAATT
TGAATTACTTAGAAAGGGATGAAGTTGAGACACAAAGAAAGAATATAATAGATATATTGCCTTAGCTTGTAATTC
ACAGTTTAGTTGTTTCAAAGAGCAATATCATTGGAATTTCGTAGTAACCAATATATATTGTGGTTATATATTTGT
GGGGAGTGTGTATATGTATTTGATATGTTGATATTTTTCTTCAAAAGACAGATGATAGAGCTAAAACGATTTTTT
GTCGATATGTTGCGCTTCGAAGTAGCTGAACCTGAGTGATAATGTCTTCATCTATTGGACAAGTAGGGAAGAAGC
TAGCTCGCCGTATGCAAATACATAAAGTTCCATGTTTCACTTCTTCTAGGGGTGAATAGACGAGGACACACGATA
CAGTTTGTCATGTTAATCTTTCAAGCTCAAAAAATATAAAAATTGTATGAAAATCTGATACGGTAAATCTATATA
TTCTTTTGCATGTTTTTAAAGGAGCACTTATCTTTATTTGTTATAGAAATTTATTATTTAAAAGAAATTCGTCTT
TATAGAAATGTGCATGTGTTTTTTTTATTAGTAACACATTTTCATGCAACAGGAATCTTCGCTTATCTGTAGTGT
ACATCTCATGAGCCCATATTCATAGTTGTAAAGCAACACCTTCTCTGCAGCTTTTTCTTTCCTATTGTTTTTTGT
ATCCATAGTTCACATCTACACACTATATAAGGCTATTACAAACAATGATACAATATATGTTCTCATATAATTTGA
TCTTAATTTTCAAGATATTTAAGGTGGGAAAATATCGAGATAGTATACAATGGAAATCGTTACCACGTGCATGCC
AATCTTTAGTAGAAGAAGATTCCTAGATTCCAACAAGAACATGCGCATTTTTTCACGAAAGGCTGGAAAGTTTAA
TATATAATGGTCGAAAAAATAGGCATAATCTATTTAAGTACTAGCTTAAGTGTTTTACGCTAGATCTGGTGTTGA
CACTTGTACACCTGGTTTTAAAGTTGCAGAGAAAACTAGATTAATGCTCAAATTGTAGTAAGTAGATTGGATAAT
TTACACTCGGAAAAAACAAGTTTTTTCAGAAGAATTAGACTGTTGCTCACTTGAGAAAAATCTAGCTAGCTTTGT
TAATTGAGGATAGTTAGTTATAGTATTTGAATGACATTAAGGTGTTTGAATATCGCATGAATTGTCGCCTTGGTA
TTTGGGCAATTACCGGTCATTATTTTGATCTTTGTGATGTCAACTAATCGATCCCAACCATATATATCATAAACT
ACTGTCTCTTAATTGATCCATTGTGTTTTTAATTTTATCATTACTTTTTTTAACTAGTTGAGAGAATGATTATCT
AACCCCTTTCATATTCAGAAAACTTAGGTTTTGAGTTTTTGCTTTATTTGCTGCTCTCTTGAATTAATAGAGCTC
TCGTATGCGATTGACTTTGGGTGATTTTTTTTTGTTGGAAAAGCAAATTTCATTGACCAATATAACTAAACTAAC
ACACTACTTCCATGTGGTTAAGGGCCGGGATCATTTATTATTTGCATCTTTTATACGACAATGACAAGGATTCGT
TCTCATTACTCCATCCCATTTTGTGGTATGTAAAATCCTAACTTTTTTACTGCATTGATACAACATTTTATTTTA
GCATTAGATCAATACGATGGACATATGTCATCAAGTGAGGGGGTAGAAGTTTGCAACTAATGTTTTTGTGCTCAT
GAATCAATCTCTTCTAGGTAACATTATTTCACTCTTTTCTATAGCGAATACTGCAATGCTTTTACGTTTTAACTA
AACATGACTAAACCAAATACCAAAAAAAAAACGAGTAACACTTTGGTGGTTCCTATCTAAGTTGACCGGATCACT
CGATTTGATAAAAAAAATTATAGTAAAAGTTTTTGTTTTTGAATACAAATTATATATTTGACTAAAATTCGAAAC
TTAACCGAAACTAAAACCTTAGCAGATAATCTATAGTATAGTAGATTCATAAACCGAAAAAACAAAAATCTAAAC
CTAAACTGAGTAATTACTAATGGTGGATCCTTAAAACAAAGCCATAATTTGCAATTGCAACAAATGTTTTTGTTT
GGAGTAAAAACCAAAAAAAAAATGATGGGGAGCAACTAAAAATGTAAAATGGTCCCAATCTAATCGCATAAGTTG
GGAAGATAGTTAGTAGTTCTCCTACTCCCAACTACATCCTCTTCAATGCTCAATCTCGTCTTCTTCTTCCTCTCT
ATTTAATTTCCCTTGTCTCTCTATCTCTCTCAATTTCCTCATCTGGGTCTTCCTCGTTTGCTCCGCTTAAGCACC
SEQ ID NO: 67 pAtWSD1_At5g37300
ATCACAAATGATCAGTTCCAGTGTCATTAGACTTGAGCGTTAGGGAGTTTTTAAGAGGACAAAATCTATTCTGTC
TTCCCGAAAACCACTATGACCGTGAGGGGGAAACAACTTCGGGAAGATGGTTAAAGGGACATACCGTTCATCGGT
GGTCTAATCGATCTTTTTCTCGCAGGTTATTCTCGAGTTCTCGCACACATAAAAGTTTTTAATCGTATGAAAGTT
CCCGAAAATTTGACAAAAACTTAATTTTTTACAATTATAAAATTTCATTTAAAAAAAAACTTGTTTATAATTTGC
TTTCTTCCTATTAATCCTTTGATTCCTAGACTTATTTTGCTTTCTTGCTCCTAAATTAATTGAGTTGCTCTCTTG
GTTCGATTTAAATTGTTTGATCGACATCGATCTGGAGTGAATTTGGAGGAAGTGTAGACCCTAAAAGGTCGTATC
TCAAACTCGATGTTGGCATGTGATGCAAGAATTTGACTTACAAGTGGGTGTAGATCAAGGGTTGATGTTAATCTA
CTATCCGATGTAATTCGATACGTTCCATTGATGTGAGGAGGGGCCAATGTTCATGCTTAAGCCTCTAGTGTTATC
ACCGAACTCAAGACAATTGAATGCTGCAAGATTGTGTACTAAGAGTATGCAGACTGATTTTACGTGTCAAGACTT
TCCACTGCGATTTCAACCACAGTGTACGTATGTTTTCAGTGACAATAATACGGTATTATTAATACCCGACTCTTT
CACAAACTTTGGAAATTAGTTTGAGTTCAATTCAAAATCGCATTTAAAAAAATAATAAAAAAATTACATGAAACC
CACAATATTTGGTATGAAATTTAATATGGTAGTTAGTAACCTCGTAGGTACAACATTCATCTTCTTGCTGCCGAC
ATCATAATTGTTCATAAATATTTTTGAACACGTACATATAATGTTCATGGACCAAATCTGATCAGACTCCTAATT
AGATGAATTGTGTGGTCGATTTTGAGATGTACTTTTTGAATCATAATTCTTTCAACTACTCGAAATAAATTTCCC
TCACCATCCCAAGACTACTAGAATAGCATATTCTTAAATATAAATTATTATATAACCAAACTATTATTCTTTTTA
AGATTATCACTGGATGTTACGTTGAAGTTATAAGACATGATATATATCACTCAATTTCAGAAATGAGTAGAGTTC
TTAGTGCATAGTTTTTCTTAAACCTACAAGGCTACAATGAATAAAGCACGACACCATACTGTTCTCAAAATATTT
GTATATAGTGGAACTCGGTTGAAACATATATATACCAGTAAAGTTATTTTTGTTTTTGTTAATAAAAGAGTAACT
CGTGAACTTGAAATCAAGGAGCTAATAAAAAATTATTTTTGTTTGAATTTAACTTTTTCAAAACCTAACACGAAA
ATCAGAAATTCTGAGGGGTTAGAATGGTTCTCACTTGTTTACGTTTCTACGTCCAAAATACTCATTGAGTTTTCA
CATTCTCAAGTTTTGGAAAAATTAACAATTTCGTTTATACCACTAACATGTTTAAAACTATAATTATTCTAAAAC
TTTAATTATCAAGTTCATTTCGTTTAGCTAAAATTTATTCTGTTATATAGTTATTTATTGGATATAAACCCTAAA
TTTAGTGTTTTGATGCCACACGTAAGCTGATTTTCAAATCACAACTTTATAAATTCCCTCATTAATTTATCTTCT
AATGCATCTATCAACAATAACGTTATTTATATATGTTGTCTATATCTATTAATCTAAATTTGTTACAAACAATTT
AAGTCAGCACTTATCGGTTTTATGTCTACATCATACATTATGTAGGTTGTACGATGACGTGGGTTGTAGCTAGTT
AGGTATAAAAATTTACTTTTAAAAGAAAAACATAATCTCCTCATCAAAAGCTTTTAATTGAATTTAATAGCCAGG
GCCAAATATGCCAACAATATACACCAAAAAACATATTTGTCGCTAGAACAGTGCTAATTAAGGGACTCACATACT
ATTAGACATTAGTGCATGTCAGCCAATAGGCTTATTGTATTTTCAACATGTTGGCCCATGTCAGCCAATAGGCTT
ATAGTATTTGCTAAAAATAAAAATATATATATTTGCTCAAGTGTTGGTGTTGCATGGTTCAATATTTATCAAAAC
TCAAAAGAATTGTTGACTAACTTTACATATTAACAAGACTAATCATTTTTCTGGTTTGTCCTTAATTTTAAATAT
GTAAACCACTCATTTATGATTTCTCCAATTTAGAAAGAGTGAGATGTGAAGTTTCTCCCAAAGAAAGAAAAAAAC
TACAGGTAAATAAAGTGTATACTGCTTTCCAAAATATGTCGAATTATCACAAAAATAAAACGAATTTATAATTAA
ACATCTCGTTATGGATTTTGTATAGAAATTTATTGGCCTACAACTGTTAATAGACATATTATTTGGCATTGTACG
TAATCTAGATTAGCCAAACTATATATAGAACATCATGTTTAGCTGATTTTTCAAAAAAAAAAAAAAAAAAATCAT
GCGTAGACGAAATGCAAGTTTTAATTTAATTTTATTGCTTTCTCTGGTTAGCTCTAGAGGTCTTTAATCCACCGA
TCTTAAAAAAAAATCCAACTTTCTACTTTCTCTTTCTAAATGAAACAATTCCATATTTTTCATTTCATTTATACA
TCATACACTTAATTAATGATATTCTCTTTCTAGTAATTAGTACATTAATTTAAATTTATCAAGAAAATGCTGTTG
AACATATATAATCAATTATTATGAAATGAAATTTATAATGAAATGATTTGTTTGATAGAAAGAATAAATTATAAA
CTACTTAAGAGACCGACGAAGATAGCAACGACAAAACCCTAACCTTATACCAACCCTCATTTAATACCAATAAAA
AAAGAGGGACAAAAAAAATCAATGCTATATAATCGAAGTATCCACTACAAAGGTTGGAGAATCCTTAAACTGACT
SEQ ID NO: 68 pAtMAH1_CYP96A15_At1g57750
GTACAAAAAAAAGAACAAAATCTATCATCATTTAATTCGTTGCTTTATTTAAATAAAAAACACAAAAACTTGAAA
TCATATACTTTATATGATAAATTTAAAATCTATATTAGTAGTATAAATTTAAAATAATTTAAGGGACGGTATACC
ACGAGTGGGATCCTAAAATGATTGGATTTATTATGATAATAATTTATTTTTTTGAATTTTATAACACTAATAATT
TAGCTAACGAGATAATATATACGGATTTATATGGGATGACGGGTTTTGATACATATTGATAAAGAAAAGTTTATC
ACGGATGAAATCTTAAAATTAACAATCAAAATACAATTATACAACCTTAAACACAAAAAAAAATATTCAATATTA
ACAAAACAAATATAACTTAAAATTTAAAAACTTTAGCTATAAAAAATCGGTCCACAATATATTGCGGATAAAATA
TTCAAAATTTTAAATGTATAAAAATATATCTTTTGTATTAATACTTCAAAAAGTGAAGCCTAAACAATAGTAATT
ATATACTAAATAATAAAGAAATAGATATCTATTTAAAATACAAATTAATATGATATATTTTTAGTTAATCTTTAA
AAAAAAATATTACATTTAAAAAACAATATTACATTTTAAAGAAAAATATAGTCCCGCGGTATACCGCGGGTTAAT
TTCTAGTTTTAGATTATAACTGAAGTTGTTACTTACAAAACAAATACAGATTTAAAAAAAAAAAACTAATTATCC
AGACGAAACAAAGAGGATCATAATTATTAGTACAAAAATCTAAGATATAAACAGTAGTAGATAAATATTACCCAC
TCATAGTATAAACAGTAGAGGATAACTTTTGGCATTGATGGACGTAATAAAAACCAAAATGTTGTCATACTAACC
AATTAACTACTAACTAGAAACCAGACTAGCCAGGCTGTAGGGAAGAACAACATTGATTGATGTAGTGCTACATAT
CGATTAACTACTAACTAGAAACCAGACTAACCAGTCCATCATTAATATGTATAAATACATATTGTATATACATAT
TTTTAACAAATGATAAGATAATAGTAGTATTAGTTTTTTTTGGTGAGTAATAGACTAATAGTATTAGTTTTTACA
ACAGTTTAAATTGTTCAAACAAAATAAAACGATGTAGTCTATATAAATAAGCAAAACAAGGTTCCCGGCGGTATA
ATATACGCATGGTCGATCAATGAAAATTATGGATCTAATCAGTAAAACATTATTATTTCTTACGTAATTACAATA
GTAATTTATCTCAAAGGTAAAAAATTATTATAAATAAGTAAAATACAAAATCTAAATAGCAATGAAAAGAAGAAA
AAAAAAACATATTTCTCATAAAACAATTTACGTAGTTAAGAAGATTTTTTTTTTTCACAAAAAAGTAGTTAAGAA
GATTTTATCTTTCATTTAATTGTAGTGGAAAGATGTTTTTTCTCGCATAACATGTCATTTTTATGTGAGTGGCTA
ATTTATTAAATTGGTTAGATTTTAGTAGTTTAGATTTAGGGGTTAGTTTTGTTAAAAAATTATATGATAAATTAG
AGGGCCACAAAAAACATATTTTGAAATTTAATACGTCATGACTCTTAAAACAGTAAAATTTAATACGTCATGACT
CTTAAATTTAGCCGTTGGATGATGAATATGCACGACTTTTGTGATGTACATTATTAACAAGTTATGAACTAATTA
GCAATGACAATATACGTCATAATATACGGTATAAAATAATTCAGACATATTTTACCTAACAATATAGCGATGAAT
GATGTATGTGGTAACTTATTGACAAATTTAATATGTCGGTAAATATAATCAGTCACTAATATAACAACGGATATG
CGATGATAATAGGTTCTAGGTCTTTAGCAATGAGATAAGCGAGATATTTATGTAACATTAGTCTTTAAATTTTAT
ATATATGTCTTTTCTATTTTATTTTATTTTTTGAATGAAAATGTCTTCTTTATTCGTAATTTTAAACTCACTGGT
GGTGGATATATTGTTATGTCCCCAATTCGTCTGGCAACTCTCGTATATTAGTGAGAAAAATTTGTCCATTATTTA
CTGCACTATTACCCTGTGTTAATTTTTTGTATTGAAATTGTTTTTTAGTAATTCACGTCATATAGCGAATGATTC
TTTAATTTTAAAAATTCAGTCTTAAGTTTACAAATTAAATAACGCTACTGTAACCAACTCTGTACGACCAACATG
TTCGAGTTTTTGTATATACGGCCATATATGTACATATTTTACTATAAAGCGAAAAAATCCATAAATTATTTAATT
AATATATAAAGGTGCCATTCTATTTCCAATGTGCTTAGGAAAATGCAGAACCTCGTGCTATATCTCTGTCGCCAC
GTGCAAATATAACAATATGAAATAGAACTAGCAAATCTTGAAATCTAACTCTTAAGACTAATTCAAGCACATACG
TAGAGAAAGTTGACCAACGGTTATCAGCATTTTAACATGGACCTTATCAACATTTTAACAAAGTCCACAAACAAC
CAGTCTTACAATCGCATTGGTACAAGATAATCGAATTCATCTTCCATATAACAAAACCTAAACCTTGGTGTGAAA
AGGAGAAGATATGTATGTTAAAGGCCGCCTATGCCTCTGGTTTGGGGTATATGATTCTAAGATTAGGGTTTGAAT
ATTTTCGTTAGCCTGCCATGAGATATATTTATGTGATAATTAGAGCCTCTTATGCATTAATGCATAACCGACTAG
ATCATGTGGTATTCAGCTAATCAGTACACACAAGACAAAGTAGTAAATGAGTTTGATGAAGACTGTGGTCTGATA
ATTCCTATCAACGTTAAATCTGTCGGGGCCAGGCAGCCAGCAACATTTTGCCTAACAACGCTCTGAATTCAATTG
AACCTAGGCTATATAATAGCAGGCTAACTTAACTAAGAGTTTTGTATCTATACTCATAAATCCTTTTGTCTAAAA
SEQ ID NO: 69 pAtWBC11_ABCG11_DSO_COF1_At1g17840
GATGCGACGGATACAAATAAACGTATCATTTCACTTTTCTCGTCTTTGCGGTGGTTTAACTTTTGTTTGTGTGAT
AACTAATCTATCATATTGAATGATTTTAAGAACGAAAAGGTAAGTAAAAGCTATTTTTGAAAAATACAGTAGTAT
GTATTCGTTTCTTCTTCCTCAGTCTGTAAGTAAACACGGATTCAATAGTGAAAAGAAAAAAGAAAACACGGATTC
AATTATTGAGCACTAATTGGTTAAAGGCCCATTATGTTCCATTTTGTTTTTTGGGCTTTGACTCTTTTGTCACAC
ATGCGTTGACGTAGTAGAATTTTATAAACATTTTAGCCCAATATAACATTCTTTCAGCATCTTGATCCTTCCTGT
ATGATAAAATTAAAAATCGCCATGAATCTTTGGAAATCAACAAAGGGCCAGAACTTATAAGAATATTTGTTTTGA
TTATTAAGGTCTATAAATGTCTGTCAAACAAAAATGCATGTATTGTCTATATCATCAAAGTATGAATATCCAATA
CTGTTAAAATATTTATTTATCCGCATAAATACAAATACCTCTGAATATAGTTTTTATTTGGATTAAATACTAGAA
TTTAATTAAGAATTAATTGTCTAAATAACCCTAAAAAATAAAATAAAATTACAAACAGGCATTTACGTAACGTAA
TGACTTACAGAAACAAGCACTTTTATCTTTGCAAAATAACGGATTTACCTTCATTCATCCACAATATTACAATAA
TAGCTGAGACTTTTGACGACGACTATGGTGAACTCCGGCCATAGAAACTCAAATGACGGTGTGATAAACGAAAAA
TCGTGAAAGACATGACTCGAATCTGAAACAGTTGTGTAAGGATCTCTCTGTCACCGTTGGCATATAAATACCATT
GTTTCTCTCCTTTAGATTCCAAATTTCTGTTTTAATCACACTCTCTGTTGTTGCAGTTTAGATCTATTTCTGAAA
TCTCAATAGAAATCTTTCCCGATTCGTTTGTATGTGATCTGGTTTTTGTAAATAGCCATTATCGGGAGAAATTAT
GTTGAAATCAATTTCTCCTTTGTCACGTCGAGATCAACTTCTCTGATATTTTCCTTCATCATTTCCTATTACATC
TCACAACTCTAAGAATATCTTCTCTGCAATCTCAATGTCAATCACAATTCATCAATTCCAAACACAAATCTTTTT
CTTCTCCTAACTATCACAAAATTAGATGAGTGTTTATGATTAGAACCCTTGGGTTCCATATATACTATCTCATTA
GGGTTTAGATTTTGCTTGTATTCTCTTAATTTTGTTTGATTAAAATCGATTTCAAGGTTTCTGTAAATAAATAAA
TACAAATTGAAGAAGAAGAACGATGGAGAAGAAGCATGAACAATGAAACAGGGGCAAATCGTAAGTTGGCATATC
TGAAAGTTTCTTTTTTCAAATCCCATCTCAAAAATGGTTACCGAGAGGAAGCAAATGTTTTTGGTCGACGGAGTA
ACGTGGTGGTGGTGGTCGAGAAAGAAGAAAGAAGGAGATGATACAGATGACAGGGGTAAAACAGTCTTTTTCCCT
AGCACAAAGTACTCTTTTTCAAAATCCAACACAGAAAAACCTAAATTCAAAGATTTGGCAAAAAAAAGCCCTGTT
TTGACAATAAACCCTTTAATTAGTTGAAACAATTATAGAAATAAAAGGAATATTTGGCCACAAAATAAATAAATA
GTTTTAGCAAAGGTGTGTTTTCCCCTGTGCATTACTCAATCATAAATTTTACAAAAATTTATAGTCAATGAGATC
CATAAGAATACAGAGAAATTTTGAATTGGTTAGAAATATGTTTGTTTTTTTCGATAACTTAACTAACAAAATCGA
AAGTCTTATGAATTCAAAGATATTATTTAGTTTCCAATAATTATTTTACTTTTGATTTTTTTTTTGTTGAATTAA
TCATTAATGACAAATGGACCTACGAAGAATGCCATCAAGGGTCCTTATTAACAAGTACCAACAAATCAACAATAG
GCTCGTACTCCTTTCTAATTTTTCGGCACTTTTTTGTCAACAATATTTCAGCATTTATATACATGGTTTTATAAT
TAAAAGACAAATTATTATTTGTAACATATGTCTCCTACTACACCGAAAATTACCGGAAAAAAAAACATAATGTAC
ATATCTCAAAAGACAAAAAATTAAATAAAAACTTCAATTTCAAAGTAGTAACTTTCATTTTAACAAAAAAAAAAA
GTAGTAACTTTCATTCAAGCATATAATTTTTCTGTAAGATGCTTCATAACTAACTTTTAATCATTTTCATTACCA
TTAAGCAAAAGCTATAAAATCCCATAATTCATATACATAACAAAATAACAATCTTCCATCAAATAACTTTTATCT
ATGATATTAAAAAAAAAGAGAGGCCAGATTTGGGGGTGGTCTATTCATACTAAATGCAAACCATCTATCGACTAC
CAGACTGCATTAAATGTAACAAGTAACAACAACCAAAAAAAAAGATGAAAATTCACCACATCAACAGTATTGGAC
CCCACTTTCTTCTTAATTTTTCATTCAATTCTTCTCCCAACCATCTCTTTGCTGTCTTCATAACTATCTCATTTC
TTGTACGAATCAAGAATTTTTTTTCATTTTTGAAACCAAATAAATAAATAAAAATCTCATTATCTTGTTAGAGAT
AAAAAGAGAAACAACCAAAAAGAAAGAAGAAGAAGTGTTTGTTCTGTTTCAGTCATTCAAGTTCATGAGAATAGA
AACGAGTCCAAGTTTGTTGTTCATGCAAAATTCTCATGACTCTGTTACACTCCTGAATTTCTTCTACTTATATTC
CATTCAATAACTTTTCTTCTTCTATCAGTTCAACGTTCACACACTTTTTCCATTTCCTCGACAAACTCCTTTTTT
TCTCTCACTTTTCTCGAATTCTTTTTTTTTTTTTTTCTCTCAACGAAACATAAATCCTCTGTTTTGGTTTAAGAG
SEQ ID NO: 70 pAtKCS1_At1g01120
TATGCTTAAAAGTTAAATCTCTTTTGCAGTTCTACAAAAACATCTGGATTGGAGGATGTAGTCTGGAATACTGAT
GCAGCCGTTGAAGAACAAAAGCAAAAAAAAAAAAAAATAGCAAGTTGTTCAATTCAAAGTTTTTAACTAAAGTTG
AATGTATAATGAATAGCAAAAAATGCTGGAGTTGGCAAAATAGAACTATTCTTTACAAAAAGCCAAACCTAGAAG
TTGCTACAGATTTAGATCCATATGTTAGCCAATAGCCATATGCGATCATCCTCAAAACTCGGGTTGGCTTACTTT
GAGTTAGTCAGACCTATGAGTCAAAGTTCAAACAATTCAATTAGTCAAAAATACTCGGGAAGTTCGATATTTGAG
TATAGTTGAATACAAAGTTGTCACTTTATGGCTACGGTTGGCAAAGCGTTACAGTGACACAATATCGACAACAAT
TTGGAAACACACAATGATAGAATAATAGAGAGAAGCAAAAAAGTTATGTTAGCTATAAATCTACGAACCAAGTAT
ATTCTGAGTATAAAGTTTTGTTTTGTTATAAATGGGATTGGTTAATAATTTGCCAATCAAGTAGTGATTTTACTA
CAAAATACATAATGCTGGATATATAGCCAAGAATATGGATATAGATGGTCCATGTCTACGACAAAACTAGCAATG
AAATTGTGGCGGATAAAAAGACTTGATCATTTGTCGCTATTATGTTGCTATTTGTTAATCCATAGATTTATTCAA
AACAAATGACTACGATTTGGCAACAGAAACAATTGACAGAATTTATTTATCTGGTGATATGATGATATTGACATT
TTCAATATCGAATTCGTAGTTGGATTCGGATTAATCGTGACCAAATATTTATAATTCCATATATTAGCATTAATC
TTTCTTATTCGAGATTAAGAAAAAATTCGAGTAATTAAAAAAAAAAATTGTTTGAAAAACCATTAAAATCAATAA
TATTGTATTATATACAGTTATATACTGAAATTCAAATACAAGACCACGACAAAATGTTTACTCTAATGTTTCAAT
TTAATGAATCATTCGGTATTTTGAAGCATGTTAGAGCCTGGTAGTACTGTGCATGTTATACAGCCAATATCAACC
TTTTTCGCTAGTTTTATGTAAGGCTATATCGTTTATTTATGTAGCAACCATTTAAATTGTTAGTCCTTTTTAAAA
TTATTGCAAGATGTGAGTGTGTACATTCATACATTGATGACGGACTGGTTTCTAGTTAGTTGGTATTAAGCCGAC
CAACATTAATTGTTGGTTTATGTCTACACATGGGTACTAGTCTACCATTTATTATCATCATTATTTCATTTATCT
TGCAAATTAATACTTTGATCTGTTTATTGATTAATTCTCTCTATTGTTCATTATGCTGTACATAAACCTAAATAT
ATGAATTATTAGCATCTGATGAACTGGATCTTTCCAATATTGAAGTTTGAGAGTTGGTATGCGTACTGTTGAAAC
ATGTGTTTCATGCAGTAATCACGATATTATGGAAACGACGTCGCTGCCTTTTGTCTTTGTTTTCTTTTTCTCTAT
TATTTCCCAGTAATCACGATATTAGAAATGATTTAACTATTAAAAGCATTACATTTTACCAACAACTTTATCCCA
ATGAAAACAAAACAGGGACAATAAGTCCACTTTGGGACCTCAAATCCGAACATTTTATTTACCTACAAAGCTACA
AGCTACAACTACCCGTCCGAGGTCTATTTTGGACACCTTGGCACAATCAATATGAAATAACTAGAAAAATACTCG
AAAACTAACTAGATATGCAACTCCAAAACTAATTACTTCTTTATCAGATTTATAATATGAAATAAATTCATTATG
TAAAGGGTCCCTTATATAACAAACGTCTTAACGAAGACAGCAAATTTATAAATGACAATGACTACAATACTGGAA
CAGCTAATAGGGATGGTATATAATGGAGAAACAAATCGAAATGTGCTGCTATTAAGAAGTCATAGTAATTCATTA
TCATCAATTTACCATGGCCCATATTAAGGAGTTTGTATGCATGTGGGATACACATTTATACAAAGAAATAACAAA
TTTGAATATTTGGGACACGCTTTCGTCTACCCTTGTTCTTGCAATTTGGACAGTGAACGCTTCTCATGAATGAGA
GGTACGTTTCTCGAACACGTTCTCCCTTTAAATTTACTATTTACAACATATTATATCAACTTACCCGTCGCTGAC
CCAGTGCATTTGCACTACGTGCAGCCAACCTGCTTATAATATTCAATTGATATATAATTTTATAACTTGCCCTCA
CTCAATTTTGGATAACCTACATACACTTGCTAATAAAAACATCATGGATGTCTAAAACGTTTAAGTTTAATTATA
AGGAAATATATAACAAACATGTCATATTTTGTTGTGTTGTAGTGGTATAATTAACAAGTTATGTTGTTTATCAAA
AACTTCAAAAAAAAAAAAAATTAGGTTACACCAAAATAGAATGTTATGATGCCCAAATATATGTGTGAAAAAAAA
GATAAACCAAATTTATGATCTACCATATGACTTATGAGATTCAGCTAGGTTTTGTTAGACTTTTGTAACTTGCAT
TTCGTGTGAACAAAACATGAAAAATAATCTGACTACTTAATTTAATATCAAGGTCCCATATAAGTAGTTGAGCTA
ATAGTATTATCTCGGAACCACCCATCATTAAATTCGAATATTATTCCATCTTTTTTTTTTTTTTGTTAAGTCATT
ACTATTATTAATCTTTAAAATATTTTTATCTTTCTTGCACTCTATATATCTCTCCATCCCTACTTGCCCTCATTT
CACCAAAATCCTCCTAATTCACCAATTTTAGAATTCCCATTCTCATTTTCTCTCTCTTTCTATTTCTCCAAATTT
GCCTAAAGATTTTACATTTTATCTTCAATCCTTCCTTGATTTACGTTTGAAATATTTCGACCCAAAACTATACTG
SEQ ID NO: 71 pAtKCS2_DAISY_At1g04220
CGTTGCGTTTCATAAAAATTGTAAACAGCTTTCCAACAGACTTTTTTTTCTTAATAGGCTTTAAAGCCCAACGTC
ATAACTGTCTTTATTAAACATGATAAACGGTAGCATAAAATCTAGACGAAAAATCAACATTGTTAGACATGTAAA
TCATATGTTAATAGATTTTTGATTATTGGGTACAGAATTTGTGGTTGTTATAGCTACATGCATTTATATATGTAG
TACTATTCAACTTTTCTTGTTGTTACATGATTTCTATACGCTTGATTCGCATTAATCACACTTGTTCTATTCATT
TGTTCCTTGTTTAGCTATCACCATTCGATCTCGTATATGTACCCAACCCAAGTACTGAGTAACTATTTTTATAAA
TTTATCCAAGTACGAGTGAATCAAGTATTTATTTTTTTAGACCAACTTTTATTCTCTACATATACTTGAATCTTG
GGATACGTATAATATAAACCAACCCTCTTTAGATTTTAGGATTGTATTTTTGTGCATACACAGAAATCTGAGCAT
AGACAAATGGATAGAAACCACATTTGCTTTATTTCTATAGAGTTTGGACATTTTTTGGCTATGATTTTGTAAAAT
TGTTGTGTGCTCCGTGTTAGCGTTTGCCGAATGTGACACTAATTTTGATCGAACAAACAAAAAACTGAACATAAA
TTAATTAATTTGCACACATGTTTAACATCTATAAAAAAATTAAAAAAAATTGCACACATAAATATAGACAAATGT
TTGGAATAAGTAAAACATGAATCCAAGTGTTATTAAAATCAGTATTTTGTTACTACAATTGCACCAACAAAATTG
TATGTTCAAAAATTTAGCGCAGTACGAATCATTAATAACTTGTCCCTAACTACTAACTATATTCAGATGTATGCC
TAACTTGTAAATGTTACTCATGGTTGAGTAGTTGAATGTTGAAGAGCTTACGTTATAATTAAATGCTCGCTTTGA
TTGATGCAAACAATTTCTTCACTATAATAAGCGAAAATAATAGAAATAATAAAGATGGAAAACTAACAATTAATA
GTAGAACTATATATATATATATCCACATATAAATTCTTTTTTTTTTTGAGGGGTGGTTTAAAACTAGCTATTTAG
CTGGATGCTCGGCTATTCGGATTTGTGTAAAATACTTCTAAATATTGAGCAATAGGTAAATATAGTGAATATTTA
ACCATGACAAACAATAAAACACACACACATCAGAGGAAACTTGGGAATGAACGTGAACAATAAAAAAAAAATTAA
TAATGATAATAATAGTGGGCATAGAGATGGTTCTTCAACGAATTATTTCTTGCCCAAGTAGCACCGTTTGGTTAG
AAATTCTTCGACATTTATAGTTCAATTAAAAATTACTACTCCATAATTTTGGGTGACTTACCACACAATCTTTCC
ACAAATCTTCGACACGCAATTCACAACAGTATCATAGTTTATTATCACACTTTAGTAATTTATGCCAAACCTCAC
TACGCAAGCAAACTATGATTCATTAGATGTGTGGACAATTACGACGACAGCAAAAAAATAATATGTGTGGACACA
CAATGACAAACTCACATTGAACTATCTTGAAATAAATTTATACTAAATCGTAGCGAATATATAATCAGTAAATAG
AAGTTAAACTGAAGATCATAATCAACTAAAATGAATATCGTAATCAAATTGATGATAATAATGATTTCAGACACA
ATAATGGTGACGGTGTTGATGATGAAAAGGATCTGTGGCATAACAACTGATTGAATTCAATGCATCTTCATGGGT
TTTATTCCGTTAGCTTTATCTTTCACGTCTTTATATTCTTTAAACTTTGTTATGTTATTGACATTTAAATTCATT
TGTATGCATGTTATGAAGAGATCGTATAACTACAAAAATATAAAGGTAAATCTTTCATTTTAAAAAGAAAATGTT
TCTATACGTGTAAATAATTAGTAAATAACACATATTTCCTCCTGCGCGTAGCAGTTCAACAAACTCTCTTCTTCG
CGTAGCTAGAAGCTGCAATAACATAAAGAAATGATATATTTATAAATTATGTGAAAATGAAAAAAAATATTAGTC
CGCAATATCCACGTATCATTATGCGCCACGTAAACAAAGTGGACCCAGAATAACCCTTGTTTCCAGTAGAAGGAA
TCAATCAGTGGCAAGAAAGAAAATAATGAATTTCATTAAATCGCCACTCTCGAGCCATCAAAGTATCAAACAATT
GATTGGAGTTAGTTGTATCCTTCGCCTACAAATTTTTCCTTACTCTGTTTTATAAGTATATAATAGTTCCACTAA
AATCATTTTGGATACAACCTCAATAGATCTCAGCGTATTGTTTCCTGTATGTTCAATGAATCCATCCTCCATGCA
ATCTGGAGACTGGAGTATAAAATAACACGTATGGTATGGTATTTATTTCTCATAGCTAGTTCCTTAACAGTTGGA
AGAATTTATACATGACCCGGCCCTTTAAAACCTATCCCTACTACAAGACATTTGGGTTACTTTCCATCGACTTTT
GAATGTGCATCAGGCATGAAAATCCTTTAATTATGCATATTTTATAATCAATGCTTAAATTACTATAAACACAAA
AATTGTAGTGATATATTAGAAAGATAATTTAAATTGTTACATTGAAAAGATAATAAAAAATTATAAATAGACATC
TGATATAAAAATGGATGAAGTATAGCATATTAAAAAAACATATGTTTTTGGTCAAAACAGAATCAATGCATAGTT
AGCTCACCGCTACAACAATAACCATAGGGACTACGTACCATCCATAACTACATTTTCTTAAATTGCATCCTCTTT
CTAAAATTTTGCCTATAAATTCACAATAACACTTCAACTTTTTAGGCCATAAGTTATCTCTTTCTCTACAATAAG
CAATAAATCTCACCTCCCTTTTTTTTTTTTTTGTCTCGCTACTTTTGATTATCATTTAAAACCAAAAAACCTACC
SEQ ID NO: 72 pAtFATB_At1g08510
CACTATCCACATTAAGATTTCAATAAAATGTCTCAAAATTTATTAGTATGAATTTCTAATATGATATTTATTGTT
TTATAAATTTATTAATTAACCTAGAAAAATAATTAAAGAAGTTTTTCTTATTAATTGGAGAGTTACGTTTTTATT
CAATTTATATCTTTTAGGAACAAATAGATAGCGAAGGATTAAATTTTGCATAATAAATCATGAAATCTCAAGGTT
TAATTAAGTCAAGAGTATGGGAAATGCGAATTCAATCGCTTGGATGTAGTAAACAAGTGGTAACTAATATAGACC
ACAAACATTATTAGCTTTGACTATAAAATTGATCCTCAAGGTGAAAAGTAGGCTAACTCCTATAAATCTCCAAGG
CAACATCCTTAATTTTGAGAGTCATTCATAAGAAGAATTGTGTTCATAACTTTTTTTACTTGATCAGAAAATAAA
CTATGCAATAAGTAATAACTAGGTTTTCAAAAGGCATAATCATCTGTACTTCTTTGTATATGAAAAATAAATATG
CAAATGCAACTAAAGGTTTTGAATCATCTTTTTTGTTTAATTAAGACTTTTTAATGTAAACCAAATTAAACACGA
TATATATTCAAAATGAAAACAGAATTATCAACGCTGCTGTTTCTCCTGACTTAATTCTTGACTCGTCTCCAAGCA
AATTAAAGCGTCCAAATGAGAGATTTAATAATAATCAAAGATAAGTAATATCATCAATGACGAAATAGTCAACAT
AAAATAAAGTTTATTGTGATCTAGTCGATGTCTACTGTACTTTCTCAATGGTTATACTATTATTGGTCCCAGGCT
CCTCTTGTAGCGTATGCGAGGACTAATAGGCTGCTCTCATGGCTCATTTTAGAATTGTAAGTACGTTTCGCTTAA
GTCTCCTTCTCCATATGGCTCTTCGGAAGTACTAGTAGATCACATGCTCTACAAAGTTTTATACCGTCTCCTTCA
AGACGTATGCTAAGCATGCTTGTGCGAGTGTGCATCTCCCACACGTCTCTCGATCGTGGTAGTTATCTATGTCCC
TAGTATATGTGTTGTCAAACTTTTATTGATCTACGCTTGCCATCATCTCATGCATGCATGTGTCTCACATTTATT
ATGATCGTCAAATTTAACCACGAATATGGAGCAAACAAGAGACGGATATGGTCCTTGTAAAGATTGGCAATCATA
GTCAATTCCATGAACCTGGTCCTCAATAGCTTATCATATCTTCTTGTTAGCCTCTTATACGCTACCAAAATGATA
GCTGAAGGCAAAATTGATCTCTCCAAACTGGTAATTATATATTGTGATGTTCTTGTTGGGAAGTGTATCTACCTA
CTCTCCTAAACATTTGGATTGGCTATATTGTTTCGATTTGAAAAAAATAACACGTTGCTTTTGTATAATAACATT
ATTTGTATAGATTACTTGGTACGTAAAATTTTGAAGAAGTTAATTTAAATTATGTTTTCGGTGGAATAAATATAT
AAATCCGAAGGCTGACTTTACACGTTCGACAAAGTTTTATGGTTAAACTAAAAAAATGAGAAAAAGCTAGAGTTA
GGTTTAGTTTAAATGTAGTTTTCGTTAGTCTTAATAAATACAAGTAAGATAGAGATTGATGCACATGTACCTTGT
TGTCTCTGCCCACTTATTATTACACAGCGTCGGTGCCAGTATTGACAACAAGCTTGCTCACACATTCGGATATGT
GTCTTTTTCTCTCCAATTACAAATTTGATTGGTTCGATTTTTCAAAAAAAAATTTCCAAGTCCAAAGGAAATTTC
CACGGAAGCGCAAATCAAGTTTTGCGTGCTAAATGAAATTCAAAGAAAATTTGTAGTCCATATGAATAAAAATTG
AATAAATTTGCCTAATTAGGTTAACCAATCTTGTAACAAGAAAAGGGCAAAATGGGAAATATAGAAAATGAAATA
AAAATGTGTCGTTGTTGTTTTGAAACTATGGAGAAGAAGAGGAGAGGCAATTATATAAAAACCGACTCTCATATC
CACATATATCTCTCTCTCACCCACCTTATAGAATTTTCTCGCCCTATTTTTCTCTCAACACCAAAAATTTGTATT
TTTTTTTTACTATTGTTTACCCCAAAAAGTATTGAGAATCGCAAAAAAACTGAATTGTAAAAAATATTTGAGAAA
GAGAAATACAAAAAAAAACAAAAAAAAAGTTTTAAAGAAAAGGCGAAGGAGAGATCGTGAGAGACAGAGAGGCTG
CCGAAGAAAGGAGAATTTAGGGTTTGGAGACTTTTGTTGAAATTTGGCGTTACGTTTTTTTTGTATTTCCTCAAC
AAATTTCTCATCTGCTCTCTTCATATTGGTAAGATCTCTCTCTCTTTCATTTTTTTGTTTTTTTTGGGTTCTTCT
TCTCTTTGTGTAATTTCTGAATCTCTTCGTCGCTGGTTCGGATTCTTCATCTGATTGATGTTTGATTGAAGAATG
ATGTTTGAATTACATTTTTTCTAATACAAGTTTTTGTTTTTCTTTTGCGTTTCTACGATCTGGTTTTACCGGCTT
TAGCTTTTTCTCGCTTCTGTTCTTTGTTGTTTTGTATTTCAGATCTGGTGTTTTTTTTCTTACCTGCATCAAATT
GGTTTCTACCAAAACTTCGGAAACCTCTTTTGGCAAATGTTGAATCTTTGAATACAATGACGATTTACATAATAG
TCTCAGTGGCCGAACTGGATTATCTTACAATTTACGCAATAACAAAAAGTTTTTTTTTTTTTTTTTTTTTTGTGT
GTGTGTGTGGTGTGTTGAAGATTTTTAGTGTTTGTTTACTTCGTTTATGGAAGTCCTTTTCCTCTTCTGCCATTT
TTGTAGTTAACTACAAATTATACCTACTTTAGGAAGATCCTCCTGCTAGTAGCTAAAAGATGTAGCATTTATTTT
ATTATCACTCACTTGAGCTAACTTTTTTCGATCTTTATTTGGTGGCAGTGTCTTTGAACGCTTCATCTCCTCGTC
SEQ ID NO; 73 pAtLACS1_At2g47240
AGGTTGGAAAAATTTAATGATACATTGTACGTTTTCTAGGTTTTGTTCTAAATGCTCATAAACCTACTTGGATTT
GGAAATAAATGAGAGCTTACATATATAGAACAATACTCTATTTCCACAAAAATTATTTGCTACTCATATACAAAT
GTAGGTTTAGAAAAAATCCACTTTAGCAAAAAGAGATGGGAAATTAATGAAAATTCACTTGACTTTCTTTGTTTT
GTGAGCAATTTTCGATTTCAAAGTAATGGTCGGACTTAGACTTTTTCCTGTTCTTTTTTGCTGACTTAATAATAA
GCTGAAACTTAACAGTTAGAAGTTGGAACAAGATATAAATAAAAAAGCTAAGTTGTCAAACATAAAGTTGAAAAT
GATTTGGTGATGTCAATTAAATAAAAAGTTGAAAACTAATCATTTGTATTTGACCGGCGGTTACTTGGAATAAGA
CTAAGAATTATTGTACTTTCTATTTAATGGGTTTGCTAAGTTTTTTATCAGATCATTTTAAGCAAAATATCGATA
ACTTTTCGAAGAGATTAGTTGAAAATACTAAGCATTATATATATTTAAATATGATGGATCAGTATATATAGTTTT
GAAAATCACACTGCAGGATCCGTTGGAATTGGTGGCTGAGAACTTTTATGATCTTCTTAAAATTTTAATCCGTAA
ATAAATTATACGAGTTGGATTGGTTCGCGTCTTCTAATGCGTATTGACATCTATAATAAAAGCTATCAGAAAAGT
TGCTGATCTTTTTCTGTATCTTATTAAATATGTAGACCCGAGCAGAGAAATGTGGATCAATTAATAAATATCATG
TACGGATGAACAAGAGAGAAGAAAAAAGTATAATAATACTATAGAGCGAAAAGTATTGCATGATGGAGAAACTCT
CAACAATTTCAAACATGAACGACTTCTTACAAAACAATGTATCTTTAGTTGTCACAATCTTTGAAATTCTACAAA
TAGAGAAGACGGAAAAAGATGAAAATGAAATTAGTGTTTTTCTTTGGCAAGGTCCTTGGATCTCTTAAAAGTGAT
TAGATCAACGGTAATGTTTTAGATATCCTACATGTGCATTGATTCGATAAAAGCATTTAAATAGTGAATATTACA
AACAAAAATAGATACTTACATCATGAAATAGAAACAAAATCAAACTGGGTTGCTACTTACCAGTAATCACGTGAC
GGTAACTGGCTAACTGCAATAGTATATCCAAAACAACTTTAAGAAATGGATACGAAATGTTTTAGTTTTGGGAAA
ATGTCAAATAAAGATTTCGTCACAAATCCATCAATCCAGTATAGAACACTTTTATCAAAATGATTTTGCAAATTG
ATTCTTAGAGTACGATACAAGTTGCAAACTGGAAAAATAATTTTGCAGTTGCAACAAATAATAATAAATGATTGT
GCAAATTGATTCTCAAATTAAGTAGGAAACAAGTTGTAGTAGATATAGACTGTTTTTACATGATGTTAATTCCAA
AAGGATTCATTGCCATACAAATGGTTTTCTCATAGAGGACGTAATATCAATTAATGGAAAATAGTAGTTGAAGAT
TTATAGAAGAAAAAAAAAACTTTCCGCAAACAACACTATATATATATATATTGGATCAAAAATAAAAAGAGTTAA
ATGAAAGAAATACACAGACCTGGCCTGTATCAACCAACTCATATATTATTATATAAACCAACATCCATTTCTCAG
CTCCTCATCAAATTATCTTCTTTCTCTTTCTCTCTGATCATTTCCAAATTATTTCTAAGCTAAGAAAATCTTTAA
AAATAACAAAGCTTCTAAAATAATAATCCGACCTACCGGATTCAAACCATTGATCTGAAGATCATCTATAAACCA
GTCCTTGATCTATAAACTAAAATAAGGTCGGTTGCTTAATCTTTGTTACCCTTTTATTTTTGTGGTTATAATTAT
ATTATCTTCATTTTGTTTCCTTTTATGATTAATCGATCATATCAGGATAGATATTTCGTTAAGTTTATAGTTTAA
ATCCAGAATTTTATGTTTAGAAAAGAGAATGGGAAGAGTTGAAGTGTTTAACGTCTATGAATATTCCTCACGTCA
AGAAAAAAGAAAAAAAACTATGAATGGTCCTCTCTCGATGTCCTTACATTGGTTAGAGAGCGTTGCAACTACAAT
ACATACATCACTTTCATCATATTTATTGTCTTTTTCAGAGGTTTTCATATTTCGACTGAATTCTTCAATTTGCAA
TATTATGAATAATAGAGAAGATGATGAAAAAAGGATAAAGAGAGTGAAGCCAGGGTTATGCATGCTCTCACGTTT
AGGGCGTGACTCTAAACTAGCGATTTAGTTTACGGTATTATTAAAAATAATATTAGTCACATAATATTTGTAATT
TTAGTCGAAATTAATGACAGAAAAATACTAGACGCTAGGATTTGTACTTCCTTGAATCCTTGGAAAATATACGTA
TGTAATTAATATTATTCATTATCAAACCACAAATACGGAAACCAAGTTTGTGTCAAATCCAAATGGAGTAGTTTG
GTATCTTAGGTTGTTGTTTATCATAAATGAGAAACTTTGGGGTTGCACTCGATCGGTATGGTTAGGTGGACCGGA
CTGGTGACAATTAATGCCATTGATCTATAACTGTTAGATTATAATCTAAAGGTTCCATTGCAGAACAATTCATCA
AACTTTAATATCTATGTATTTATGTATGTTCCCAACTCACAGAATTTCACGGAATTGCAAAACTATGATAAATTC
TGCTACCTTAATGAATACACTATGAACTTGACTAAAAAAACAAGAATAAAGTATGATAAATTCCCTCATTTTATA
TTTCTCTTATCTATTTCACATTTTTGCTTGAAAATTTGTACCATTAATCTCTCTATTCGGCAGTGTTAAATTTTA
TTATTTAACTCCGTCGGTTGGATTCATTTTAAATTTCTCTTCAGGGAAGAAAAAAACAAGAGAGAGAAATAGAGA
SEQ ID NO: 74 pAtLACS2_At1g49430
CGATGGTTTCGATGAGCTTAATAGCTCCACGTCCTTTGGAACAGAGTTTGTCCATCACTTTCTCAGGACTGCAGC
AAACCACTTTGATTGTGACCGTGTTGGTTTCTTCTTCGAACTTTTGGTCTCTTACTTCTTTTGTTTTCCCAAAAA
AATCACATAAAACAAATTATTATTATGAGTTTTTAATTTCAAACCAAAAGAGACGTAAAAGGAGAGAAAGATATA
ACAATAACATACGAGGTATGCTCGAAAGAGCCTTCTTGACTTTCGAGAAATTCTTTTCGTTACTAAGAGGCTCCG
TCTTCAATTTCATCCATGTTACCTAGTTTAATTTCATCATATAATGTTCTCATAGCTTTTAATACAACTTTTGTA
ACAATTAGTAGTAACGGTTTCAAAGAGAAGCACAAAAAAGAAAAAGAAGAGAGCGTTTATTTACCTTCTCCTTAC
CCTTCTCGGCCATGGCTTTGAAAAGAAAAATATTTTCAGTGTAATTGCTTGTATATACTAACACTACTATGCAGC
TATGCTATATATTTGGAGTTTCACGAGAGGGTTTCCAAGGAAAAAGAAGTTGAATATTGTACAATTTACTTTTCT
CTTAGAATGTATAATTTCCATAGCTATTATTCCATATTACATTGTCCTAAAACAATGTAAATTTTCTTTTCTTTT
TTTGTTTAAAGTTTTTCCTATCTTGTTTTTTTAGTTGAAATTTCAAATTTCCTTTTGTTAACAAAAAGAAAATAT
TACCAAAAAACAAATGATGGGGCTTAGTGGGGGTTCTAAAGAGAGAAACAAAGCATAGTTCTATTCTTTTGGAAG
AGGACTAAACCCTGCTGTTTTTTTCGGAGCGAGGATTTACGAGTTTTGGCCAAGAACTTTCGGTTTTTTTTTTTT
TTTTGAATAGTCCTAGAACTTTCGGTTTTTTAAAAAATAATACTAATGTAATAGAAAAAAAATGGTGTTTCTTCG
GAGAAAAATGACAACGGGTGGCAAACCACCTTCGAAGCTATCCCTTGAGCATGTGCACGCCAGTGTACTTAAGTG
AGACATTAAGTTCCAAATCAATGCGAACGAGCTGAGAGGAGGAGATCCCATTACGACTGTGTCGACTCAAATTTC
TCTTCTTCCATAAGTAATAGATCACTGATTGGAACCACCTTGTGATACAAAACATTATAATCCCATTTGATGTAT
TTTACAGAGATTAGATGAGAAACCGTGTCTGACCAATGATAGTCGGCTAGAATGTGAATGTGTAAGAACACACAA
AACAAAATAAACCTTGGTGGAAAAATTATTATTCAAGCGTTAATCGTGAGAAGAAAATAATATTCTTTCTTAAAG
TATTGTGAAAACATAAACTTTTCAAAATCTAAAATATGTTAAATATTTATTTGAATTTACTTTGTAGTTTAACCG
TGTAAAAACTTTTTACCTATAAAAAAGAAAAAAAAATAATAGGGATAAGTTTTTTTTATATATACCCATAATAAT
TAAAACATCGTTCAAATTTGGATATTTTCCTTATAAAAAGTTAAAGAAAAAGCATGTTTTTGTAAATTTAATTCA
TAGAAAATGTCTGAAATTTTCCATTATTTATAAAGATTATAGATTCGCCGAAAATTAAATTGTTAAGGCTGGGGA
AGAATTATTGTTCACGCCAACAATCACGTTATGACATAAGATTCGAAAGTTAGCGTTAAATTTGATGTTTCCACT
GTTACCAAGCAAAAAAATAAACATGTCAAACAAAAGAAAATGGACTACAAATTTGATAGAAAAAAATGTGAATAA
AATAACTGTAGTCGTTTATTTACTTAGAGTTGTATACATATTGACTAAACACAATAAGAAAACAATTTTATAGAA
TACTATAGGTCTTAAAAGATAGTATATTTTATTTTCATTTGATGAAATTACATATTTGACTATGTTTCTAATTGA
AAAAAAAAAAATTTATTAAAATATCCATAAAAATGAAATGATAAAATTTATTAAAATTAAATGGGCTGACCTTGT
AATAATATAGGGAGCAGGTCGGAGACTCAGAGAGTGAGCTTTTCTTGCTCGTCCATTTATTAATAATTTCATTAA
AGAGAAAATCTGTAGAGTTGACACTTTTTAAAGTCTAATTATTAGGGATATATACGTAATTTGGTTTGAAAATAA
ATATCAAAATCTAGCTAACTTGATATCAGTATGGGTAAAAGATAGGAGTACCTTTTGTTGTTGATTTAATTGACG
ATCTAGTGTTAACCCAGAGAATTCTAATTAGATTAGCCTCTTTCTCTCATCCGAAAGTTGAAGTTTAATAAGTTA
ATCTGATATTTGTAAATAACTTGAACAGTAAATCATAAACAAAGAAAAACTGATTTGGTTGCACAATTTAATTAG
TGAGGTGAGGTCTATCACAATCACTGGTTACTATAGTGATAAAGATCCTGATTATGACAGTGACTGTGAGCTGGT
GAGGTCTAAGAGTACAAAAACAAATGATCTAAGTTATCAAACTATAAACTATATAATTCGAAATATATTAATTTT
GTTAAATCCAAAACCAAATTGCATGCAAATGGTCAGTTTGTAAATAGTCTACGTTTGGTCCAATTACTCTTTACT
CTTAAGGTTCTTTACTATATTCAAACCCACTAAATCTACTTGCATATAACACATAGAGACACAAAAATTAGTATT
TAAAACATTTAGATAACAAACAAAAGAAAGAGAGGCTCCTAATAAAGGCGCAATTAAAAACCGGTCAGAAATACA
CCAATTGTGAGACTCAAGAAGTTATTGCTCAACTACTTCCTCTAACTCATAACCACAATCTCCAAACTTCACCAC
CGATTCACAGAGACTTTTATTAACCTTCAAAACATTATTTATTATTCCAATCTTCAAAGAGAGACACTCACACTC
CTTATCTTCTTCATTCCTCCATCCTATTTTTCTTCTTCGCCAAAGTGTTATTCCTCAACTTTATATATTACACAA
SEQ ID NO: 75 pAtCYP86A4_At1g01600
ACGATATGCTTTCTTCATCTTCTTCTGATCAAATCAATCGCCTCGCTGTTTCCGTCGAGGGACCCTACGGCCCTA
GTTCCACTGATTTCCTACGGTAATTCACTTAAATCACAAACAGAGAAGTTCAACATTCTGTTTCTTCCGACACAA
GTAACATTGATGCTCTGTTTTTCTTCCAACACAAGTAAAATGCTCTGTTTTTCTTTAAGTGCAGACATGAATCTC
TGGTGATGGTGAGTGGAGGCAGTGGAATTACACCGTTTATCTCCATAGTCCGCGACCTATTCTACATGAGTTCAA
CGCACAAATGTAAAATCCCCAAGATGACCCTGATCTGCGCGTTCAAAAACTCTTCCGACCTATCCATGCTCGACC
TAATTCTACCCACCTCTGGTCTCACCACCGACATGGCCTCGTTTGTAGATATCCAGATCAAAGCCTTCGTCACCC
GAGAAGAGAAAACATCAGTAAAAGAATCAACTCACAATAGGAATATCATCAAAACCCGACATTTCAAACCAAACG
TCTCAGACCAGCCCATCTCACCAATCCTCGGGCCCAACTCTTGGCTCTGTCTTGCAGCCATCCTCTCTTCTTCCT
TCATGATCTTTATTGTCATCATCGCAATCATCACGAGATATCACATTCACCCAATCGATCAAAACTCGGAAAAGT
ACACTTGGGCTTATAAATCGCTCATTTACCTTGTATCAATCTCCATTACCGTGGTGACTACTTCTACGGCTGCTA
TGTTGTGGAACAAAAAAAAGTACTATGCGAAAAATGACCAATATGTCGACAACTTATCTCCGGTGATTATCGAGA
GCTCGCCGCAGCAATTGATATCGCAATCTACCGACATTCATTACGGAGAGAGGCCTAACCTTAACAGTAAGTCTA
TTTATAACATTTTCATAGAGTAAAATGCTGAGTTCTAACTCTGTTTATTCTATTGTGTGAAACAGAGCTTCTAGT
TGGTTTAAAGGGTTCGAGCGTGGGAATTCTTGTGTGTGGTCCGAAGAAGATGAGACAAAAGGTAGCGAAGATCTG
TTCTTTTGGTTCTGCTGAGAATCTTCACTTTGAATCTATTAGTTTTAGCTGGTGAATTTTAAATTCAGACTGATG
TTTTATCATAGATGTTTGGGTTACAAAAGAACTAGGTGGCTTCGAGAGTTCATAACTCTAAACATGTCCTAGTAC
TAATTTATCCGTCTCCGGTCTCCTAGTAGACTAGTACTAATATTAAAATGTGAAGCGATATATGAGTCACATGTT
GGCAGGATTTAAAGTTTGACAGATCTATATGCGGTGGATACGAGTTCGGTTAGCTGAAATTTGTAGAACAAGCAT
TTATGTCATGAACGTTTCTGAATGGTTTTATAATGGTAATTAGTGAATGGTTTAAGACATTCTTTAGGCAGTCAT
GTAGTTTTGTAATATCAAGGAAAATTGGCTTGGCTCTGAATATGATTTTTTTTTCTGAATTGGCTTGTTCGAATC
CTCAAGATGATTTTTTGAAATGTAAAGTTGCATTTTCTGATATAATTTTTTTTTGTTGCCTTTTATAAATAATCA
TCTTTTCTTTAACATTTGGTTGCCTTTTATTAAAAATCATCTTTTCTGATTTTTTTTGGTCAACTAATGTAACAC
CTTTTCTTTAACATTTTTTTTAAAAAAATAAAACGCAAATGAAGTCATGGTAACCCTTTTGACTTTCACAATACC
ATTCAGTGACATCTTTCTTACTTAATATTCCGCTGTCATGTCCCACTTTGGTTAACATTTGAGGAATTTCATCAG
TAAAAATCGAAAAGATATAAAGGCATCAAAGGAGAAACCTTAAAAAGTCAAAATAGAGACACAAGAAGTGTTTCT
TTGTAGGCTTAGATGAGAAGAAGAAACTAAGGAAACTGTATTCCTAAAGTAAATACTACTTCCTTCATTCCTATG
ATAATCAATTATTAAAAGATATGGAGTCGCTTACCAAATCATAAAACCGGTAACCCAAATCTAGGAAATTTTATT
GTGGAATAAATAACAAAGCCTTTATTTTATTTAAATAGAACAAGATAAAACACCAAAAATAATAGCTATGTTCTG
AAGTCTTGACCAAACTACTGATACGGGATCTCACCTAGATTACTTACAATGTAGGTGTAAACCGAGTTTTTAACC
AAACTAAAACGAATTAAGCGACAATTAGTTACGGAAGCTTAAACTCTACATAGATTGATAGATTAAAAAACCCTT
GAACAAAAAAAGAATCCAAATTCAGCTTGAAGTAGATTCAACCAAACCGAAATAATTTTTTAAAAATATGAATTT
TGTTTCAGTTTGAATTTCATTTGATTTTCCAAAAATTCACAAACCAAACCAAAAACTGAATATCACTTGTAGCTG
TACCTATAAATTATTAGAGCATTAAATCTATCTCACCACCGCCAAACTCAACGTGTGTTAATCTTTTAACCCTAA
ACTTCTAAATTAAAAAATAGACGACTTTTTTACTTATTTCTTCTTCTTCCCCACCATCTTTTTTTGTGCTCTATG
TTCTCAACTAATCTCATTTAATTCACCACATACAGCTGCATCAAACATATATATATATGCATGTATTTATGTACT
TATACACATACATACATATGATCATTTAATGAAATTCATTAAATGAAACTATTCCTTTCGCTGCAAAATTAGGTA
CCAGTAAAGCATAAACACCACTTTACCACTTCTTGAATTCCAAATCTGAAAGCTCTGTCATTATATATGTATAAA
CCAATTTAAACATATACATGTCTCACCGATAACAACGGAGGTTCATTAATGAGGGTTTGATTATGATACTTACGA
GTGACAGATTGTGAATATCAAAGAGAAGCTTCTTCCTCCCTTTTTTCTCCCTATAAATTTCCGTAGAAAAATCAT
TCATTCGCATCTCTTCTCTTCTCAAAAATCACCACATTTCTTTACTTCTCTGGGAAACAATAATAAAAGAGCTAC
SEQ ID NO: 76 pAtCYP86A7_At1g63710
CTTCCTTTTATAGCTCCACCTTCATCTCCAGCTTCATTTTTCCAATCAGAGCCACCTTCTGCTACACAATCACCT
GTTGGAATCCTCTCTTTTAGTCCTTTGCCTTGTAACAACCGTCCTTCCATCTTCGCCATTGGACCTTACGCTCAT
GAAACTCAATTGGTATCTCCTCCGGTTTTCTCAACTTACACTACTGAACCATCTTCAGCTCCAATCACACCTCCT
CTTGATGACTCATCTATCTACTTAACCACCACAACACCTTCTTCACCTGAAGTGCCTTTTGCTCAGCTCTTTAAC
TCGAACCATCAAACCGGTAGCTATGGTTATAAGTTCCCAATGTCTTCTAGTTATGAGTTTCAGTTTTACCAACTT
CCTCCTGGTAGTCCACTTGGTCAGCTTATTTCCCCGAGCCCTGGTTCTGGTCCAACTTCTCCTTTTCCCGATGGA
GAAACCTCGCTGTTCCCTCACTTTCAAGTCTCTGATCCTCCAAAACTGTTGAGTCCAAAGACTGCTGGTGTTACA
ACTCCTTGTAAAGAGCAGAAGATTGTAAGACCGCATAAACCGGTTTCATTCGATCTTGATGCTGATCATGTCATT
AGATGCGTGGATCAGAAGCTAAGAACAACGTTCCCTGAAGCATCATCAGATCAAGAATCAATGAATCATTCGTCT
CTCGGGTCCAATAAGGAATTCAATTTCGGCACGGATGAGAAACATTTGACCGTTGATGAACATAGATCAGCTTCG
CCGAAGAACAGCAATGATTGGTCTTTTTTCCCTGTGATGCAGTCAGGTACACTAAGCTAACCTTCATCAGAAGAA
TAGAAATCTGAAATTTAGATATCGATTCGGACAAATATCTTGTTCAAGATTCAAGAACAATTATAGAATTTTTAG
ATGATTCTGTTCAGGATCTTAAGGATATTTTCTTGTCTCTCTTTTTGGTTTTGTAATAAATATTTGGCATCGTTA
GTTGTTGTATATGGCTACTCTTTATGTAGTTTTTTGTTTTTGTGAATACACATTTGATCGCATTTGTAAATAAAA
TTTAATCAGTTTCTTCGGAGAAATTCCATTAAATTTATAGGTATTGTTTACTATTTGATCTTCTGTTCTGTGTAT
TGTTGTGACTAGAGTTTTGTTATTGTTAGAGCCAATAAAAGGTGAGATCTATAGCATAAAGGTAGTGAATGTTTC
TCCTTATTTGTTATCTTAGATTCAACCATGATATGCAATGTAAAGGAGAAAAAGAAAAAAGAGAGTAAACATTTG
AGTATACAATTAACAAGCAACTCAAGACTGTTAACAAGTTTTATTCACCAAATCGATATACCGGTCAGGTTTTAT
AAGAAACATCACATCACAACCACTAAGTCCACTACAATTTGAAGCTTTGAGCCCTGTAAACAAGGATTATACATG
ACAAGTTAGAATGGGCCAGCTTAAATTACTAAGTCCACTCTCAGCATTCCGCAAGCCCACTTGGAATCCAGCTTC
AAGTTCTCATAGGCGCTTTTGGTTCAATTAATCCCACCTAAAACCAGTATGCAATTGGAGAGTTAATAAATCTTG
TAGGTTTAACTCTTGTTAGGAGGATAAATTAGATGATTTAAATTTCCTTATGTCTTTTTAAAAAAAAAAAATACA
ATTGAGTCTTGTCAATATGTATCTCTTTTTTTTTTTTTTTTTTTTTTACAGCAACGTTAACTACTAGTCAGCAAA
AAACACATAGGTTAAAATATGTCGAGTCTCATAATGTGAAAACAAAAGAAAATACAAAGATTGAAAAAGAAACAA
CGACCCCCACTTTTAAACCCAATTAATGCCGTATTTTAAGTTGAAGTGCCGCTTTCCTCAAATCTCACTATTGTT
TCTCTTATTCTCTATCATTCATATTTCATAGCCAACAAAGATTATGGTTTAGAGCATTTACTGATTTTATGTCAA
TTATAAACATGTCTTTTTTATCAAACGTTGTGAATAATCGATTACTTATTATATGGTGCTATTTATAAACTTACT
GCATTTCTTTTTCTTCAAATATTCCGTTATAAAGGTGTTGAGAATTTTGTTTATAAAATTTTAGAATTACTGATT
ACTGTACATACATGTGTTTGTGGTATTTTGTTAATTATTGTCAATTATCAAAACACGAAATGTTGTCTTATTAAC
CCTTTTTCTGAATTATTGTGTTCTTGTGGAGGATATATTATATAGGTCGACATAATTTATTCGGATCACGCGTGG
AAATTATATTTTGCCAAAACGAAAGTAGGAATTATATGATTCATGTATCATGTTATTGCTATTTTTATTTGATAC
TGTATTTTTTATAAATAAAGTTTTTTGGCTCACCAACTTAAGTGGCACTAATGGCATTGGCCCCTTTAAGCTGTA
AGTTACCATTAATTTATGTTGCCAAAATATATAGCTGTCATTATGTTGCCTTAGTATGAATATATTTTTCTATCA
CACAATATCAATAGATATATGGTTATGACCATATCAACTTGTCCCATCAACATCTCAATCAAAACATTTGACATT
ATATAATTAACCAATTGATTTTAGGATAGGACTCTACAAACTTTTATTTAAACAATTTTCGCCTGAATAAAATCA
TGTGTTTAATTAGGAAGGTGATTATGCACAAACTCATAAGTGAGGAAATAAAAGGTTGAGATATAGTAGGTGAAT
TAATATATACAATATTAGGTTATTATAAACACAATGACAACCACAAAACTCTACACAAACAAACACATGACATGC
AAATGCAACTATACGTCCCCAATTTTTTGGTTGGGAAGAATAATCAACAACAAACTTAGTTGCCTTATTAATTGC
TTATACTTTTCTTTTCTCTTTTTAGATTTCATTTTCACATTTTTTAATCTATAAAACATTAATATGATTATCTTC
CATTTCACACACACACTCACACTTCAATTCTCTTCTTATAGATTCTGCTCTCACCTCCAGACTAAAATATAAAAA
SEQ ID NO: 77 pAtLCR_CYP86A5_At2g45970
CCATTTCACAAAGATGGTAAAAACCGTAAAATGCTGATGAAATCGCAAAATCATTTTTTTTACTACCAAAGAGAA
CCATTGTTACACCGATACAAGCTTTAGCTTTAGCATGTGTCGCAGTAGTCGAGACAAAATATAAATCCGTCGTTC
TTTAGTTTCGGTGGTAAACGAGAGAATAAATGACTAATGATATAGATAATTAAGTAAACATGTGATTATGCGTAT
TGAATTATTAGCCTCTAGAGTGGTTGAGTGTTGTCTGAATTACTATTAATTACCATGTGAATAAGCGACGTGGTA
ACTAACTATCATAATGATTCATTCATTATTTTAGCTACTTTTCAATATGATTTTTTTTATATCTCCTTTTGATTC
TATATATGGTGGTCTTAAATGCCAAATGAGACAAGTAACAAGTAAAGTGACCAAAACAATTTCACTTAAATGACC
AGTTTTACGTGGGACAACCCACGGAATCTAAAGATTTCTATGTTGTTTATTACTTAACGTGATAGGTTAAGTTTG
TTTGTTAAGTCGTAAGAATCGTAACCATGGACTCATTACCATTAATGTGCCAAATTCCGAATAGATTTTGTAGTC
ATATAAAGTCATTGCGTTTATTCAATAAACAAGTCATAGCTATCGAGAATTCCTTCATTTTATTTTATTTTTTTG
GTAAATAGTGCATGGGATGGATATACTTGATGCACCTAATCTAACTATTTTATTGTCTATTATTTATGTAACTTC
CCGTACGTAACTGTCAATTTCAAGTATAAATATTAATTAATTTGTAAAGATATTATTATTTAAAATGATCTACAA
AGTGTATTGTATATGTTTTTATTTTGCCGTTGATGGGAAGGTAACCTAATTAGATAACTTTATATTCTCTATCGA
TTTGTGTCCTTTGTCTTAAGTTTTAACCTAATATTGCATATTTAACCACATAAATACACTAACATGTGTAAACAT
TCACATTCTGCAAGGATTTTTTTGTTTTTAAAAGAACTTGAAACAAAATAAAAGAAAAGGATCAACTGAAAACCA
ACTGTCAATCAAGTTCATTTAAAACTCACCTTGTTTCATACAGCTGTAATGAAAAATAATAGTAATGGATTTCGA
AGTATTTTAAAGTTCCAACCGGGTATTCTGCAATGCTTGGTTTGAAATTTGAAAAAGTTGACTATGGTTGGTCAA
TGCATTCATGCTTTGATGACAAAAATCAAAATTATATATATAAAAAATAGTTTTCTTCCTTCACAATTATCCACT
ATAGGGTTTATATATACTATATACTCTTTAATTAATTCTCCGATAGTTGTTCCTTTTGAAAAGGGTTGAATTTTC
TGATAGTTTCGTTAACTTAATCGTTTTTCTTCTATGCATTAATTTTTCGAAAATTGAACTCCAATGCAGTACATG
ACTACATCGTATTTTAATATTTTCCCACAGCTTCTAATAATATTACAACGGGGGGTTTAAATTCAAAATTTAAAA
GATTTTAATCGTCGACATGTGAAAATGTAGTATTAGGAGTGTGTTTATATATCCCATGTACACATCACGAGTTCT
CGTATGAGTATGTGTTGGTTCTTACATTAATCAATTTATCACGATCACGCATTAAGAAGGCGACACATTATTATA
TTGTAGACATTGAATACTTGTAACGTCCTTTTTAAAAATATATTGACAAATATGTCACCAAACCCAAAAGCAACC
TACATATGTTGTGTAGTTGAGCAAAACTATCATTGGTGAGTTTGTTCACCTATACCCTAACAATCTCAAATCTAA
AACCCTAATTACTCATCCCTATACTCTATTGTATCACAAAAATAGGATTCTATAATTTTATACACGCACATATAC
TTTGTCAAAGCGTATCATAAAAAAATGAAATAAAAATCAACGAAATAACGTGATATGGCTAGTAATATAGTACGT
AGTCTTACGACAATAGTTTAACTTATACGTGAAAAAGTGGGATAACATGAGTTTTCATATTTTTGTCATCAAATG
ATATGAAATAGAATAACCGAACATAGATCATTTTTTCACTAATTGAGAAGAAGAGAATTAGAGACTATCGACTCG
TACGAGTGGTGAAACCCCTGTCATTTTGCCGTTATACAATCTACTGACCATGTCCATGACTAATAATACATCTAT
CAACAACCATCATGTCCCTTGAACCGACACCGAATTACAAAACCCTAACTTAATATCTTCAAAACATGCAATTAA
TAGACACGAGAATAAGATAAAATGAGGAGGCAACATCCCATGCTTACTTTGTGACTTCTTTTACACGATTTTCTA
ATACATTTTAAAAATACTACTTGACATCTCTCCATTTTTGGGGGGTTTTGAATTAAATTGTATATATAAATGGTT
CATGCTTGGACATTGAAAACCTGTGGGTCATTTAGTCATAGATCTCTCTCTATCTTCCTTCCTCCTCCTCTTACT
AGGCTCCTCAACTAAACGTCTTCAATGCTCTCTCTCTCTCTCTCTCCATTCTTTTACGACCTCATAGACTTGAAA
ATTTACCATTCTATTTTTCAAAACCTTTATATTGATGAGATTAATATATAAAGTCTCCAACCACCTAGCTACCAG
CCTAGAGCAGGAACATTAATGGTGGTGCACACCATCTCATGCTTCACATACCCTAAAGTTCTTCACTCCAAGTTT
ATGAGGAGATCTAGAGAGATAATCATATAGAGAAAGAGAGATCTCCAGAGAAAATCCATTGAAGGGAAAAAAACA
GTCTGTCTTTCATAATTGCTCTTTCTATAAATCTTCTCTCTTGTGTTCTTACCTTTCTTCTAAAACCATTCTCTC
TCTCTTTAACACTTCATCACTCTTTACGGAAAATTTCTTATTTTCACCACCTGAGCCATCTCTATTTTCGTCAAA
ACTTCTCAACTTTTGTGCCAAAACGTCATTTCTTCTGAGGGAATAAACACAAAAGAACAATTTTCCCGGAAAATC
SEQ ID NO: 78 pAtKCS10_FDH_At2g26250
GTCGTGTTTCGTTTGTGGATTTTGTTTTCTCATGATTTTTAAAATCCCATGTTTACACGATCATAACGTTTCGTC
AATTTTAAAACGATAACTCTATAAGTAATAGCGTGTACCTTCATCGTATTTCTTTCGACGTCGTAATTATGATCA
CCATTCACCATTTTCCTTCTTTTTTCTTTTTAGGGTATATAACGCAACTATATAAAGCACACGTACTTAAAGAAA
CCCATTTGGGCATGTGTTGTCCTTGCTGTGGATCACATGCATAAGTCTGCTTTTTCTTTTTAATATGAATTGTTT
TTGACTTCATATATAGGACCAATTCAAACATTTATATTTAAAGAATATTTTACTCAGTATTATTACTTTCTTTTG
TAATGATTTACAATTTTGTGCCACTCGTATGTATAGCGTTTTAAAGAGGTTACTTACCAGTAGTTTTTGTCCAAA
TTTTATATGTGAAATAACCCTTCTCGGAAGGCTACCACATTTCCATTAAATTATTGGAAAACTTTTACAAAATGT
GACAATTCGATCGGAAGGCTACTATCTCATAGGTATTGAGGCAGGATAGTCACTAAGTTAATGATTCTGCATCGG
TCACAAACGTGGTGATCGACGTGAAAAGATTGAGCCCTCGCAAAAGTGAGATGGTGCTTTGTCAAACTTTTGCGA
TAAGTGATCATGATAATTACAAATTAAACTTCATTTTTGCCGTTTATGTGTCACTTAAAATTATGTTTTATTTTA
GTTTTCTTAAATGAAAAAAGAGAGTTTATATATATATATATATATATATGAATCAAAATGTTAGTTAGTGGATCT
TTTGATTCTGAAAAATTGTTGGATATTTGTTTCCTCGCTTAAATTGGTATTTATAGTAGAGTTTGAATCGAGGAT
TTGAATAGGTTCAAGAATTATTTTACCCTTTGGAAGCGTCATCTCGCTTTGAGTTCTACTACGATGTTATGGTTA
GACTCCCACGGTGGGAGTTCCCATTTACTCTTAAATGCAACTGAACGGGCTTTACTATTTGATGCAATGATACAA
AGAACCTTCAGATTTAAACAATGAGACGATCGCAACACAAGAGAATAATGTTACTGATCCTTCTACAACAAACAC
TGCACCAAGGCCTAGTTTGCTTGATTGAATGAAGTATACAAAGCATAGACACTTATCACATTTAAAGTATTTGTA
ATATGGCAATTTCGTATGTGTTAACACATTTCTCAAATTTTAAAAATCGTTTATATGGATCTTATAAAATATGGT
AGTAATAATTTAGGTGGGGGATTGGGTCATTATTTATTTCTCTTCTATTTTGCTACTGCATTAACTAATATTAGG
CTAAAAGTTCCTTAAATAATAAAGCTTTTTTATGCGTAATGTAGCCGCCCTCATTTATATAGGTCTCACCACCAA
TCGATTAATTGTAAGTAACAGAAATTGTTGATTATAGTTAAATGAGTCGTTGATAATTTGTTTAAATTTGTAAAC
TCTTCAGGCAAAATTTATATTATGTGAACAAAATTGCCATATATTTCATATCATGAATGAATTTGGTCTAAACAT
ACTCCATACTGTCAAGGTTTAAAAAACATTATATTCATACAATCATACCATGAATGAATTTGTGTAAAGCTGTAA
ACTCTTCTTGCATCCTAGCTATTTTCCTCTTCTTCTTTTTTCTTTTTCTGACTAGTCATATAGATTTTTTTTTTG
ATTTTTATTTGATCGTTGGAGAATCTTCAAAAGATGACTCCACTCATCTTTTCAAAAACTACCTAGACCATATAC
AGGGCTCTCCGTCTACAACAATTAGCGTCCGGAAAACGTAAAGAAGTCTACAACAATTAGCGTCCTCGTGGATCA
GTATCGTGACATTTGTGTCATGGAACCTATCCTTGGCAGTCGAAGTTAGGCTCACTCCCAACGGTCTAACTTCTA
AGTGCTCACTAAAATATGAAAAACTAATGTTCTCGAACAAACACCACCTATAATGACTCAACCCTCCATAAAATT
CGAAAACTGATCTTAAACAAAAATATATATAAGTGACGTAAAACATAAATTTATGTATATCTATTAAATAATAAA
TTAGTGAAGTAAAACATAAACTTAATCGCATAAAAGTTTAACTTTATTTTGCTTCTCAATAGATTTTGACTACAT
AATAGACGTGGATTTTAGTCAAACACAAGCGATATTGCAAAATTTCCGATATATATATATACACGTCTTTTTCAC
TAATAGAAAAGTAAACATATCAGCTCATCTTTTCTCTATTGGCAGTTCTCATCATCTTAATTGAAAATTTATGTT
TCCACTTTTTTTAGCTTAGATACAAACTCAACTTTGTAAAAGATTTTCTTGTTGCATATTAAATTTTACATCACA
ACAAAAAGAAAAAAAGAAATAAACAAAGGAGATCAAATAGGTTGTACATAAATTAAGCAATCATACAACATCAAA
TCAACAGCTAAAAAGTTCAGTACAATCTTTACTTCCGAATTTACTTCATTGTCTTGACATCATGTTCTAAACAAA
AACCAAACCAAAGTTCTCCACAAACCTGTCGGTTTTTAACCAAACCAAAGTTACTAGCTTAATTCAAAAAAAAAA
AAAAACAGAACTGATTCAGAAACCCCAAATTGACAAATTGTCAAATTGTCAAAAAGTGGTCATCTTTATCCTTTG
ACATCTGGTTAGTATAAAAAATTGTAATAAATGCAAACATAAGATCCAAAACTACACCTTATTAGAACGGAGGAA
TTAAGAGTGACATAACTCACTCCAACTACGACCAGTACAAGTAGTTGGCGACATTAACTACCTCTCACCAACCAC
CAAACCCAATCCCCACAATATTACCATTACTCTCATATAACTACACATATTCATATTTACATTTTTTGCCAACAC
AACTCCTTATAAGATATACACTTCATCAACCTATAGATCTCACTCACATAATCAACCTACAAAACAAAAACAAGA
SEQ ID NO: 79 pAtCER60_KCS5_At1g25450
GGAAAACAAAAAAAAGCTTCAATAAACATAAGCCAGTTTATTTTATCAAAAATGTTATGACAAATAATGTAAACA
TGAGATATGTTTTGACCAGATTGTTGTTGTCCAGACGAACAGAAACTCGAGATGCGATGTGGAAGACTCCATCAA
CACCGTTGACTGCTTCGTCAAAGCTTCCCTCTATCGTTAGATCAGCTTCGAAGATCTTCAACCTTTCTTTTGCTC
CTTTTAAGTCCCACAAGAAACCCACCTTTTCTTCATCTTCTTAATAACAAGGAAAGAAAATTACAGAATCATTTA
AGAAACCAAAAAAAAAAACTTGTGGAAGCAAACCTAAGAATCTTTGGAGGATTCTAGGTAAAGTACCTGAGTCTC
TAACAGTTGTTCTTACGTAATGACCAAATTCTAGAAGTGACTTGATAACATGAGAGGCTATAAAGCTGGTTCCTC
CGGTAACCAAGTACTCTGCCATTTTATTAGGCTCCTCTGATTTTGTGTAACATTGACAAACTAGATCATGCGATT
AGATGGTCTAATTTTATTTTATTTTGTGTTAGAAGGATGTTCAATCCTGGTTGATTTAGTTTATGTTTGACTTTT
TTTTATGTAGTCTTTGAATAATAGTAGTTACATCTAACTTTCATAAAGACTTGGATTAGTATAACCTAATTATAA
GCTTTGGGCTTGAAGCAAATATAATGAACTGAATGTGGGCTTTTAAAGTATCCAAAGAGGTTTATGAAATGTGAT
ATATTTTTTTTGGTCTTCAATCTCTTGAAAATTGGTTAGCAATCCATTGGCAGTTGAAGGCTCTCTCAGAGACCT
TGACTAGAAGGCCTCAAAGGCAAAGGAAGGTGGAAGGAAAAATCAAAATTCCCGGAGCTGAAAGTTGACACAAAG
GAATCAACCTAGTTCTTTCTTGTCTTAGCTTTTTTGAAGGGAAATGAAAATTGACACAAAAGAAGGTGGAAGGAA
AAATATGATGCTTGTCCTTTATTGGACATATCATTTTGCAGAATATATATTCTTCCTAGCATTTTGCATGCATTG
TGTCTAGGTTGTCATGTTGTGGCTGGTTTACTAAGTTCCTAGTACAATGAACAAAGCGCCAGAGCTGAAAGTTGA
AATAACAATCGTCAACATCCCAACCGCCTTAACATTAAAACCAACCAAAAAAACAAAAACACATCCCAACCGAAA
GCAGCAATCCAGGAAGATTAGTTTATGTAAATGACAAACATAAATATGAAACCGTCACATAGTTGAGAGTCGCCG
TGAATATTTACTTTTTAAATAAAAAGATATGGTTAGCCGCTGGTTGGTCTGACACCCCATGTATCATTATACTCA
TTATCCGTATGAAGGACCATTTTTTCATATAAGGATATACACGTTTGTAACGTATGCAGAGCCAACACCAAGTAT
ATAACTTGTACTGTCGTCTCTGATTCTGTTAAAGCGGTTAACTAGATTATTGACTATGATAATTCAATGTAACAC
CAATTTATGCCCCGGACAAAAAAAAAATGGTGAAACCAAAATATTTGATTTGCATCCCAGTTACTATTTCCCTGC
AATGATATGGGGGCGGCGGTTTGGAGATATCGCCAATGCCACAATTTGTCGTAAAAACAGTTAAACATTTTACTC
ACTAGTCACTCCCCACAAAAAAAACGATACTTAGTTCCACTCTCTACTACTACAAAACTTGTATACATTAAAAAA
CGATTTTTATTGAGTAGACATTAATGATCGGCTTATAAGTCTAAACAAGTAATGTGACAGAATTCACGTGAACAA
ATGCTCGAGTGGGATGTTTTTATTGCTTCTCAAGTTGGTGATCTTTAATATCGCCAAGATTAGGTTGTGCATTAA
ATTTGGATTACTTTTTATAAATTAAATCAAGATATGGATATGATAAACTTTCTCTCTTTTCTTTTAGTTAACATA
CTTTATAGACTTTATACTACATTAGATTAATTGCAGATTTAAACAATGTTTAAAATTACAAAAACGTTAAATGTC
ATATTTTCCATATAAATTTTGTTTTACGTAAAAGCAATTTACATGAATTTTGATAAAATCATGAAAAAACAGAAG
CGGTGTCCAAGATTGGGGATGAATGAGAAAACACCGCACTGATAGTAACTGAAAAGAATGTAACCAGTCTCACGA
TGGGGATGGCGGAGAGAAAGAGAGAGACAAGACAGTAGAGAAAAGAAGGTACGACGGAGAGATGAAGGTTGGACA
GAGAGAACAAAACGAGAACGGCAATGGCCGGGAAAAAAGGAGAGTAGGGAGGAAAAAAAAAAGAATTTTGTTTTT
TTAGCTTTTGTAGGGTTTTTTGTTAGTCGTAAAATATAAATTATAATATCAAAGAAAATAATGAGTTAATTTTAA
AGAAGGTTAGATGATATTTTTCACAAGTTTTTGTTGATATAGTAAACCCACAATTTTATTTTGTCATTCAAAGTA
AATTTTCAACAAAATATAGCTGTTGCTGGTCACATTTTAACGGGAAAAAATAATTCTTGTACCTGGATGTATAAC
CTCTGAACCTTGTATTACAAATTTACAAAACACATTGATCTCAATCAGACCGAACATGTATATCGAGTCAACTGA
CAAATCTCTTTTCCTACTCTTGTTACATATTCTTTCTAGGAAAAAAAAATGCTTTCAGGCTCAATGAAATGAATT
AAAACAGCAAAAGGTTGGTAAAAGTCTTTTTCCCCATAATCATTTGCTCCATAATTGACGTTTGTCTGGCAACAA
CTGGAAACACAGATATAGAAAAAAAGATTGGACAGTAAATCATAAAAAAAAAAAAGCAAAATTAAATAGAAAGAC
GAAGCGAGAGAAAGAGCGTTGAGTGGCGTGAGAGTTTGAGTCCGAAAATTAACTCCTCTCCCATCTGCCAACTTT
CCTCTGTTTCTCCTTTATATCCCAAACCTATTCTTCCTCCACACCTCATCATAGGCATAGCAAAACTCTTCCAAA

Illustrative Myb 96 protein sequences and accession numbers:

SEQ ID NO: 80 Myb96_At5g62470_NP_201053
MGRPPCCEKIGVKKGPWTPEEDIILVSYIQEHGPGNWRSVPTHTGLRRCSKSCRLRW
TNYLRPGIKRGNFTEHEEKTIVHLQALLGNRWAAIASYLPERTDNDIKNYWNTHLKK
KLKKINESGEEDNDGVSSSNTSSQKNHQSTNKGQWERRLQTDINMAKQALCEALSL
DKPSSTLSSSSSLPTPVITQQNIRNFSSALLDRCYDPSSSSSSTTTTTTSNTTNPYPSGVY
ASSAENIARLLQDFMKDTPKALTLSSSSPVSETGPLTAAVSEEGGEGFEQSFFSFNSM
DETQNLTQETSFFHDQVIKPEITMDQDHGLISQGSLSLFEKWLFDEQSHEMVGMALA
GQEGMF
SEQ ID NO: 81 AtMyb94_At3g47600_NP190344
MGRPPCCDKIGVKKGPWTPEEDIILVSYIQEHGPGNWRSVPTHTGLRRCSKSCRLRW
TNYLRPGIKRGNFTEHEEKMILHLQALLGNRWAAIASYLPERTDNDIKNYWNTHLK
KKLKKMNDSCDSTINNGLDNKDFSISNKNTTSHQSSNSSKGQWERRLQTDINMAKQ
ALCDALSIDKPQNPTNFSIPDLGYGPSSSSSSTTTTTTTTRNTNPYPSGVYASSAENIAR
LLQNFMKDTPKTSVPLPVAATEMAITTAASSPSTTEGDGEGIDHSLFSFNSIDEAEEKP
KLIDHDINGLITQGSLSLFEKWLFDEQSHDMIINNMSLEGQEVLF
SEQ ID NO: 82 ThMyb96_BAJ34253
MGRPPCCEKTGVKKGPWTPEEDIILVSYIQEHGPGNWRSVPTNTGLKRCSKSCRLRW
TNYLRPGIKRGNFTEHEEKMIVHLQALLGNRWAAIASYLPERTDNDIKNYWNTHLK
KKLKKINEFGEEDNDGFSSSNTSSQKQHQSSNKGQWERRLQTDINMAKQALCEALSL
DKPSSSTLSPSSSPLSPVIVPQNIPSFSSALLDRCYDLSSSSSSTTTTTTTTITSNTTTNPY
PSGVYASSAENIARLLQDFMKDTPKALTLTSSSPVSETGPLSAAACEEGGEGFEQSFFS
FNSMEETQNLTQETRFFHDQESKPVISMDQDHGLISQGSLSLLEKWLFDENMVGMAL
EGQEAMF
SEQ ID NO: 83 VvMyb30_XP_002284926
MGRPPCCDKIGVKKGPWTPEEDIILVSYIQEHGPGNWRAVPTSTGLLRCSKSCRLRW
TNYLRPGIKRGNFTDQEEKTIIHLQALLGNRWAAIASYLPQRTDNDIKNYWNTHLKK
KLKKFPTGVDDHNQDGFSISKGQWERRLQTDIHMAKQALCEALSIDTSSSLPDLKSS
NGYNPNTRPVQASTYASSAENIAKLLEGWMRNSPKSTRTNSEATQNSKNSSEGATTP
DALDSLFSFNSSNSDLSLSNDETANFTPETILFQDESKPNLETQVPLTMIEKWLFDEGA
ATQEQEDLIDMSLEDTAQLF
SEQ ID NO: 84 PtMyb081_XP_002323853
MGRPPCCDKIGVKKGPWTPEEDIILVSYIQEHGPGNWRAVPTSTGLLRCSKSCRLRW
TNYLRPGIKRGNFTDHEEKMIIHLQALLGNRWAAIASYLPQRTDNDIKNFWNTHLKK
KLRKLQAGQEGQSRDGLSSTGSQQISRGQWERRLQTDINMARQALCEALSPGKPSSL
LTGLKPSCGYEKPATEPIYASSTENISRLLKGWMISGPKQSLKNSTTQNSFIDTAGADS
LSSEGTPDKADKNGTGLSQAFESLFGFDSFDSSNSDFSQSMSPDTGLFQDESKPNSSA
QVPLSLIERWLFDEGAMQGKDYINEVTIDEDNLF
SEQ ID NO: 85 MtMyb_MTR4g108430_XP_003609059
MGRPPCCEKLGIKKGPWTPEEDIILVSYIQQHGPGNWRSVPTNTGLMRCSKSCRLRW
TNYLRPGIKRGNFTDHEEKMIVHLQALLGNRWAAIASYLPQRTDNDIKNYWNTHLK
RKMNKDQSSTDEGVDQESRSQLPNKGQWERRLQTDIHMAKQALSEALSLQHNPTTL
GTLPDQMKPSSSFSHSHEHPPNLNIPSPYASSYENISRLMETWMKSPNSSAETNSSSIFS
NMQGSSCSEGAQSTTQDHHGLNSSKSDYASRFRSSHEGNNSFNLNTKEGLFFHQEER
INIKANMETHVPLTLLEKWLFEDGGASHECHEELINMSLEGTTSDFF
SEQ ID NO: 86 MtMyb_MTR_3g039990_XP_003599668
MVRPPCCEKIGVKKGPWTPEEDIILVSYIQEHGPGNWRSVPTTTGLMRCSKSCRLRW
TNYLRPGIKRGNFNDHEEKMIIHLQALLGNRWAAIASYLPERTDNDIKNYWNTHLRK
KLKKINQTGDENEVEENSIPQVKGQWERRLQTDIHMAKQALCEALSLDKPTPILAEN
QTSPYASNTENIARLLEKWMKKPENSVETTNSGNSIMVVTGSGSREGGQNTIACKQK
DQAFDSLVSFNSLNSDCSQSVSVEEKNFLAMDSCFFQYQSKPNQETQDPLMFMENW
PFDDEAAQCNEDLMNVSMEENTPGLF
SEQ ID NO: 87 CmMyb60_ABK59039
MGRPPCCDKIGIKKGPWTPEEDIILVSYIQEHGPGNWRAVPTNTGLLRCSKSCRLRWT
NYLRPGIKRGNFTDQEEKMIIHLQALLGNRWAAIASYLPQRTDNDIKNYWNTHLKK
KVKKLQLAAAGCSEDNSQYRDELASASSQQISRGQWERRLQTDIHMAKQALCAALS
PDKASILSELKPANGFISYTKPAVQAPTYASSTENIAKLLKGWARNAQKSASSNSGVT
DQNSINNNVNHIAGAESASSEETPSKVASNSTGIELSEAFESLFGFESFDSSNSTDLSQS
VTPESSTFQDYESKQLLLDPSASADDDQMPQLSLIEKWLFDDQGAKDYLNDLKLDD
HEDTDMF
SEQ ID NO: 88 BdMyb_XP_003574549
MGRPPCCDQAGVKKGPWTPEEDLMLVSYIQEHGPGNWRAVPTNTGLMRCSKSCRL
RWTNYLRPGIKRGNFTDQEEKLIVHLQALLGNRWAAIASYLPERTDNDIKNYWNTH
LKRKLKKMSAAAGEDGAAAATAGGAEAKSRATAPKGQWERRLQTDIHTARQALR
DALSLDTTAPAPPKPAPMERSSKGAVYASSAENIARLLEGWMRPGEGKASSGGSGSG
SRSSASVVSAEGASASHSGTAPTPEGSTVTSKTKDEVAVAAPPAFSMLENWLFDDGM
GMGHNGIGDVGLDDVPLGDPSEFF
SEQ ID NO: 89 BdMyb_XP_00356188
MGRPPCCDKDGVKKGPWTPEEDIILVSYVQDHGPGNWRAVPPNTGLMRCSKSCRLR
WTNYLRPGIRRGNFSEQEEKHIVQLQALLGNRWAAIASYLPDRTDNDIKNYWNTHL
KKKLLHRTSTATPAPAPTTHKDQNNNKGQWERRLQTDIHLARQALREALSLDTAST
SATPGPAAYALSAQNVSRMLDDWAVAADSASSEVTECSGGSTASNGTLWSSLLGRE
STGAAAAGVEDPAALSAIESWLLLDDGTDRQQPPEQEQSGGQLLP
SEQ ID NO: 90 TaMyb_AEV91147
MGRPPCCDKEGVKKGPWTPEEDLVLVSYVQEHGPGNWRAVPTRTGLMRCSKSCRL
RWTNYLRPGIKRGNFTDQEEKLIVHLQALLGNRWAAIASYLPERTDNDIKNYWNTH
LKRNLQAGGDAAAKPAAQRPASSSKGQWERRLQTDINMARRALREALTTLDDIKRQ
QPDAADGVNGPAAAGADSGSPAASSSSAASLSQCSPSAAGPYVLTTANISRMLDGW
ASKGRSAVPAADSPSGSSASEVSYGSGAAARALGSAFEYDRKPAVLAPDQTQLNAIE
TWLFADDNSNNDHHGHGGGGSGLLGVPATLGYPF
SEQ ID NO: 91 OSMyb_Os09g0414300_NP_001063167
MGRPPCCDKVGVKKGPWTPEEDLMLVSYIQEHGAGNWRAVPTNTGLMRCSKSCRL
RWTNYLRPGIKRGNFTEQEEKLIVHLQALLGNRWAAIASYLPERTDNDIKNYWNTH
LKKKLKKMQAAGGGEDSGAASEGGGGRGDGDGGGKSVKAAAPKGQWERRLQTDI
HTARQALRDALSLDHPDPSPATAAAAATPAGSSAAYASSADNIARLLQGWMRPGGG
GGGNGKGPEASGSTSTTATTQQQPQCSGEGAASASASASQSGAAAAATAQTPECSTE
TSKMATGGGAGGPAPAFSMLESWLLDDGGMGLMDVVPLGDPSF
SEQ ID NO: 92 OSMyb_Os08g0437200_NP_001175597
MGRPPCCVKAEVKKGPWTPEEDLMLVAYVQEHGPGNWRAVPTNTGLMRCSKSCR
LRWTNYLRPGIKRGNFTDQEEKLIVHLQALLGNRWAAIASYLPERTDNDIKNYWNT
HLKKKLKKMSATGGGGDDGEGGGAGEVKTRAAAPKGQWERRLQTDIHTARQALR
DALSLDPSPPAKPLDSSSGATAPPSSQAATSYASSAENIARLLEGWMRPGGGGGKTTT
TPSSGSRSSAASVLSGEASHSGGATAPTPDGSTVTSKTKDEETAGAPPPPPPPAFSMLE
SWLLDDGMGHGEVGLMDVVVPLGDPSEFF
SEQ ID NO: 93 ZmMyb_NP_001132068
MGRPPCCEKAGLKKGPWSPEEDLLLVSYVQEHGPGNWRAVPCSTGLMRCSKSCRLR
WTNYLRPGIKRGSFSDQEEKLIIHLQELLGNKWSAIASYLPERTDNDIKNYWNTHLK
KKLAKTGARESGASAKTTKKSDRAAAPKGQWERRLQTNIHTARQALREALSMDDT
APPAIKPEPLPLPLGQLPAPASQAMYASSIDNIARLLEGWMRPSVSGNASAESMSSFS
AFSGGGDGASASHIGTAHTPEGFTGTRKEEGAGPGPASLPMFENWLLDDGMGNGDA
SLICVPLADPCEFF
SEQ ID NO: 94 Illustrative REF4 promoter At2g48110; NP_566125 (encoded protein
sequene)
TTTCGCAGGCCCTAATTAAGACATTCAAGAAAACAAGAAGAAGCATAAGAAGAGGCCTAAATGCCCAGAGAATT
AAACAAATGGGCCTTTAACAATATTTTAAACAACACTGAACAATAGATGAGATCTCAACTTCGAAAAGCTAAAGC
CTTAATATAAAACCAAACATCAATCTCTCGCCGTTAAGGTTACATCTGTCGCCGCCGCTGAGACCGCCACTTAAG
GCCGTCTCCTCCGCTCTATCCATATCCAAAACAGCTATAAAGATAAACTTCCAGAGCTTGGTGAAGGAGCAGCAA
CCCTAGTTTTCAATCCCTAAAGGTGAAGTTTTTGTTCTTCTTTTTCTTTGATTCTACTCTTTTTTCGTCACAATA
GCGCAGTAGCGATCTGGGTTGGCGTTTTTATTGACGGATTTTGGTGGGTTTGGGTTCGTGTTAGATTTTACTTTC
ACAGATTCAGGTATGTATTTGATCTTATTTAGCTTTGGTAATACTCAAACCAACTAGTATTTGATCTTAATTAGG
TGACGTTGATATTTTGTGTGTTGTGGGTCAATCATCCACTCTAGGTTGATCTGCGTCTATGTTTAGTCCTAATTT
TGAAGAATCATTCTAATTGCTGATGTTTATTTATGACAATTTTATGTCTTACTTCGAATTTAGCTAAGATAATAA
ATCTAGACGAGTGCCTTTATAAACAGTTTATGTATGAACAATGCTTCGCTTGTGGGATTGTTGATTTCACTACAT
TTGATTAAGAATGTACATACTATTACAAATGTTATCGATTAAAAACGTCATTTTCAGATATTAGTTCTCCAGCGA
GTTGACACAAAACCCGATTACGTTTCATCCGGCGACTCGCTTTGATATTCCATGGATTCTCAGTTGAATCCTTCC
AAGAGACGCAAGATAAGGTTCGCGTCTCTCATCTTTTCCCCAGTTTTTTTTCTTATTCGAATCACTTAAGGCTTG
AGAAATTAGTGGTTAGGGTGTGTAAATTGCGTTTGATATAGTGTCAGGCACAGATGCGTTGCGTGCTATAAGATG
TTCAATAGACGAGAACACCTCGTTGAGCACATGAAGATTTCCTACCACTCACTTCACCAGCCTCGCTGTGGGGTT
TGCCTCAAGCACTGTAAATCCTTCGAATCCGTGAGGGAACACCTTAACGGTATGTTTGTATTTGTATTCGTTTTC
CACCACACATCGTATGAATATGGTGGTTGTTGATGTTTGTTTGATTCCGTCTTTCCAACTATTTCAGTTCCAGAC
CATCTTTCCAAAGGAAACTGCAAAGCCATTTTCACTAAACGAGGCTGTACTCTCTGTCTTCAAATCTTTGAGGAG
GCCTTTGCTCTCGCCGAGCATAAAAACAAGTGTCACCTCTCCCCACCTCGTCCTCTTGTAAGTTTTGTTGGGAAT
TATTTAGATAATGTGGACTATATATGCTCTGCCGCCTCCAATATTCCCGAGTCTATTCTTTCTGATTATTGAAAT
ATCAGCAGTTTTCCCCTTAAATGATCTGATTAGTGCTTTATTCATATCAGGGAACATCTACCCAAAGGAATCCTT
CTAGTTCACTTGCTGGTTCACGTCTCAAGGCTATGGCACTTGACTGTGAAATGGTTGGTGGTGGTGCTGATGGGA
CTATTGATCAGTGCGCATCGGTTTGCCTGGTTGACGATGACGAGAATGTGATCTTCTCCACTCACGTTCAACCAC
TGCTCCCTGTCACCGATTACAGGTTCTGCTTGTGGACCATTTGTGCTTGTTTTGTTTTATAATTCTTCCTTTTAA
ATCTCACCCCGCCTCATGTTCAGGCACGAGATAACTGGATTGACTAAGGAAGATTTGAAGGATGGTATGCCACTT
GAGCATGTACGAGAGAGAGTTTTTTCGTTCTTGTGCGGTGGACAGAATGATGGGGCTGGAAGGCTTCTTCTTGTT
GGTCATGACCTTAGGCATGATATGAGTTGCTTGAAGCTTGAATACCCTAGCCATTTGTTGAGGTAACTAACTGAC
CCGTTTTTGTTGACTCTTTGCTTGAAATCTAATGTAATTGCTATGCTTCACCTCAGAGACACAGCAAAATACGTG
CCGTTGATGAAGACAAATCTTGTAAGCCAATCGCTCAAGTACCTCACAAAGTCATATCTCGGGTAAGTTATGCTT
GGGCTTTGATTTGTTGACATTGGATTATGAAAACTTGGAACATGAGATAGAAACTGGTTTTGTTTGTATGTGTAG
ATACAAGATCCAGTGCGGGAAGCACGAGGTTTACGAGGACTGTGTATCTGCGATGAGACTGTACAAGAGAATGCG
GGATCAAGAGCATGTTTGTAGTGGAAAGGCAGAAGGGAATGGTCTGAACTCGCGGAAACAGAGCGATCTAGAGAA
GATGAATGCGGAAGAGCTGTACCAAAAATCAACGTCAGAGTACCGGTGCTGGTGCCTTGACCGACTCAGCAATCC
ATGAAATGAAATCCCAATTTCTCTTTTAAAAGAAAGCTCCTTCTCTTCCTCCCTTAAGTCTCTTTTTGTTCGATG
GGAGGATTAAGAAGACTTGTTAAGAGCTTTTCCGGCTAGTTTTTGGATAATCAAATATGATATGGCTTCATTCAA
CAACAACTCTCCCTTTGGGAACTCGTATAAAAAGTTACTACTTAAGCTTCAAATCTGTGTAACTAAAATGGATAA
AGTCTGATGCATCTGAGGTCTTGGAATCTGTTGCTCATAGTTGTGGATACCAGCAACATCAAACATTATTACTTG
TTTACCAAACCCACTCTCAGCTTCCATTTCATCAGTTTGAAGTCAAGTGGATAAAAAGACTGTCTACATAACTCA
AATACTGTAGTATTATATTCACATATAAATGAGAAATTGACATTTCCTCTAGAAAAAGAAAAGCAATCGTGTGAG
AAGAAACAAGTAAAAGGCTGAGGAAGAAGACGACTATTAGTCCCCGTCGAACTTGTTTCTCCCGGTCGTCCAACA
A
SEQ ID NO: 95 Illustrative RFR1 promoter At3g23590; NP_189001 (encoded protein
sequence)
AAATGATTTGTTTTGTGAATAGTTGATTCCAGCTAATGTGGTGAAAGACAATCATCTTACGTTTGGTGATTGTAT
CAACTACATTGATGAAGAAGGGAACTTAGTAGGACAAAGAGGGAATAAGCAGATGATAGGATTTGCTTCAAAGGA
TGCATGGCGACATAATTTGCAGAAGGATTAGACATAATAACATGAAGATACTGTCAAGTATGAGCTCCTTCATGA
ACTTAAGTACGTTCATAATTCCACTTGATCCACAACTTTGGGCTATTGGTATAAAAAGCTAACTTTATCTAGATT
ACATGCTACATCTCAATTCAAGATGTCCATTTATTAAAGCAAAGGCATGACTTTATCTTCGACTAGAAACTGGTG
ATGGACCATCCTCAACCTCCACTAGCATCATCTCCACCTACTCCTAACCACTAACCCAATTATCCTTTTCACCTT
TTCCTCTCAACACTATCCTATTTTTAGGTGATTTAGGGAAAAGAGAAATACTACACTTTGATGTAATTTTTTTTT
TAATTGAATTGAGCTTTGAATACTATGGTTAATATGGATGAAAGAAAACACACGTTCCTACTAATTTCTTTTTCT
AAATGATGACGTTTTCTCAAGTTACTTATCGTAGTCTAATCGGTTATTGACAAAAATGATCTAGACGTTTGAAAA
TCATTGGCCAACCTCTTCACTAGATAGAATTTTTATTTCTAACCATTACCTACCTGATACACACACCAAGTTCTG
AATCTAGAGATGATCTTGTCTCATTTGTTTATAGACTAAAGAGTATATAAAAGCTGGATTCATGTGGGTCATCCA
TCATTAATCGTGTTCTTCAATTCACCAACTTCGTAGGTTCCGCATCTTTTGTACTTTCTTTCTATTTTTAATCTT
GCAATATCAAAAATAAATATATACACCTTTTAAAAAAATTCTGAATATGCATATGGTAATGGTATCATCAGAAAA
CATTGAATATTGAATCGTCTATCTACCAATCTCTTCAAGTTGTGAATTTTATTTATCAAAAAAAAAAAAAAAATC
AACTGAAACGTCAATGTTATGTTTTGCTTCAATCTAAAGAAAGTTGTACCAAAAATAAAGTACTTTTTTACCAAG
AAATTTCGGATTGTAGCCAAAATATGTCTTGTTTTATTTTTTTTGTTCACCAAAAATATGTTAAACTAGTTTTTT
GACTTTTTCCCCCATGCGTTGTTGTTGTTATTCGGACGACCAATATTTAATACAGTATTCAAGTGTAGCTAAGCT
ACTGATTCCAGATAAAGTAAAAAAATGTCTTTGATACCAACTTCACTTTGACCAATTATCGAACTCTTTAATTTC
ATAATATATAGCTTTGACAAATATCCCATCAGAATTCATAAATCGCCACATGAATCTTATAATTGTCAACTATGT
GCAGTCCAATATTAATATCATGGAAAACTAGATTACTAGCATTTAATTTCTTAATTGTATTTAGGTTATAGACTT
AACATGGTTTTTCTTCTAGAGCATTTTCACTACATTTTTTTCTTTCTATGTTATTATTTTATGTTTTACTTATTG
TTACATCCATTTACAACGATGTTGGATAAAATATTTTTAACTGTTCAGTCCGTAAATGACTAGCTAAATTTTTAC
GGATATATTTTTACATAAAAAAAGAAATTATTTTTACGATTTACCAATTTAACTCTTCTTTCTCTCTAAGTTTTC
ATTTATTAAAAATGATAGTTTTTTTAAGAAAGTTATTTGTATTTATTTATTTAAAATTGGATGCAAAAAATTACA
AAACTTGGAAGAAATATTGTTAACCCATCTTTATCAGTATAGAATTTCTGATTACTCCCTGCGTTGAAAATAAGA
AGATCCATAGAACGTGGTAATGTTTTTTTTTTTTTTTTTCTGGCAAGAAATGGTAATGTTAATCAATTAGACTCA
TGACTAAAGCTGGAAAATTTGACTAAAATGGTATTATCTTGCATTATTACCTAAAAGCTCTTCTTGCATTACTCT
TTCATGCAAAATTGCCTCCAAAAGTCATACTTCGAAATCATGGCTTTGTAACTCACCCTTGCCACCATCTATCTA
AACTTATTATTGTTTAAAACAAAATTGATTTTCAAAGAAAAAAAAAATATAAACTCCCACCGGTCAATAACTTTT
AATTTTCACTTCAATTATTCATAAAATAAATATATACACCTTGATTCTTTCTTAATAACTGGAGTTATTTTGAAT
TTAAATTACTTTTGAAATACAAAAGAAAGTAAGATAATTTTGCCATTTAAAATGTCTACAACAATTATGTTAACA
CTTAACATACATTTTTAGAACCAAATGGTAATGGTCCACTAGTTAAAAGATTTCTTATGAATGTCTTTTTATGCA
GAGTTCGAATTTTCTCACAACGATTTTAACCATTTTCGTCACTCGCAAAATTTTTAGTGGATAACAAAAAAAAAA
AAAAGTTGAAATTTAATGGAAATTTAGGAGATTTGTCCAATTTTTAATTTTCCCGATTAATTTAAAAAATCTACT
TTACGCCTTTTATTGACAAAGATGCATATTAACTATATATATATATATATATATATATATATATATATATATATA
TTATAAACTAAATTTTGTAGAACCCATCAGAAGAAATTTGTACTTTTAACTTTAAAATCAATTAAAAAATTTCAA
TTCAAATAAAAAAAAATCCAAAATTGTTTATAAATTCAAGAAATATAACACACAAGTTGACATAAACAGTGGAGA
GATTTGTGAAATGTGAACTCTTTTGACGTCTTTCTCATATTTCGTCTCGGGACAATTCACACGCACTGAATCATC
TCTCTCACGAGGACTATTCAGCCATTTGCAAACGCACACACAAACGCACACGCGTTTATTTTTTTTTCGCCAAAT
CAAATCTGAAGAGTTCCTGTATCTTTTAACCGCTCTTCTTCTTCTTCAGAGAGCTTCGTTGATTGAACGGAAAAA
Legend: At: Arabidopsis thaliana; Th: Thellungiella halophila; Mt: Medicago truncatula; Pt: Populus trichocarpa; Vv: Vitis vinifera; Cm: Citrus macrophylla; Bd: Brachypodium distachyon; Ta: Triticum aestivum; Os: Oryza sativa; Zm: Zea mays

1. A method of engineering a plant to increase the production of a biosynthetic product in a desired tissue, the method comprising:
introducing an expression cassette into the plant, wherein the expression cassette comprises a polynucleotide encoding a transcription factor that regulates production of the biosynthetic product operably linked to a heterologous promoter, wherein the heterologous promoter is a promoter that induces gene expression of a gene that is a downstream target of the transcription factor in the desired tissue; and
culturing the plant under conditions in which the transcription factor is expressed.

2. The method of 1, wherein the promoter is a tissue-specific secondary wall promoter and the transcription factor induces expression of secondary wall biosynthetic products.

3. The method of claim 2, wherein the transcription factor is NAC secondary wall-thickening promoting factor 1 (NST1), NST2, NST3, secondary wall-associated NAC domain protein 2 (SND2), SND3, MYB domain protein 103 (MYB103), MBY85, MYB46, MYB83, MYB58, or MYB63 and/or the tissue-specific secondary wall promoter is an IRX1, IRX3, IRX5, IRX8, IRX9, IRX14, IRX7, IRX10, GAUT13, GAUT14, or CESA4 promoter.

4. (canceled)

5. The method of 1, wherein the transcription factor induces expression of wax and/or cutin.

6. The method of claim 5, wherein the transcription factor is a shine (SHN) transcription factor selected from SHN1 (also known as WIN1), SHN2, SHN3, SHN4, or SHN5; or MYB 96; and/or the promoter is a CER1, CER2, CER3, CER4, CER5, CER6, CER10, WSD1, Mah1, WBC11, KCS1, KCS2, FATB, LACS1, LACS2, CYP864A, CYP86A7, CYP86A5, KCS10, or KCS5 promoter.

7.-8. (canceled)

9. A method of engineering a plant having lignin deposition that is substantially localized to the vessels of xylem tissue of the plant, the method comprising:
introducing an expression cassette into the plant, wherein the plant is modified to have a reduced level of expression of a lignin biosynthesis enzyme; and further, wherein the expression cassette comprises a polynucleotide encoding the lignin biosynthesis enzyme operably linked to a heterologous vessel-specific promoter; and
culturing the plant under conditions in which the lignin biosynthesis enzyme is expressed.

10. The method of claim 9, wherein the lignin biosynthesis enzyme is PAL, C4H, 4CL, HCT, C3H, or CCR1.

11. (canceled)

12. The method of claim 9, wherein the promoter is a VND1, VND2, VND3, VND4, VND5, VND6, VND7, VNI2, REF4, or RFR1 promoter.

13. The method of claim 9, wherein the level of expression of the lignin biosynthesis enzyme in the modified plant is reduced by contacting the plant with an antisense oligonucleotide that silences expression of the gene encoding the lignin biosynthesis enzyme.

14. The method of claim 9, wherein the modified plant in which the polynucleotide operably linked to the heterologous promoter is expressed has a mutation in the gene encoding the lignin synthesis enzyme that decreases expression of the enzyme.

15. (canceled)

16. A plant engineered by the method of claim 1, or a progeny of the plant.

17.-18. (canceled)

19. A plant cell comprising a polynucleotide encoding a lignin biosynthesis enzyme operably linked to a heterologous vessel-specific promoter, wherein the plant cell is engineered by the method of claim 9.

20. The plant cell of claim 19, wherein the lignin biosynthesis enzyme is PAL, C4H, 4CL, HCT, C3H, or CCR1; and/or the promoter is a VND1, VND2, VND3, VND4, VND5, VND6, VND7, VNI2, REF4, or RFR1 promoter.

21. (canceled)

22. A plant comprising the plant cell of claim 19, wherein the plant has lignin deposition that is substantially localized to the vessels of xylem tissue of the plant.

23. (canceled)

24. A method of obtaining an increased amount of soluble sugars from a plant in a saccharification reaction, the method comprising:
subjecting the plant of claim 16 to a saccharification reaction, thereby increasing the amount of soluble sugars that can be obtained from the plant as compared to a wild-type plant.

25. A method of engineering a plant having increased secondary cell wall deposition, the method comprising:
introducing an expression cassette into the plant, wherein the expression cassette comprises a polynucleotide encoding a transcription factor that regulates the production of secondary cell wall in woody tissue operably linked to a heterologous promoter, wherein the promoter enhances expression of a gene that is a downstream target of the transcription factor; and
culturing the plant under conditions in which the transcription factor is expressed.

26. The method of claim 25, wherein the transcription factor is NST1, NST2, NST3, MYB103, MYB85, MYB46, MYB83, MYB58, or MYB63; and or the promoter is an IRX1, IRX3, IRX5, IRX8, IRX9, IRX14, IRX7, or IRX10 promoter.

27.-29. (canceled)

30. The method of claim 25, wherein the plant in which the polynucleotide operably linked to the heterologous promoter is expressed is a wild-type plant.

31. The method of claim 25, wherein the plant in which the polynucleotide operably linked to the heterologous promoter is expressed is an engineered plant having lignin deposition that is substantially localized to the vessels of xylem tissue of the plant.

32. (canceled)

33. A plant engineered by the method of claim 25, or a progeny of the plant.

34.-41. (canceled)

42. A method of increasing bioenergy production from biomass derived from a plant, the method comprising:
harvesting biomass from the plant of claim 33; and
subjecting the biomass to a conversion reaction, thereby increasing bioenergy production as compared to a wild-type plant.

43. A method of increasing stem, straw or timber strength from plants during growth, the method comprising
cultivating the plant of claim 33 to produce seed, wherein resistance lodging is improved as compared to a wild type plant.

44. A method of obtaining a plant having improved resistance to mechanical stress, the method comprising:
cultivating the plant of claim 33, thereby obtaining a plant having improved resistance to mechanical stress compared to a wildtype plant that has not been genetically modified to increase secondary cell wall production.

45. A method of engineering a plant having xylan deposition that is substantially localized to the vessels of xylem tissue of the plant, the method comprising:
introducing an expression cassette into the plant, wherein the plant is modified to have a reduced level of expression of a xylan biosynthesis enzyme; and further, wherein the expression cassette comprises a polynucleotide encoding the xylan biosynthesis enzyme operably linked to a heterologous vessel-specific promoter; and
culturing the plant under conditions in which the xylan biosynthesis enzyme is expressed.

46. The method of claim 45, wherein the xylan biosynthesis enzyme is irregular xylem 8 (IRX8), IRX14, IRX14-like, IRX9, IRX9-like, IRX7, IRX10, IRX10-like, IRX15, IRX15-like, F8H, or PARVUS; and/or the promoter is a VND1, VND2, VND3, VND4, VND5, VND6, VND7, VNI2, REF4, or RFR1 promoter.

47. (canceled)

48. The method of claim 45, wherein the level of expression of the xylan biosynthesis enzyme in the modified plant is reduced by contacting the plant with an antisense oligonucleotide that silences expression of the gene encoding the xylan biosynthesis enzyme.

49. The method of claim 45, wherein the modified plant in which the polynucleotide operably linked to the heterologous promoter is expressed has a mutation in the gene encoding the xylan synthesis enzyme that decreases expression of the enzyme.

50. (canceled)

51. A plant engineered by the method of claim 45, or a progeny of the plant.

52.-58. (canceled)

59. A method of obtaining an increased amount of soluble sugars from a plant in a saccharification reaction, the method comprising:
subjecting the plant of claim 51 to a saccharification reaction, thereby increasing the amount of soluble sugars that can be obtained from the plant as compared to a wild-type plant.

60. A method of obtaining an increased C6/C5 sugars ratio from a plant after a hydrolysis reaction, the method comprising:
subjecting the plant of claim 51 to chemical or enzymatic hydrolysis, thereby increasing the amount of C6 sugars compared to C5 sugars that can be obtained from the plant as compared to a wild-type plant.

61. A method of engineering a plant having xylan O-acetylation that is substantially localized to the vessels of xylem tissue of the plant, the method comprising:
introducing an expression cassette into the plant, wherein the plant is modified to have a reduced level of expression of an enzyme responsible for xylan O-acetylation; and further, wherein the expression cassette comprises a polynucleotide encoding the xylan O-acetylation enzyme operably linked to a heterologous vessel-specific promoter; and
culturing the plant under conditions in which the xylan O-acetylation enzyme is expressed.

62. The method of claim 61, wherein the xylan O-acetylation enzyme is an RWA protein.

63. The method of claim 61, wherein the promoter is a VND1, VND2, VND3, VND4, VND5, VND6, VND7, VNI2, REF4, or RFR1 promoter.

64. The method of claim 61, wherein the level of expression of the xylan O-acetylation enzyme in the modified plant is reduced by contacting the plant with an antisense oligonucleotide that silences expression of the gene encoding the xylan O-acetylation enzyme.

65. The method of claim 61, wherein the modified plant in which the polynucleotide operably linked to the heterologous promoter is expressed has a mutation in the gene encoding the xylan O-acetylation enzyme that decreases expression of the enzyme.

66. (canceled)

67. A plant engineered by the method of claim 61, or a progeny of the plant.

68.-74. (canceled)

75. A method of obtaining an increased amount of soluble sugars from a plant in a saccharification reaction, the method comprising:
subjecting the plant of claim 67 to a saccharification reaction, thereby increasing the amount of soluble sugars that can be obtained from the plant as compared to a wild-type plant.
